(19) United States
(12) Patent Application Publication
(10) Pub. No.: US 2025/0289867 A1
(43) Pub. Date: Sep. 18, 2025

(54) MULTIVALENT PARTICLES COMPOSITIONS AND METHODS OF USE

(71) Applicant: Achelois BioPharma, Inc., Redwood City, CA (US)

(72) Inventors: Chang-Zheng CHEN, Palo Alto, CA (US); Guoqiang DONG, Redwood City, CA (US); Yiling LUO, South San Francisco, CA (US); Hua ZHOU, San Mateo, CA (US); Tian-Qiang SUN, San Francisco, CA (US); Michael CHEN, Palo Alto, CA (US)

(21) Appl. No.: 18/861,304

(22) PCT Filed: May 2, 2023
(86) PCT No.: PCT/US2023/066483

§ 371 Date: Oct. 29, 2024

Related U.S. Application Data
(60) Provisional application No. 63/338,155, filed on May 4, 2022.
(60) Provisional application No. 63/426,288, filed on Nov. 17, 2022.

(51) Int. Cl. C07K 14/705; A61P 31/14; C07K 14/005

(52) U.S. Cl. CPC C07K 14/70596; A61P 31/14; C07K 14/005; C07K 2319/03; C12N 2740/15023; C12N 2760/20222; C12N 2770/20022; C12N 2770/20034

(57) ABSTRACT

Provided herein are multivalent particles and compositions of multivalent particles for blocking viral infection.

### CROSS REFERENCE

This application claims the benefit of U.S. Provisional Application No. 63/338,155, filed May 4, 2022, and U.S. Provisional Application No. 63/426,288, filed Nov. 17, 2022, each of which is incorporated herein by reference in their entirety.

### SEQUENCE LISTING

The instant application contains a Sequence Listing in electronic format. The Sequence Listing is provided as a file entitled 48295-712_601_SL.xml, created on Apr. 26, 2023, which is 42,940 bytes in size. The information in the electronic format of the Sequence Listing is incorporated by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Disclosed herein, in one aspect, is a composition of multivalent particle that comprises a virus-capturing polypeptide on a surface of the particle wherein the displayed virus-capturing polypeptide is expressed at a valency of at least 10 copies on the surface of the particle and in an oligomerized format wherein the displayed virus-capturing polypeptide forms multivalent interactions with a viral spike protein which provide tighter binding through avidity than a soluble version of the displayed virus-capturing polypeptide to the viral spike protein. In some embodiments, the viral spike protein is from severe acute respiratory syndrome coronavirus 1 (SARS-CoV-1), severe acute respiratory syndrome coronavirus 2 (SARS-CoV-2), Middle East respiratory syndrome-related coronavirus (MERS-CoV), respiratory syncytial virus (RSV), human immunodeficiency virus (HIV), hepatitis B virus (HBV), hepatitis D virus (HDV), or combinations thereof. In some embodiments, the displayed virus-capturing polypeptide comprises a receptor for the virus entry, a ligand, a secreted protein, an antibody, or an engineered protein. In some embodiments, the receptor comprises a viral entry receptor or a viral attachment receptor. In some embodiments, the receptor is both a viral entry receptor and a viral attachment receptor. In some embodiments, the displayed virus-capturing polypeptide comprises an extracellular domain of the receptor. In some embodiments, the displayed virus-capturing polypeptide comprises angiotensin-converting enzyme 2 (ACE2), transmembrane serine protease 2 (TRMPSS2), dipeptidyl peptidase 4 (DPP4), cluster of differentiation 4 (CD4), C-C chemokine receptor type 5 (CCR5), C-X-C chemokine receptor type 4 (CXCR4), cluster of differentiation 209 (CD209), or C-type lectin domain family 4 member M (CLEC4M). In some embodiments, the displayed virus-capturing polypeptide comprises an amino acid sequence of at least 90% sequence identity to the amino acid sequence according to any one of SEQ ID NOs: 1-4. In some embodiments, the displayed virus-capturing polypeptide is part of a fusion protein, wherein the fusion protein comprises a transmembrane polypeptide. In some embodiments, the transmembrane polypeptide comprises a spike glycoprotein transmembrane region, a mammalian membrane protein, an envelope protein, a nucleocapsid protein, or a cellular transmembrane protein. In some embodiments, the transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD 114, Baboon endogenous virus (BaEV), glycoprotein (GP41), or glycoprotein 120 (GP120). In some embodiments, the VSVG transmembrane region comprises full length VSVG transmembrane region or a truncated VSVG transmembrane region. In some embodiments, the transmembrane polypeptide comprises the VSVG transmembrane region and a VSVG cytoplasmic tail. In some embodiments, the transmembrane polypeptide comprises an amino acid sequence at least about 90% identical to the amino acid sequence according to any one of SEQ ID NOs: 19-27. In some embodiments, the fusion protein further comprises an oligomerization domain. In some embodiments, the oligomerization domain comprises a dimerization domain, a trimerization domain, or a tetramerization domain. In some embodiments, the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain. In some embodiments, the oligomerization domain comprises an amino acid sequence that has at least 90% sequence identity to an amino acid sequence according to any one of SEQ ID NOs: 5-18, and 28. In some embodiments, the fusion protein is expressed at least about 50 copies on a surface of the multivalent particle. In some embodiments, the fusion protein is expressed at least about 75 copies on a surface of the multivalent particle. In some embodiments, the fusion protein is expressed at least about 100 copies on a surface of the multivalent particle. In some embodiments, the fusion protein is expressed at least about 150 copies on a surface of the multivalent particle. In some embodiments, the fusion protein is expressed at least about 200 copies on a surface of the multivalent particle. In some embodiments, the multivalent particle further comprises a second displayed virus-capturing polypeptide on a surface of the particle that binds to the viral protein, wherein the second displayed virus-capturing polypeptide is expressed at least about 10 copies on the surface of the multivalent particle. In some embodiments, the second displayed virus-capturing polypeptide comprises a receptor for the viral protein. In some embodiments, the receptor comprises a viral entry receptor or a viral attachment receptor. In some embodiments, the receptor is both a viral entry receptor and a viral attachment receptor. In some embodiments, the second displayed virus-capturing polypeptide comprises an extracellular domain of the receptor. In some embodiments, the second displayed virus-capturing polypeptide comprises a ligand or a secreted protein. In some embodiments, the second displayed virus-capturing polypeptide comprises ACE2, TRMPSS2, DPP4, CD4, CCR5, CXCR4, CD209, or CLEC4M. In some embodiments, the second displayed virus-capturing polypeptide comprises an amino acid sequence of at least 90% sequence identity to the amino acid sequence according to any one of SEQ ID NOs: 1-4. In some embodiments, the second displayed virus-capturing polypeptide is part of a second fusion protein, wherein the second fusion protein comprises a second transmembrane polypeptide. In some embodiments, the second transmembrane polypeptide comprises a spike glycoprotein transmembrane region, a mammalian membrane protein, an envelope protein, a nucleocapsid protein, or a cellular transmembrane protein. In some embodiments, the second transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD114, Baboon endogenous virus (BaEV), glycoprotein (GP41), or glycoprotein 120 (GP120). In some embodiments, the VSVG transmembrane region of the second transmembrane polypeptide comprises full length VSVG transmembrane region or a truncated VSVG transmembrane region. In some embodiments, the second transmembrane polypeptide comprises the VSVG transmembrane region and a VSVG cytoplasmic tail. In some embodiments, the second transmembrane polypeptide comprises an amino acid sequence at least about 90% identical to the amino acid sequence according to any one of SEQ ID NOs: 19-27. In some embodiments, the second fusion protein further comprises a second oligomerization domain. In some embodiments, the second oligomerization domain comprises a dimerization domain, a trimerization domain, or a tetramerization domain. In some embodiments, the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain. In some embodiments, the oligomerization domain comprises an amino acid sequence that has at least 90% sequence identity to an amino acid sequence according to any one of SEQ ID NOs: 5-18, and 28. In some embodiments, the second fusion protein is expressed at least about 50 copies on a surface of the multivalent particle. In some embodiments, the second fusion protein is expressed at least about 75 copies on a surface of the multivalent particle. In some embodiments, the second fusion protein is expressed at least about 100 copies on a surface of the multivalent particle. In some embodiments, the second fusion protein is expressed at least about 150 copies on a surface of the multivalent particle. In some embodiments, the second fusion protein is expressed at least about 200 copies on a surface of the multivalent particle. In some embodiments, the multivalent particle does not comprise viral genetic material. In some embodiments, the multivalent particle is a viral-like a particle. In some embodiments, the multivalent particle is an extracellular vesicle. In some embodiments, the multivalent particle is an exosome. In some embodiments, the multivalent particle is an ectosome.

Disclosed herein, in another aspect, is a particle that comprises a ACE2 polypeptide on a surface of the particle wherein the ACE2 polypeptide is expressed at a valency of at least 10 copies on the surface of the particle and in an oligomerized format wherein the ACE2 polypeptide forms multivalent interactions with a viral spike protein which provide tighter binding through avidity than a soluble version of the ACE2 polypeptide to the viral spike protein. In some embodiments, the ACE2 polypeptide comprises a wildtype ACE2, or a H2A mutant of ACE2. In some embodiments, the viral spike protein comprises a wildtype SARS CoV-1 spike protein, a mutant of SARS CoV-1 spike protein, a wildtype SARS CoV-2 spike protein, or a mutant of SARS CoV-2 spike protein that binds to ACE2. In some embodiments, the viral spike protein comprises the spike protein of original SARS CoV-2, the spike protein of Beta variant of SARS CoV-2, the spike protein of Delta variant of SARS CoV-2, the spike protein of B.1.351 Beta variant of SARS CoV-2, the spike protein of B.1.617.2 Delta variant of SARS CoV-2, the spike protein of SARS CoV-2 USA-WA1/2020 strain, the D614G mutant of SARS CoV-2 spike protein, the N439K mutant of SARS CoV-2 spike protein, the N501Y mutant of SARS CoV-2 spike protein, the E484K mutant of SARS CoV-2 spike protein, the E484Q+L452R mutant of SARS CoV-2 spike protein, the spike protein of Omicron variant of SARS CoV-2, or the E484K+N501Y mutant of spike protein of the B.1.351 South Africa strain of SARS CoV-2. In some embodiments, the particle binds to the viral spike protein to effectively neutralize a virus comprising the viral spike protein. In some embodiments, the virus comprises a SARS CoV-1 virus, or a SARS CoV-2 virus. In some embodiments, the virus comprises the original SARS CoV-2 virus, Delta variant of SARS CoV-2, Beta variant of SARS CoV-2, B.1.351 Beta variant of SARS CoV-2, B.1.617.2 Delta variant of SARS CoV-2, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant of SARS CoV-2, or B.1.351 South Africa strain of SARS CoV-2 comprising the E484K+N501Y mutant of spike protein. In some embodiments, the ACE2 polypeptide is part of a fusion protein, wherein the fusion protein comprises a transmembrane polypeptide. In some embodiments, the transmembrane polypeptide comprises a spike glycoprotein transmembrane region, a mammalian membrane protein, an envelope protein, a nucleocapsid protein, or a cellular transmembrane protein. In some embodiments, the transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD 114, Baboon endogenous virus (BaEV), glycoprotein (GP41), or glycoprotein 120 (GP120). In some embodiments, the VSVG transmembrane region comprises full length VSVG transmembrane region or a truncated VSVG transmembrane region. In some embodiments, the transmembrane polypeptide comprises the VSVG transmembrane region and a VSVG cytoplasmic tail. In some embodiments, the transmembrane polypeptide comprises an amino acid sequence at least about 90% identical to the amino acid sequence according to any one of SEQ ID NOs: 19-27. In some embodiments, the fusion protein further comprises an oligomerization domain. In some embodiments, the oligomerization domain comprises a dimerization domain, a trimerization domain, or a tetramerization domain. In some embodiments, the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain. In some embodiments, the oligomerization domain comprises an amino acid sequence that has at least 90% sequence identity to an amino acid sequence according to any one of SEQ ID NOs: 5-18, and 28. In some embodiments, the particle is a viral-like a particle. In some embodiments, the particle is an extracellular vesicle. In some embodiments, the particle is an exosome. In some embodiments, the particle is an ectosome.

Disclosed herein, in another aspect, is a particle that comprises a DPP4 polypeptide on a surface of the particle wherein the DPP4 polypeptide is expressed at a valency of at least 10 copies on the surface of the particle and in an oligomerized format wherein the DPP4 polypeptide forms multivalent interactions with a viral protein which provide tighter binding through avidity than a soluble version of the DPP4 polypeptide to the viral protein. In some embodiments, the viral protein comprises a MERS coronavirus spike protein. In some embodiments, the particle binds to the viral protein to effectively neutralize a virus comprising the viral protein. In some embodiments, the virus comprises a MERS coronavirus. In some embodiments, the DPP4 polypeptide is part of a fusion protein, wherein the fusion protein comprises a transmembrane polypeptide. In some embodiments, the transmembrane polypeptide comprises a spike glycoprotein transmembrane region, a mammalian membrane protein, an envelope protein, a nucleocapsid protein, or a cellular transmembrane protein. In some embodiments, the transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD114, Baboon endogenous virus (BaEV), glycoprotein (GP41), or glycoprotein 120 (GP120). In some embodiments, the transmembrane polypeptide comprises an amino acid sequence at least about 90% identical to the amino acid sequence according to any one of SEQ ID NOs: 19-27. In some embodiments, the fusion protein further comprises an oligomerization domain. In some embodiments, the oligomerization domain comprises a dimerization domain, a trimerization domain, or a tetramerization domain. In some embodiments, the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain. In some embodiments, the oligomerization domain comprises an amino acid sequence that has at least 90% sequence identity to an amino acid sequence according to any one of SEQ ID NOs: 5-18, and 28. In some embodiments, the particle is a viral-like a particle. In some embodiments, the particle is an extracellular vesicle. In some embodiments, the particle is an exosome. In some embodiments, the particle is an ectosome.

Disclosed herein, in another aspect, is a multivalent particle that comprises at least 10 copies of a ACE2 polypeptide on the surface of the particle wherein the particle is self-assembled from the expression of a fusion protein that contains the ACE2 polypeptide sequence and an oligomerization domain sequence from a vector that is transfected into a cell.

Disclosed herein, in another aspect, is a multivalent particle that comprises at least 10 copies of a DPP4 polypeptide on the surface of the particle wherein the particle is self-assembled from the expression of a fusion protein that contains the DPP4 polypeptide sequence and an oligomerization domain sequence from a vector that is transfected into a cell.

Disclosed herein, in another aspect, is a method of treating a viral infection in a subject in need thereof comprising a multivalent particle displaying a ACE2 polypeptide on a surface of a particle, wherein the displayed ACE2 molecule on the particle forms multivalent interaction to a viral protein which provides tighter binding through avidity than a soluble version of the display polypeptide to the viral protein and administering the multivalent particle to the subject.

Disclosed herein, in another aspect, is a method of treating a viral infection in a subject in need thereof comprising a multivalent particle displaying a DPP4 polypeptide on a surface of a particle, wherein the displayed DPP4 molecule on the particle forms multivalent interaction to a viral protein which provides tighter binding through avidity than a soluble version of the display polypeptide to the viral protein and administering the multivalent particle to the subject.

Disclosed herein, in another aspect, is a method of inducing immunity against a viral infection in a subject comprising administering to the subject a composition disclosed herein or a particle disclosed herein. In some embodiments, the administering further comprises administering to the subject a virus comprising the viral protein, wherein the viral infection comprises an infection by the virus. In some embodiments, the method further comprises mixing the composition disclosed herein or the particle disclosed herein with the virus before the administering. In some embodiments, the method further comprises, before the administering, incubating the composition disclosed herein or the particle disclosed herein with the virus after the mixing, wherein the composition disclosed herein or the particle disclosed herein neutralizes the virus. In some embodiments, the composition disclosed herein or the particle disclosed herein forms a complex with the virus. In some embodiments, the complex is in an immune cell after the administering. In some embodiments, the immune cell comprises a macrophage. In some embodiments, the immune cell comprises a dendritic cell. In some embodiments, the viral infection comprises an infection by SARS CoV-2, SARS CoV-1, MERS CoV, respiratory syncytial virus, HBV, HDV, or HIV. In some embodiments, the viral infection comprises an infection by the original SARS CoV-2 virus, Delta variant of SARS CoV-2, Beta variant of SARS CoV-2, SARS CoV-2 B.1.351 Beta variant, SARS CoV-2 B.1.617.2 Delta variant, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant SARS CoV-2, or the B.1.351 South Africa strain of SARS CoV-2 comprising the E484K+N501Y mutant of spike protein. In some embodiments, the multivalent particle neutralizes a virus comprising the viral spike protein when the displayed virus-capturing polypeptide forms multivalent interactions with the viral spike protein. In some embodiments, the neutralizing potency of the multivalent particle against the virus increases when the displayed virus-capturing polypeptide form oligomers on the surface of the multivalent particle. In some embodiments, the neutralizing potency of the multivalent particle against the virus is correlated with the copy number of the displayed virus-capturing polypeptide expressed on the surface of the multivalent particle. In some embodiments, the neutralizing potency of the multivalent particle against the virus increases when the copy number of the displayed virus-capturing polypeptide expressed on the surface of the multivalent particle increases. In some embodiments, the neutralizing potency comprises: (a) a suppression of a viral infection; and/or (b) a number of viral particles that bind to a single multivalent particle.

Disclosed herein, in another aspect, is a method for immunizing a subject against a viral infection comprising administering to the subject: (a) a composition disclosed herein or a particle disclosed herein; and (b) a virus comprising the viral spike protein, wherein the viral infection comprises an infection by the virus. In some embodiments, the virus comprises a live virus. In some embodiments, the virus comprises an inactivated or dead virus. In some embodiments, the viral infection comprises an infection by SARS CoV-2, SARS CoV-1, MERS CoV, respiratory syncytial virus, HBV, HDV, or HIV. In some embodiments, the viral infection comprises an infection by the original SARS CoV-2 virus, Delta variant of SARS CoV-2, Beta variant of SARS CoV-2, B.1.351 Beta variant of SARS CoV-2, B.1.617.2 Delta variant of SARS CoV-2, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant of SARS CoV-2, or B.1.351 South Africa strain of SARS CoV-2 comprising the E484K+N501Y mutant of spike protein. In some embodiments, the administering comprises administering through inhalation. In some embodiments, the administering comprises administering intranasally. In some embodiments, the administering comprises administering intravenously.

Disclosed herein, in another aspect, is a method for treating a viral infection in a subject comprising administering to the subject a composition disclosed herein or a particle disclosed herein. In some embodiments, the administering induces immunity in the subject against the viral infection. In some embodiments, the viral infection comprises an infection by SARS CoV-2, SARS CoV-1, MERS CoV, respiratory syncytial virus, HBV, HDV, or HIV. In some embodiments, the viral infection comprises an infection by the original SARS CoV-2 virus, Delta variant of SARS CoV-2, Beta variant of SARS CoV-2, B.1.351 Beta variant of SARS CoV-2, B.1.617.2 Delta variant of SARS CoV-2, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant of SARS CoV-2, or B.1.351 South Africa strain of SARS CoV-2 comprising the E484K+N501Y mutant of spike protein.

Disclosed herein, in another aspect, is multivalent particle comprising a fusion protein that comprises a mammalian polypeptide that binds to a viral protein and a transmembrane polypeptide wherein the fusion protein is expressed at least about 10 copies on a surface of the multivalent particle. In some embodiments, the viral protein is from SARS-CoV-1, SARS-CoV-2, MERS-CoV, respiratory syncytial virus, HBV, HDV, HIV, or combinations thereof. In some embodiments, the mammalian polypeptide comprises a receptor that has binding specificity for the viral protein. In some embodiments, the receptor comprises a viral entry receptor or a viral attachment receptor. In some embodiments, the receptor is both a viral entry receptor and a viral attachment receptor. In some embodiments, the mammalian polypeptide comprises an extracellular domain of the receptor. In some embodiments, the mammalian polypeptide comprises a ligand or a secreted protein. In some embodiments, the mammalian polypeptide comprises ACE2, TRMPSS2, DPP4, CD4, CCR5, CXCR4, CD209, or CLEC4M. In some embodiments, the mammalian polypeptide comprises an amino acid sequence of at least 90% sequence identity to an amino acid sequence according to SEQ ID NO: 1. In some embodiments, the mammalian polypeptide comprises an amino acid sequence of at least 90% sequence identity to an amino acid sequence according to SEQ ID NO: 2. In some embodiments, the transmembrane polypeptide anchors the fusion protein to a bilayer of the multivalent particle. In some embodiments, the transmembrane polypeptide comprises a spike glycoprotein transmembrane region, a mammalian membrane protein, an envelope protein, a nucleocapsid protein, or a cellular transmembrane protein. In some embodiments, the transmembrane polypeptide comprises a VSVG transmembrane region, spike protein S1 transmembrane region, spike protein S2 transmembrane region, Sindbis virus envelope (SINDBIS) protein, hemagglutinin envelope protein from measles virus, envelope glycoprotein of measles virus fusion (F) protein, RD114, BaEV, GP41, or GP120. In some embodiments, the VSVG transmembrane region comprises full length VSVG transmembrane region or a truncated VSVG transmembrane region. In some embodiments, the transmembrane polypeptide comprises the VSVG transmembrane region and a VSVG cytoplasmic tail. In some embodiments, the transmembrane polypeptide comprises an amino acid sequence at least about 90% identical to that set forth in SEQ ID NO: 3. In some embodiments, the transmembrane polypeptide comprises an amino acid sequence at least about 90% identical to that set forth in SEQ ID NO: 4. In some embodiments, the fusion protein is expressed at least about 50 copies on a surface of the multivalent particle. In some embodiments, the fusion protein is expressed at least about 75 copies on a surface of the multivalent particle. In some embodiments, the fusion protein is expressed at least about 100 copies on a surface of the multivalent particle. In some embodiments, the fusion protein is expressed at least about 150 copies on a surface of the multivalent particle. In some embodiments, the fusion protein is expressed at least about 200 copies on a surface of the multivalent particle. In some embodiments, the mammalian polypeptide comprises ACE2 and the transmembrane polypeptide comprises a VSVG transmembrane region. In some embodiments, the mammalian polypeptide comprises ACE2 and the transmembrane polypeptide comprises a spike protein S2 transmembrane region. In some embodiments, the mammalian polypeptide comprises ACE2 and the transmembrane polypeptide comprises a surface glycoprotein transmembrane region of an enveloped virus. In some embodiments, the mammalian polypeptide comprises DPP4 and the transmembrane polypeptide comprises hemagglutinin envelope protein from measles virus. In some embodiments, the hemagglutinin envelope protein from measles virus is a variant of the hemagglutinin envelope protein from measles virus. In some embodiments, the multivalent particle further comprises a second fusion protein that comprises a second mammalian polypeptide that binds to the viral protein and a second transmembrane polypeptide wherein the second fusion protein is expressed at least about 10 copies on the surface of the multivalent particle. In some embodiments, the second mammalian polypeptide comprises a receptor that has binding specificity for the viral protein. In some embodiments, the receptor comprises a viral entry receptor or a viral attachment receptor. In some embodiments, the receptor is both a viral entry receptor and a viral attachment receptor. In some embodiments, the second mammalian polypeptide comprises an extracellular domain of the receptor. In some embodiments, the second mammalian polypeptide comprises a ligand or a secreted protein. In some embodiments, the second mammalian polypeptide comprises ACE2, TRMPSS2, DPP4, CD4, CCR5, CXCR4, CD209, or CLEC4M. In some embodiments, the second mammalian polypeptide comprises an amino acid sequence of at least 90% sequence identity to an amino acid sequence according to SEQ ID NO: 1. In some embodiments, the second mammalian polypeptide comprises an amino acid sequence of at least 90% sequence identity to an amino acid sequence according to SEQ ID NO: 2. In some embodiments, the second transmembrane polypeptide comprises a transmembrane anchoring protein. In some embodiments, the second transmembrane polypeptide comprises a spike glycoprotein transmembrane region, a mammalian membrane protein, an envelope protein, a nucleocapsid protein, or a cellular transmembrane protein. In some embodiments, the second transmembrane polypeptide comprises VSVG transmembrane region, spike protein S1 transmembrane region, spike protein S2 transmembrane region, Sindbis virus envelope (SINDBIS) protein, hemagglutinin envelope protein from measles virus, envelope glycoprotein of measles virus fusion (F) protein, RD 114, BaEV, GP41, or GP120. In some embodiments, the VSVG transmembrane region comprises full length VSVG transmembrane region or a truncated VSVG transmembrane region. In some embodiments, the transmembrane polypeptide comprises a VSVG transmembrane region and a VSVG cytoplasmic tail. In some embodiments, the second transmembrane polypeptide comprises an amino acid sequence at least about 90% identical to that set forth in SEQ ID NO: 3. In some embodiments, the second transmembrane polypeptide comprises an amino acid sequence at least about 90% identical to that set forth in SEQ ID NO: 4. In some embodiments, the second fusion protein is expressed at least about 50 copies on a surface of the multivalent particle. In some embodiments, the second fusion protein is expressed at least about 75 copies on a surface of the multivalent particle. In some embodiments, the second fusion protein is expressed at least about 100 copies on a surface of the multivalent particle. In some embodiments, the second fusion protein is expressed at least about 150 copies on a surface of the multivalent particle. In some embodiments, the second fusion protein is expressed at least about 200 copies on a surface of the multivalent particle. In some embodiments, the second mammalian polypeptide comprises ACE2 and the second transmembrane polypeptide comprises VSVG transmembrane region. In some embodiments, the second mammalian polypeptide comprises ACE2 and the second transmembrane polypeptide comprises spike protein S2 transmembrane region. In some embodiments, the second mammalian polypeptide comprises ACE2 and the second transmembrane polypeptide comprises a surface glycoprotein of an enveloped virus. In some embodiments, the second mammalian polypeptide comprises DPP4 and the second transmembrane polypeptide comprises hemagglutinin envelope protein from measles virus. In some embodiments, the hemagglutinin envelope protein from measles virus is a variant of the hemagglutinin envelope protein from measles virus. In some embodiments, the mammalian polypeptide comprises a viral entry receptor and the second mammalian polypeptide comprises a viral attachment receptor. In some embodiments, the mammalian polypeptide comprises ACE2, the transmembrane polypeptide comprises VSVG transmembrane region, spike protein S1 transmembrane region, spike protein S2 transmembrane region, or a surface glycoprotein of an enveloped virus, the second mammalian polypeptide comprises a heparan sulfate proteoglycan, and the second transmembrane polypeptide comprises VSVG transmembrane region, spike protein S1 transmembrane region, spike protein S2 transmembrane region, or a surface glycoprotein of an enveloped virus. In some embodiments, the mammalian polypeptide comprises CD4 and the second mammalian peptide comprises, CCR5, CXCR4, or both. In some embodiments, the multivalent particle comprises an IC50 of less than 5 picomolar (pM) in a neutralization assay. In some embodiments, the multivalent particle comprises an IC50 of less than 2.5 picomolar (pM) in a neutralization assay. In some embodiments, the multivalent particle comprises an IC50 of less than 1 picomolar (pM) in a neutralization assay. In some embodiments, the multivalent particle does not comprise viral genetic material. In some embodiments, the multivalent particle is synthetic. In some embodiments, the multivalent particle is recombinant. In some embodiments, the multivalent particle is a viral-like a particle. In some embodiments, the multivalent particle is an extracellular vesicle. In some embodiments, the multivalent particle is an exosome. In some embodiments, the multivalent particle is an ectosome. In some embodiments, the fusion protein further comprises an oligomerization domain. In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the trimerization domain comprises human C-propeptide of α1(I) collagen. In some embodiments, the oligomerization domain is a tetramerization domain. In some embodiments, the tetramerization domain comprises an influenza neuraminidase stem domain. In some embodiments, the oligomerization domain comprises an amino acid sequence that has at least 95% sequence identity to an amino acid sequence according to SEQ ID NOs: 5-18, or 28. In some embodiments, when the fusion protein is expressed on the surface of the multivalent particle, the oligomerization domain is outside of the multivalent particle. In some embodiments, when the fusion protein is expressed on the surface of the multivalent particle, the oligomerization domain is outside of the multivalent particle and adjacent to a signal peptide. In some embodiments, when the fusion protein is expressed on the surface of the multivalent particle, the oligomerization domain is inside of the multivalent particle. In some embodiments, when the fusion protein is expressed on the surface of the multivalent particle, the oligomerization domain is inside of the multivalent particle and adjacent to the transmembrane polypeptide. In some embodiments, the fusion protein comprises a signal peptide. In some embodiments, domains of the fusion protein are arranged from the N-terminus to the C-terminus in the following orders: (a) signal peptide, extracellular domain of a viral entry receptor which binds to a surface protein of a virus, oligomerization domain, transmembrane polypeptide, and cytosolic domain; (b) signal peptide, extracellular domain of a viral entry receptor which binds to a surface protein of a virus, transmembrane polypeptide, oligomerization domain, and cytosolic domain; or (c) signal peptide, oligomerization domain, extracellular domain of a viral entry receptor, transmembrane polypeptide, and cytosolic domain.

Disclosed herein, in another aspect, is a composition comprising a first nucleic acid sequence encoding a multivalent particle comprising a fusion protein that comprises an extracellular domain of a viral entry receptor that binds to a viral protein and a transmembrane polypeptide wherein the fusion protein is expressed at least about 10 copies on a surface of the multivalent particle when the multivalent particle is expressed; and an excipient. In some embodiments, the viral protein is from SARS-CoV-1, SARS-CoV-2, MERS-CoV, respiratory syncytial virus, HBV, HDV, HIV, or combinations thereof. In some embodiments, the composition further comprises a second nucleic acid sequence that encodes one or more packaging viral proteins. In some embodiments, the one or more packaging viral proteins is a lentiviral protein, a retroviral protein, an adenoviral protein, or combinations thereof. In some embodiments, the one or more packaging viral proteins comprises gag, pol, pre, tat, rev, or combinations thereof. In some embodiments, the composition further comprises a third nucleic acid sequence that encodes a replication incompetent viral genome, a reporter, a therapeutic molecule, or combinations thereof. In some embodiments, the viral genome is derived from vesicular stomatitis virus, measles virus, Hepatitis virus, influenzavirus, or combinations thereof. In some embodiments, the reporter is a fluorescent protein or luciferase. In some embodiments, the fluorescent protein is green fluorescent protein. In some embodiments, the therapeutic molecule is an immune modulating protein, a cellular signal modulating molecule, a proliferation modulating molecule, a cell death modulating molecule, or combinations thereof. In some embodiments, the mammalian polypeptide comprises a receptor that has binding specificity for the viral protein. In some embodiments, the receptor comprises a viral entry receptor or a viral attachment receptor. In some embodiments, the receptor is both a viral entry receptor and a viral attachment receptor. In some embodiments, the mammalian polypeptide comprises an extracellular domain of the receptor. In some embodiments, the mammalian polypeptide comprises a ligand or a secreted protein. In some embodiments, the mammalian polypeptide comprises ACE2, TRMPSS2, DPP4, CD4, CCR5, CXCR4, CD209, or CLEC4M. In some embodiments, the mammalian polypeptide comprises an amino acid sequence of at least 90% sequence identity to an amino acid sequence according to SEQ ID NO: 1. In some embodiments, the mammalian polypeptide comprises an amino acid sequence of at least 90% sequence identity to an amino acid sequence according to SEQ ID NO: 2. In some embodiments, the transmembrane polypeptide comprises a transmembrane anchoring protein. In some embodiments, the transmembrane polypeptide comprises a spike glycoprotein transmembrane region, a mammalian membrane protein, an envelope protein, a nucleocapsid protein, or a cellular transmembrane protein. In some embodiments, the transmembrane polypeptide comprises VSVG transmembrane region, spike protein S1 transmembrane region, spike protein S2 transmembrane region, Sindbis virus envelope (SINDBIS) protein, hemagglutinin envelope protein from measles virus, envelope glycoprotein of measles virus fusion (F) protein, RD114, BaEV, GP41, or GP120. In some embodiments, the VSVG transmembrane region comprises full length VSVG transmembrane region or a truncated VSVG transmembrane region. In some embodiments, the transmembrane polypeptide comprises a VSVG transmembrane region and a VSVG cytoplasmic tail. In some embodiments, the transmembrane polypeptide comprises an amino acid sequence at least about 90% identical to that set forth in SEQ ID NO: 3. In some embodiments, the transmembrane polypeptide comprises an amino acid sequence at least about 90% identical to that set forth in SEQ ID NO: 4. In some embodiments, the fusion protein further comprises an oligomerization domain. In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the trimerization domain comprises human C-propeptide of α1(I) collagen. In some embodiments, the oligomerization domain is a tetramerization domain. In some embodiments, the tetramerization domain comprises an influenza neuraminidase stem domain. In some embodiments, the oligomerization domain comprises an amino acid sequence that has at least 95% sequence identity to an amino acid sequence according to SEQ ID NOs: 5-18, or 28. In some embodiments, when the fusion protein is expressed on the surface of the multivalent particle, the oligomerization domain is outside of the multivalent particle. In some embodiments, when the fusion protein is expressed on the surface of the multivalent particle, the oligomerization domain is outside of the multivalent particle and adjacent to a signal peptide. In some embodiments, when the fusion protein is expressed on the surface of the multivalent particle, the oligomerization domain is inside of the multivalent particle. In some embodiments, when the fusion protein is expressed on the surface of the multivalent particle, the oligomerization domain is inside of the multivalent particle and adjacent to the transmembrane polypeptide. In some embodiments, the fusion protein is expressed at least about 50 copies on a surface of the multivalent particle when it is expressed. In some embodiments, the fusion protein is expressed at least about 75 copies on a surface of the multivalent particle when itis expressed. In some embodiments, the fusion protein is expressed at least about 100 copies on a surface of the multivalent particle when it is expressed. In some embodiments, the fusion protein is expressed at least about 150 copies on a surface of the multivalent particle when it is expressed. In some embodiments, the fusion protein is expressed at least about 200 copies on a surface of the multivalent particle when it is expressed. In some embodiments, the mammalian polypeptide comprises ACE2 and the transmembrane polypeptide comprises VSVG transmembrane region. In some embodiments, the mammalian polypeptide comprises ACE2 and the transmembrane polypeptide comprises spike protein S2 transmembrane region. In some embodiments, the mammalian polypeptide comprises ACE2 and the transmembrane polypeptide comprises a surface glycoprotein of an enveloped virus. In some embodiments, the mammalian polypeptide comprises DPP4 and the transmembrane polypeptide comprises hemagglutinin envelope protein from measles virus. In some embodiments, the hemagglutinin envelope protein from measles virus is a variant of the hemagglutinin envelope protein from measles virus. In some embodiments, the composition further comprises a fourth nucleic acid sequence encoding a second fusion protein that comprises a second mammalian polypeptide that binds to the viral protein and a second transmembrane polypeptide wherein the second fusion protein is expressed at least about 10 copies on the surface of the multivalent particle when it is expressed. In some embodiments, the second mammalian polypeptide comprises a receptor that has binding specificity for the viral protein. In some embodiments, the receptor comprises a viral entry receptor or a viral attachment receptor. In some embodiments, the receptor is both a viral entry receptor and a viral attachment receptor. In some embodiments, the second mammalian polypeptide comprises an extracellular domain of the receptor. In some embodiments, the second mammalian polypeptide comprises a ligand or a secreted protein. In some embodiments, the second mammalian polypeptide comprises ACE2, TRMPSS2, DPP4, CD4, CCR5, CXCR4, CD209, or CLEC4M. In some embodiments, the second mammalian polypeptide comprises an amino acid sequence of at least 90% sequence identity to an amino acid sequence according to SEQ ID NO: 1. In some embodiments, the second mammalian polypeptide comprises an amino acid sequence of at least 90% sequence identity to an amino acid sequence according to SEQ ID NO: 2. In some embodiments, the second transmembrane polypeptide comprises a transmembrane anchoring protein. In some embodiments, the second transmembrane polypeptide comprises a spike glycoprotein transmembrane region, a mammalian membrane protein, an envelope protein, a nucleocapsid protein, or a cellular transmembrane protein. In some embodiments, the second transmembrane polypeptide comprises VSVG transmembrane region, spike protein S1 transmembrane region, spike protein S2 transmembrane region, Sindbis virus envelope (SINDBIS) protein, hemagglutinin envelope protein from measles virus, envelope glycoprotein of measles virus fusion (F) protein, RD114, BaEV, GP41, or GP120. In some embodiments, the VSVG transmembrane region comprises full length VSVG transmembrane region or a truncated VSVG transmembrane region. In some embodiments, the VSVG transmembrane region comprises a VSVG transmembrane region and a VSVG cytoplasmic tail. In some embodiments, the second transmembrane polypeptide comprises an amino acid sequence at least about 90% identical to that set forth in SEQ ID NO: 3. In some embodiments, the second transmembrane polypeptide comprises an amino acid sequence at least about 90% identical to that set forth in SEQ ID NO: 4. In some embodiments, the second fusion protein further comprises an oligomerization domain. In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the trimerization domain comprises human C-propeptide of α1(I) collagen. In some embodiments, the oligomerization domain is a tetramerization domain. In some embodiments, the tetramerization domain comprises an influenza neuraminidase stem domain. In some embodiments, the oligomerization domain comprises an amino acid sequence that has at least 95% sequence identity to an amino acid sequence according to SEQ ID NOs: 5-18, or 28. In some embodiments, when the second fusion protein is expressed on the surface of the multivalent particle, the oligomerization domain is outside of the multivalent particle. In some embodiments, when the second fusion protein is expressed on the surface of the multivalent particle, the oligomerization domain is outside of the multivalent particle and adjacent to a signal peptide. In some embodiments, when the second fusion protein is expressed on the surface of the multivalent particle, the oligomerization domain is inside of the multivalent particle. In some embodiments, when the second fusion protein is expressed on the surface of the multivalent particle, the oligomerization domain is inside of the multivalent particle and adjacent to the transmembrane polypeptide. In some embodiments, the second fusion protein is expressed at least about 50 copies on a surface of the multivalent particle when itis expressed. In some embodiments, the second fusion protein is expressed at least about 75 copies on a surface of the multivalent particle when it is expressed. In some embodiments, the second fusion protein is expressed at least about 100 copies on a surface of the multivalent particle when it is expressed. In some embodiments, the second fusion protein is expressed at least about 150 copies on a surface of the multivalent particle when it is expressed. In some embodiments, the second fusion protein is expressed at least about 200 copies on a surface of the multivalent particle when it is expressed. In some embodiments, the second mammalian polypeptide comprises ACE2 and the second transmembrane polypeptide comprises VSVG transmembrane region. In some embodiments, the second mammalian polypeptide comprises ACE2 and the second transmembrane polypeptide comprises spike protein S2 transmembrane region. In some embodiments, the second mammalian polypeptide comprises ACE2 and the second transmembrane polypeptide comprises a surface glycoprotein of an enveloped virus. In some embodiments, the second mammalian polypeptide comprises DPP4 and the second transmembrane polypeptide comprises hemagglutinin envelope protein from measles virus. In some embodiments, the hemagglutinin envelope protein from measles virus is a variant of the hemagglutinin envelope protein from measles virus. In some embodiments, the mammalian polypeptide comprises a viral entry receptor and the second mammalian polypeptide comprises a viral attachment receptor. In some embodiments, the mammalian polypeptide comprises ACE2, the transmembrane polypeptide comprises VSVG transmembrane region, spike protein S1 transmembrane region, spike protein S2 transmembrane region, or a surface glycoprotein of an enveloped virus, the second mammalian polypeptide comprises a heparan sulfate proteoglycan, and the second transmembrane polypeptide comprises VSVG transmembrane region, spike protein S1 transmembrane region, spike protein S2 transmembrane region, or a surface glycoprotein of an enveloped virus. In some embodiments, the mammalian polypeptide comprises CD4 and the second mammalian peptide comprises, CCR5, CXCR4, or both. In some embodiments, the first nucleic acid sequence, the second nucleic acid sequence, and the third nucleic acid sequence are within a same vector. In some embodiments, the first nucleic acid sequence, the second nucleic acid sequence, and the third nucleic acid sequence are within different vectors. In some embodiments, the first nucleic acid sequence, the second nucleic acid sequence, the third nucleic acid sequence, and the fourth nucleic acid sequence are within a same vector. In some embodiments, the first nucleic acid sequence, the second nucleic acid sequence, third nucleic acid sequence, and the fourth nucleic acid sequence are within different vectors. In some embodiments, the nucleic acid sequence that encodes the first fusion protein and the second fusion protein and the second nucleic acid sequence and the third nucleic acid sequence are mRNAs. In some embodiments, the nucleic acid sequence that encodes the first fusion protein and the second fusion protein and the second nucleic acid sequence and the third nucleic acid sequence are DNA. In some embodiments, the composition comprises a vector, wherein the vector is a lentivirus vector, an adenovirus vector, or an adeno-associated virus vector.

Disclosed herein, in another aspect, is a pharmaceutical composition comprising a multivalent particle disclosed herein and a pharmaceutically acceptable excipient.

Disclosed herein, in another aspect, is a method of treating a viral infection in a subject in need thereof, comprising administering to the subject a multivalent particle disclosed herein or a composition disclosed herein. In some embodiments, the multivalent particle is administered intravenously. In some embodiments, the multivalent particle is administered through inhalation. In some embodiments, the multivalent particle is administered through intranasal delivery. In some embodiments, the multivalent particle is administered by an intraperitoneal injection. In some embodiments, the multivalent particle is administered by a subcutaneous injection. In some embodiments, the viral infection comprises an infection by SARS CoV-2, SARS CoV-1, MERS CoV. In some embodiments, the composition is administered intravenously. In some embodiments, the composition is administered through inhalation. In some embodiments, the composition is administered by an intraperitoneal injection. In some embodiments, the composition is administered by a subcutaneous injection. In some embodiments, the composition comprises a liposome. In some embodiments, the composition comprises an adeno-associated virus (AAV). In some embodiments, the composition comprises a lipid nanoparticle. In some embodiments, the composition comprises a polymer. In some embodiments, the SARS CoV-2, SARS CoV-1, MERS CoV are effectively neutralized in vivo by the multivalent particle or the composition. In some embodiments, the multivalent particle or the composition inhibits a respiratory symptom of the viral infection. In some embodiments, the multivalent particle or the composition induces robust immunity against different strains of the viral infection. In some embodiments, the viral infection comprises infection by SARS CoV-2, and the multivalent particle or the composition induces robust immunity against Delta variant of SARS CoV-2.

Disclosed herein, in another aspect, is a method of producing immunity against a viral infection in a subject in need thereof, comprising administering to the subject a multivalent particle disclosed herein or a composition disclosed herein and a virus of the viral infection. In some embodiments, the multivalent particle is administered intravenously. In some embodiments, the multivalent particle is administered through inhalation. In some embodiments, the multivalent particle is administered through intranasal delivery. In some embodiments, the multivalent particle is administered by an intraperitoneal injection. In some embodiments, the multivalent particle is administered by a subcutaneous injection. In some embodiments, the viral infection comprises an infection by SARS CoV-2, SARS CoV-1, MERS CoV, respiratory syncytial virus, HBV, HDV, or HIV. In some embodiments, the composition is administered intravenously. In some embodiments, the composition is administered through inhalation. In some embodiments, the composition is administered by an intraperitoneal injection. In some embodiments, the composition is administered by a subcutaneous injection. In some embodiments, the composition comprises a liposome. In some embodiments, the composition comprises an adeno-associated virus (AAV). In some embodiments, the composition comprises a lipid nanoparticle. In some embodiments, the composition comprises a polymer. In some embodiments, the SARS CoV-2, SARS CoV-1, MERS CoV, respiratory syncytial virus, HBV, HDV, or HIV are effectively neutralized in vivo by the multivalent particle or the composition. In some embodiments, the multivalent particle or the composition inhibits a respiratory symptom of the viral infection. In some embodiments, the multivalent particle or the composition induces robust immunity against different strains of the viral infection. In some embodiments, the viral infection comprises infection by SARS CoV-2, and the multivalent particle or the composition induces robust immunity against Delta variant of SARS CoV-2.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the design, characterization, and function of ACE-2 antiviruses.

FIG. 2 shows the design, characterization, and function of decoy antiviruses displaying enzymatically inactive ACE2.

FIG. 3 shows the neutralizing activities and toxicities of monomeric WT-VGTM and oligomeric H2A-VGTM as determined in a SARS CoV-2 live virus neutralization assay.

FIG. 4 shows that post-exposure intranasal delivery of H2A-D4VG Antivirus recued lethal infection of hACE2 transgenic mice by the SARS CoV-2 Washington strain.

FIG. 5 shows that post-exposure intranasal delivery of H2A-D4VG Antivirus recued lethal infection of hACE2 transgenic mice by the SARS CoV-2 delta variant.

FIG. 6 shows that infected mice rescued by H2A-antivirus treatment had broad immunity against SARS CoV-2 variants.

FIG. 7A shows the design of uptake analyses of ACE2-antivirus and SARS CoV-2 pseudovirus complexes by lung alveolar macrophages and dendritic cells.

FIG. 7B shows FACS staining and gating strategy for defining the uptake of ACE2-antivirus and SARS CoV-2 pseudovirus complexes by lung alveolar macrophages and dendritic cells.

FIG. 7C shows the relative uptake of dye-labeled SARS CoV-2 pseudovirus alone or ACE2-antivirus and pseudovirus complexes by lung alveolar macrophages.

FIG. 7D shows the relative uptake of dye-labelled SARS CoV-2 pseudovirus alone or ACE2-antivirus and pseudovirus complexes by dendritic cells.

FIG. 8 shows the neutralizing potentials of ACE2-VGTM antivirus in a delayed pseudovirus neutralization assay (PNA).

FIG. 9 shows the stability of H2A-D4VG antivirus stored at −80° C., 4° C., and 25° C.

FIG. 10 shows the pharmacokinetics of ACE2-antivirus following intranasal dosing.

FIG. 11A shows a schematic of pseudotyped lentiviral particles with a fusion protein consisting of the ACE2 extracellular domain and the membrane anchoring segment of a viral envelop protein.

FIG. 11B shows quantitative Western blot analysis of ACE2 valency of different multivalent particles.

FIG. 11C shows the particle size distribution of ACE2-VGTM MVPs as determined by Tunable Resistive Pulse Sensing Analysis using a qNano instrument.

FIG. 11D shows representative Electron Microscopy images of ACE2-VGTM MVPs at nominal magnification of 150,000×.

FIG. 12A shows results of a microneutralization assay using 293T/ACE2 cells as target cells.

FIG. 12B shows the maximum inhibition of pseudovirus infection by different multivalent particles.

FIG. 12C shows stoichiometric ratios between the neutralizing decoy ACE2-MVPs and the pseudovirus particles as determined by P24 ELISA assays.

FIG. 12D shows results of a microneutralization assay using a decoy ACE2-MVP and two neutralizing antibodies.

FIG. 13A shows neutralization of lentiviruses pseudotyped with SARS CoV-1 spike (CoV-1 PVPs).

FIG. 13B shows neutralizing activities of ACE2-MVPs in a CoV-1 PVP neutralization using VERO-E6 cells as target cells.

FIG. 13C shows results of a microneutralization assay against Cov-1, Cov-2 WT and Cov-2 D614G pseudotyped viruses using 293T/ACE2 cells as target cells.

FIG. 13D shows results of a microneutralization assay against Cov-1, Cov-2 WT and Cov-2 D614G pseudotyped viruses using H1573/ACE2 cells as target cells.

FIG. 13E shows a comparison of the neutralizing activities of the ACE2-VGTM MVPs against a variety of SARS CoV-2 variants in pseudovirus infection assay using 293 T/ACE2 cells as target cells.

FIG. 14A shows a schematic of a decoy DPP4-MVP with fusion protein comprising a hemagglutinin envelope protein from measles virus (HCA18) and the DPP4 extracellular domain.

FIG. 14B depicts quantitative Western blot analysis of HCA-DPP4 valency of different multivalent particles.

FIG. 14C depicts the neutralizing activities of DDP4-MVPs were tested against lentiviruses pseudotyped with MERS spike (MERS-PVPs) in a microneutralization assay using H1650 cells as target cells.

FIG. 14D shows the design and production of NA75-DPP4 MVPs.

FIG. 14E shows the neutralizing activities of NA75-DPP4 MVPs determined in a MERS pseudovirus infection assay using H1650 cells as target cells.

FIG. 15 shows decoy-MVPs displaying enzymatic-inactive H2A-ACE2 with a reduced neutralizing activity against CoV-2 pseudovirus.

FIG. 16A shows the structure of post-fusion VSV-G with D4 domain as the trimerization domain.

FIG. 16B shows schematics illustrating the oligomerized ACE2-displaying constructs with ACE2 extracellular domain fused to the VSVG transmembrane domain (ACE2-VGTN) for monomeric display or to the D4 post-fusion trimerization domain and VSVG transmembrane domain (ACE2-D4VG) for trimeric display.

FIG. 16C shows the copy number of ACE2 molecules on the decoy-MVPs determined by quantitative Western-blot analyses.

FIG. 16D shows representative TRPS analysis of ACE2-D4VG MVPs.

FIG. 16E shows a representative Electron Microscopy image of H2A/ACE2-D4VG MVPs at nominal magnification of 150,000×.

FIG. 17A shows the neutralizing activities of the monomeric and trimeric wild-type ACE2-MVPs and enzymatically-inactive H2A/ACE2 MVPs as determined in a SARS CoV-2 pseudovirus infection assay using 293T/ACE2 cells as target cells.

FIG. 17B shows the neutralizing activities of the monomeric and trimeric wild-type ACE2 MVPs and enzymatically-inactive H2A/ACE2 MVPs as determined in a SARS CoV-1 pseudovirus infection assay using VERO-E6 cells as target cells.

FIG. 17C compares the neutralizing activities of the H2A/ACE2-D4VGMVPs against a variety of SARS CoV-2 variants in pseudovirus infection assay using 293T/ACE2 cells as target cells.

FIG. 18A shows the antiviral activity of monomeric wild-type ACE2-MVP:WT-VGTM in a premixed live SARS CoV-2 virus neutralization assay.

FIG. 18B shows the antiviral activity of trimeric enzymatically-inactive H2A/ACE2-MVP:H2A-D4VG were determined in a SARS CoV-2 live virus neutralization assay.

FIG. 19A shows the neutralizing activity of trimeric H2A/ACE2-MVPs against live wild-type SARS CoV-2

FIG. 19B shows the neutralizing activity of trimeric H2A/ACE2-MVPs against South Africa variant SARS CoV-2 determined via PRNT assay.

FIG. 20A shows the effect of trimeric H2A/ACE2-MVPs treatment on weight loss.

FIG. 20B shows the effect of trimeric H2A/ACE2-MVPs treatment on viral load in lung.

FIG. 21A shows the effect of trimeric H2A/ACE-MVPs treatment on survival of SARS CoV-2 infected hACE2 transgenic mice.

FIG. 21B shows the effects of the weight loss in hACE2 transgenic mice infected with the original WA strain of SARS CoV-2.

FIG. 21C shows the effect of trimeric H2A/ACE2-MVPs treatment on survival of SARS CoV-2 Delta variant infected hACE2 transgenic mice.

FIG. 21D shows the effects of the weight loss in hACE2 transgenic mice infected with the SARS CoV-2 Delta variant.

FIG. 22A shows the effect of SARS CoV-2 re-challenge on the body weight of infected hACE2 transgenic mice.

FIG. 22B shows the effect of SARS CoV-2 re-challenge on the survival of infected hACE2 transgenic mice.

FIG. 22C shows the effect of Delta variant re-challenge on the body weight of infected hACE2 transgenic mice.

FIG. 22D shows the effect of Delta variant re-challenge on the survival of infected hACE2 transgenic mice.

FIG. 23A shows the particle size distribution of EV-based ACE2-D4VG MVP determined by Tunable Resistive Pulse Sensing Analysis using a qNano instrument.

FIG. 23B shows the neutralizing activity of EV-based ACE2-D4VG MVP determined in a SARS CoV-2 pseudovirus infection assay using 293 T/ACE2 cells as target cells.

FIG. 23C shows the neutralizing activity of EV-based ACE2-D4VG MVPs determined in a SARS CoV-2 live virus neutralization assay.

FIG. 23D shows the cytotoxicity of EV-based ACE2-D4VG MVPs in the live virus neutralization assay.

FIG. 24A shows vector design for a monomeric display vector expressing a fusion protein consisting of a protein linked to the VSVG transmembrane and intracellular domains.

FIG. 24B shows vector design for a trimeric display vector expressing a fusion protein consisting of a protein linked to the D4 post-fusion trimerization domain of VSVG, followed by the transmembrane and intracellular domains of VSVG.

FIG. 25A shows monomeric decoy-MVP production by pseudo-typing ACE2 receptors on the lentiviral-based viral-like particles with viral genome.

FIG. 25B shows Monomeric decoy-MVP production by pseudo-typing ACE2 receptors on the lentiviral-based viral-like particles without viral genome.

FIG. 25C shows monomeric decoy-MVP production by pseudo-typing extracellular vesicles with ACE2 receptors.

FIG. 26A shows trimeric decoy-MVP production by pseudo-typing ACE2 receptors onto the lentiviral-based viral-like particles with viral genome.

FIG. 26B shows trimeric decoy-MVP production by pseudo-typing ACE2 receptors onto the lentiviral-based viral-like particles without viral genome.

FIG. 26C shows trimeric decoy-MVP production by pseudo-typing extracellular vesicles with ACE2 receptors.

FIG. 27A shows mixed monomeric and trimeric decoy-MVP production by pseudo-typing viral-entry receptors onto the lentiviral-based viral-like particles with viral genome.

FIG. 27B shows mixed monomeric and trimeric decoy-MVP production by pseudo-typing viral-entry receptors onto the lentiviral-based viral-like particles without viral genome.

FIG. 27C shows mixed monomeric and trimeric decoy-MVP production by pseudo-typing extracellular vesicles with viral-entry receptors.

FIG. 28A shows the decoy receptor display configuration with the D4 trimerization domain located outside of the decoy-MVP and adjacent to the transmembrane domain.

FIG. 28B shows the decoy receptor display configuration with the D4 trimerization domain located inside of the decoy-MVP and adjacent to the transmembrane domain.

FIG. 28C shows the decoy receptor display configuration with the D4 trimerization domain located outside of the decoy-MVP and after the signal peptide.

FIG. 28D shows the D4 truncations for trimeric display of decoy receptors on decoy-MVPs.

FIG. 28E shows the neutralizing activities of ACE2-D4VG MVPs with varied D4 location and length determined in a SARS CoV-2 pseudovirus infection assay using 293T/ACE2 cells as target cells.

FIG. 29A shows the decoy receptor display configuration with the oligomerization domain located outside of the decoy-MVP and adjacent to the transmembrane domain.

FIG. 29B shows the decoy receptor display configuration with the oligomerization domain located inside of the decoy-MVP and adjacent to the transmembrane domain.

FIG. 29C shows the decoy receptor display configuration with the oligomerization domain located outside of the decoy-MVP and after the signal peptide.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### Multivalent Particles

The COVID-19 pandemic has caused tremendous losses in human life and economic activities. Current strategies such as antibody therapies for neutralizing viruses are not entirely effective. This is in part due to viruses being able to adapt strategies to effectively gain entry of host cells while evading the control by host immune systems. Nearly all viruses utilize a multivalent strategy for attachment and entry of host cells. Each virion display hundreds of copies of spike proteins, which can simultaneously interact with multiple copies of host cell receptors and attachment proteins.

In the case of coronaviruses, SARS CoV-2 virions display hundreds of copies of trimeric spike proteins, and utilize local trimeric as well as global multivalent interactions between spike and host cell proteins for attachment and entry. For example, host cell receptors angiotensin-converting enzyme 2 (ACE2) and dipeptidyl peptidase 4 (DPP4) are used as entry receptors for SARS CoV-1/2 and MERS coronaviruses, respectively. The densely packed spike proteins on the virions enable them to interact with multiple copies of ACE2 or DPP4 on the host cell surface. The boost in functional affinity that viruses receive through multivalent interactions is exponential, and nearly all enveloped and non-enveloped viruses use this multivalent strategy for attachment and host-cell entry. This provides a tremendous advantage to viruses. Most notably, the multivalent strategy enables viruses to turn relatively weak monovalent interactions with millimolar binding affinities into super-strong multivalent interactions with functional affinities in the nanomolar to picomolar range, in turn creating a high threshold for low or monovalent binders, such as neutralizing antibodies and recombinant protein inhibitors, to overcome. Moreover, viruses harness high mutation rates and multivalent binding to host cells to facilitate immune evasion. Spike mutagenesis and novel glycosylation patterns can effectively disrupt the neutralizing function of antibodies and other low-valency viral-blocking agents with little impact on viral attachment and entry. The current development of viral neutralization molecules does not address the multivalent nature of virions and host cell interaction. Mutations that are resistant to current combinations of clinically-tested neutralization antibodies have emerged and render existing therapies ineffective or less effective.

Given that trimeric and multivalent spike presentation on virions underlies SASR CoV-2's ability to escape immune control through rapid mutagenesis, here we describe multivalent particles (MVPs) displaying multiple copies of viral entry receptors, such as ACE2 and DPP4, that mirror the trimeric multivalent pattern of spike proteins on the virions. We showed that the MVPs effectively counteracts the multivalent interactions between viruses and host cell proteins and have improved potency against viruses such as coronavirus. Multivalent particles (MVPs) disclosed herein display thousands copies of oligomeric viral-capturing protein mirroring the viral spike patterns and therefore are also referred to as antiviruses. The terms of MVPs and antiviruses are used interchangeably throughout the disclosure. An antivirus is a MVP disclosed herein that displays thousands copies of oligomeric viral-capturing protein and has enhanced antiviral activity and/or effects. Most importantly, the MVPs are insensitive to spike mutagenesis and therefore are variant-proof neutralizing therapeutics. Finally, treatment of SARS CoV-2 infection in representative animal models can effectively rescue lethal infection and induced robust immunity against dominant SARS CoV-2 strains including the Delta variant

Disclosed herein, in one aspect, is a composition of multivalent particle. In some embodiments, the multivalent particle comprises a virus-capturing polypeptide on a surface of the particle. In some embodiments, the displayed virus-capturing polypeptide is expressed at a valency of at least 10 copies on the surface of the particle. In some embodiments, the displayed virus-capturing polypeptide is expressed in an oligomerized format on the surface of the particle. In some embodiments, the virus-capturing polypeptide displayed on the surface of the particle forms multivalent interactions with a viral spike protein which provide tighter binding through avidity than a soluble version of the displayed virus-capturing polypeptide to the viral spike protein. Disclosed herein, in some embodiments, is a composition of multivalent particle that comprises a virus-capturing polypeptide on a surface of the particle wherein the displayed virus-capturing polypeptide is expressed at a valency of at least 10 copies on the surface of the particle and in an oligomerized format wherein the displayed virus-capturing polypeptide forms multivalent interactions with a viral spike protein which provide tighter binding through avidity than a soluble version of the displayed virus-capturing polypeptide to the viral spike protein. In some embodiments, the viral spike protein is from severe acute respiratory syndrome coronavirus 1 (SARS-CoV-1), severe acute respiratory syndrome coronavirus 2 (SARS-CoV-2), Middle East respiratory syndrome-related coronavirus (MERS-CoV), respiratory syncytial virus, HBV, HDV, or human immunodeficiency virus (HIV), or combinations thereof. In some embodiments, the displayed virus-capturing polypeptide comprises a receptor for the virus entry, a ligand, a secreted protein, an antibody, or an engineered protein. In some embodiments, the receptor comprises a viral entry receptor or a viral attachment receptor. In some embodiments, the receptor is both a viral entry receptor and a viral attachment receptor. In some embodiments, the displayed virus-capturing polypeptide comprises an extracellular domain of the receptor. In some embodiments, the displayed virus-capturing polypeptide comprises angiotensin-converting enzyme 2 (ACE2), transmembrane serine protease 2 (TRMPSS2), dipeptidyl peptidase 4 (DPP4), cluster of differentiation 4 (CD4), C-C chemokine receptor type 5 (CCR5), C-X-C chemokine receptor type 4 (CXCR4), cluster of differentiation 209 (CD209), or C-type lectin domain family 4 member M (CLEC4M). In some embodiments, the displayed virus-capturing polypeptide comprises an amino acid sequence of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% sequence identity to the amino acid sequence according to any one of SEQ ID NOs: 1-4. In some embodiments, the displayed virus-capturing polypeptide comprises an amino acid sequence according to any one of SEQ ID NOs: 1-4. In some embodiments, the displayed virus-capturing polypeptide is part of a fusion protein. In some embodiments, the fusion protein comprises a transmembrane poly peptide. In some embodiments, the transmembrane polypeptide comprises a spike glycoprotein transmembrane region, a mammalian membrane protein, an envelope protein, a nucleocapsid protein, or a cellular transmembrane protein. In some embodiments, the transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD114, Baboon endogenous virus (BaEV), glycoprotein (GP41), or glycoprotein 120 (GP120). In some embodiments, the VSVG transmembrane region comprises full length VSVG transmembrane region or a truncated VSVG transmembrane region. In some embodiments, the transmembrane polypeptide comprises the VSVG transmembrane region and a VSVG cytoplasmic tail. In some embodiments, the transmembrane polypeptide comprises an amino acid sequence at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% identical to the amino acid sequence according to any one of SEQ ID NOs: 19-27. In some embodiments, the transmembrane polypeptide comprises an amino acid sequence according to any one of SEQ ID NOs: 19-27. In some embodiments, the fusion protein further comprises an oligomerization domain. In some embodiments, the oligomerization domain comprises a dimerization domain, a trimerization domain, or a tetramerization domain. In some embodiments, the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain. In some embodiments, the oligomerization domain comprises an amino acid sequence that has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% sequence identity to an amino acid sequence according to any one of SEQ ID NOs: 5-18, and 28. In some embodiments, the oligomerization domain comprises an amino acid sequence of any one of SEQ ID NOs: 5-18, and 28. In some embodiments, the fusion protein is expressed at least about 10 copies on a surface of the multivalent particle. In some embodiments, the fusion protein is expressed at least about 15 copies on a surface of the multivalent particle. In some embodiments, the fusion protein is expressed at least about 20 copies on a surface of the multivalent particle. In some embodiments, the fusion protein is expressed at least about 25 copies on a surface of the multivalent particle. In some embodiments, the fusion protein is expressed at least about 30 copies on a surface of the multivalent particle. In some embodiments, the fusion protein is expressed at least about 35 copies on a surface of the multivalent particle. In some embodiments, the fusion protein is expressed at least about 40 copies on a surface of the multivalent particle. In some embodiments, the fusion protein is expressed at least about 45 copies on a surface of the multivalent particle. In some embodiments, the fusion protein is expressed at least ab out 50 copies on a surface of the multivalent particle. In some embodiments, the fusion protein is expressed at least about 75 copies on a surface of the multivalent particle. In some embodiments, the fusion protein is expressed at least about 100 copies on a surface of the multivalent particle. In some embodiments, the fusion protein is expressed at least about 150 copies on a surface of the multivalent particle. In some embodiments, the fusion protein is expressed at least about 200 copies on a surface of the multivalent particle. In some embodiments, the fusion protein is expressed at least about 300 copies on a surface of the multivalent particle. In some embodiments, the fusion protein is expressed at least about 400 copies on a surface of the multivalent particle. In some embodiments, the fusion protein is expressed at least about 500 copies on a surface of the multivalent particle. In some embodiments, the multivalent particle further comprises a second displayed virus-capturing polypeptide on a surface of the particle. In some embodiments, the second displayed virus-capturing polypeptide binds to the viral protein. In some embodiments, the second displayed virus-capturing polypeptide is expressed at least about 10 copies on the surface of the multivalent particle. In some embodiments, the second displayed virus-capturing polypeptide comprises a receptor for the viral protein. In some embodiments, the receptor comprises a viral entry receptor or a viral attachment receptor. In some embodiments, the receptor is both a viral entry receptor and a viral attachment receptor. In some embodiments, the second displayed virus-capturing polypeptide comprises an extracellular domain of the receptor. In some embodiments, the second displayed virus-capturing polypeptide comprises a ligand or a secreted protein. In some embodiments, the second displayed virus-capturing polypeptide comprises ACE2, TRMPSS2, DPP4, CD4, CCR5, CXCR4, CD209, or CLEC4M. In some embodiments, the second displayed virus-capturing polypeptide comprises an amino acid sequence of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% sequence identity to the amino acid sequence according to any one of SEQ ID NOs: 1-4. In some embodiments, the second displayed virus-capturing polypeptide comprises an amino acid sequence according to any one of SEQ ID NOs: 1-4. In some embodiments, the second displayed virus-capturing polypeptide is part of a second fusion protein. In some embodiments, the second fusion protein comprises a second transmembrane polypeptide. In some embodiments, the second transmembrane polypeptide comprises a spike glycoprotein transmembrane region, a mammalian membrane protein, an envelope protein, a nucleocapsid protein, or a cellular transmembrane protein. In some embodiments, the second transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD114, Baboon endogenous virus (BaEV), glycoprotein (GP41), or glycoprotein 120 (GP120). In some embodiments, the VSVG transmembrane region of the second transmembrane polypeptide comprises full length VSVG transmembrane region or a truncated VSVG transmembrane region. In some embodiments, the second transmembrane polypeptide comprises the VSVG transmembrane region and a VSVG cytoplasmic tail. In some embodiments, the second transmembrane polypeptide comprises an amino acid sequence at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% identical to the amino acid sequence according to any one of SEQ ID NOs: 19-27. In some embodiments, the second transmembrane polypeptide comprises an amino acid sequence of any one of SEQ ID NOs: 19-27. In some embodiments, the second fusion protein further comprises a second oligomerization domain. In some embodiments, the second oligomerization domain comprises a dimerization domain, a trimerization domain, or a tetramerization domain. In some embodiments, the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain. In some embodiments, the oligomerization domain comprises an amino acid sequence that has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% sequence identity to an amino acid sequence according to any one of SEQ ID NOs: 5-18, and 28. In some embodiments, the oligomerization domain comprises an amino acid sequence according to any one of SEQ ID NOs: 5-18, and 28. In some embodiments, the second fusion protein is expressed at least about 10 copies on a surface of the multivalent particle. In some embodiments, the second fusion protein is expressed at least about 15 copies on a surface of the multivalent particle. In some embodiments, the second fusion protein is expressed at least about 20 copies on a surface of the multivalent particle. In some embodiments, the second fusion protein is expressed at least about 25 copies on a surface of the multivalent particle. In some embodiments, the second fusion protein is expressed at least about 30 copies on a surface of the multivalent particle. In some embodiments, the second fusion protein is expressed at least about 35 copies on a surface of the multivalent particle. In some embodiments, the second fusion protein is expressed at least about 40 copies on a surface of the multivalent particle. In some embodiments, the second fusion protein is expressed at least about 45 copies on a surface of the multivalent particle. In some embodiments, the second fusion protein is expressed at least about 50 copies on a surface of the multivalent particle. In some embodiments, the second fusion protein is expressed at least about 75 copies on a surface of the multivalent particle. In some embodiments, the second fusion protein is expressed at least about 100 copies on a surface of the multivalent particle. In some embodiments, the second fusion protein is expressed at least about 150 copies on a surface of the multivalent particle. In some embodiments, the second fusion protein is expressed at least about 200 copies on a surface of the multivalent particle. In some embodiments, the second fusion protein is expressed at least about 300 copies on a surface of the multivalent particle. In some embodiments, the second fusion protein is expressed at least about 400 copies on a surface of the multivalent particle. In some embodiments, the second fusion protein is expressed at least about 500 copies on a surface of the multivalent particle. In some embodiments, the multivalent particle does not comprise viral genetic material. In some embodiments, the multivalent particle is a viral-like a particle. In some embodiments, the multivalent particle is an extracellular vesicle. In some embodiments, the multivalent particle is an exosome. In some embodiments, the multivalent particle is an ectosome.

Disclosed herein, in another aspect, is a particle that comprises a ACE2 polypeptide on a surface of the particle. In some embodiments, the ACE2 polypeptide is expressed at a valency of at least 10 copies on the surface of the particle. In some embodiments, the ACE2 polypeptide is expressed in an oligomerized format on the surface of the particle. In some embodiments, the ACE2 polypeptide on the surface of the particle forms multivalent interactions with a viral spike protein. In some embodiments, the multivalent interactions provide tighter binding through avidity than a soluble version of the ACE2 polypeptide to the viral spike protein. Disclosed herein, in some embodiments, is a particle that comprises a ACE2 polypeptide on a surface of the particle wherein the ACE2 polypeptide is expressed at a valency of at least 10 copies on the surface of the particle and in an oligomerized format wherein the ACE2 polypeptide forms multivalent interactions with a viral spike protein which provide tighter binding through avidity than a soluble version of the ACE2 polypeptide to the viral spike protein. In some embodiments, the ACE2 polypeptide comprises a wildtype ACE2. In some embodiments, the ACE2 polypeptide comprises a mutant of ACE2. In some embodiments, the ACE2 polypeptide comprises a H2A mutant of ACE2. In some embodiments, the viral spike protein comprises a wildtype SARS CoV-1 spike protein, a mutant of SARS CoV-1 spike protein, a wildtype SARS CoV-2 spike protein, or a mutant of SARS CoV-2 spike protein that binds to ACE2. In some embodiments, the viral spike protein comprises the spike protein of original SARS CoV-2, the spike protein of Beta variant of SARS CoV-2, the spike protein of Delta variant of SARS CoV-2, the spike protein of B.1.351 Beta variant of SARS CoV-2, the spike protein of B.1.617.2 Delta variant of SARS CoV-2, the spike protein of SARS CoV-2 USA-WA1/2020 strain, the D614G mutant of SARS CoV-2 spike protein, the N439K mutant of SARS CoV-2 spike protein, the N501Y mutant of SARS CoV-2 spike protein, the E484K mutant of SARS CoV-2 spike protein, the E484Q+L452R mutant of SARS CoV-2 spike protein, the spike protein of Omicron variant of SARS CoV-2, or the E484K+N501Y mutant of spike protein of B.1.351 South Africa strain of SARS CoV-2. In some embodiments, the particle binds to the viral spike protein to effectively neutralize a virus comprising the viral spike protein. In some embodiments, the virus comprises a SARS CoV-1 virus, or a SARS CoV-2 virus. In some embodiments, the virus comprises the original SARS CoV-2 virus, Delta variant of SARS CoV-2, Beta variant of SARS CoV-2, B.1.351 Beta variant of SARS CoV-2, B.1.617.2 Delta variant of SARS CoV-2, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant of SARS CoV-2, or B.1.351 South Africa strain of SARS CoV-2 comprising the E484K+N501Y mutant of spike protein. In some embodiments, the ACE2 polypeptide is part of a fusion protein, wherein the fusion protein comprises a transmembrane polypeptide. In some embodiments, the transmembrane polypeptide comprises a spike glycoprotein transmembrane region, a mammalian membrane protein, an envelope protein, a nucleocapsid protein, or a cellular transmembrane protein. In some embodiments, the transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD 114, Baboon endogenous virus (BaEV), glycoprotein (GP41), or glycoprotein 120 (GP120). In some embodiments, the VSVG transmembrane region comprises full length VSVG transmembrane region or a truncated VSVG transmembrane region. In some embodiments, the transmembrane polypeptide comprises the VSVG transmembrane region and a VSVG cytoplasmic tail. In some embodiments, the transmembrane polypeptide comprises an amino acid sequence at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% identical to the amino acid sequence according to any one of SEQ ID NOs: 19-27. In some embodiments, the transmembrane polypeptide comprises an amino acid sequence according to any one of SEQ ID NOs: 19-27. In some embodiments, the fusion protein further comprises an oligomerization domain. In some embodiments, the oligomerization domain comprises a dimerization domain, a trimerization domain, or a tetramerization domain. In some embodiments, the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain. In some embodiments, the oligomerization domain comprises an amino acid sequence that has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% sequence identity to an amino acid sequence according to any one of SEQ ID NOs: 5-18, and 28. In some embodiments, the oligomerization domain comprises an amino acid sequence according to any one of SEQ ID NOs: 5-18, and 28. In some embodiments, the particle is a viral-like a particle. In some embodiments, the particle is an extracellular vesicle. In some embodiments, the particle is an exosome. In some embodiments, the particle is an ectosome.

Disclosed herein, in another aspect, is a particle that comprises a DPP4 polypeptide on a surface of the particle. In some embodiments, the DPP4 polypeptide is expressed at a valency of at least 10 copies on the surface of the particle. In some embodiments, the DPP4 polypeptide expressed in an oligomerized format on the surface of the particle. In some embodiments, the DPP4 polypeptide on the surface of the particle forms multivalent interactions with a viral protein. In some embodiments, the multivalent interactions provide tighter binding through avidity than a soluble version of the DPP4 polypeptide to the viral protein. Disclosed herein, in some embodiments, is a particle that comprises a DPP4 polypeptide on a surface of the particle wherein the DPP4 polypeptide is expressed at a valency of at least 10 copies on the surface of the particle and in an oligomerized format wherein the DPP4 polypeptide forms multivalent interactions with a viral protein which provide tighter binding through avidity than a soluble version of the DPP4 polypeptide to the viral protein. In some embodiments, the viral protein comprises a MERS coronavirus spike protein. In some embodiments, the particle binds to the viral protein to effectively neutralize a virus comprising the viral protein. In some embodiments, the virus comprises a MERS coronavirus. In some embodiments, the DPP4 polypeptide is part of a fusion protein. In some embodiments, the fusion protein comprises a transmembrane polypeptide. In some embodiments, the transmembrane polypeptide comprises a spike glycoprotein transmembrane region, a mammalian membrane protein, an envelope protein, a nucleocapsid protein, or a cellular transmembrane protein. In some embodiments, the transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD114, Baboon endogenous virus (BaEV), glycoprotein (GP41), or glycoprotein 120 (GP120). In some embodiments, the transmembrane polypeptide comprises an amino acid sequence at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% identical to the amino acid sequence according to any one of SEQ ID NOs: 19-27. In some embodiments, the transmembrane polypeptide comprises an amino acid sequence according to any one of SEQ ID NOs: 19-27. In some embodiments, the fusion protein further comprises an oligomerization domain. In some embodiments, the oligomerization domain comprises a dimerization domain, a trimerization domain, or a tetramerization domain. In some embodiments, the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain. In some embodiments, the oligomerization domain comprises an amino acid sequence that has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% sequence identity to an amino acid sequence according to any one of SEQ ID NOs: 5-18, and 28. In some embodiments, the oligomerization domain comprises an amino acid sequence according to any one of SEQ ID NOs: 5-18, and 28. In some embodiments, the particle is a viral-like a particle. In some embodiments, the particle is an extracellular vesicle. In some embodiments, the particle is an exosome. In some embodiments, the particle is an ectosome.

Disclosed herein, in another aspect, is a multivalent particle that comprises at least 10 copies of a ACE2 polypeptide on the surface of the particle wherein the particle is self-assembled from the expression of a fusion protein that contains the ACE2 polypeptide sequence and an oligomerization domain sequence from a vector that is transfected into a cell.

Disclosed herein, in another aspect, is a multivalent particle that comprises at least 10 copies of a DPP4 polypeptide on the surface of the particle wherein the particle is self-assembled from the expression of a fusion protein that contains the DPP4 polypeptide sequence and an oligomerization domain sequence from a vector that is transfected into a cell.

Disclosed herein, in another aspect, is a method of treating a viral infection in a subject in need thereof comprising a multivalent particle displaying a ACE2 polypeptide on a surface of a particle, wherein the displayed ACE2 molecule on the particle forms multivalent interaction to a viral protein which provides tighter binding through avidity than a soluble version of the display polypeptide to the viral protein and administering the multivalent particle to the subject. In some embodiments, the ACE2 polypeptide comprises a wildtype ACE2. In some embodiments, the ACE2 polypeptide comprises a mutant of ACE2. In some embodiments, the ACE2 polypeptide comprises a H2A mutant of ACE2. In some embodiments, the viral protein comprises a viral spike protein. In some embodiments, the viral spike protein comprises a wildtype SARS CoV-1 spike protein, a mutant of SARS CoV-1 spike protein, a wildtype SARS CoV-2 spike protein, or a mutant of SARS CoV-2 spike protein that binds to ACE2. In some embodiments, the viral spike protein comprises the spike protein of original SARS CoV-2, the spike protein of Beta variant of SARS CoV-2, the spike protein of Delta variant of SARS CoV-2, the spike protein of B.1.351 Beta variant of SARS CoV-2, the spike protein of B.1.617.2 Delta variant of SARS CoV-2, the spike protein of SARS CoV-2 USA-WA1/2020 strain, the D614G mutant of SARS CoV-2 spike protein, the N439K mutant of SARS CoV-2 spike protein, the N501Y mutant of SARS CoV-2 spike protein, the E484K mutant of SARS CoV-2 spike protein, the E484Q+L452R mutant of SARS CoV-2 spike protein, the spike protein of Omicron variant of SARS CoV-2, or the E484K+N501Y mutant of spike protein of B.1.351 South Africa strain of SARS CoV-2. In some embodiments, the particle binds to the viral spike protein to effectively neutralize a virus comprising the viral spike protein. In some embodiments, the virus comprises a SARS CoV-1 virus, or a SARS CoV-2 virus. In some embodiments, the virus comprises the original SARS CoV-2 virus, Delta variant of SARS CoV-2, Beta variant of SARS CoV-2, B.1.351 Beta variant of SARS CoV-2, B.1.617.2 Delta variant of SARS CoV-2, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant of SARS CoV-2, or B.1.351 South Africa strain of SARS CoV-2 comprising the E484K+N501Y mutant of spike protein.

Disclosed herein, in another aspect, is a method of treating a viral infection in a subject in need thereof comprising a multivalent particle displaying a DPP4 polypeptide on a surface of a particle, wherein the displayed DPP4 molecule on the particle forms multivalent interaction to a viral protein which provides tighter binding through avidity than a soluble version of the display polypeptide to the viral protein and administering the multivalent particle to the subject. In some embodiments, the viral protein comprises a MERS coronavirus spike protein. In some embodiments, the particle binds to the viral protein to effectively neutralize a virus comprising the viral protein. In some embodiments, the virus comprises a MERS coronavirus.

Disclosed herein, in another aspect, is a method of inducing immunity against a viral infection in a subject comprising administering to the subject a composition disclosed herein or a particle disclosed herein. In some embodiments, the administering further comprises administering to the subject a virus comprising a viral protein. In some embodiments, the viral protein binds to a particle disclosed herein. In some embodiments, the viral infection comprises an infection by the virus comprising the viral protein. In some embodiments, the method further comprises mixing the composition disclosed herein or the particle disclosed herein with the vims before the administering. In some embodiments, the method further comprises, before the administering, incubating the composition disclosed herein or the particle disclosed herein with the virus after the mixing, wherein the composition disclosed herein or the particle disclosed herein neutralizes the virus. In some embodiments, the composition disclosed herein or the particle disclosed herein forms a complex with the virus. In some embodiments, the complex is in an immune cell after the administering. In some embodiments, the complex is taken up by an immune cell of the subject after the administering. In some embodiments, the virus is in the immune cell after the administering. In some embodiments, the virus is taken up by the immune cell after the administering. In some embodiments, the amount of the complex in the immune cell is more than the amount of the virus in the immune cell. In some embodiments, an immune cell preferentially takes up the complex formed between the particle disclosed herein and the virus compared with the virus alone. In some embodiments, the immune cell comprises a T cell, a B cell, a natural killer (NK) cell, a neutrophil, a monocyte, a dendritic cell, or a macrophage. In some embodiments, the immune cell comprises a macrophage. In some embodiments, the immune cell comprises a dendritic cell. In some embodiments, the viral infection comprises an infection by SARS CoV-2, SARS CoV-1, MERS CoV, respiratory syncytial virus, HBV, HDV, or HIV. In some embodiments, the viral infection comprises an infection by a mutant of SARS CoV-2, a mutant of SARS CoV-1, a mutant of MERS CoV, a mutant of RSV, a mutant of HBV, a mutant of HDV, or a mutant of HIV. In some embodiments, the viral infection comprises an infection by the original SARS CoV-2 virus, Delta variant of SARS CoV-2, Beta variant of SARS CoV-2, SARS CoV-2 B.1.351 Beta variant, SARS CoV-2 B.1.617.2 Delta variant, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant SARS CoV-2, or the B.1.351 South Africa strain of SARS CoV-2 comprising the E484K+N501Y mutant of spike protein. In some embodiments, the composition disclosed herein comprises a multivalent particle, as disclosed herein. In some embodiments, the multivalent particle disclosed herein comprises the displayed virus-capturing polypeptide on the surface of the multivalent particle. In some embodiments, the displayed virus-capturing polypeptide on the surface of the multivalent particle forms multivalent interactions with the viral spike protein which provide tighter binding through avidity than a soluble version of the displayed virus-capturing polypeptide to the viral spike protein. In some embodiments, the multivalent particle neutralizes a virus comprising the viral spike protein when the displayed virus-capturing polypeptide forms multivalent interactions with the viral spike protein. In some embodiments, the neutralizing potency of the multivalent particle against the virus increases when the displayed virus-capturing polypeptide form oligomers on the surface of the multivalent particle. In some embodiments, the neutralizing potency of the multivalent particle against the virus increases when the displayed virus-capturing polypeptide form oligomers on the surface of the multivalent particle compared with a multivalent particle comprising a monomeric form of the displayed virus-capturing polypeptide. In some embodiments, the neutralizing potency of a multivalent particle comprising an oligomeric form of the displayed virus-capturing polypeptide is higher than the neutralizing potency of a multivalent particle comprising a monomeric form of the displayed virus-capturing polypeptide. In some embodiments, an oligomeric form or an oligomer comprises a dimeric form, a trimeric form, or a tetrameric form. In some embodiments, the neutralizing potency of the multivalent particle against the virus is correlated with the copy number of the displayed virus-capturing polypeptide expressed on the surface of the multivalent particle. In some embodiments, the neutralizing potency of the multivalent particle against the virus increases when the copy number of the displayed virus-capturing polypeptide expressed on the surface of the multivalent particle increases. In some embodiments, the neutralizing potency comprises a suppression of a viral infection. In some embodiments, the neutralizing potency comprises a number of viral particles that bind to a single multivalent particle.

Disclosed herein, in another aspect, is a method for immunizing a subject against a viral infection comprising administering to the subject: (a) a composition disclosed herein or a particle disclosed herein; and (b) a virus comprising a viral spike protein, wherein the viral infection comprises an infection by the virus. In some embodiments, the composition disclosed herein comprises a particle, as disclosed herein. In some embodiments, the particle disclosed herein comprises the displayed virus-capturing polypeptide on the surface of particle. In some embodiments, the displayed virus-capturing polypeptide on the surface of the particle forms multivalent interactions with the viral spike protein which provide tighter binding through avidity than a soluble version of the displayed virus-capturing polypeptide to the viral spike protein. In some embodiments, the virus comprises a live virus. In some embodiments, the virus comprises an inactivated or dead virus. In some embodiments, the viral infection comprises an infection by SARS CoV-2, SARS CoV-1, MERS CoV, respiratory syncytial virus, HBV, HDV, HIV, or a mutant thereof. In some embodiments, the viral infection comprises an infection by the original SARS CoV-2 virus, Delta variant of SARS CoV-2, Beta variant of SARS CoV-2, B.1.351 Beta variant of SARS CoV-2, B.1.617.2 Delta variant of SARS CoV-2, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant of SARS CoV-2, or B.1.351 South Africa strain of SARS CoV-2 comprising the E484K+N501Y mutant of spike protein. In some embodiments, the administering comprises administering through inhalation. In some embodiments, the administering comprises administering intranasally. In some embodiments, the administering comprises administering intravenously.

Disclosed herein, in another aspect, is a method for treating a viral infection in a subject comprising administering to the subject a composition disclosed herein or a particle disclosed herein. In some embodiments, the composition disclosed herein comprises a particle, as disclosed herein. In some embodiments, the particle disclosed herein comprises the displayed virus-capturing polypeptide on the surface of particle. In some embodiments, the displayed virus-capturing polypeptide on the surface of the particle forms multivalent interactions with the viral spike protein which provide tighter binding through avidity than a soluble version of the displayed virus-capturing polypeptide to the viral spike protein. In some embodiments, the administering induces immunity in the subject against the viral infection. In some embodiments, the viral infection comprises an infection by SARS CoV-2, SARS CoV-1, MERS CoV, respiratory syncytial virus, HBV, HDV, HIV, or a mutant thereof. In some embodiments, the viral infection comprises an infection by the original SARS CoV-2 virus, Delta variant of SARS CoV-2, Beta variant of SARS CoV-2, B.1.351 Beta variant of SARS CoV-2, B.1.617.2 Delta variant of SARS CoV-2, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant of SARS CoV-2, or B.1.351 South Africa strain of SARS CoV-2 comprising the E484K+N501Y mutant of spike protein.

Disclosed herein are compositions comprising a decoy-antivirus, wherein the decoy-antivirus comprises a multivalent particle (MVP) displaying at least about 10 copies of a decoy viral entry receptor on a surface of the MVP, wherein at least one decoy viral entry receptor comprises an oligomerized format, wherein the at least one decoy viral entry receptor forms a multivalent interaction with an oligomeric multivalent spike protein on a virus. In some embodiments, the decoy viral entry receptor is a recombinant fusion protein comprising a decoy viral entry receptor, an oligomerization domain, a transmembrane domain, and a cytosolic domain, wherein when the recombinant fusion protein is expressed on a surface of an enveloped particle, and wherein the recombinant fusion protein is displayed in an oligomeric format.

Disclosed herein are compositions comprising an ACE2-antivirus, wherein the ACE2-antivirus comprises a multivalent particle (MVP) displaying at least about 10 copies of ACE2 on a surface of the MVP in an oligomerized format, wherein the ACE2 forms a multivalent interaction with an oligomeric multivalent spike protein on a SARS Coronavirus. In some embodiments, the displayed peptide is a recombinant ACE2 fusion protein comprising a ACE2 decoy, an oligomerization domain, a transmembrane domain, and a cytosolic domain, wherein when the recombinant ACE2 fusion protein is expressed on a surface of an enveloped particle, and wherein the recombinant ACE2 fusion protein is displayed in an oligomeric format.

Disclosed herein are compositions and methods to counter the multivalent binding strategy seen in viruses to turn weak molecular interactions into strong functional affinity. The increased avidity can create an insurmountable threshold for low-valency or monovalent binders, such as neutralizing antibodies. Spike mutagenesis is likely to have a greater impact on the binding affinity of spike and neutralizing antibody interactions than that of multivalent interactions between multivalent spikes and entry receptors. Thus, multivalence can underpin a coronavirus' ability to select for future super variants while simultaneously disrupting immune control by neutralizing antibodies.

Disclosed herein are the design and preparation decoy-antivirus by genetically programming multivalent particles (MVPs) to display multiple copies of viral entry-receptors mirroring spike patterns, dubbed decoy-Antivirus. The decoy-antivirus platform disclosed herein may be employed to generate highly potent, variant-proof neutralizing therapeutics against existing and future pandemic viruses. Instead of chasing constantly evolving spikes, the decoy-antiviruses of the disclosure are highly potent, variant-proof neutralizing therapeutics using defined entry receptors. The data disclosed herein demonstrate that the antiviruses can effectively counter the multivalent interactions between viral spikes and host cells. The decoy-antiviruses of the disclosure can be used for future zoonic viruses, and pathogenic human viruses, such as influenza, coronaviruses, hepatitis viruses, dengue virus, and human immunodeficiency virus (HIV).

Disclosed herein are methods to use decoy-antivirus as a treatment vaccine to induce antiviral immunity against a respiratory virus. In some embodiments, the respiratory virus is a coronavirus, influenza, respiratory syncytial virus, or a human Rhinoviruses. In some embodiments, an infected patent is treated with an inhaled or intranasal decoy-antivirus to induce protective immunity against future infections.

Disclosed herein are methods to use ACE2-antivirus as a treatment vaccine to induce antiviral immunity against a SARS coronavirus through treatment with an ACE2-antivirus. In some embodiments, a patient infected with a SARS coronavirus utilizing ACE2 as an entry receptor can be treated with an inhaled or intranasal ACE2-antivirus to induce protective immunity against future infections.

Disclosed herein are methods of using a DDP4-antivirus as a treatment vaccine to induce antiviral immunity against MERS through treatment with a DDP4-antivirus. In some embodiments, an infected patient can be treated with an inhaled or intranasal DDP4-antivirus to induce protective immunity against future infections.

Disclosed herein are methods to induce protective immunity using a decoy-antivirus inactivated vaccine, wherein the decoy-antivirus inactivated vaccine comprises a virus and a decoy-antivirus, wherein the virus is a virus utilizing an entry receptor. In some embodiments, the decoy-antivirus inactivated vaccine is administered intranasally. In some embodiments, the decoy-antivirus inactivated vaccine is administered through inhalation. In some embodiments, the decoy-antivirus inactivated vaccine is administered intramuscularly.

Disclosed herein are methods to induce protective immunity using an ACE2-antivirus inactivated vaccine, wherein a live SARS CoV-2 is mixed with an ACE2-antivirus to generate a ACE2-antivirus inactivated vaccine, wherein the SARS CoV-2 is a variant utilizing SARS CoV-2 as an entry receptor. In some embodiments, the decoy-antivirus inactivated vaccine is administered intranasally. In some embodiments, the decoy-antivirus inactivated vaccine is administered through inhalation. In some embodiments, the decoy-antivirus inactivated vaccine is administered intramuscularly.

Disclosed herein are methods to induce protective immunity using a DDP4-antivirus inactivated vaccine, wherein live MERS is mixed with a DPP4-antivirus to generate a DPP4-antivirus inactivated vaccine, wherein the MERS is a variant utilizing DPP4 as an entry receptor. In some embodiments, the DDP4-antivirus inactivated vaccine is administered intranasally. In some embodiments, the DDP4-antivirus inactivated vaccine is administered intramuscularly.

Disclosed herein are compositions of antibody-based antiviruses (Ab-antivirus) comprising a multivalent particle (MVP) displaying at least about 10 copies of a spike-binding antibody on a surface of the MVP, wherein at least one spike-binding antibody on the MVP comprises an oligomerized format, wherein the at least one spike-binding antibody forms a multivalent interaction with an oligomeric multivalent spike protein on a virus. In some embodiments, the spike-binding antibody is a recombinant fusion protein comprising a spike-binding antibody, an oligomerization domain, a transmembrane domain, and a cytosolic domain, wherein when the recombinant fusion protein is expressed on a surface of an enveloped particle, and wherein the recombinant fusion protein is displayed in an oligomeric format.

Disclosed herein are the design and preparation Ab-antivirus by genetically programming a multivalent particle (MVP) to display a plurality of spike-binding antibody mirroring spike patterns, dubbed Ab-Antivirus. The Ab-antivirus platform disclosed herein may be employed to generate highly potent, variant-resistant neutralizing therapeutics against a virus. Instead of chasing constantly evolving spikes, the Ab-antiviruses of the disclosure are highly potent, variant-proof neutralizing therapeutics using defined entry receptors. In some embodiments, the virus is a zoonic virus, a pathogenic human virus (e.g., influenza, influenza, coronavirus, hepatitis virus, dengue virus, and human immunodeficiency virus (HIV)).

Disclosed herein are methods of using an Ab-antivirus as a treatment vaccine to induce antiviral immunity against a respiratory virus through treatment with the Ab-antivirus, wherein infected patients are treated with inhaled or intranasal decoy-antiviruses to induce protective immunity against future infections. In some embodiments, the respiratory virus is a coronavirus, influenza, RSV, or a human rhinovirus.

Disclosed herein are methods of inducing protective immunity using an Ab-antivirus inactivated vaccine, wherein the virus is mixed with a corresponding Ab-antivirus to generate decoy-antivirus inactivated vaccine, wherein Ab-antivirus inactivated vaccine are delivered intranasal or intramuscular, wherein the virus is any viruses utilizing an entry receptor.

Described herein, in some embodiments, are MVPs displaying the ACE2 entry receptors as neutralizing decoys for SARS CoV-1/2. In some embodiments, the ACE2 MVPs inhibit the infection of the SARS CoV-2 viruses with a sub-picomolar IC₅₀ in pseudo-virus and live-virus neutralization assays. In some embodiments, the ACE2 MVPs are more potent than a ACE2 recombinant protein or a therapeutic neutralizing antibody. In some embodiments, each ACE2 MVP neutralizes at least about 10 pseudotyped SARS CoV-2 virions, and MVPs with higher ACE2 density can inhibit virus infection more completely. In some embodiments, the ACE2 MVPs of the disclosure can neutralize SARS CoV-2 variants and SARS CoV-1 at sub-picomolar IC₅₀s, and are thus broadly neutralizing against evolving SARS Coronaviruses utilizing ACE2 as an entry receptor. In some embodiments, the ACE2 MVPs are insensitive to spike mutagenesis and therefore are variant-proof neutralizing therapeutics. In some embodiments, MVPs displaying dipeptidyl peptidase 4 (DPP4-MVPs), the entry receptor for MERS CoV, can inhibit the infection of MERS pseudovirus at a picomolar IC₅₀. In some embodiments, the ACE2 MVPs are effective in rescue animals from lethal SARS CoV-2 infection. In some embodiments, treatment of SARS CoV-2 infection with the ACE2 MVPs are effective in inducing robust immunity against dominant SARS CoV-2 strains including the Delta variant.

Described herein, in some embodiments, are multivalent particles comprising a fusion protein that comprises a transmembrane polypeptide and a mammalian polypeptide that binds to a viral protein. In some embodiments, the viral protein is from SARS-CoV-1, SARS-CoV-2, MERS-CoV, respiratory syncytial virus (RSV), HBV, HDV, HIV, or combinations thereof. In some embodiments, the viral protein is from SARS-CoV-2. In some embodiments, the viral protein is from MERS-CoV. In some embodiments, the viral protein is from SARS-CoV-1.

Various multivalent particles are contemplated herein. In some embodiments, the multivalent particle is synthetic. In some embodiments, the multivalent particle is recombinant. In some embodiments, the multivalent particle does not comprise viral genetic material. In some embodiments, the multivalent particle is a viral-like particle or virus-like particle. As used herein, viral-like particle and virus-like particle interchangeably. In some embodiments, the viral-like particle is synthetic. In some embodiments, the viral-like particle is recombinant. In some embodiments, the viral-like particle does not comprise viral genetic material. In some embodiments, the multivalent particle is an extracellular vesicle. In some embodiments, the multivalent particle is an exosome. In some embodiments, the multivalent particle is an ectosome.

Multivalent particles as described herein, in some embodiments, comprise a fusion protein, wherein the fusion protein is expressed at multiple copies on a surface of the multivalent particle. In some embodiments, the fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the multivalent particle. In some embodiments, the fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the multivalent particle. In some embodiments, the fusion protein is expressed at least or about 10 copies on a surface of the multivalent particle. In some embodiments, the fusion protein is expressed at least or about 25 copies on a surface of the multivalent particle. In some embodiments, the fusion protein is expressed at least or about 50 copies on a surface of the multivalent particle. In some embodiments, the fusion protein is expressed at least or about 75 copies on a surface of the multivalent particle. In some embodiments, the fusion protein is expressed at least or about 100 copies on a surface of the multivalent particle. In some embodiments, the fusion protein is expressed at least or about 125 copies on a surface of the multivalent particle. In some embodiments, the fusion protein is expressed at least or about 150 copies on a surface of the multivalent particle. In some embodiments, the fusion protein is expressed at least or about 175 copies on a surface of the multivalent particle. In some embodiments, the fusion protein is expressed at least or about 200 copies on a surface of the multivalent particle. In some embodiments, the fusion protein is expressed at least or about 225 copies on a surface of the multivalent particle. In some embodiments, the fusion protein is expressed at least or about 250 copies on a surface of the multivalent particle. In some embodiments, the fusion protein is expressed at least or about 275 copies on a surface of the multivalent particle. In some embodiments, the fusion protein is expressed at least or about 300 copies on a surface of the multivalent particle.

In some embodiments, the multivalent particle is a viral-like particle. The viral-like particle as described herein, in some embodiments, comprise a fusion protein, wherein the fusion protein is expressed at multiple copies on a surface of the viral-like particle. In some embodiments, the fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the viral-like particle. In some embodiments, the fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the viral-like particle. In some embodiments, the fusion protein is expressed at least or about 10 copies on a surface of the viral-like particle. In some embodiments, the fusion protein is expressed at least or about 25 copies on a surface of the viral-like particle. In some embodiments, the fusion protein is expressed at least or about 50 copies on a surface of the viral-like particle. In some embodiments, the fusion protein is expressed at least or about 75 copies on a surface of the viral-like particle. In some embodiments, the fusion protein is expressed at least or about 100 copies on a surface of the viral-like particle. In some embodiments, the fusion protein is expressed at least or about 125 copies on a surface of the viral-like particle. In some embodiments, the fusion protein is expressed at least or about 150 copies on a surface of the viral-like particle. In some embodiments, the fusion protein is expressed at least or about 175 copies on a surface of the viral-like particle. In some embodiments, the fusion protein is expressed at least or about 200 copies on a surface of the viral-like particle. In some embodiments, the fusion protein is expressed at least or about 225 copies on a surface of the viral-like particle. In some embodiments, the fusion protein is expressed at least or about 250 copies on a surface of the viral-like particle. In some embodiments, the fusion protein is expressed at least or about 275 copies on a surface of the viral-like particle. In some embodiments, the fusion protein is expressed at least or about 300 copies on a surface of the viral-like particle.

In some embodiments, the multivalent particle is an extracellular vesicle. The extracellular vesicle as described herein, in some embodiments, comprise a fusion protein, wherein the fusion protein is expressed at multiple copies on a surface of the extracellular vesicle. In some embodiments, the fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the extracellular vesicle. In some embodiments, the fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the extracellular vesicle. In some embodiments, the fusion protein is expressed at least or about 10 copies on a surface of the extra cellular vesicle. In some embodiments, the fusion protein is expressed at least or about 25 copies on a surface of the extracellular vesicle. In some embodiments, the fusion protein is expressed at least or about 50 copies on a surface of the extracellular vesicle. In some embodiments, the fusion protein is expressed at least or about 75 copies on a surface of the extracellular vesicle. In some embodiments, the fusion protein is expressed at least or about 100 copies on a surface of the extracellular vesicle. In some embodiments, the fusion protein is expressed at least or about 125 copies on a surface of the extracellular vesicle. In some embodiments, the fusion protein is expressed at least or about 150 copies on a surface of the extracellular vesicle. In some embodiments, the fusion protein is expressed at least or about 175 copies on a surface of the extracellular vesicle. In some embodiments, the fusion protein is expressed at least or about 200 copies on a surface of the extracellular vesicle. In some embodiments, the fusion protein is expressed at least or about 225 copies on a surface of the extracellular vesicle. In some embodiments, the fusion protein is expressed at least or about 250 copies on a surface of the extracellular vesicle. In some embodiments, the fusion protein is expressed at least or about 275 copies on a surface of the extracellular vesicle. In some embodiments, the fusion protein is expressed at least or about 300 copies on a surface of the extracellular vesicle.

In some embodiments, the multivalent particle is an exosome. The exosome as described herein, in some embodiments, comprise a fusion protein, wherein the fusion protein is expressed at multiple copies on a surface of the exosome. In some embodiments, the fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the exosome. In some embodiments, the fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the exosome. In some embodiments, the fusion protein is expressed at least or about 10 copies on a surface of the exosome. In some embodiments, the fusion protein is expressed at least or about 25 copies on a surface of the exosome. In some embodiments, the fusion protein is expressed at least or about 50 copies on a surface of the exosome. In some embodiments, the fusion protein is expressed at least or about 75 copies on a surface of the exosome. In some embodiments, the fusion protein is expressed at least or about 100 copies on a surface of the exosome. In some embodiments, the fusion protein is expressed at least or about 125 copies on a surface of the exosome. In some embodiments, the fusion protein is expressed at least or about 150 copies on a surface of the exosome. In some embodiments, the fusion protein is expressed at least or about 175 copies on a surface of the exosome. In some embodiments, the fusion protein is expressed at least or about 200 copies on a surface of the exosome. In some embodiments, the fusion protein is expressed at least or about 225 copies on a surface of the exosome. In some embodiments, the fusion protein is expressed at least or about 250 copies on a surface of the exosome. In some embodiments, the fusion protein is expressed at least or about 275 copies on a surface of the exosome. In some embodiments, the fusion protein is expressed at least or about 300 copies on a surface of the exosome.

In some embodiments, the multivalent particle is an ectosome. The ectosome as described herein, in some embodiments, comprise a fusion protein, wherein the fusion protein is expressed at multiple copies on a surface of the ectosome. In some embodiments, the fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the ectosome. In some embodiments, the fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the ectosome. In some embodiments, the fusion protein is expressed at least or about 10 copies on a surface of the ectosome. In some embodiments, the fusion protein is expressed at least or about 25 copies on a surface of the ectosome. In some embodiments, the fusion protein is expressed at least or about 50 copies on a surface of the ectosome. In some embodiments, the fusion protein is expressed at least or about 75 copies on a surface of the ectosome. In some embodiments, the fusion protein is expressed at least or about 100 copies on a surface of the ectosome. In some embodiments, the fusion protein is expressed at least or about 125 copies on a surface of the ectosome. In some embodiments, the fusion protein is expressed at least or about 150 copies on a surface of the ectosome. In some embodiments, the fusion protein is expressed at least or about 175 copies on a surface of the ectosome. In some embodiments, the fusion protein is expressed at least or about 200 copies on a surface of the ectosome. In some embodiments, the fusion protein is expressed at least or about 225 copies on a surface of the ectosome. In some embodiments, the fusion protein is expressed at least or about 250 copies on a surface of the ectosome. In some embodiments, the fusion protein is expressed at least or about 275 copies on a surface of the ectosome. In some embodiments, the fusion protein is expressed at least or about 300 copies on a surface of the ectosome.

In some embodiments, the multivalent particle is a replication competent virus. The replication competent virus as described herein, in some embodiments, comprise a fusion protein, wherein the fusion protein is expressed at multiple copies on a surface of the replication competent virus. In some embodiments, the fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the replication competent virus. In some embodiments, the fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the replication competent virus. In some embodiments, the fusion protein is expressed at least or about 10 copies on a surface of the replication competent virus. In some embodiments, the fusion protein is expressed at least or about 25 copies on a surface of the replication competent virus. In some embodiments, the fusion protein is expressed at least or about 50 copies on a surface of the replication competent virus. In some embodiments, the fusion protein is expressed at least or about 75 copies on a surface of the replication competent virus. In some embodiments, the fusion protein is expressed at least or about 100 copies on a surface of the replication competent virus. In some embodiments, the fusion protein is expressed at least or about 125 copies on a surface of the replication competent virus. In some embodiments, the fusion protein is expressed at least or about 150 copies on a surface of the replication competent virus. In some embodiments, the fusion protein is expressed at least or about 175 copies on a surface of the replication competent virus. In some embodiments, the fusion protein is expressed at least or about 200 copies on a surface of the replication competent virus. In some embodiments, the fusion protein is expressed at least or about 225 copies on a surface of the replication competent virus. In some embodiments, the fusion protein is expressed at least or about 250 copies on a surface of the replication competent virus. In some embodiments, the fusion protein is expressed at least or about 275 copies on a surface of the replication competent virus. In some embodiments, the fusion protein is expressed at least or about 300 copies on a surface of the replication competent virus.

Multivalent particles as described herein, in some embodiments, comprise a second fusion protein, wherein the second fusion protein is expressed at multiple copies on a surface of the multivalent particle. In some embodiments, the second fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the multivalent particle. In some embodiments, the second fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the multivalent particle. In some embodiments, the second fusion protein is expressed at least or about 10 copies on a surface of the multivalent particle. In some embodiments, the second fusion protein is expressed at least or about 25 copies on a surface of the multivalent particle. In some embodiments, the second fusion protein is expressed at least or about 50 copies on a surface of the multivalent particle. In some embodiments, the second fusion protein is expressed at least or about 75 copies on a surface of the multivalent particle. In some embodiments, the second fusion protein is expressed at least or about 100 copies on a surface of the multivalent particle. In some embodiments, the second fusion protein is expressed at least or about 125 copies on a surface of the multivalent particle. In some embodiments, the second fusion protein is expressed at least or about 150 copies on a surface of the multivalent particle. In some embodiments, the second fusion protein is expressed at least or about 175 copies on a surface of the multivalent particle. In some embodiments, the second fusion protein is expressed at least or about 200 copies on a surface of the multivalent particle. In some embodiments, the second fusion protein is expressed at least or about 225 copies on a surface of the multivalent particle. In some embodiments, the second fusion protein is expressed at least or about 250 copies on a surface of the multivalent particle. In some embodiments, the second fusion protein is expressed at least or about 275 copies on a surface of the multivalent particle. In some embodiments, the second fusion protein is expressed at least or about 300 copies on a surface of the multivalent particle.

The viral-like particle as described herein, in some embodiments, comprise a second fusion protein, wherein the second fusion protein is expressed at multiple copies on a surface of the viral-like particle. In some embodiments, the second fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the viral-like particle. In some embodiments, the second fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the viral-like particle. In some embodiments, the second fusion protein is expressed at least or about 10 copies on a surface of the viral-like particle. In some embodiments, the second fusion protein is expressed at least or about 25 copies on a surface of the viral-like particle. In some embodiments, the second fusion protein is expressed at least or about 50 copies on a surface of the viral-like particle. In some embodiments, the second fusion protein is expressed at least or about 75 copies on a surface of the viral-like particle. In some embodiments, the second fusion protein is expressed at least or about 100 copies on a surface of the viral-like particle. In some embodiments, the second fusion protein is expressed at least or about 125 copies on a surface of the viral-like particle. In some embodiments, the second fusion protein is expressed at least or about 150 copies on a surface of the viral-like particle. In some embodiments, the second fusion protein is expressed at least or about 175 copies on a surface of the viral-like particle. In some embodiments, the second fusion protein is expressed at least or about 200 copies on a surface of the viral-like particle. In some embodiments, the second fusion protein is expressed at least or about 225 copies on a surface of the viral-like particle. In some embodiments, the second fusion protein is expressed at least or about 250 copies on a surface of the viral-like particle. In some embodiments, the second fusion protein is expressed at least or about 275 copies on a surface of the viral-like particle. In some embodiments, the second fusion protein is expressed at least or about 300 copies on a surface of the viral-like particle.

The extracellular vesicle, as described herein, in some embodiments, comprise a second fusion protein, wherein the second fusion protein is expressed at multiple copies on a surface of the extracellular vesicle. In some embodiments, the second fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the extracellular vesicle. In some embodiments, the second fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the extracellular vesicle. In some embodiments, the second fusion protein is expressed at least or about 10 copies on a surface of the extracellular vesicle. In some embodiments, the second fusion protein is expressed at least or about 25 copies on a surface of the extracellular vesicle. In some embodiments, the second fusion protein is expressed at least or about 50 copies on a surface of the extracellular vesicle. In some embodiments, the second fusion protein is expressed at least or about 75 copies on a surface of the extracellular vesicle. In some embodiments, the second fusion protein is expressed at least or about 100 copies on a surface of the extracellular vesicle. In some embodiments, the second fusion protein is expressed at least or about 125 copies on a surface of the extracellular vesicle. In some embodiments, the second fusion protein is expressed at least or about 150 copies on a surface of the extracellular vesicle. In some embodiments, the second fusion protein is expressed at least or about 175 copies on a surface of the extracellular vesicle. In some embodiments, the second fusion protein is expressed at least or about 200 copies on a surface of the extracellular vesicle. In some embodiments, the second fusion protein is expressed at least or about 225 copies on a surface of the extracellular vesicle. In some embodiments, the second fusion protein is expressed at least or about 250 copies on a surface of the extracellular vesicle. In some embodiments, the second fusion protein is expressed at least or about 275 copies on a surface of the extracellular vesicle. In some embodiments, the second fusion protein is expressed at least or about 300 copies on a surface of the extracellular vesicle.

The exosome, as described herein, in some embodiments, comprise a second fusion protein, wherein the second fusion protein is expressed at multiple copies on a surface of the exosome. In some embodiments, the second fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the exosome. In some embodiments, the second fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the exosome. In some embodiments, the second fusion protein is expressed at least or about 10 copies on a surface of the exosome. In some embodiments, the second fusion protein is expressed at least or about 25 copies on a surface of the exosome. In some embodiments, the second fusion protein is expressed at least or about 50 copies on a surface of the exosome. In some embodiments, the second fusion protein is expressed at least or about 75 copies on a surface of the exosome. In some embodiments, the second fusion protein is expressed at least or about 100 copies on a surface of the exosome. In some embodiments, the second fusion protein is expressed at least or about 125 copies on a surface of the exosome. In some embodiments, the second fusion protein is expressed at least or about 150 copies on a surface of the exosome. In some embodiments, the second fusion protein is expressed at least or about 175 copies on a surface of the exosome. In some embodiments, the second fusion protein is expressed at least or about 200 copies on a surface of the exosome. In some embodiments, the second fusion protein is expressed at least or about 225 copies on a surface of the exosome. In some embodiments, the second fusion protein is expressed at least or about 250 copies on a surface of the exosome. In some embodiments, the second fusion protein is expressed at least or about 275 copies on a surface of the exosome. In some embodiments, the second fusion protein is expressed at least or about 300 copies on a surface of the exosome.

The ectosome, as described herein, in some embodiments, comprise a second fusion protein, wherein the second fusion protein is expressed at multiple copies on a surface of the ectosome. In some embodiments, the second fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the ectosome. In some embodiments, the second fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the ectosome. In some embodiments, the second fusion protein is expressed at least or about 10 copies on a surface of the ectosome. In some embodiments, the second fusion protein is expressed at least or about 25 copies on a surface of the ectosome. In some embodiments, the second fusion protein is expressed at least or about 50 copies on a surface of the ectosome. In some embodiments, the second fusion protein is expressed at least or about 75 copies on a surface of the ectosome. In some embodiments, the second fusion protein is expressed at least or about 100 copies on a surface of the ectosome. In some embodiments, the second fusion protein is expressed at least or about 125 copies on a surface of the ectosome. In some embodiments, the second fusion protein is expressed at least or about 150 copies on a surface of the ectosome. In some embodiments, the second fusion protein is expressed at least or about 175 copies on a surface of the ectosome. In some embodiments, the second fusion protein is expressed at least or about 200 copies on a surface of the ectosome. In some embodiments, the second fusion protein is expressed at least or about 225 copies on a surface of the ectosome. In some embodiments, the second fusion protein is expressed at least or about 250 copies on a surface of the ectosome. In some embodiments, the second fusion protein is expressed at least or about 275 copies on a surface of the ectosome. In some embodiments, the second fusion protein is expressed at least or about 300 copies on a surface of the ectosome.

The replication competent virus, as described herein, in some embodiments, comprise a second fusion protein, wherein the second fusion protein is expressed at multiple copies on a surface of the replication competent virus. In some embodiments, the second fusion protein is expressed at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the replication competent virus. In some embodiments, the second fusion protein is expressed at least or about 5 to about 400, about 20 to about 400, about 10 to about 300, about 20 to about 300, about 20 to about 200, about 50 to about 150, about 20 to about 100, or about 50 to about 100 copies on a surface of the replication competent virus. In some embodiments, the second fusion protein is expressed at least or about 10 copies on a surface of the replication competent virus. In some embodiments, the second fusion protein is expressed at least or about 25 copies on a surface of the replication competent virus. In some embodiments, the second fusion protein is expressed at least or about 50 copies on a surface of the replication competent virus. In some embodiments, the second fusion protein is expressed at least or about 75 copies on a surface of the replication competent virus. In some embodiments, the second fusion protein is expressed at least or about 100 copies on a surface of the replication competent virus. In some embodiments, the second fusion protein is expressed at least or about 125 copies on a surface of the replication competent virus. In some embodiments, the second fusion protein is expressed at least or about 150 copies on a surface of the replication competent virus. In some embodiments, the second fusion protein is expressed at least or about 175 copies on a surface of the replication competent virus. In some embodiments, the second fusion protein is expressed at least or about 200 copies on a surface of the replication competent virus. In some embodiments, the second fusion protein is expressed at least or about 225 copies on a surface of the replication competent virus. In some embodiments, the second fusion protein is expressed at least or about 250 copies on a surface of the replication competent virus. In some embodiments, the second fusion protein is expressed at least or about 275 copies on a surface of the replication competent virus. In some embodiments, the second fusion protein is expressed at least or about 300 copies on a surface of the replication competent virus.

Described herein, in some embodiments, are multivalent particles comprising improved binding properties. In some embodiments, the multivalent particle comprises a binding affinity (e.g., K_{D}) to the viral protein of less than 100 pM, less than 200 pM, less than 300 pM, less than 400 pM, less than 500 pM, less than 600 pM, less than 700 pM, less than 800 pM, or less than 900 pM In some embodiments, the multivalent particle comprises a K_{D} of less than 1 nM, less than 1.2 nM, less than 2 nM, less than 5 nM, or less than 10 nM. In some instances, the multivalent particle comprises a K_{D} of less than 1 nM. In some instances, the multivalent particle comprises a K_{D} of less than 1.2 nM. In some instances, the multivalent particle comprises a K_{D} of less than 2 nM. In some instances, the multivalent particle comprises a K_{D} of less than 5 nM. In some instances, the multivalent particle comprises a K_{D} of less than 10 nM.

In some embodiments, the multivalent particle comprises an IC50 of less than 20 picomolar (pM) in a neutralization assay. In some embodiments, the multivalent particle comprises an IC50 of less than 15 picomolar (pM) in a neutralization assay. In some embodiments, the multivalent particle comprises an IC50 of less than 10 picomolar (pM) in a neutralization assay. In some embodiments, the multivalent particle comprises an IC50 of less than 5 picomolar (pM) in a neutralization assay. In some embodiments, the multivalent particle comprises an IC50 of less than 2.5 picomolar (pM) in a neutralization assay. In some embodiments, the multivalent particle comprises an IC50 of less than 1 picomolar (pM) in a neutralization assay. In some embodiments, the multivalent particle comprises an IC50 of less than 0.5 picomolar (pM) in a neutralization assay.

### Mammalian Polypeptides

Described herein, in some embodiments, are multivalent particles comprising a fusion protein that comprises a mammalian polypeptide that binds to a viral protein and a transmembrane polypeptide, wherein the mammalian polypeptide comprises a receptor that has binding specificity for the viral protein. In some embodiments, the receptor comprises a viral entry receptor or a viral attachment receptor. In some embodiments, the receptor is both a viral entry receptor and a viral attachment receptor. In some embodiments, the mammalian polypeptide comprises an extracellular domain of the receptor. In some embodiments, the mammalian polypeptide is a Type I receptor. In some embodiments, the mammalian polypeptide is a Type II receptor. In some embodiments, the mammalian polypeptide is a multi-span transmembrane protein. In some embodiments, the mammalian polypeptide is a de novo designed viral-binding protein. In some embodiments, the de novo designed viral-binding protein comprises using phage display or yeast display. In some embodiments, the mammalian polypeptide comprises a ligand or a secreted protein.

In some embodiments, the mammalian polypeptide comprises ACE2, TRMPSS2, DPP4, CD4, CCR5, CXCR4, CD209, or CLEC4M. In some embodiments, the mammalian polypeptide comprises ACE2. In some embodiments, the mammalian polypeptide comprises DPP4.

In some embodiments, the mammalian polypeptide comprises an amino acid sequence of at least 75% sequence identity to an amino acid sequence according to SEQ ID NO: 1. In some embodiments, the mammalian polypeptide comprises an amino acid sequence of at least 80% sequence identity to an amino acid sequence according to SEQ ID NO: 1. In some embodiments, the mammalian polypeptide comprises an amino acid sequence of at least 85% sequence identity to an amino acid sequence according to SEQ ID NO: 1. In some embodiments, the mammalian polypeptide comprises an amino acid sequence of at least 90% sequence identity to an amino acid sequence according to SEQ ID NO: 1. In some embodiments, the mammalian polypeptide comprises an amino acid sequence of at least 95% sequence identity to an amino acid sequence according to SEQ ID NO: 1. In some embodiments, the mammalian polypeptide comprises an amino acid sequence of at least 98% sequence identity to an amino acid sequence according to SEQ ID NO: 1. In some embodiments, the mammalian polypeptide comprises an amino acid sequence of at least 99% sequence identity to an amino acid sequence according to SEQ ID NO: 1.

In some embodiments, the mammalian polypeptide comprises an amino acid sequence of at least 75% sequence homology to an amino acid sequence according to SEQ ID NO: 1. In some embodiments, the mammalian polypeptide comprises an amino acid sequence of at least 80% sequence homology to an amino acid sequence according to SEQ ID NO: 1. In some embodiments, the mammalian polypeptide comprises an amino acid sequence of at least 85% sequence homology to an amino acid sequence according to SEQ ID NO: 1. In some embodiments, the mammalian polypeptide comprises an amino acid sequence of at least 90% sequence homology to an amino acid sequence according to SEQ ID NO: 1. In some embodiments, the mammalian polypeptide comprises an amino acid sequence of at least 95% sequence homology to an amino acid sequence according to SEQ ID NO: 1. In some embodiments, the mammalian polypeptide comprises an amino acid sequence of at least 98% sequence homology to an amino acid sequence according to SEQ ID NO: 1. In some embodiments, the mammalian polypeptide comprises an amino acid sequence of at least 99% sequence homology to an amino acid sequence according to SEQ ID NO: 1.

In some instances, the mammalian polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 consecutive amino acids of SEQ ID NO: 1.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, the mammalian polypeptide comprises an amino acid sequence of at least 75% sequence identity to an amino acid sequence according to SEQ ID NO: 2. In some embodiments, the mammalian polypeptide comprises an amino acid sequence of at least 80% sequence identity to an amino acid sequence according to SEQ ID NO: 2. In some embodiments, the mammalian polypeptide comprises an amino acid sequence of at least 85% sequence identity to an amino acid sequence according to SEQ ID NO: 2. In some embodiments, the mammalian polypeptide comprises an amino acid sequence of at least 90% sequence identity to an amino acid sequence according to SEQ ID NO: 2. In some embodiments, the mammalian polypeptide comprises an amino acid sequence of at least 95% sequence identity to an amino acid sequence according to SEQ ID NO: 2. In some embodiments, the mammalian polypeptide comprises an amino acid sequence of at least 98% sequence identity to an amino acid sequence according to SEQ ID NO: 2. In some embodiments, the mammalian polypeptide comprises an amino acid sequence of at least 99% sequence identity to an amino acid sequence according to SEQ ID NO: 2.

In some embodiments, the mammalian polypeptide comprises an amino acid sequence of at least 75% sequence homology to an amino acid sequence according to SEQ ID NO: 2. In some embodiments, the mammalian polypeptide comprises an amino acid sequence of at least 80% sequence homology to an amino acid sequence according to SEQ ID NO: 2. In some embodiments, the mammalian polypeptide comprises an amino acid sequence of at least 85% sequence homology to an amino acid sequence according to SEQ ID NO: 2. In some embodiments, the mammalian polypeptide comprises an amino acid sequence of at least 90% sequence homology to an amino acid sequence according to SEQ ID NO: 2. In some embodiments, the mammalian polypeptide comprises an amino acid sequence of at least 95% sequence homology to an amino acid sequence according to SEQ ID NO: 2. In some embodiments, the mammalian polypeptide comprises an amino acid sequence of at least 98% sequence homology to an amino acid sequence according to SEQ ID NO: 2. In some embodiments, the mammalian polypeptide comprises an amino acid sequence of at least 99% sequence homology to an amino acid sequence according to SEQ ID NO: 2.

In some instances, the mammalian polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, or more than 720 consecutive amino acids of SEQ ID NO: 2.

Described herein, in some embodiments, are multivalent particles comprising a fusion protein that comprises a mammalian polypeptide that binds to a viral protein and a transmembrane polypeptide, wherein the multivalent particles further comprises a second fusion protein that comprises a second mammalian polypeptide that binds to the viral protein and a second transmembrane polypeptide. In some embodiments, the second mammalian polypeptide comprises a receptor that has binding specificity for the viral protein. In some embodiments, the receptor comprises a viral entry receptor or a viral attachment receptor. In some embodiments, the receptor is both a viral entry receptor and a viral attachment receptor. In some embodiments, the second mammalian polypeptide comprises an extracellular domain of the receptor. In some embodiments, the second mammalian polypeptide is a Type I receptor. In some embodiments, the second mammalian polypeptide is a Type II receptor. In some embodiments, the mammalian polypeptide is a multi-span transmembrane protein. In some embodiments, the mammalian polypeptide is a de novo designed viral-binding protein. In some embodiments, the de novo designed viral-binding protein comprises using phage display or yeast display. In some embodiments, the second mammalian polypeptide comprises a ligand or a secreted protein.

In some embodiments, the second mammalian polypeptide comprises ACE2, TRMPSS2, DPP4, CD4, CCR5, CXCR4, CD209, or CLEC4M. In some embodiments, the second mammalian polypeptide comprises ACE2. In some embodiments, the second mammalian polypeptide comprises DPP4.

In some embodiments, the second mammalian polypeptide comprises an amino acid sequence of at least 75% sequence identity to an amino acid sequence according to SEQ ID NO: 1. In some embodiments, the second mammalian polypeptide comprises an amino acid sequence of at least 80% sequence identity to an amino acid sequence according to SEQ ID NO: 1. In some embodiments, the second mammalian polypeptide comprises an amino acid sequence of at least 85% sequence identity to an amino acid sequence according to SEQ ID NO: 1. In some embodiments, the second mammalian polypeptide comprises an amino acid sequence of at least 90% sequence identity to an amino acid sequence according to SEQ ID NO: 1. In some embodiments, the second mammalian polypeptide comprises an amino acid sequence of at least 95% sequence identity to an amino acid sequence according to SEQ ID NO: 1. In some embodiments, the second mammalian polypeptide comprises an amino acid sequence of at least 98% sequence identity to an amino acid sequence according to SEQ ID NO: 1. In some embodiments, the second mammalian polypeptide comprises an amino acid sequence of at least 99% sequence identity to an amino acid sequence according to SEQ ID NO: 1.

In some embodiments, the second mammalian polypeptide comprises an amino acid sequence of at least 75% sequence homology to an amino acid sequence according to SEQ ID NO: 1. In some embodiments, the second mammalian polypeptide comprises an amino acid sequence of at least 80% sequence homology to an amino acid sequence according to SEQ ID NO: 1. In some embodiments, the second mammalian polypeptide comprises an amino acid sequence of at least 85% sequence homology to an amino acid sequence according to SEQ ID NO: 1. In some embodiments, the second mammalian polypeptide comprises an amino acid sequence of at least 90% sequence homology to an amino acid sequence according to SEQ ID NO: 1. In some embodiments, the second mammalian polypeptide comprises an amino acid sequence of at least 95% sequence homology to an amino acid sequence according to SEQ ID NO: 1. In some embodiments, the second mammalian polypeptide comprises an amino acid sequence of at least 98% sequence homology to an amino acid sequence according to SEQ ID NO: 1. In some embodiments, the second mammalian polypeptide comprises an amino acid sequence of at least 99% sequence homology to an amino acid sequence according to SEQ ID NO: 1.

In some instances, the second mammalian polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 consecutive amino acids of SEQ ID NO: 1.

In some embodiments, the second mammalian polypeptide comprises an amino acid sequence of at least 75% sequence identity to an amino acid sequence according to SEQ ID NO: 2. In some embodiments, the second mammalian polypeptide comprises an amino acid sequence of at least 80% sequence identity to an amino acid sequence according to SEQ ID NO: 2. In some embodiments, the second mammalian polypeptide comprises an amino acid sequence of at least 85% sequence identity to an amino acid sequence according to SEQ ID NO: 2. In some embodiments, the second mammalian polypeptide comprises an amino acid sequence of at least 90% sequence identity to an amino acid sequence according to SEQ ID NO: 2. In some embodiments, the second mammalian polypeptide comprises an amino acid sequence of at least 95% sequence identity to an amino acid sequence according to SEQ ID NO: 2. In some embodiments, the second mammalian polypeptide comprises an amino acid sequence of at least 98% sequence identity to an amino acid sequence according to SEQ ID NO: 2. In some embodiments, the second mammalian polypeptide comprises an amino acid sequence of at least 99% sequence identity to an amino acid sequence according to SEQ ID NO: 2.

In some embodiments, the second mammalian polypeptide comprises an amino acid sequence of at least 75% sequence homology to an amino acid sequence according to SEQ ID NO: 2. In some embodiments, the second mammalian polypeptide comprises an amino acid sequence of at least 80% sequence homology to an amino acid sequence according to SEQ ID NO: 2. In some embodiments, the second mammalian polypeptide comprises an amino acid sequence of at least 85% sequence homology to an amino acid sequence according to SEQ ID NO: 2. In some embodiments, the second mammalian polypeptide comprises an amino acid sequence of at least 90% sequence homology to an amino acid sequence according to SEQ ID NO: 2. In some embodiments, the second mammalian polypeptide comprises an amino acid sequence of at least 95% sequence homology to an amino acid sequence according to SEQ ID NO: 2. In some embodiments, the second mammalian polypeptide comprises an amino acid sequence of at least 98% sequence homology to an amino acid sequence according to SEQ ID NO: 2. In some embodiments, the second mammalian polypeptide comprises an amino acid sequence of at least 99% sequence homology to an amino acid sequence according to SEQ ID NO: 2.

In some instances, the second mammalian polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, or more than 720 consecutive amino acids of SEQ ID NO: 2.

### Oligomerization Domains

In some embodiments, the multivalent particle comprises an oligomerization domain. In some embodiments, the fusion protein comprises an oligomerization domain. In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the trimerization domain comprises human C-propeptide of α1(I) collagen. In some embodiments, the oligomerization domain is a tetramerization domain. In some embodiments, the tetramerization domain comprises an influenza neuraminidase stem domain.

TABLE 1
Exemplary Oligomerization Domain Sequences

| | Oligo- | | SEQ ID |
|---|---|---|---|
| Domain | merization | Amino Acid Sequence | NO: |
| D4 Variation 1 | Trimer | IGTALQVKMPKSHKAIQADGWMCHASK | 5 |
| | | WVTTCDFRWYGPKYITHSIRSFTPSVEQ | |
| | | CKESIEQTKQGTWLNPGFPPQSCGYATV | |
| | | TDAEAVIVQVTPHHVLVDEYTGEWVDS | |
| | | QFINGKCSNYICPTVHNSTTWHSDYKVK | |
| | | GLCDSNLISMDI | |
| D4 Variation 2 | Trimer | IQADGWMCHASKWVTTCDFRWYGPKY | 6 |
| | | ITHSIRSFTPSVEQCKESIEQTKQGTWLNP | |
| | | GFPPQSCGYATVIDAEAVIVQVTPHHVL | |
| | | VDEYTGEWVDSQFINGKCSNYICPTVHN | |
| | | STTWHSDYKVKGLCDSNL | |
| D4 Variation 3 | Trimer | IQADGWMCHASKWVTTCDFRWYGPKY | 7 |
| | | ITHSIRSFTPSVEQCKESIEQTKQGTWLNP | |
| | | GFPPQSCGYATVTDAEAVIVQVTPHHVL | |
| | | VDEYTGEWVDSQFINGKCSNYICPTVHN | |
| | | STT | |
| D4 Variation 4 | Trimer | IQADGWMCHASKWVTTCDFRWYGPKY | 8 |
| | | ITHSIRSFTPSVEQCKESIEQTKQGTWLNP | |
| | | GFPPQSCGYATVIDAEAVIVQVTPHHVL | |
| | | VDEYTGEWVDSQFING | |
| D4 Variation 5 | Trimer | IQADGWMCHASKWVTTCDFRWYGPKY | 9 |
| | | ITHSIRSFTPSVEQCKESIEQTKQGTWLNP | |
| | | GFPPQSCGYATVIDAEAVIVQVTPHHVL | |
| Foldon | Trimer | GYIPEAPRDGQAYVRKDGEWVLLSTFL | 10 |
| Leucine Zipper | Dimer | RMKQLEDKVEELLSKQYHLENEVARLK | 11 |
| V1 | | KLVGER | |
| Leucine Zipper | Dimer | RMKQLEDKVEELLSKNYHLENEVARLK | 12 |
| V2 | | KLVGER | |
| Neuraminidase | Tetramer | MNPNQKIITIGSICLVVGLISLILQIGNIISI | 13 |
| Stem V1 | | WISHSIQT | |
| Neuraminidase | Tetramer | MNPNQKIITIGSICMVTGIVSLMLQIGNM | 14 |
| Stem V2 | | ISIWVSHSIHTGNQHQSEPISNTNFLTEKA | |
| | | VASVKLAGNSSLCPIN | |
| Dengue E Fusion | Trimer | KLCIEAKISNTTTDSRCPTQGEATLVEEQ | 15 |
| V1 | | DTNFVCRRTFVDRGHGNGCGLFGKGSLI | |
| | | TCAKFKCVTKL | |
| Dengue E Fusion | Trimer | IELLKTEVTNPAVLRKLCIEAKISNTTTDS | 16 |
| V2 | | RCPTQGEATLVEEQDTNFVCRRTFVDRG | |
| | | HGNGCGLFGKGSLITCAKFKCVTKL | |
| Dengue E Fusion | Trimer | KLCIEAKISNTTTDSRCPTQGEATLVEEQ | 17 |
| V3 | | DTNFVCRRTFVDRGHGNGCGLFGKGSLI | |
| | | TCAKFKCVTKLEGKIVQYENLKYSVI | |
| Dengue E Fusion | Trimer | EAKISNTTTDSRCPTQGEATLVEEQDTNF | 18 |
| V4 | | VCRRTFVDRGHGNGCGLFGKGSLITCAK | |
| | | FK | |
| human C- | Trimer | ETGHHHHHHSADEPMDFKINTDEIMTSL | 28 |
| propeptide of | | KSVNGQIESLISPDGSRKNPARNCRDLKF | |
| α1(I) collagen | | CHPELKSGEYWVDPNQGCKLDAIKVFC | |
| | | NMETGETCISANPLNVPRKHWWTDSSA | |
| | | EKKHVWFGESMDGGFQFSYGNPELPED | |
| | | VLDVQLAFLRLLSSRASQQITYHCKNSIA | |
| | | YMDQASGNVKKALKLMGSNEGEFKAE | |
| | | GNSKFTYTVLEDGCTKHTGEWSKTVFE | |
| | | YRTRKAVRLPIVDIAPYDIGGPDQEFGV | |
| | | DVGPVCFL | |

In some embodiments, the oligomerization domain comprises an amino acid sequence disclosed in Tablet 1, or an amino acid sequence that is substantially identical to an amino acid sequence in Table 1 (e.g. 80%, 85%, 90%, 9500, 96%, 9700, 98%, 9900 sequence identity). In some instances, the oligomerization domain comprises an amino acid sequence comprising at least a portion having at least or about 10, 20, 30, 40, 50, 60, 70, 80, 90, 100, 110, 120, 130 consecutive amino acid sequences of any sequence according to Tablet 1. In some embodiments, the oligomerization domain comprises an amino acid sequence that has at least 95% sequence identity to an amino acid sequence according to any one of SEQ ID NOs: 5-18 and 28.

### Transmembrane Polypeptides

Described herein, in some embodiments, are multivalent particles comprising a fusion protein that comprises a mammalian polypeptide that binds to a viral protein and a transmembrane polypeptide. In some embodiments, the transmembrane polypeptide comprises the transmembrane domain of a Vesicular Stomatitis virus glycoprotein (VSV-G). In some embodiments, the transmembrane polypeptide comprises the transmembrane domain and cytosolic domain of a Vesicular Stomatitis virus glycoprotein (VSV-G). In some embodiments, the transmembrane polypeptide comprises the transmembrane domain of a Dengue E protein. In some embodiments, the transmembrane polypeptide comprises the transmembrane domain and cytosolic domain of a Dengue E protein. In some embodiments, the transmembrane polypeptide comprises the transmembrane domain of influenza Hemagglutinin (HA). In some embodiments, the transmembrane polypeptide comprises the transmembrane domain and cytosolic domain of influenza Hemagglutinin (HA). In some embodiments, the transmembrane polypeptide comprises the transmembrane domain of HIV surface glycoprotein GP120 or GP41. In some embodiments, the transmembrane polypeptide comprises the transmembrane domain and cytosolic domain of HIV surface glycoprotein GP120 or GP41. In some embodiments, the transmembrane domain comprises the transmembrane polypeptide of measles virus surface glycoprotein hamagglutinin (H) protein. In some embodiments, the transmembrane polypeptide comprises the transmembrane domain and cytosolic domain of measles virus surface glycoprotein hamagglutinin (H) protein. In some embodiments, the transmembrane polypeptide comprises the transmembrane domain of influenza Neuraminidase (NA). In some embodiments, the transmembrane polypeptide comprises the transmembrane domain and cytosolic domain of influenza Neuraminidase (NA).

TABLE 2
Exemplary Transmembrane Polypeptide Sequences

| Domain | Amino Acid Sequence | SEQ ID NO: |
|---|---|---|
| VSV-G | IASFFFIIGLIIGLFLVLRVGI | 19 |
| Transmembrane | | |
| (TM) V1 | | |
| VSV-G | PIELVEGWFSSWKSSIASFFFIIGLIIGLFL | 20 |
| Transmembrane | VLRVGI | |
| (TM) V2 | | |
| VSV-G | DDESLFFGDTGLSKNPIELVEGWFSSWK | 21 |
| Transmembrane | SSIASFFFIIGLIIGLFLVLRVGIH | |
| (TM) V3 | | |
| VSV-G | GMLDSDLHLSSKAQVFEHPHIQDAASQL | 22 |
| Transmembrane | PDDESLFFGDTGLSKNPIELVEGWFSSW | |
| (TM) V4 | KSSIASFFFIIGLIIGLFLVLRVGI | |
| VSV-G | HLCIKLKHTKKRQIYTDIEMNRLGK | 23 |
| Cytosolic Tail | | |
| (CT) | | |
| Influenza | IITIGSVCMTIGMANLILQIGNI | 24 |
| Neuraminidase | | |
| TM (N1) | | |
| Influenza | LAIYSTVASSLVLVVSLGAISFW | 25 |
| Hemagglutinin | | |
| TM (H1) | | |
| Dengue E | AYGVLFSGVSWTMKIGIGILLTWLGLNS | 26 |
| Protein TM | RSTSLSMTCIAVGMVTLYLGVMVQ | |
| HIV gp TM | FIMIVGGLVGLRIVFAVLSIV | 27 |

In some embodiments, the transmembrane polypeptide comprises an amino acid sequence disclosed in Table 2, or an amino acid sequence that is substantially identical to an amino acid sequence in Table 2 (e.g. 80%, 85%, 900%, 9500, 9600, 9700, 98%, 9900 sequence identity). In some instances, the transmembrane polypeptide comprises an amino acid sequence comprising at least a portion having at least or about 10, 20, 30, 40, 50, 60, 70, 80, 90, 100, 110, 120, 130 consecutive amino acid sequences of any sequence according to Table 2.

Described herein, in some embodiments, are multivalent particles comprising a fusion protein that comprises a mammalian polypeptide that binds to a viral protein and a transmembrane polypeptide. In some embodiments, the transmembrane polypeptide anchors the fusion protein to a lipid bilayer of the multivalent particle. In some embodiments, the transmembrane polypeptide comprises a spike glycoprotein, a mammalian membrane protein, an envelope protein, a nucleocapsid protein, or a cellular transmembrane protein. In some embodiments, the transmembrane polypeptide comprises the transmembrane domain of VSVG, spike protein S1, spike protein S2, Sindbis virus envelope (SINDBIS) protein, hemagglutinin envelope protein from measles virus, envelope glycoprotein of measles virus fusion (F) protein, RD 114, BaEV, GP41, or GP120. In some embodiments, the transmembrane polypeptide comprises VSVG transmembrane region. In some embodiments, the VSVG transmembrane region comprises full length VSVG transmembrane region or a truncated VSVG transmembrane region. In some embodiments, the transmembrane polypeptide comprises a VSVG transmembrane region and a VSVG cytoplasmic tail. In some embodiments, the hemagglutinin envelope protein from measles virus is a variant of the hemagglutinin envelope protein from measles virus. In some instances, the variant is HCΔ18.

In some embodiments, the transmembrane polypeptide comprises an amino acid sequence of at least 75% sequence identity to an amino acid sequence according to SEQ ID NO: 3. In some embodiments, the transmembrane polypeptide comprises an amino acid sequence of at least 80% sequence identity to an amino acid sequence according to SEQ ID NO: 3. In some embodiments, the transmembrane polypeptide comprises an amino acid sequence of at least 85% sequence identity to an amino acid sequence according to SEQ ID NO: 3. In some embodiments, the transmembrane polypeptide comprises an amino acid sequence of at least 90% sequence identity to an amino acid sequence according to SEQ ID NO: 3. In some embodiments, the transmembrane polypeptide comprises an amino acid sequence of at least 95% sequence identity to an amino acid sequence according to SEQ ID NO: 3. In some embodiments, the transmembrane polypeptide comprises an amino acid sequence of at least 98% sequence identity to an amino acid sequence according to SEQ ID NO: 3. In some embodiments, the transmembrane polypeptide comprises an amino acid sequence of at least 99% sequence identity to an amino acid sequence according to SEQ ID NO: 3.

In some embodiments, the transmembrane polypeptide comprises an amino acid sequence of at least 75% sequence homology to an amino acid sequence according to SEQ ID NO: 3. In some embodiments, the transmembrane polypeptide comprises an amino acid sequence of at least 80% sequence homology to an amino acid sequence according to SEQ ID NO: 3. In some embodiments, the transmembrane polypeptide comprises an amino acid sequence of at least 85% sequence homology to an amino acid sequence according to SEQ ID NO: 3. In some embodiments, the transmembrane polypeptide comprises an amino acid sequence of at least 90% sequence homology to an amino acid sequence according to SEQ ID NO: 3. In some embodiments, the transmembrane polypeptide comprises an amino acid sequence of at least 95% sequence homology to an amino acid sequence according to SEQ ID NO: 3. In some embodiments, the transmembrane polypeptide comprises an amino acid sequence of at least 98% sequence homology to an amino acid sequence according to SEQ ID NO: 3. In some embodiments, the transmembrane polypeptide comprises an amino acid sequence of at least 99% sequence homology to an amino acid sequence according to SEQ ID NO: 3.

In some instances, the transmembrane polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or more than 490 consecutive amino acids of SEQ ID NO: 3.

In some embodiments, the transmembrane polypeptide comprises an amino acid sequence of at least 75% sequence identity to an amino acid sequence according to SEQ ID NO: 4. In some embodiments, the transmembrane polypeptide comprises an amino acid sequence of at least 80% sequence identity to an amino acid sequence according to SEQ ID NO: 4. In some embodiments, the transmembrane polypeptide comprises an amino acid sequence of at least 85% sequence identity to an amino acid sequence according to SEQ ID NO: 4. In some embodiments, the transmembrane polypeptide comprises an amino acid sequence of at least 90% sequence identity to an amino acid sequence according to SEQ ID NO: 4. In some embodiments, the transmembrane polypeptide comprises an amino acid sequence of at least 95% sequence identity to an amino acid sequence according to SEQ ID NO: 4. In some embodiments, the transmembrane polypeptide comprises an amino acid sequence of at least 98% sequence identity to an amino acid sequence according to SEQ ID NO: 4. In some embodiments, the transmembrane polypeptide comprises an amino acid sequence of at least 99% sequence identity to an amino acid sequence according to SEQ ID NO: 4.

In some embodiments, the transmembrane polypeptide comprises an amino acid sequence of at least 75% sequence homology to an amino acid sequence according to SEQ ID NO: 4. In some embodiments, the transmembrane polypeptide comprises an amino acid sequence of at least 80% sequence homology to an amino acid sequence according to SEQ ID NO: 4. In some embodiments, the transmembrane polypeptide comprises an amino acid sequence of at least 85% sequence homology to an amino acid sequence according to SEQ ID NO: 4. In some embodiments, the transmembrane polypeptide comprises an amino acid sequence of at least 90% sequence homology to an amino acid sequence according to SEQ ID NO: 4. In some embodiments, the transmembrane polypeptide comprises an amino acid sequence of at least 95% sequence homology to an amino acid sequence according to SEQ ID NO: 4. In some embodiments, the transmembrane polypeptide comprises an amino acid sequence of at least 98% sequence homology to an amino acid sequence according to SEQ ID NO: 4. In some embodiments, the transmembrane polypeptide comprises an amino acid sequence of at least 99% sequence homology to an amino acid sequence according to SEQ ID NO: 4.

In some instances, the transmembrane polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 800, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, or more than 1250 consecutive amino acids of SEQ ID NO: 4.

Described herein, in some embodiments, are multivalent particles comprising a fusion protein that comprises a mammalian polypeptide that binds to a viral protein and a transmembrane polypeptide, wherein the multivalent particles further comprises a second fusion protein that comprises a second mammalian polypeptide that binds to the viral protein and a second transmembrane polypeptide. In some embodiments, the second transmembrane polypeptide comprises the transmembrane region of a spike glycoprotein, a mammalian membrane protein, an envelope protein, a nucleocapsid protein, or a cellular transmembrane protein. In some embodiments, the second transmembrane polypeptide comprises the transmembrane region of VSVG, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120. In some embodiments, the second transmembrane polypeptide comprises VSVG transmembrane region. In some embodiments, the VSVG transmembrane region comprises full length VSVG transmembrane region or a truncated VSVG transmembrane region. In some embodiments, the transmembrane polypeptide comprises a VSVG transmembrane region and a VSVG cytoplasmic tail. In some embodiments, the hemagglutinin envelope protein from measles virus is a variant of the hemagglutinin envelope protein from measles virus. In some instances, the variant is HCΔ18.

In some embodiments, the second transmembrane polypeptide comprises an amino acid sequence of at least 75% sequence identity to an amino acid sequence according to SEQ ID NO: 3. In some embodiments, the second transmembrane polypeptide comprises an amino acid sequence of at least 80% sequence identity to an amino acid sequence according to SEQ ID NO: 3. In some embodiments, the second transmembrane polypeptide comprises an amino acid sequence of at least 85% sequence identity to an amino acid sequence according to SEQ ID NO: 3. In some embodiments, the second transmembrane polypeptide comprises an amino acid sequence of at least 90% sequence identity to an amino acid sequence according to SEQ ID NO: 3. In some embodiments, the second transmembrane polypeptide comprises an amino acid sequence of at least 95% sequence identity to an amino acid sequence according to SEQ ID NO: 3. In some embodiments, the second transmembrane polypeptide comprises an amino acid sequence of at least 98% sequence identity to an amino acid sequence according to SEQ ID NO: 3. In some embodiments, the second transmembrane polypeptide comprises an amino acid sequence of at least 99% sequence identity to an amino acid sequence according to SEQ ID NO: 3.

In some embodiments, the second transmembrane polypeptide comprises an amino acid sequence of at least 75% sequence homology to an amino acid sequence according to SEQ ID NO: 3. In some embodiments, the second transmembrane polypeptide comprises an amino acid sequence of at least 80% sequence homology to an amino acid sequence according to SEQ ID NO: 3. In some embodiments, the second transmembrane polypeptide comprises an amino acid sequence of at least 85% sequence homology to an amino acid sequence according to SEQ ID NO: 3. In some embodiments, the second transmembrane polypeptide comprises an amino acid sequence of at least 90% sequence homology to an amino acid sequence according to SEQ ID NO: 3. In some embodiments, the second transmembrane polypeptide comprises an amino acid sequence of at least 95% sequence homology to an amino acid sequence according to SEQ ID NO: 3. In some embodiments, the second transmembrane polypeptide comprises an amino acid sequence of at least 98% sequence homology to an amino acid sequence according to SEQ ID NO: 3. In some embodiments, the second transmembrane polypeptide comprises an amino acid sequence of at least 99% sequence homology to an amino acid sequence according to SEQ ID NO: 3.

In some instances, the second transmembrane polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or more than 490 consecutive amino acids of SEQ ID NO: 3.

In some embodiments, the second transmembrane polypeptide comprises an amino acid sequence of at least 75% sequence identity to an amino acid sequence according to SEQ ID NO: 4. In some embodiments, the second transmembrane polypeptide comprises an amino acid sequence of at least 80% sequence identity to an amino acid sequence according to SEQ ID NO: 4. In some embodiments, the second transmembrane polypeptide comprises an amino acid sequence of at least 85% sequence identity to an amino acid sequence according to SEQ ID NO: 4. In some embodiments, the second transmembrane polypeptide comprises an amino acid sequence of at least 90% sequence identity to an amino acid sequence according to SEQ ID NO: 4. In some embodiments, the second transmembrane polypeptide comprises an amino acid sequence of at least 95% sequence identity to an amino acid sequence according to SEQ ID NO: 4. In some embodiments, the second transmembrane polypeptide comprises an amino acid sequence of at least 98% sequence identity to an amino acid sequence according to SEQ ID NO: 4. In some embodiments, the second transmembrane polypeptide comprises an amino acid sequence of at least 99% sequence identity to an amino acid sequence according to SEQ ID NO: 4.

In some embodiments, the second transmembrane polypeptide comprises an amino acid sequence of at least 75% sequence homology to an amino acid sequence according to SEQ ID NO: 4. In some embodiments, the second transmembrane polypeptide comprises an amino acid sequence of at least 80% sequence homology to an amino acid sequence according to SEQ ID NO: 4. In some embodiments, the second transmembrane polypeptide comprises an amino acid sequence of at least 85% sequence homology to an amino acid sequence according to SEQ ID NO: 4. In some embodiments, the second transmembrane polypeptide comprises an amino acid sequence of at least 90% sequence homology to an amino acid sequence according to SEQ ID NO: 4. In some embodiments, the second transmembrane polypeptide comprises an amino acid sequence of at least 95% sequence homology to an amino acid sequence according to SEQ ID NO: 4. In some embodiments, the second transmembrane polypeptide comprises an amino acid sequence of at least 98% sequence homology to an amino acid sequence according to SEQ ID NO: 4. In some embodiments, the second transmembrane polypeptide comprises an amino acid sequence of at least 99% sequence homology to an amino acid sequence according to SEQ ID NO: 4.

In some instances, the second transmembrane polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 800, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, or more than 1250 consecutive amino acids of SEQ ID NO: 4.

### Mammalian Polypeptide and Transmembrane Polypeptide Combinations

Described herein, in some embodiments, are multivalent particles comprising a fusion protein that comprises a mammalian polypeptide that binds to a viral protein and a transmembrane polypeptide. In some embodiments, the mammalian polypeptide is a Type I receptor. In some embodiments, the mammalian polypeptide is a Type II receptor.

In some embodiments, the mammalian polypeptide comprises ACE2, TRMPSS2, DPP4, CD4, CCR5, CXCR4, CD209, or CLEC4M and the transmembrane polypeptide comprises the transmembrane region of VSVG, spike protein S1, spike protein S2, Sindbis virus envelope (SINDBIS) protein, hemagglutinin envelope protein from measles virus, envelope glycoprotein of measles virus fusion (F) protein, RD 114, BaEV, GP41, or GP120.

In some embodiments, the mammalian polypeptide comprises ACE2 and the transmembrane polypeptide comprises VSVG transmembrane region. In some embodiments, the mammalian polypeptide comprises ACE2 and the transmembrane polypeptide comprises spike protein S1 transmembrane region. In some embodiments, the mammalian polypeptide comprises ACE2 and the transmembrane polypeptide comprises spike protein S2 transmembrane region. In some embodiments, the mammalian polypeptide comprises ACE2 and the transmembrane polypeptide comprises the transmembrane region of a surface glycoprotein of an enveloped virus. In some embodiments, the mammalian polypeptide comprises ACE2 and the transmembrane polypeptide comprises the transmembrane region of Sindbis virus envelope (SINDBIS) protein. In some embodiments, the mammalian polypeptide comprises ACE2 and the transmembrane polypeptide comprises BaEV transmembrane region. In some embodiments, the mammalian polypeptide comprises ACE2 and the transmembrane polypeptide comprises GP41 transmembrane region. In some embodiments, the mammalian polypeptide comprises ACE2 and the transmembrane polypeptide comprises GP120 transmembrane region.

In some embodiments, the mammalian polypeptide comprises CD4 and the transmembrane polypeptide comprises VSVG transmembrane region. In some embodiments, the mammalian polypeptide comprises CD4 and the transmembrane polypeptide comprises spike protein S1 transmembrane region. In some embodiments, the mammalian polypeptide comprises CD4 and the transmembrane polypeptide comprises spike protein S2 transmembrane region. In some embodiments, the mammalian polypeptide comprises CD4 and the transmembrane polypeptide comprises the transmembrane region of a surface glycoprotein of an enveloped virus. In some embodiments, the mammalian polypeptide comprises CD4 and the transmembrane polypeptide comprises Sindbis virus envelope (SINDBIS) protein transmembrane region. In some embodiments, the mammalian polypeptide comprises CD4 and the transmembrane polypeptide comprises BaEV transmembrane region. In some embodiments, the mammalian polypeptide comprises CD4 and the transmembrane polypeptide comprises GP41 transmembrane region. In some embodiments, the mammalian polypeptide comprises CD4 and the transmembrane polypeptide comprises GP120 transmembrane region.

In some embodiments, the mammalian polypeptide comprises DPP4 and the transmembrane polypeptide comprises hemagglutinin envelope protein from measles virus. In some embodiments, the hemagglutinin envelope protein from measles virus is a variant of the hemagglutinin envelope protein from measles virus. In some embodiments, the variant is HCΔ18. In some embodiments, the mammalian polypeptide comprises DPP4 and the transmembrane polypeptide comprises envelope glycoprotein of measles virus fusion (F) protein.

In some embodiments, the mammalian polypeptide comprises TRMPSS2 and the transmembrane polypeptide comprises hemagglutinin envelope protein from measles virus. In some embodiments, the hemagglutinin envelope protein from measles virus is a variant of the hemagglutinin envelope protein from measles virus. In some embodiments, the variant is HCΔ18. In some embodiments, the mammalian polypeptide comprises TRMPSS2 and the transmembrane polypeptide comprises envelope glycoprotein of measles virus fusion (F) protein.

In some embodiments, the mammalian polypeptide comprises CD209 and the transmembrane polypeptide comprises hemagglutinin envelope protein from measles virus. In some embodiments, the hemagglutinin envelope protein from measles virus is a variant of the hemagglutinin envelope protein from measles virus. In some embodiments, the variant is HCΔ18. In some embodiments, the mammalian polypeptide comprises CD209 and the transmembrane polypeptide comprises envelope glycoprotein of measles virus fusion (F) protein.

In some embodiments, the mammalian polypeptide comprises CLEC4M and the transmembrane polypeptide comprises hemagglutinin envelope protein from measles virus. In some embodiments, the hemagglutinin envelope protein from measles virus is a variant of the hemagglutinin envelope protein from measles virus. In some embodiments, the variant is HCΔ18. In some embodiments, the mammalian polypeptide comprises CLEC4M and the transmembrane polypeptide comprises envelope glycoprotein of measles virus fusion (F) protein.

Described herein, in some embodiments, are multivalent particles comprising a fusion protein that comprises a mammalian polypeptide that binds to a viral protein and a transmembrane polypeptide, wherein the multivalent particles further comprise a second mammalian polypeptide and second transmembrane polypeptide. In some embodiments, the second mammalian polypeptide is a Type I receptor. In some embodiments, the second mammalian polypeptide is a Type II receptor.

In some embodiments, the second mammalian polypeptide comprises ACE2, TRMPSS2, DPP4, CD4, CCR5, CXCR4, CD209, or CLEC4M and the second transmembrane polypeptide comprises the transmembrane region of VSVG, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120.

In some embodiments, the second mammalian polypeptide comprises ACE2 and the second transmembrane polypeptide comprises VSVG transmembrane region. In some embodiments, the second mammalian polypeptide comprises ACE2 and the second transmembrane polypeptide comprises spike protein S1 transmembrane region. In some embodiments, the second mammalian polypeptide comprises ACE2 and the second transmembrane polypeptide comprises spike protein S2 transmembrane region. In some embodiments, the second mammalian polypeptide comprises ACE2 and the second transmembrane polypeptide comprises the transmembrane region of a surface glycoprotein of an enveloped virus. In some embodiments, the second mammalian polypeptide comprises ACE2 and the second transmembrane polypeptide comprises Sindbis virus envelope (SINDBIS) protein transmembrane region. In some embodiments, the second mammalian polypeptide comprises ACE2 and the second transmembrane polypeptide comprises BaEV transmembrane region. In some embodiments, the second mammalian polypeptide comprises ACE2 and the second transmembrane polypeptide comprises GP41 transmembrane region. In some embodiments, the second mammalian polypeptide comprises ACE2 and the second transmembrane polypeptide comprises GP120 transmembrane region.

In some embodiments, the second mammalian polypeptide comprises CD4 and the second transmembrane polypeptide comprises VSVG transmembrane region. In some embodiments, the second mammalian polypeptide comprises CD4 and the second transmembrane polypeptide comprises spike protein S1 transmembrane region. In some embodiments, the second mammalian polypeptide comprises CD4 and the second transmembrane polypeptide comprises spike protein S2 transmembrane region. In some embodiments, the second mammalian polypeptide comprises CD4 and the second transmembrane polypeptide comprises the transmembrane region of a surface glycoprotein of an enveloped virus. In some embodiments, the second mammalian polypeptide comprises CD4 and the second transmembrane polypeptide comprises Sindbis virus envelope (SINDBIS) protein transmembrane region. In some embodiments, the second mammalian polypeptide comprises CD4 and the second transmembrane polypeptide comprises BaEV transmembrane region. In some embodiments, the second mammalian polypeptide comprises CD4 and the second transmembrane polypeptide comprises GP41 transmembrane region. In some embodiments, the second mammalian polypeptide comprises CD4 and the second transmembrane polypeptide comprises GP120 transmembrane region.

In some embodiments, the second mammalian polypeptide comprises DPP4 and the second transmembrane polypeptide comprises hemagglutinin envelope protein from measles virus. In some embodiments, the hemagglutinin envelope protein from measles virus is a variant of the hemagglutinin envelope protein from measles virus. In some embodiments, the variant is HCΔ18. In some embodiments, the second mammalian polypeptide comprises DPP4 and the second transmembrane polypeptide comprises envelope glycoprotein of measles virus fusion (F) protein.

In some embodiments, the second mammalian polypeptide comprises TRMPSS2 and the second transmembrane polypeptide comprises hemagglutinin envelope protein from measles virus. In some embodiments, the hemagglutinin envelope protein from measles virus is a variant of the hemagglutinin envelope protein from measles virus. In some embodiments, the variant is HCΔ18. In some embodiments, the second mammalian polypeptide comprises TRMPSS2 and the second transmembrane polypeptide comprises envelope glycoprotein of measles virus fusion (F) protein.

In some embodiments, the second mammalian polypeptide comprises CD209 and the second transmembrane polypeptide comprises hemagglutinin envelope protein from measles virus. In some embodiments, the hemagglutinin envelope protein from measles virus is a variant of the hemagglutinin envelope protein from measles virus. In some embodiments, the variant is HCΔ18. In some embodiments, the second mammalian polypeptide comprises CD209 and the second transmembrane polypeptide comprises envelope glycoprotein of measles virus fusion (F) protein.

In some embodiments, the second mammalian polypeptide comprises CLEC4M and the second transmembrane polypeptide comprises hemagglutinin envelope protein from measles virus. In some embodiments, the hemagglutinin envelope protein from measles virus is a variant of the hemagglutinin envelope protein from measles virus. In some embodiments, the variant is HCΔ18. In some embodiments, the second mammalian polypeptide comprises CLEC4M and the second transmembrane polypeptide comprises envelope glycoprotein of measles virus fusion (F) protein.

In some embodiments, the mammalian polypeptide comprises ACE2, the transmembrane polypeptide comprises VSVG transmembrane region, spike protein S2 transmembrane region, or a surface glycoprotein of an enveloped virus, the second mammalian polypeptide comprises a heparan sulfate proteoglycan, and the second transmembrane polypeptide comprises VSVG transmembrane region, spike protein S2 transmembrane region, or a surface glycoprotein of an enveloped virus. In some embodiments, the mammalian polypeptide comprises CD4 and the second mammalian peptide comprises, CCR5, CXCR4, or both.

Described herein, in some embodiments, are multivalent particles comprising a fusion protein that comprises a mammalian polypeptide that binds to a viral protein and a transmembrane polypeptide, wherein the multivalent particles further comprise, wherein the multivalent particles further comprise an oligomerization domain.

In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the trimerization domain comprises human C-propeptide of α1(I) collagen. In some embodiments, the oligomerization domain is a tetramerization domain. In some embodiments, the tetramerization domain comprises an influenza neuraminidase stem domain.

In some embodiments, when the fusion protein is expressed on the surface of the multivalent particle, the oligomerization domain is outside of the multivalent particle. In some embodiments, when the fusion protein is expressed on the surface of the multivalent particle, the oligomerization domain is outside of the multivalent particle and adjacent to a signal peptide. In some embodiments, when the fusion protein is expressed on the surface of the multivalent particle, the oligomerization domain is outside of the multivalent particle and adjacent to the transmembrane domain. In some embodiments, when the fusion protein is expressed on the surface of the multivalent particle, the oligomerization domain is inside of the multivalent particle. In some embodiments, when the fusion protein is expressed on the surface of the multivalent particle, the oligomerization domain is inside of the multivalent particle and adjacent to the transmembrane polypeptide.

In some embodiments, the fusion protein comprises a signal peptide.

In some embodiments, domains of the fusion protein are arranged from the N-terminus to the C-terminus in the following orders: (a) signal peptide, mammalian polypeptide, oligomerization domain, transmembrane polypeptide, and cytosolic domain; (b) signal peptide, mammalian polypeptide, transmembrane polypeptide, oligomerization domain, and cytosolic domain; or (c) signal peptide, oligomerization domain, mammalian polypeptide, transmembrane polypeptide, and cytosolic domain. In some embodiments, domains of the fusion protein are arranged from the N-terminus to the C-terminus in the following order: signal peptide, mammalian polypeptide, oligomerization domain, transmembrane polypeptide, and cytosolic domain. In some embodiments, domains of the fusion protein are arranged from the N-terminus to the C-terminus in the following order: signal peptide, mammalian polypeptide, transmembrane polypeptide, oligomerization domain, and cytosolic domain. In some embodiments, domains of the fusion protein are arranged from the N-terminus to the C-terminus in the following order: signal peptide, oligomerization domain, mammalian polypeptide, transmembrane polypeptide, and cytosolic domain.

Disclosed herein are fusion proteins comprising a transmembrane polypeptide, a cytosolic domain, a mammalian polypeptide, and an oligomerization domain wherein when the fusion protein is expressed on the surface of a multivalent particle, the fusion protein is displayed in an oligomeric format.

TABLE 3
Exemplary Fusion Protein Sequences

| Fusion Protein | Amino Acid Sequence | SEQ ID NO: |
|---|---|---|
| ACE2 fused with | MSSSSWLLLSLVAVTAAQSTIEEQAKTFLDKFNHEAED | 29 |
| VSVG | LFYQSSLASWNYNTNITEENVQNMNNAGDKWSAFLK | |
| transmembrane | EQSTLAQMYPLQEIQNLTVKLQLQALQQNGSSVLSED | |
| domain (VGTM) | KSKRLNTILNTMSTIYSTGKVCNPDNPQECLLLEPGLNE | |
| | IMANSLDYNERLWAWESWRSEVGKQLRPLYEEYVVL | |
| | KNEMARANHYEDYGDYWRGDYEVNGVDGYDYSRGQ | |
| | LIEDVEHTFEEIKPLYEHLHAYVRAKLMNAYPSYISPIG | |
| | CLPAHLLGDMWGRFWTNLYSLTVPFGQKPNIDVTDA | |
| | MVDQAWDAQRIFKEAEKFFVSVGLPNMTQGFWENSM | |
| | LTDPGNVQKAVCHPTAWDLGKGDFRILMCTKVTMDD | |
| | FLTAHHEMGHIQYDMAYAAQPFLLRNGANEGFHEAV | |
| | GEIMSLSAATPKHLKSIGLLSPDFQEDNETEINFLLKQA | |
| | LTIVGTLPFTYMLEKWRWMVFKGEIPKDQWMKKWW | |
| | EMKREIVGVVEPVPHDETYCDPASLFHVSNDYSFIRYY | |
| | TRTLYQFQFQEALCQAAKHEGPLHKCDISNSTEAGQKL | |
| | FNMLRLGKSEPWTLALENVVGAKNMNVRPLLNYFEPL | |
| | FTWLKDQNKNSFVGWSTDWSPYADQSIKVRISLKSAL | |
| | GDKAYEWNDNEMYLFRSSVAYAMRQYFLKVKNQMIL | |
| | FGEEDVRVANLKPRISFNFFVTAPKNVSDIIPRTEVEKAI | |
| | RMSRSRINDAFRLNDNSLEFLGIQPTLGPPNQPPVSSRG | |
| | MLDSDLHLSSKAQVFEHPHIQDAASQLPDDESLFFGDT | |
| | GLSKNPIELVEGWFSSWKSSIASFFFIIGLIIGLFLVLRVG | |
| | IHLCIKLKHTKKRQIYTDIEMNRLGK | |
| ACE2 fused with | MSSSSWLLLSLVAVTAAQSTIEEQAKTFLDKFNHEAED | 30 |
| S2 | LFYQSSLASWNYNTNITEENVQNMNNAGDKWSAFLK | |
| transmembrane | EQSTLAQMYPLQEIQNLTVKLQLQALQQNGSSVLSED | |
| domain (S2TM) | KSKRLNTILNTMSTIYSTGKVCNPDNPQECLLLEPGLNE | |
| | IMANSLDYNERLWAWESWRSEVGKQLRPLYEEYVVL | |
| | KNEMARANHYEDYGDYWRGDYEVNGVDGYDYSRGQ | |
| | LIEDVEHTFEEIKPLYEHLHAYVRAKLMNAYPSYISPIG | |
| | CLPAHLLGDMWGRFWTNLYSLTVPFGQKPNIDVTDA | |
| | MVDQAWDAQRIFKEAEKFFVSVGLPNMTQGFWENSM | |
| | LTDPGNVQKAVCHPTAWDLGKGDFRILMCTKVTMDD | |
| | FLTAHHEMGHIQYDMAYAAQPFLLRNGANEGFHEAV | |
| | GEIMSLSAATPKHLKSIGLLSPDFQEDNETEINFLLKQA | |
| | LTIVGTLPFTYMLEKWRWMVFKGEIPKDQWMKKWW | |
| | EMKREIVGVVEPVPHDETYCDPASLFHVSNDYSFIRYY | |
| | TRTLYQFQFQEALCQAAKHEGPLHKCDISNSTEAGQKL | |
| | FNMLRLGKSEPWTLALENVVGAKNMNVRPLLNYFEPL | |
| | FTWLKDQNKNSFVGWSTDWSPYADQSIKVRISLKSAL | |
| | GDKAYEWNDNEMYLFRSSVAYAMRQYFLKVKNQMIL | |
| | FGEEDVRVANLKPRISFNFFVTAPKNVSDIIPRTEVEKAI | |
| | RMSRSRINDAFRLNDNSLEFLGIQPTLGPPNQPPVSDIG | |
| | GGSVASQSIIAYTMSLGAENSVAYSNNSIAIPTNFTISVT | |
| | TEILPVSMTKTSVDCTMYICGDSTECSNLLLQYGSFCTQ | |
| | LNRALTGIAVEQDKNTQEVFAQVKQIYKTPPIKDFGGF | |
| | NFSQILPDPSKPSKRSFIEDLLFNKVTLADAGFIKQYGD | |
| | CLGDIAARDLICAQKFNGLTVLPPLLTDEMIAQYTSALL | |
| | AGTITSGWTFGAGAALQIPFAMQMAYRENGIGVTQNV | |
| | LYENQKLIANQFNSAIGKIQDSLSSTASALGKLQDVVN | |
| | QNAQALNTLVKQLSSNFGAISSVLNDILSRLDKVEAEV | |
| | QIDRLITGRLQSLQTYVTQQLIRAAEIRASANLAATKMS | |
| | ECVLGQSKRVDFCGKGYHLMSFPQSAPHGVVFLHVTY | |
| | VPAQEKNFTTAPAICHDGKAHFPREGVFVSNGTHWFV | |
| | TQRNFYEPQIITTDNTFVSGNCDVVIGIVNNTVYDPLQP | |
| | ELDSFKEELDKYFKNHTSPDVDLGDISGINASVVNIQKE | |
| | IDRLNEVAKNLNESLIDLQELGKYEQYIKWPWYIWLGF | |
| | IAGLIAIVMVTIMLCCMTSCCSCLKGCCSCGSCCKFDE | |
| | DDSEPVLKGVKLHYTYTDIEMNRLGK | |
| HCΔ-DPP4 | MGSRIVINREHLMIDRPYVLLAVLFVMFLSLIGLLAIAG | 31 |
| | IRLHRAAIYTAEIHKSLSTNLDVTNSIEHQVKDVLTPLF | |
| | KIIGDEVGLRTPQRFTDLVKFISDKIKFLNPDREYDFRD | |
| | LTWCINPPERIKLDYDQYCADVAAEELMNALVNSTLL | |
| | ETRTTNQFLAVSKGNCSGPTTIRGQFSNMSLSLLDLYL | |
| | GRGYNVSSIVTMTSQGMYGGTYLVEKPNLSSKRSELS | |
| | QLSMYRVFEVGVIRNPGLGAPVFHMTNYLEQPVSNDL | |
| | SNCMVALGELKLAALCHGEDSITIPYQGSGKGVSFQLV | |
| | KLGVWKSPTDMQSWVPLSTDDPVIDRLYLSSHRGVIA | |
| | DNQAKWAVPTTRTDDKLRMETCFQQACKGKIQALCE | |
| | NPEWAPLKDNRIPSYGVLSVDLSLTVELKIKIASGFGPLI | |
| | THGSGMDLYKSNHNNVYWLTIPPMKNLALGVINTLE | |
| | WIPRFKVSPALFNVPIKEAGGDCHAPTYLPAEVDGDVK | |
| | LSSNLVILPGQDLQYVLATYDTSAVEHAVVYYVYSPSR | |
| | SFSYFYPFRLPIKGVPIELQVECFTWDQKLWCRHFCVL | |
| | ADSESGGHITHSGMVGMGVSCTVTREGGGSKGTDDAT | |
| | ADSRKTYTLTDYLKNTYRLKLYSLRWISDHEYLYKQE | |
| | NNILVFNAEYGNSSVFLENSTFDEFGHSINDYSISPDGQ | |
| | FILLEYNYVKQWRHSYTASYDIYDLNKRQLITEERIPNN | |
| | TQWVTWSPVGHKLAYVWNNDIYVKIEPNLPSYRITWT | |
| | GKEDIIYNGITDWVYEEEVFSAYSALWWSPNGTFLAY | |
| | AQFNDTEVPLIEYSFYSDESLQYPKTVRVPYPKAGAVN | |
| | PTVKFFVVNTDSLSSVTNATSIQITAPASMLIGDHYLCD | |
| | VTWATQERISLQWLRRIQNYSVMDICDYDESSGRWNC | |
| | LVARQHIEMSTTGWVGRFRPSEPHFTLDGNSFYKIISNE | |
| | EGYRHICYFQIDKKDCTFITKGTWEVIGIEALTSDYLYY | |
| | ISNEYKGMPGGRNLYKIQLSDYTKVTCLSCELNPERCQ | |
| | YYSVSFSKEAKYYQLRCSGPGLPLYTLHSSVNDKGLRV | |
| | LEDNSALDKMLQNVQMPSKKLDFIILNETKFWYQMILP | |
| | PHFDKSKKYPLLLDVYAGPCSQKADTVFRLNWATYLA | |
| | STENIIVASFDGRGSGYQGDKIMHAINRRLGTFEVEDQI | |
| | EAARQFSKMGFVDNKRIAIWGWSYGGYVTSMVLGSG | |
| | SGVFKCGIAVAPVSRWEYYDSVYTERYMGLPTPEDNL | |
| | DHYRNSTVMSRAENFKQVEYLLIHGTADDNVHFQQSA | |
| | QISKALVDVGVDFQAMWYTDEDHGIASSTAHQHIYTH | |
| | MSHFIKQCFSLPAAARGSGLNDIFEAQKIEWHE | |
| NA75-DPP4 | KGTDDATADSRKTYTLTDYLKNTYRLKLYSLRWISDH | 32 |
| | EYLYKQENNILVFNAEYGNSSVFLENSTFDEFGHSIND | |
| | YSISPDGQFILLEYNYVKQWRHSYTASYDIYDLNKRQL | |
| | ITEERIPNNTQWVTWSPVGHKLAYVWNNDIYVKIEPNL | |
| | PSYRITWTGKEDIIYNGITDWVYEEEVFSAYSALWWSP | |
| | NGTFLAYAQFNDTEVPLIEYSFYSDESLQYPKTVRVPY | |
| | PKAGAVNPTVKFFVVNTDSLSSVTNATSIQITAPASMLI | |
| | GDHYLCDVTWATQERISLQWLRRIQNYSVMDICDYDE | |
| | SSGRWNCLVARQHIEMSTTGWVGRFRPSEPHFTLDGN | |
| | SFYKIISNEEGYRHICYFQIDKKDCTFITKGTWEVIGIEA | |
| | LTSDYLYYISNEYKGMPGGRNLYKIQLSDYTKVTCLSC | |
| | ELNPERCQYYSVSFSKEAKYYQLRCSGPGLPLYTLHSS | |
| | VNDKGLRVLEDNSALDKMLQNVQMPSKKLDFIILNET | |
| | KFWYQMILPPHFDKSKKYPLLLDVYAGPCSQKADTVF | |
| | RLNWATYLASTENIIVASFDGRGSGYQGDKIMHAINRR | |
| | LGTFEVEDQIEAARQFSKMGFVDNKRIAIWGWSYGGY | |
| | VTSMVLGSGSGVFKCGIAVAPVSRWEYYDSVYTERYM | |
| | GLPTPEDNLDHYRNSTVMSRAENFKQVEYLLIHGTAD | |
| | DNVHFQQSAQISKALVDVGVDFQAMWYTDEDHGIASS | |
| | TAHQHIYTHMSHFIKQCFSLP | |
| H374A and | MSSSSWLLLSLVAVTAAQSTIEEQAKTFLDKFNHEAED | 33 |
| H378A | LFYQSSLASWNYNTNITEENVQNMNNAGDKWSAFLK | |
| (H2A)/ACE2- | EQSTLAQMYPLQEIQNLTVKLQLQALQQNGSSVLSED | |
| VGTM | KSKRLNTILNTMSTIYSTGKVCNPDNPQECLLLEPGLNE | |
| | IMANSLDYNERLWAWESWRSEVGKQLRPLYEEYVVL | |
| | KNEMARANHYEDYGDYWRGDYEVNGVDGYDYSRGQ | |
| | LIEDVEHTFEEIKPLYEHLHAYVRAKLMNAYPSYISPIG | |
| | CLPAHLLGDMWGRFWTNLYSLTVPFGQKPNIDVTDA | |
| | MVDQAWDAQRIFKEAEKFFVSVGLPNMTQGFWENSM | |
| | LTDPGNVQKAVCHPTAWDLGKGDFRILMCTKVTMDD | |
| | FLTAHAEMGAIQYDMAYAAQPFLLRNGANEGFHEAV | |
| | GEIMSLSAATPKHLKSIGLLSPDFQEDNETEINFLLKQA | |
| | LTIVGTLPFTYMLEKWRWMVFKGEIPKDQWMKKWW | |
| | EMKREIVGVVEPVPHDETYCDPASLFHVSNDYSFIRYY | |
| | TRTLYQFQFQEALCQAAKHEGPLHKCDISNSTEAGQKL | |
| | FNMLRLGKSEPWTLALENVVGAKNMNVRPLLNYFEPL | |
| | FTWLKDQNKNSFVGWSTDWSPYADQSIKVRISLKSAL | |
| | GDKAYEWNDNEMYLFRSSVAYAMRQYFLKVKNQMIL | |
| | FGEEDVRVANLKPRISFNFFVTAPKNVSDIIPRTEVEKAI | |
| | RMSRSRINDAFRLNDNSLEFLGIQPTLGPPNQPPVSSRG | |
| | MLDSDLHLSSKAQVFEHPHIQDAASQLPDDESLFFGDT | |
| | GLSKNPIELVEGWFSSWKSSIASFFFIIGLIIGLFLVLRVG | |
| | IHLCIKLKHTKKRQIYTDIEMNRLGK | |
| WT/ACE2- | MSSSSWLLLSLVAVTAAQSTIEEQAKTFLDKFNHEAED | 34 |
| D4VG | LFYQSSLASWNYNTNITEENVQNMNNAGDKWSAFLK | |
| | EQSTLAQMYPLQEIQNLTVKLQLQALQQNGSSVLSED | |
| | KSKRLNTILNTMSTIYSTGKVCNPDNPQECLLLEPGLNE | |
| | IMANSLDYNERLWAWESWRSEVGKQLRPLYEEYVVL | |
| | KNEMARANHYEDYGDYWRGDYEVNGVDGYDYSRGQ | |
| | LIEDVEHTFEEIKPLYEHLHAYVRAKLMNAYPSYISPIG | |
| | CLPAHLLGDMWGRFWTNLYSLTVPFGQKPNIDVTDA | |
| | MVDQAWDAQRIFKEAEKFFVSVGLPNMTQGFWENSM | |
| | LTDPGNVQKAVCHPTAWDLGKGDFRILMCTKVTMDD | |
| | FLTAHHEMGHIQYDMAYAAQPFLLRNGANEGFHEAV | |
| | GEIMSLSAATPKHLKSIGLLSPDFQEDNETEINFLLKQA | |
| | LTIVGTLPFTYMLEKWRWMVFKGEIPKDQWMKKWW | |
| | EMKREIVGVVEPVPHDETYCDPASLFHVSNDYSFIRYY | |
| | TRTLYQFQFQEALCQAAKHEGPLHKCDISNSTEAGQKL | |
| | FNMLRLGKSEPWTLALENVVGAKNMNVRPLLNYFEPL | |
| | FTWLKDQNKNSFVGWSTDWSPYADQSIKVRISLKSAL | |
| | GDKAYEWNDNEMYLFRSSVAYAMRQYFLKVKNQMIL | |
| | FGEEDVRVANLKPRISFNFFVTAPKNVSDIIPRTEVEKAI | |
| | RMSRSRINDAFRLNDNSLEFLGIQPTLGPPNQPPVSSRIQ | |
| | ADGWMCHASKWVTTCDFRWYGPKYITHSIRSFTPSVE | |
| | QCKESIEQTKQGTWLNPGFPPQSCGYATVIDAEAVIVQ | |
| | VTPHHVLVDEYTGEWVDSQFINGKCSNYICPTVHNSTT | |
| | WHSDYKVKGLCDSNLGMLDSDLHLSSKAQVFEHPHIQ | |
| | DAASQLPDDESLFFGDTGLSKNPIELVEGWFSSWKSSIA | |
| | SFFFIIGLIIGLFLVLRVGIHLCIKLKHTKKRQIYTDIEMN | |
| | RLGK | |
| H2A/ACE2- | MSSSSWLLLSLVAVTAAQSTIEEQAKTFLDKFNHEAED | 35 |
| D4VG | LFYQSSLASWNYNTNITEENVQNMNNAGDKWSAFLK | |
| | EQSTLAQMYPLQEIQNLTVKLQLQALQQNGSSVLSED | |
| | KSKRLNTILNTMSTIYSTGKVCNPDNPQECLLLEPGLNE | |
| | IMANSLDYNERLWAWESWRSEVGKQLRPLYEEYVVL | |
| | KNEMARANHYEDYGDYWRGDYEVNGVDGYDYSRGQ | |
| | LIEDVEHTFEEIKPLYEHLHAYVRAKLMNAYPSYISPIG | |
| | CLPAHLLGDMWGRFWTNLYSLTVPFGQKPNIDVTDA | |
| | MVDQAWDAQRIFKEAEKFFVSVGLPNMTQGFWENSM | |
| | LTDPGNVQKAVCHPTAWDLGKGDFRILMCTKVTMDD | |
| | FLTAHAEMGAIQYDMAYAAQPFLLRNGANEGFHEAV | |
| | GEIMSLSAATPKHLKSIGLLSPDFQEDNETEINFLLKQA | |
| | LTIVGTLPFTYMLEKWRWMVFKGEIPKDQWMKKWW | |
| | EMKREIVGVVEPVPHDETYCDPASLFHVSNDYSFIRYY | |
| | TRTLYQFQFQEALCQAAKHEGPLHKCDISNSTEAGQKL | |
| | FNMLRLGKSEPWTLALENVVGAKNMNVRPLLNYFEPL | |
| | FTWLKDQNKNSFVGWSTDWSPYADQSIKVRISLKSAL | |
| | GDKAYEWNDNEMYLFRSSVAYAMRQYFLKVKNQMIL | |
| | FGEEDVRVANLKPRISFNFFVTAPKNVSDIIPRTEVEKAI | |
| | RMSRSRINDAFRLNDNSLEFLGIQPTLGPPNQPPVSSRIQ | |
| | ADGWMCHASKWVTTCDFRWYGPKYITHSIRSFTPSVE | |
| | QCKESIEQTKQGTWLNPGFPPQSCGYATVIDAEAVIVQ | |
| | VTPHHVLVDEYTGEWVDSQFINGKCSNYICPTVHNSTT | |
| | WHSDYKVKGLCDSNLGMLDSDLHLSSKAQVFEHPHIQ | |
| | DAASQLPDDESLFFGDTGLSKNPIELVEGWFSSWKSSIA | |
| | SFFFIIGLIIGLFLVLRVGIHLCIKLKHTKKRQIYTDIEMN | |
| | RLGK | |

In some embodiments, the fusion protein or second fusion protein comprises an amino acid sequence of at least 7500 sequence identity to an amino acid sequence according to SEQ ID NO: 29. In some embodiments, the fusion protein or second fusion protein comprises an amino acid sequence of at least 800% sequence identity to an amino acid sequence according to SEQ ID NO: 29. In some embodiments, the fusion protein or second fusion protein comprises an amino acid sequence of at least 85% sequence identity to an amino acid sequence according to SEQ ID NO: 29. In some embodiments, the fusion protein or second fusion protein comprises an amino acid sequence of at least 90% sequence identity to an amino acid sequence according to SEQ ID NO: 29. In some embodiments, the fusion protein or second fusion protein comprises an amino acid sequence of at least 9500 sequence identity to an amino acid sequence according to SEQ ID NO: 29. In some embodiments, the fusion protein or second fusion protein comprises an amino acid sequence of at least 98% sequence identity to an amino acid sequence according to SEQ ID NO: 29. In some embodiments, the fusion protein or second fusion protein comprises an amino acid sequence of at least 99% sequence identity to an amino acid sequence according to SEQ ID NO: 29.

In some embodiments, the fusion protein or second fusion protein comprises an amino acid sequence of at least 75% sequence homology to an amino acid sequence according to SEQ ID NO: 29. In some embodiments, the fusion protein or second fusion protein comprises an amino acid sequence of at least 80% sequence homology to an amino acid sequence according to SEQ ID NO: 29. In some embodiments, the fusion protein or second fusion protein comprises an amino acid sequence of at least 85% sequence homology to an amino acid sequence according to SEQ ID NO: 29. In some embodiments, the fusion protein or second fusion protein comprises an amino acid sequence of at least 90% sequence homology to an amino acid sequence according to SEQ ID NO: 29. In some embodiments, the fusion protein or second fusion protein comprises an amino acid sequence of at least 95% sequence homology to an amino acid sequence according to SEQ ID NO: 29. In some embodiments, the fusion protein or second fusion protein comprises an amino acid sequence of at least 98% sequence homology to an amino acid sequence according to SEQ ID NO: 29. In some embodiments, the fusion protein or second fusion protein comprises an amino acid sequence of at least 99% sequence homology to an amino acid sequence according to SEQ ID NO: 29.

In some instances, the fusion protein or second fusion protein comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, or more than 720 consecutive amino acids of SEQ ID NO: 29.

In some embodiments, the fusion protein or second fusion protein comprises an amino acid sequence of at least 75% sequence identity to an amino acid sequence according to SEQ ID NO: 30. In some embodiments, the fusion protein or second fusion protein comprises an amino acid sequence of at least 80% sequence identity to an amino acid sequence according to SEQ ID NO: 30. In some embodiments, the fusion protein or second fusion protein comprises an amino acid sequence of at least 85% sequence identity to an amino acid sequence according to SEQ ID NO: 30. In some embodiments, the fusion protein or second fusion protein comprises an amino acid sequence of at least 90% sequence identity to an amino acid sequence according to SEQ ID NO: 30. In some embodiments, the fusion protein or second fusion protein comprises an amino acid sequence of at least 95% sequence identity to an amino acid sequence according to SEQ ID NO: 30. In some embodiments, the fusion protein or second fusion protein comprises an amino acid sequence of at least 98% sequence identity to an amino acid sequence according to SEQ ID NO: 30. In some embodiments, the fusion protein or second fusion protein comprises an amino acid sequence of at least 99% sequence identity to an amino acid sequence according to SEQ ID NO: 30.

In some embodiments, the fusion protein or second fusion protein comprises an amino acid sequence of at least 75% sequence homology to an amino acid sequence according to SEQ ID NO: 30. In some embodiments, the fusion protein or second fusion protein comprises an amino acid sequence of at least 80% sequence homology to an amino acid sequence according to SEQ ID NO: 30. In some embodiments, the fusion protein or second fusion protein comprises an amino acid sequence of at least 85% sequence homology to an amino acid sequence according to SEQ ID NO: 30. In some embodiments, the fusion protein or second fusion protein comprises an amino acid sequence of at least 90% sequence homology to an amino acid sequence according to SEQ ID NO: 30. In some embodiments, the fusion protein or second fusion protein comprises an amino acid sequence of at least 95% sequence homology to an amino acid sequence according to SEQ ID NO: 30. In some embodiments, the fusion protein or second fusion protein comprises an amino acid sequence of at least 98% sequence homology to an amino acid sequence according to SEQ ID NO: 30. In some embodiments, the fusion protein or second fusion protein comprises an amino acid sequence of at least 99% sequence homology to an amino acid sequence according to SEQ ID NO: 30.

In some instances, the fusion protein or second fusion protein comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, or more than 720 consecutive amino acids of SEQ ID NO: 30.

In some embodiments, the fusion protein or second fusion protein comprises an amino acid sequence of at least 75% sequence identity to an amino acid sequence according to SEQ ID NO: 31. In some embodiments, the fusion protein or second fusion protein comprises an amino acid sequence of at least 80% sequence identity to an amino acid sequence according to SEQ ID NO: 31. In some embodiments, the fusion protein or second fusion protein comprises an amino acid sequence of at least 85% sequence identity to an amino acid sequence according to SEQ ID NO: 31. In some embodiments, the fusion protein or second fusion protein comprises an amino acid sequence of at least 90% sequence identity to an amino acid sequence according to SEQ ID NO: 31. In some embodiments, the fusion protein or second fusion protein comprises an amino acid sequence of at least 95% sequence identity to an amino acid sequence according to SEQ ID NO: 31. In some embodiments, the fusion protein or second fusion protein comprises an amino acid sequence of at least 98% sequence identity to an amino acid sequence according to SEQ ID NO: 31. In some embodiments, the fusion protein or second fusion protein comprises an amino acid sequence of at least 99% sequence identity to an amino acid sequence according to SEQ ID NO: 31.

In some embodiments, the fusion protein or second fusion protein comprises an amino acid sequence of at least 75% sequence homology to an amino acid sequence according to SEQ ID NO: 31. In some embodiments, the fusion protein or second fusion protein comprises an amino acid sequence of at least 80% sequence homology to an amino acid sequence according to SEQ ID NO: 31. In some embodiments, the fusion protein or second fusion protein comprises an amino acid sequence of at least 85% sequence homology to an amino acid sequence according to SEQ ID NO: 31. In some embodiments, the fusion protein or second fusion protein comprises an amino acid sequence of at least 90% sequence homology to an amino acid sequence according to SEQ ID NO: 31. In some embodiments, the fusion protein or second fusion protein comprises an amino acid sequence of at least 95% sequence homology to an amino acid sequence according to SEQ ID NO: 31. In some embodiments, the fusion protein or second fusion protein comprises an amino acid sequence of at least 98% sequence homology to an amino acid sequence according to SEQ ID NO: 31. In some embodiments, the fusion protein or second fusion protein comprises an amino acid sequence of at least 99% sequence homology to an amino acid sequence according to SEQ ID NO: 31.

In some instances, the fusion protein or second fusion protein comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, or more than 720 consecutive amino acids of SEQ ID NO: 31.

In some embodiments, the fusion protein or second fusion protein comprises an amino acid sequence of at least 75% sequence identity to an amino acid sequence according to SEQ ID NO: 32. In some embodiments, the fusion protein or second fusion protein comprises an amino acid sequence of at least 80% sequence identity to an amino acid sequence according to SEQ ID NO: 32. In some embodiments, the fusion protein or second fusion protein comprises an amino acid sequence of at least 85% sequence identity to an amino acid sequence according to SEQ ID NO: 32. In some embodiments, the fusion protein or second fusion protein comprises an amino acid sequence of at least 90% sequence identity to an amino acid sequence according to SEQ ID NO: 32. In some embodiments, the fusion protein or second fusion protein comprises an amino acid sequence of at least 95% sequence identity to an amino acid sequence according to SEQ ID NO: 32. In some embodiments, the fusion protein or second fusion protein comprises an amino acid sequence of at least 98% sequence identity to an amino acid sequence according to SEQ ID NO: 32. In some embodiments, the fusion protein or second fusion protein comprises an amino acid sequence of at least 99% sequence identity to an amino acid sequence according to SEQ ID NO: 32.

In some embodiments, the fusion protein or second fusion protein comprises an amino acid sequence of at least 75% sequence homology to an amino acid sequence according to SEQ ID NO: 32. In some embodiments, the fusion protein or second fusion protein comprises an amino acid sequence of at least 80% sequence homology to an amino acid sequence according to SEQ ID NO: 32. In some embodiments, the fusion protein or second fusion protein comprises an amino acid sequence of at least 85% sequence homology to an amino acid sequence according to SEQ ID NO: 32. In some embodiments, the fusion protein or second fusion protein comprises an amino acid sequence of at least 90% sequence homology to an amino acid sequence according to SEQ ID NO: 32. In some embodiments, the fusion protein or second fusion protein comprises an amino acid sequence of at least 95% sequence homology to an amino acid sequence according to SEQ ID NO: 32. In some embodiments, the fusion protein or second fusion protein comprises an amino acid sequence of at least 98% sequence homology to an amino acid sequence according to SEQ ID NO: 32. In some embodiments, the fusion protein or second fusion protein comprises an amino acid sequence of at least 99% sequence homology to an amino acid sequence according to SEQ ID NO: 32.

In some instances, the fusion protein or second fusion protein comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, or more than 720 consecutive amino acids of SEQ ID NO: 32.

In some embodiments, the fusion protein or second fusion protein comprises an amino acid sequence of at least 75% sequence identity to an amino acid sequence according to SEQ ID NO: 33. In some embodiments, the fusion protein or second fusion protein comprises an amino acid sequence of at least 80% sequence identity to an amino acid sequence according to SEQ ID NO: 33. In some embodiments, the fusion protein or second fusion protein comprises an amino acid sequence of at least 85% sequence identity to an amino acid sequence according to SEQ ID NO: 33. In some embodiments, the fusion protein or second fusion protein comprises an amino acid sequence of at least 90% sequence identity to an amino acid sequence according to SEQ ID NO: 33. In some embodiments, the fusion protein or second fusion protein comprises an amino acid sequence of at least 95% sequence identity to an amino acid sequence according to SEQ ID NO: 33. In some embodiments, the fusion protein or second fusion protein comprises an amino acid sequence of at least 98% sequence identity to an amino acid sequence according to SEQ ID NO: 33. In some embodiments, the fusion protein or second fusion protein comprises an amino acid sequence of at least 99% sequence identity to an amino acid sequence according to SEQ ID NO: 33.

In some embodiments, the fusion protein or second fusion protein comprises an amino acid sequence of at least 75% sequence homology to an amino acid sequence according to SEQ ID NO: 33. In some embodiments, the fusion protein or second fusion protein comprises an amino acid sequence of at least 80% sequence homology to an amino acid sequence according to SEQ ID NO: 33. In some embodiments, the fusion protein or second fusion protein comprises an amino acid sequence of at least 85% sequence homology to an amino acid sequence according to SEQ ID NO: 33. In some embodiments, the fusion protein or second fusion protein comprises an amino acid sequence of at least 90% sequence homology to an amino acid sequence according to SEQ ID NO: 33. In some embodiments, the fusion protein or second fusion protein comprises an amino acid sequence of at least 95% sequence homology to an amino acid sequence according to SEQ ID NO: 33. In some embodiments, the fusion protein or second fusion protein comprises an amino acid sequence of at least 98% sequence homology to an amino acid sequence according to SEQ ID NO: 33. In some embodiments, the fusion protein or second fusion protein comprises an amino acid sequence of at least 99% sequence homology to an amino acid sequence according to SEQ ID NO: 33.

In some instances, the fusion protein or second fusion protein comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, or more than 720 consecutive amino acids of SEQ ID NO: 33.

In some embodiments, the fusion protein or second fusion protein comprises an amino acid sequence of at least 75% sequence identity to an amino acid sequence according to SEQ ID NO: 34. In some embodiments, the fusion protein or second fusion protein comprises an amino acid sequence of at least 80% sequence identity to an amino acid sequence according to SEQ ID NO: 34. In some embodiments, the fusion protein or second fusion protein comprises an amino acid sequence of at least 85% sequence identity to an amino acid sequence according to SEQ ID NO: 34. In some embodiments, the fusion protein or second fusion protein comprises an amino acid sequence of at least 90% sequence identity to an amino acid sequence according to SEQ ID NO: 34. In some embodiments, the fusion protein or second fusion protein comprises an amino acid sequence of at least 95% sequence identity to an amino acid sequence according to SEQ ID NO: 34. In some embodiments, the fusion protein or second fusion protein comprises an amino acid sequence of at least 98% sequence identity to an amino acid sequence according to SEQ ID NO: 34. In some embodiments, the fusion protein or second fusion protein comprises an amino acid sequence of at least 99% sequence identity to an amino acid sequence according to SEQ ID NO: 34.

In some embodiments, the fusion protein or second fusion protein comprises an amino acid sequence of at least 75% sequence homology to an amino acid sequence according to SEQ ID NO: 34. In some embodiments, the fusion protein or second fusion protein comprises an amino acid sequence of at least 80% sequence homology to an amino acid sequence according to SEQ ID NO: 34. In some embodiments, the fusion protein or second fusion protein comprises an amino acid sequence of at least 85% sequence homology to an amino acid sequence according to SEQ ID NO: 34. In some embodiments, the fusion protein or second fusion protein comprises an amino acid sequence of at least 90% sequence homology to an amino acid sequence according to SEQ ID NO: 34. In some embodiments, the fusion protein or second fusion protein comprises an amino acid sequence of at least 95% sequence homology to an amino acid sequence according to SEQ ID NO: 34. In some embodiments, the fusion protein or second fusion protein comprises an amino acid sequence of at least 98% sequence homology to an amino acid sequence according to SEQ ID NO: 34. In some embodiments, the fusion protein or second fusion protein comprises an amino acid sequence of at least 99% sequence homology to an amino acid sequence according to SEQ ID NO: 34.

In some instances, the fusion protein or second fusion protein comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, or more than 720 consecutive amino acids of SEQ ID NO: 34.

In some embodiments, the fusion protein or second fusion protein comprises an amino acid sequence of at least 75% sequence identity to an amino acid sequence according to SEQ ID NO: 35. In some embodiments, the fusion protein or second fusion protein comprises an amino acid sequence of at least 80% sequence identity to an amino acid sequence according to SEQ ID NO: 35. In some embodiments, the fusion protein or second fusion protein comprises an amino acid sequence of at least 85% sequence identity to an amino acid sequence according to SEQ ID NO: 35. In some embodiments, the fusion protein or second fusion protein comprises an amino acid sequence of at least 90% sequence identity to an amino acid sequence according to SEQ ID NO: 35. In some embodiments, the fusion protein or second fusion protein comprises an amino acid sequence of at least 95% sequence identity to an amino acid sequence according to SEQ ID NO: 35. In some embodiments, the fusion protein or second fusion protein comprises an amino acid sequence of at least 98% sequence identity to an amino acid sequence according to SEQ ID NO: 35. In some embodiments, the fusion protein or second fusion protein comprises an amino acid sequence of at least 99% sequence identity to an amino acid sequence according to SEQ ID NO: 35.

In some embodiments, the fusion protein or second fusion protein comprises an amino acid sequence of at least 75% sequence homology to an amino acid sequence according to SEQ ID NO: 35. In some embodiments, the fusion protein or second fusion protein comprises an amino acid sequence of at least 80% sequence homology to an amino acid sequence according to SEQ ID NO: 35. In some embodiments, the fusion protein or second fusion protein comprises an amino acid sequence of at least 85% sequence homology to an amino acid sequence according to SEQ ID NO: 35. In some embodiments, the fusion protein or second fusion protein comprises an amino acid sequence of at least 90% sequence homology to an amino acid sequence according to SEQ ID NO: 35. In some embodiments, the fusion protein or second fusion protein comprises an amino acid sequence of at least 95% sequence homology to an amino acid sequence according to SEQ ID NO: 35. In some embodiments, the fusion protein or second fusion protein comprises an amino acid sequence of at least 98% sequence homology to an amino acid sequence according to SEQ ID NO: 35. In some embodiments, the fusion protein or second fusion protein comprises an amino acid sequence of at least 99% sequence homology to an amino acid sequence according to SEQ ID NO: 35.

In some instances, the fusion protein or second fusion protein comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, or more than 720 consecutive amino acids of SEQ ID NO: 35.

### Compositions for Generation of Multivalent Particles

Described herein, in some embodiments, are compositions comprising a multivalent particle comprising a fusion protein that comprises a mammalian polypeptide that binds to a viral protein and a transmembrane polypeptide. In some embodiments, the compositions comprise a first nucleic acid sequence encoding the multivalent particle described herein.

Compositions for generating multivalent particles, in some embodiments, further comprise a second nucleic acid sequence that encodes one or more packaging viral proteins. In some embodiments, the one or more packaging viral proteins is a lentiviral protein, a retroviral protein, an adenoviral protein, or combinations thereof. In some embodiments, the one or more packaging viral proteins comprises gag, pol, pre, tat, rev, or combinations thereof.

Compositions for generating multivalent particles, in some embodiments, further comprise a second nucleic acid sequence that encodes an expression construct for specifically targeting the mammalian polypeptide to the surface of an extracellular vesicle. In some embodiments, the second nucleic acid sequence encodes an expression construct for specifically targeting the mammalian polypeptide to the surface of an exosome. In some embodiments, the second nucleic acid sequence encodes an expression construct for specifically targeting the mammalian polypeptide to the surface of an ectosome.

Compositions for generating multivalent particles, in some embodiments, further comprise a third nucleic acid sequence that encodes a replication incompetent viral genome, a reporter, a therapeutic molecule, or combinations thereof. In some embodiments, compositions can further comprise a third nucleic acid sequence that encodes a replication competent viral genome, a reporter, a therapeutic molecule, or combinations thereof. In some embodiments, the viral genome is derived from vesicular stomatitis virus, measles virus, Hepatitis virus, influenza virus, or combinations thereof.

In some embodiments, the reporter protein is a fluorescent protein or an enzyme. Exemplary reporter genes include, but are not limited to, acetohydroxyacid synthase (AHAS), alkaline phosphatase (AP), beta galactosidase (LacZ), beta glucuronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination. In some embodiments, the reporter is a fluorescent protein. In some embodiments, the fluorescent protein is green fluorescent protein. In some embodiments, the reporter protein emits green fluorescence, yellow fluorescence, or red fluorescence. In some embodiments, the reporter is an enzyme. In some embodiments, the enzyme is β-galactosidase, alkaline phosphatase, β-lactamase, or luciferase.

In some embodiments, the therapeutic molecule is an immune modulating protein, a cellular signal modulating molecule, a proliferation modulating molecule, a cell death modulating molecule, or combinations thereof. In some embodiments, the therapeutic molecule is an immune checkpoint molecule. Exemplary immune checkpoint molecules include, but are not limited to, CTLA4, PD1, OX40, and CD28. In some embodiments, the therapeutic molecule is an inflammatory cytokine. In some embodiments, the inflammatory cytokine comprises IL-1, IL-12, or IL-18. In some embodiments, the therapeutic molecule is a proliferation cytokine. In some embodiments, the proliferation cytokine comprises IL-4, IL-7, or IL-15. In some embodiments, the cell death molecule comprises Fas or a death receptor.

Compositions for generating multivalent particles, in some embodiments, further comprise a fourth nucleic acid sequence encoding a second fusion protein that comprises a second mammalian polypeptide and a second transmembrane polypeptide that binds to the viral protein as described herein.

In some embodiments, the first nucleic acid sequence, the second nucleic acid sequence, and the third nucleic acid sequence are within a same vector. In some embodiments, the first nucleic acid sequence, the second nucleic acid sequence, and the third nucleic acid sequence are within different vectors. In some embodiments, the first nucleic acid sequence, the second nucleic acid sequence, the third nucleic acid sequence, and the fourth nucleic acid sequence are within a same vector. In some embodiments, the first nucleic acid sequence, the second nucleic acid sequence, third nucleic acid sequence, and the fourth nucleic acid sequence are within different vectors.

Various vectors, in some embodiments, are used herein. In some embodiments, the vector is a eukaryotic or prokaryotic vector. In some embodiments, the vector is a viral vector. In some embodiments, the vector is a lentivirus vector, an adenovirus vector, or an adeno-associated virus vector. Exemplary vectors include, without limitation, mammalian expression vectors: pSF-CMV-NEO-NH2-PPT-3 XFLAG, pSF-CMV-NEO-COOH-3×FLAG, pSF-CMV-PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEF1a-mCherry-N1 Vector, pEF1a-tdTomato Vector, pSF-CMV-FMDV-Hygro, pSF-CMV-PGK-Puro, pMCP-tag(m), and pSF-CMV-PURO-NH2-CMYC; bacterial expression vectors: pSF-OXB20-BetaGal, pSF-OXB20-Fluc, pSF-OXB20, and pSF-Tac; plant expression vectors: pRI 101-ANDNA and pCambia2301; and yeast expression vectors: pTYB21 and pKLAC2, and insect vectors: pAc5.1/V5-His A and pDEST8.

### Compositions and Pharmaceutical Compositions

Described herein, in some embodiments, are compositions comprising a multivalent particle comprising a fusion protein that comprises a mammalian polypeptide that binds to a viral protein and a transmembrane polypeptide. Described herein, in some embodiments, are pharmaceutical compositions comprising a multivalent particle comprising a fusion protein that comprises a mammalian polypeptide that binds to a viral protein and a transmembrane polypeptide.

For administration to a subject, the multivalent particles as disclosed herein, may be provided in a pharmaceutical composition together with one or more pharmaceutically acceptable carriers or excipients. In some embodiments, the multivalent particles as disclosed herein, may be provided in a composition together with one or more carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, intravenous, intranasal delivery, or inhalation) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Methods of Use

Multivalent particles described herein, in some embodiments, are used to treat a viral infection. In some instances, the viral infection is caused by SARS-CoV-1. In some instances, the viral infection is caused by SARS-CoV-2. In some instances, the viral infection is caused by MERS-CoV. In some instances, the viral infection is caused by respiratory syncytial virus. In some instances, the viral infection is caused by HBV/HDV. In some instances, the viral infection is caused by HIV.

In some instances, the subject is a mammal. In some instances, the subject is a mouse, rabbit, dog, pig, cattle, or human. Subjects treated by methods described herein may be infants, adults, or children. Pharmaceutical compositions or compositions comprising multivalent particles as described herein may be administered intravenously, subcutaneously, intranasally, or inhalation. In some embodiments, the multivalent particle is administered intravenously. In some embodiments, the multivalent particle is administered through intranasal delivery. In some embodiments, the multivalent particle is administered through inhalation. In some embodiments, the multivalent particle is administered by an intraperitoneal injection. In some embodiments, the multivalent particle is administered by a subcutaneous injection.

Described herein, in some embodiments, are methods of treating an infection in a subject in need thereof comprising administering to the subject a multivalent particle described herein. In some embodiments, the infection comprises infection by SARS-CoV-1, SARS-CoV-2, MERS-CoV, respiratory syncytial virus, HBV, HDV, HIV, or combinations thereof. In some embodiments, the infection comprises infection by SARS-CoV-1. In some embodiments, the infection comprises infection by SARS-CoV-2. In some embodiments, the infection comprises infection by MERS-CoV.

In some embodiments, the multivalent particle is administered to the subject through inhalation. In some embodiments, the multivalent particle is administered to the subject through intranasal delivery. In some embodiments, the multivalent particle is administered to the subject through intratracheal delivery. In some embodiments, the multivalent particle is administered to the subject by an intraperitoneal injection. In some embodiments, the multivalent particle is administered to the subject by a subcutaneous injection. In some embodiments, the administering to the subject of the multivalent particle is sufficient to reduce or eliminate the infection as compared to a baseline measurement of the infection taken from the subject prior to the administering of the multivalent particle. In some embodiments, the reduction is at least about 1-fold, 5-fold, 10-fold, 20-fold, 40-fold, 60-fold, 80-fold, or up to about 100-fold.

Described herein, in some embodiments, are methods of treating an infection in a subject in need thereof comprising administering to the subject a composition, wherein the composition comprises a nucleic acid sequence that encodes a first fusion protein disclosed herein. Described herein, in some embodiments, are methods of treating an infection in a subject in need thereof comprising administering to the subject a composition, wherein the composition comprises a nucleic acid sequence that encodes a first fusion protein disclosed herein and a second fusion protein disclosed herein. In some embodiments, the infection comprises infection by SARS-CoV-1, SARS-CoV-2, MERS-CoV, respiratory syncytial virus, HBV, HDV, HIV, or combinations thereof. In some embodiments, the nucleic acid sequence comprises mRNA. In some embodiments, the nucleic acid sequence comprises DNA.

In some embodiments, the composition is administered to the subject through inhalation. In some embodiments, the composition is administered to the subject through intranasal delivery. In some embodiments, the composition is administered to the subject through intratracheal delivery. In some embodiments, the composition is administered to the subject by an intraperitoneal injection. In some embodiments, the composition is administered to the subject by a subcutaneous injection. In some embodiments, the administering to the subject of the composition is sufficient to reduce or eliminate the infection as compared to a baseline measurement of the infection taken from the subject prior to the administering of the composition. In some embodiments, the reduction is at least about 1-fold, 5-fold, 10-fold, 20-fold, 40-fold, 60-fold, 80-fold, or up to about 100-fold.

In some embodiments, the mRNAs that encode the first fusion protein and the second fusion protein and the second nucleic acid sequence and the third nucleic acid sequence are administered inhalation. In some embodiments, the mRNAs that encode the first fusion protein and the second fusion protein and the second nucleic acid sequence an d the third nucleic acid sequence are administered through intranasal delivery. In some embodiments, the mRNAs that encode the first fusion protein and the second fusion protein and the second nucleic acid sequence and the third nucleic acid sequence are administered intratracheal delivery. In some embodiments, the mRNAs that encode the first fusion protein and the second fusion protein and the second nucleic acid sequence and the third nucleic acid sequence are administered by an intraperitoneal injection. In some embodiments, the mRNAs that encode the first fusion protein and the second fusion protein and the second nucleic acid sequence and the third nucleic acid sequence are administered by a subcutaneous injection. In some embodiments, the administering to the subject of the composition is sufficient to reduce or eliminate the infection as compared to a baseline measurement of the infection taken from the subject prior to the administering of the composition. In some embodiments, the reduction is at least about 1-fold, 5-fold, 10-fold, 20-fold, 40-fold, 60-fold, 80-fold, or up to about 100-fold.

In some embodiments, the DNAs that encode the first fusion protein and the second fusion protein and the second nucleic acid sequence and the third nucleic acid sequence are administered inhalation. In some embodiments, the DNAs that encode the first fusion protein and the second fusion protein and the second nucleic acid sequence and the third nucleic acid sequence are administered through intranasal delivery. In some embodiments, the DNAs that encode the first fusion protein and the second fusion protein and the second nucleic acid sequence and the third nucleic acid sequence are administered intratracheal delivery. In some embodiments, the DNAs that encode the first fusion protein and the second fusion protein and the second nucleic acid sequence and the third nucleic acid sequence are administered by an intraperitoneal injection. In some embodiments, the DNAs that encode the first fusion protein and the second fusion protein and the second nucleic acid sequence and the third nucleic acid sequence are administered by a subcutaneous injection. In some embodiments, the administering to the subject of the composition is sufficient to reduce or eliminate the infection as compared to a baseline measurement of the infection taken from the subject prior to the administering of the composition. In some embodiments, the reduction is at least about 1-fold, 5-fold, 10-fold, 20-fold, 40-fold, 60-fold, 80-fold, or up to about 100-fold.

In some embodiments, the composition comprises a liposome. In some embodiments, the liposome comprises a protamine liposome. In some embodiments, the liposome comprises a cationic polymer liposome. In some embodiments, the composition comprises a lipid nanoparticle. In some embodiments, the composition comprises a cationic lipid nanoparticle. In some embodiments, the composition comprises a cationic lipid, cholesterol nanoparticle. In some embodiments, the composition comprises a cationic lipid, cholesterol, PEG nanoparticle. In some embodiments, the composition comprises with a dendrimer nanoparticle.

In some embodiments, the composition comprises an adeno-associated virus (AAV). In some embodiments, the composition comprises a polymer. In some embodiments, the composition comprises protamine. In some embodiments, the composition comprises polysaccharide particle. In some embodiments, the composition comprises a cationic polymer.

In some embodiments, the composition comprises a cationic nano-emulsion. In some embodiments, the composition comprises a transfection reagent. In some embodiments, the composition comprises a dendritic cell.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Examples

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Design and Function of ACE2-Antivirus Mirroring Viral Multivalence

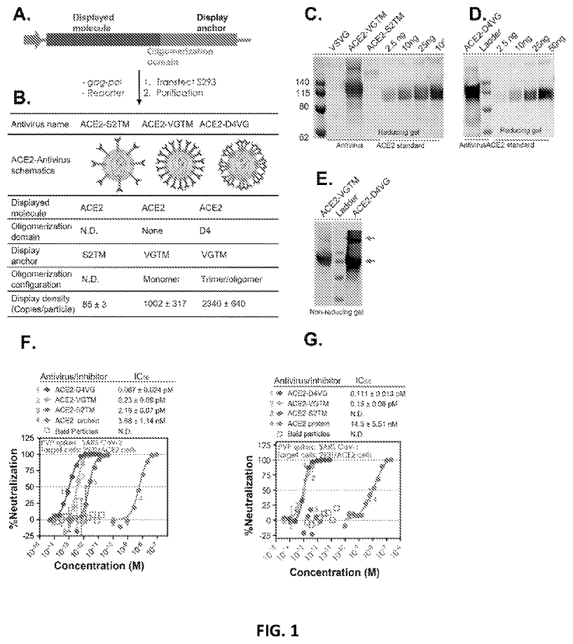

FIG. 1A-1G. Design and function of ACE2-Antiviruses. FIG. 1A illustrates a schematic of the display vectors used for pseudotyping lentivirus-based viral-like particles. FIG. 1B illustrates the design of display vectors and their corresponding multivalence and oligomerization features of the ACE2-antiviruses. FIG. 1C shows a quantitative Western-blog analysis of ACE2-S2TM antiviruses under reducing conditions. FIG. 1D shows a quantitative Western-blog analysis of ACE2-VGTM antiviruses under reducing conditions. FIG. 1E shows a Western-blot analysis of ACE2-VGTM and ACE2-D4VG antiviruses under non-reducing conditions. FIG. 1F shows the neutralizing activities of ACE2 recombinant protein and ACE2-antiviruses against SARS CoV-2 pseudovirus. FIG. 1G shows the neutralizing activities of ACE2 recombinant protein and ACE2-antiviruses against SARS CoV-1 pseudovirus.

To generate ACE2-antivirus particles mirroring multivalent spike patterns, ACE2 molecules were displayed by pseudotyping lentiviral-based viral-like particles (VLPs). Surface display vectors were generated to program VLPs with fusion proteins comprising: an ACE2 extracellular domain, an oligomerization domain, and a display anchoring domain (FIG. 1A). ACE2-Antiviruses were generated by co-transfecting the ACE2 display construct with a lentiviral packaging construct expressing essential packaging components, such as Gag-Pol and Rev proteins; and a viral genome transfer vector encoding a GFP/luciferase reporter, as disclosed herein.

ACE2 display vectors with various combinations of transmembrane anchoring peptides and oligomerization domains were designed (FIG. 1B). Tested display vectors included a display vector utilizing the S2 fusion and transmembrane domain of the SARS CoV-2 spike protein; and a display vector utilizing the transmembrane and cytosolic region of VSV-G (glycoprotein of Vesicular Stomatitis virus) as possible VLP anchoring proteins. Display vectors were also designed with no oligomerization domain, the S2 fusion domain, or the D4 post-fusion trimerization domain of VSV-G, as disclosed herein.

The fusion of ACE2 to full-length VSV-G or to HCΔ18 was also tested—a mutant version of the hemagglutinin envelope protein from measles virus (Data not shown). Among these variations, ACE2 fused to the S2 transmembrane domain (S2TM), VSV-G transmembrane domain (VGTM), or the D4 and VSV-G transmembrane domains (D4VG) produced ACE2-Antiviruses with high copy numbers of ACE2 fusion protein displayed on VLP surfaces, as determined by quantitative western blot analyses using reduced samples (FIG. 1C, 1D). The decoy-antiviruses displayed approximately 85±3 copies of ACE2-S2TM; 1100±400 copies of ACE2-VGTM; and 2700±700 copies of ACE2-VGTM per particle, respectively. Further Western blot analyses using non-reduced samples demonstrated that ACE2 was predominantly displayed as monomers on the ACE2-VGTM Antivirus, whereas over 50% of ACE2 was displayed in trimers or other oligomers on the ACE2-D4VG Antivirus (FIG. 1E). ACE2-VGTM Antiviruses were approximately 134±34 nm in diameter, as determined by Tunable Resistive Pulse Sensing (TRPS) analysis. The morphology of ACE2-VGTM MVPs was characterized by cryoEM.

The neutralizing activity of ACE2-Antiviruses was tested against SARS CoV-2 (FIG. 1F) and SARS CoV-1 (FIG. 1G) in pseudovirus neutralization assays (PNA) using 293T/ACE2 cells as target cells. ACE2-S2TM4, ACE2-VGTM, and ACE2-D4VG Antiviruses had IC₅₀s of 2.18±0.07 pM, 0.23±0.09 pM, and 0.067±0.024 pM against SARS CoV-2 pseudovirus (FIG. 1F); and IC₅₀s of N.D. (not detected), 0.13±0.06 pM and 0.111±0.013 pM, against SARS CoV-1 pseudovirus (FIG. 1G), respectively. In contrast, recombinant ACE2 protein had IC₅₀s of 3.68±1.14 nM and 14.3±5.51 nM against SARS CoV-2 and SARS CoV-1 pseudoviruses, respectively. Interestingly, the ACE2-D4VG Antivirus was a more potent neutralizer of SARS CoV-2 pseudovirus than the ACE2-VGTM Antivirus, which displayed fewer copies of ACE2. Moreover, ACE2-D4VG and ACE2-VGTM Antiviruses were over 10,000-fold more potent than ACE2 recombinant protein against SARS CoV pseudoviruses (FIGS. 1F, 1G). Notably, ACE2-VGTM Antiviruses were nearly 100-fold more potent in terms of IC₅₀ than two clinically-approved neutralizing antibodies, as disclosed herein.

### Example 2: Decoy Antivirus Displaying Enzymatically-Inactive ACE2 is Highly Potent and Variant-Proof

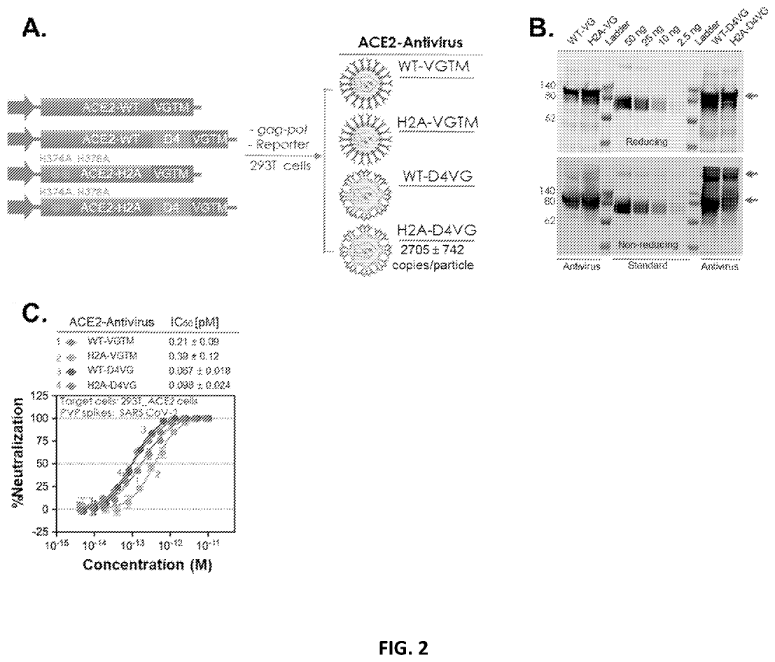

FIG. 2A illustrates the design of decoy-antiviruses with enzymatically active or enzymatically inactive ACE2 in monomeric or oligomerized configurations. FIG. 2B shows data validating multivalent ACE2 display by quantitative Western-blot analyses under both reducing and non-reducing conditions. FIG. 2C shows the neutralizing activities of ACE2-antiviruses against SARS CoV-2 pseudoviruses.

ACE2 is a critical regulator of human angiotensin systems. ACE2 lowers blood pressure by catalyzing the hydrolysis of angiotensin II, a vasoconstrictor, into angiotensin, a vasodilator. To mitigate the acute effects of ACE2 on angiotensin production and blood pressure, an enzymatically active ACE2-antivirus was designed to disrupt ACE2 enzymatic activity. However, mutations that disrupt ACE2 catalytic function, such as H374A and H378A, significantly reduce ACE2 binding affinity to the SARS CoV-2 spike protein, compromising the neutralizing potential of mutated ACE2 decoys against SARS CoV-2. Thus, monomeric and trimeric ACE2-Antiviruses pseudotyped with wild-type and H374A and H378A double mutant ACE2 (FIG. 2A) were generated, and ACE2 expression levels and oligomerization formats of these four types ACE2-antiviruses were validated by quantitative Western-blot analysis under reducing and non-reducing conditions (FIG. 2B). The neutralizing potentials of the ACE2 antiviruses were tested against SARS CoV-2 in pseudovirus neutralization assays, using 293T/ACE2 as target cells (FIG. 2C). Decoy-Antiviruses displaying trimeric WT/ACE2 and H2A/ACE2 were both highly potent inhibitors, neutralizing SARS CoV-2 pseudovirus at IC₅₀s of 0.067±0.018 pM and 0.098±0.024 pM (FIG. 2C), respectively. H2A/ACE2 mutations did not significantly affect the neutralizing activities of trimeric ACE2-Antiviruses. In comparison, decoy-Antiviruses displaying monomeric WT/ACE2 and H2A/ACE2 had about 2 to 4-fold reduced neutralizing activities against SARS CoV-2 pseudoviruses compared to their trimeric counterparts (FIG. 2C).

### Example 3: H2a-Antivirus is Highly Potent Against Live SARS CoV-2 Variants

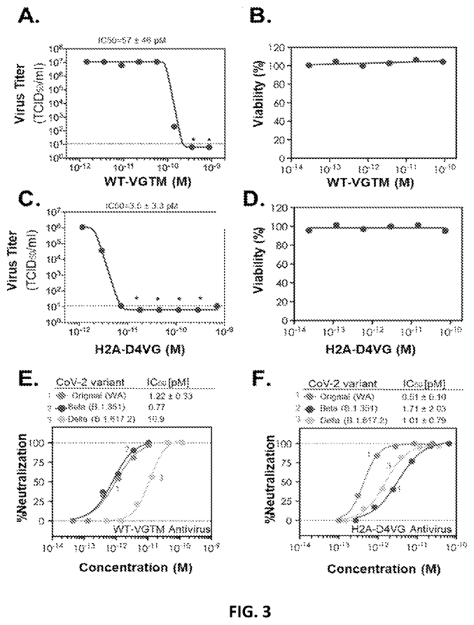

The neutralizing function of monomeric and trimeric ACE2-MVPs were further characterized against live SARS CoV-2. Both monomeric and trimeric ACE2-Antiviruses demonstrated reduced viral titers over six logs to below detection levels in micro-neutralization assays (FIG. 3A-3D). Notably, monomeric WT-VGTM antiviruses neutralized live CoV-2 virus at an IC₅₀ of 57±46 pM (FIG. 3A, 3B), whereas trimeric H2A-D4VG Antiviruses neutralized live SARS CoV-2 virus at an IC₅₀ of 3.5±3.3 pM (FIG. 3C, 3D). No cytotoxicity was observed for either kind of ACE2-Antivirus. The results demonstrated that, while both antiviruses were highly potent inhibitors, the trimeric H2A-D4VG Antiviruses were significantly more potent than monomeric WT-VGTM Antiviruses against live SARS CoV-2 virus.

The ability of decoy-Antiviruses to effectively neutralize the major SARS CoV-2 variants was examined, including the B.1.351 (beta) and B.1.617.2 (delta) variants in live virus Plaque Reduction Neutralization Tests (PRNT). Monomeric WT-VGTM Antiviruses neutralized the original USA-WA1/2020 strain, as well as the beta and delta variants at IC₅₀s of 1.22±0.33 pM, 0.77 pM, and 10.9 pM, respectively (FIG. 3E). In comparison, trimeric H2A-D4VG Antiviruses neutralized the original USA-WA1/2020 strain, beta and delta variants at IC₅₀s of 0.51±0.10 pM and 1.71±2.03 pM, 1.01±0.79 pM, respectively (FIG. 3F). Notably, both monomeric WT-Antiviruses and trimeric H2A-Antiviruses had picomolar or sub-picomolar IC₅₀s against live SARS CoV-2 variants, including the original, beta, and delta variants, in PRNT analyses. Moreover, trimeric H2A-D4VG Antiviruses consistently outperformed monomeric WT-VGTM Antiviruses in live virus neutralization assays. Both monomeric WT-VGTM Antiviruses and trimeric H2A-D4VG Antiviruses were highly potent inhibitors against existing SARS CoV-2 strains, suggesting that ACE2-Antiviruses could serve as variant-proof antivirals for all SARS Coronaviruses utilizing ACE2 as an entry receptor.

FIG. 3A-3F. ACE2-Antiviruses are highly potent against live SARS CoV-2 variants. FIG. 3A shows the neutralizing activity (virus titer) of monomeric WT-VGTM antivirus determined in a SARS CoV-2 live virus neutralization assay. FIG. 3B shows the toxicity (viability %) of monomeric WT-VGTM antivirus determined in a SARS CoV-2 live virus neutralization assay. FIG. 3C shows the neutralizing activity (virus titer) of oligomeric H2A-VGTM antivirus determined in a SARS CoV-2 live virus neutralization assay. FIG. 3D shows the toxicity (viability %) of oligomeric H2A-VGTM antivirus determined in a SARS CoV-2 live virus neutralization assay. FIG. 3E shows the neutralizing activity (% neutralization) of monomeric WT-VGTM antivirus against live SARS CoV-2 variants, as determined by a plaque reduction neutralization (PRNT) assay. FIG. 3F shows the neutralizing activity (% neutralization) of oligomeric H2A-VGTM antivirus against live SARS CoV-2 variants, as determined by a plaque reduction neutralization (PRNT) assay.

### Example 4: H2a-Antivirus Treatment Rescues Mice from Lethal Infection by SARS CoV-2 Variants

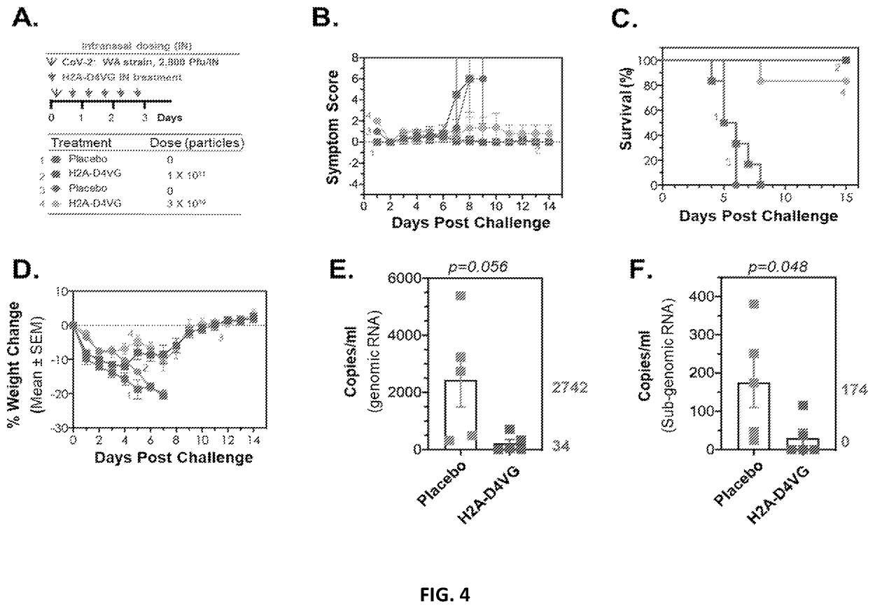

SARS CoV-2 infection causes lethality in K18-hACE2 transgenic mice and induces symptoms and pathology recapitulating many of the defining features of severe COVID-19 infection in humans. High levels of viral infection in lungs, with spread to the brain and other organs, is observed in infected mice, coinciding with massively upregulated inflammatory cytokines and infiltration of monocytes, neutrophils and activated T cells. The K18-hACE2 model was used to test the efficacy of vaccine and therapeutics in protecting against lethal SARS-CoV-2 infection. The ability of intranasally delivered ACE2-Antivirus to rescue hACE2 transgenic mice from SARS CoV-2 infection. K18-hACE2 mice were challenged with 2800 pfu of the SARS CoV-2 (Strain USA-WA1/2020, BEA resource, NR-52281) and treated with 5 doses of H2A-D4VG Antivirus at 3×10¹⁰ or 1×10¹¹ particles per dose, delivered intranasally (IN) (FIG. 4A). The first dose was given 4 hours post-infection, and subsequent doses were given twice daily at day 1 and 2 post-infection. Mice treated with high dose H2A-D4VG antivirus had no noticeable respiratory symptoms, while a few mice treated at the lower dose exhibited minor respiratory symptoms (FIG. 4B). All mice in the high dose group and 5 of 6 mice in the low dose group survived, while all mice in the placebo group succumbed to the virus at around day 6 post-infection (FIG. 4C). In comparison to the placebo group, mice in the treatment group experienced modest and transitory weight loss (FIG. 4D). Supporting the potent treatment effects of H2A-D4VG Antiviruses, viral loads were reduced to background levels as indicated by genomic and sub-genomic qPCR analyses (FIG. 4E, 4F) of oral swab samples collected at day-3 post-infection.

FIG. 4A-4F. Post-exposure intranasal delivery of H2A-D4VG Antivirus recues lethal infection of hACE2 transgenic mice by the SARS CoV-2 Washington strain. FIG. 4A shows the intranasal treatment dosing regimen of mice challenged with a lethal dose of SARS CoV-2 Washington strain. FIG. 4B shows the effect of H2A-D4VG antivirus treatment on respiratory symptoms. FIG. 4C shows the effect of H2A-D4VG antivirus treatment on survival. FIG. 4D shows the effect of H2A-D4VG antivirus treatment on weight loss. FIG. 4E shows the effect of treatment with H2A-D4VG antivirus treatment on the viral loads in the nasal swabs of treated mice determined by quantitative PCR analyses of SARS CoV-2 genomic RNAs. FIG. 4F shows the effect of H2A-D4VG antivirus treatment on the viral loads in the nasal swabs of treated mice determined by quantitative PCR analyses of SARS CoV-2 sub-genomic RNAs.

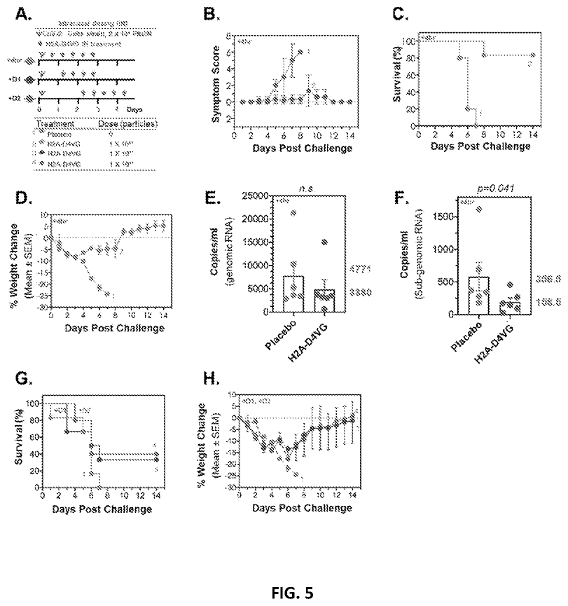

The ability of H2A-D4VG Antiviruses to rescue mice from lethal infection by the delta variant of SARS CoV-2 was studied at a higher challenge dose. K18-hACE2 mice were challenged with 2×10⁴ pfu of SARS CoV-2 delta strain, about 7-fold more than the challenge dose used in wild type study (Example 3). Mice were then treated with total 5 doses of intranasal delivered H2A-D4VG Antivirus at 1×10¹¹ particles per IN dose starting at 4 hours (+4 hr group), day 1 (+D1 group) and day 2 (+D2 group) post-challenge. Subsequent doses were given twice a day post-infection (FIG. 5A). One out of the six mice from the +4 hr early treatment group developed respiratory symptoms (FIG. 5B) and succumbed to infection (FIG. 5C). In contrast, all control mice developed rapid onset of respiratory symptoms after day-5 post-challenge and succumbed to infection (FIG. 5B, 5C). Notably, in comparison to the placebo group, mice in the treatment group experienced modest and transitory weight loss (FIG. 5D). Supporting the potent treatment effects of H2A-D4VG Antiviruses against the delta variant, viral loads in treated mice were significantly reduced, as indicated by genomic and sub-genomic qPCR analyses (FIG. 5E, 5F) of oral swab samples collected at day-3 post-challenge. Two out of six mice in the +D1 or +D2 delayed treatment groups were rescued from lethal infection (FIG. 5G, 5H). Together, the results demonstrated that H2A-D4VG Antiviruses effectively reduced viral loads and rescued mice from lethal infection by original and delta variant SARS CoV-2, indicating that ACE2-Antivirus may be a variant-proof therapeutic for SARS Coronaviruses utilizing ACE2 as an entry receptor. The therapeutic effects were dependent on the viral challenge dose and the treatment dosage.

FIG. 5A-5F. Post-exposure intranasal delivery of H2A-D4VG Antivirus recues lethal infection of hACE2 transgenic mice by the SARS CoV-2 delta variant. FIG. 5A shows intranasal treatment doses and regimens of mice challenged with the lethal dose of SARS CoV-2 delta variant. FIG. 5B shows the effect of H2A-D4VG antivirus treatment of the infected mice on their respiratory symptoms. FIG. 5C shows the effect of H2A-D4VG antivirus treatment of the infected mice on survival. FIG. 5D shows the effect of H2A-D4VG antivirus treatment of the infected mice on weight loss. FIG. 5E shows the effect of H2A-D4VG antivirus treatment on the viral loads in the nasal swabs of treated mice determined by quantitative PCR analyses of SARS CoV-2 genomic RNAs. FIG. 5F shows the effect of H2A-D4VG antivirus treatment on the viral loads in the nasal swabs of treated mice determined by quantitative PCR analyses of SARS CoV-2 sub-genomic RNAs.

### Example 5: ACE2-Antivirus Treatment Leads to Broad Immunity Against SARS CoV-2 Variants

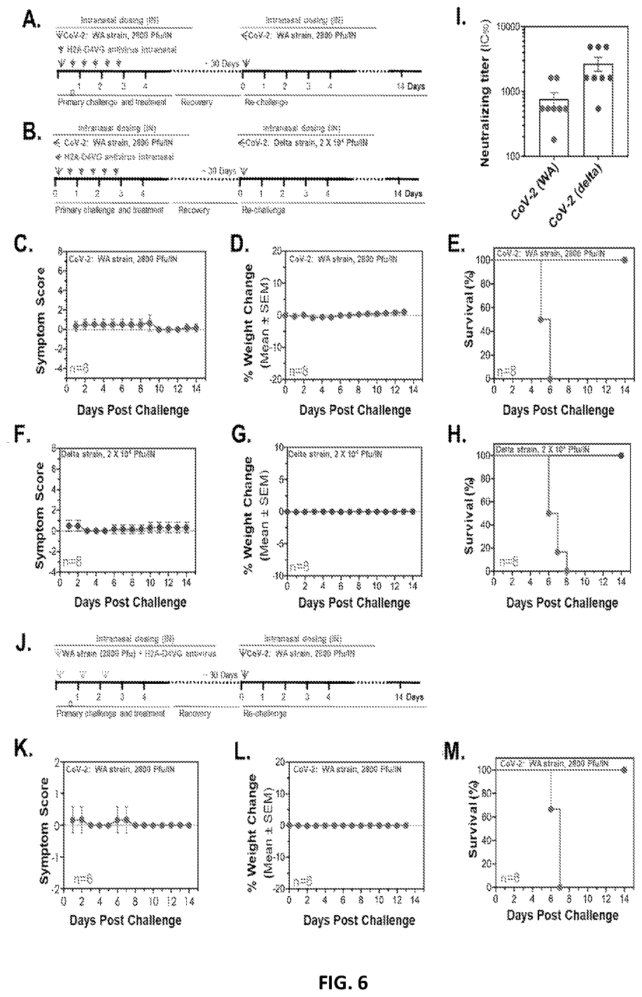

Rechallenge studies were performed to determine whether infected mice can develop protective immunity against future infection following ACE2-Antivirus treatment. The studies were performed to determine whether particle complexes of ACE2-Antivirus and coronavirus virions could be effectively taken up by antigen-presenting immune cells, and whether treated mice could demonstrate minimal noticeable respiratory symptoms. K18-hACE2 mice rescued by ACE2-antivirus treatment from primary infection (2800 pfu of the original Washington strain) were rested for 30 days. Mice were then rechallenged with either 2800 pfu of live SARS CoV-2 WA strain (FIG. 6A) or with 20,000 pfu of the SARS CoV-2 delta strain (FIG. 6B). No significant respiratory symptoms, weight loss, or death were observed in mice rechallenged with either the original WA strain (FIG. 6C-6E) or the delta variant (FIG. 6F-6H). Neutralizing titers were then measured from serum collected from these mice. The serum contained median levels of 540 or 1620 TCID₅₀ neutralizing titers against the original SARS CoV-2 and delta variants, respectively (FIG. 6I). Notably, the half-life of ACE2-antivirus in the lung was determined to be approximately 12 hours (Data not shown). Thus, the protective effect against rechallenge by SARS CoV-2 variants was unlikely to be caused by the residual ACE2-antivirus at 30 days post-primary infection and treatment, and was more likely the results of the protective immunity induced by ACE2-antivirus treatment against SARS CoV-2 variants.

To further explore the possibility of protective immunity induced by ACE2-antivirus treatment against SARS CoV-2 variants, 2800 pfu live SARS CoV2 (WA) was pre-incubated with ACE2-antivirus (1×10¹¹ particles) in vitro for 30 minutes. Mice were then re-immunized with the ACE2-antivirus “inactivated” SARS CoV-2 through three consecutive intranasal doses (FIG. 6I). As expected, the mice did not develop any respiratory symptoms or had any weight-loss or death (Data not shown). More importantly, the mice were then rechallenged with 2800 pfu of live SARS CoV-2 WA strain (FIG. 6I). No significant respiratory symptoms (FIG. 6J), weight loss (FIG. 6K), or death (FIG. 6L) were observed.

FIG. 6A-6M. Infected mice rescued by H2A-antivirus treatment have broad immunity against SARS CoV-2 variants. FIG. 6A shows doses and regimens of ACE2-antivirus treatment of primary infection and subsequent rechallenge with the original SARS CoV-2 Washington strain. FIG. 6B shows doses and regimens of ACE2-antivirus treatment of primary infection and subsequent rechallenge with the delta variant. FIG. 6C shows the effect of the Washington strain re-infection on respiratory symptoms of hACE2 mice rescued from primary infection. FIG. 6D shows the effect of the Washington strain re-infection on weight loss of hACE2 mice rescued from primary infection. FIG. 6E shows the effect of the Washington strain re-infection on survival of hACE2 mice rescued from primary infection. FIG. 6F shows the effect of the delta strain re-infection on respiratory symptoms in hACE2 mice rescued from primary infection. FIG. 6G shows the effect of the delta strain re-infection on weight loss in hACE2 mice rescued from primary infection. FIG. 6H shows the effect of the delta strain re-infection on survival in hACE2 mice rescued from primary infection. FIG. 6I shows authentic virus neutralization at 14 days post-challenge, and is expressed as the half-maximal effective concentration (IC₅₀) in the serum of rechallenged hACE2 mice. FIG. 6J illustrates immunization of mice with ACE2-antivirus “inactivated” SARS CoV-2 WA strain and subsequent rechallenge. FIG. 6K shows the effect of SARS CoV-2 WA strain re-infection on respiratory symptoms of hACE2 mice immunized with ACE2-antivirus “inactivated” SARS CoV-2. FIG. 6L shows the effect of SARS CoV-2 WA strain re-infection on weight loss of hACE2 mice immunized with ACE2-antivirus “inactivated” SARS CoV-2. FIG. 6M shows the effect of SARS CoV-2 WA strain re-infection on survival of hACE2 mice immunized with ACE2-antivirus “inactivated” SARS CoV-2.

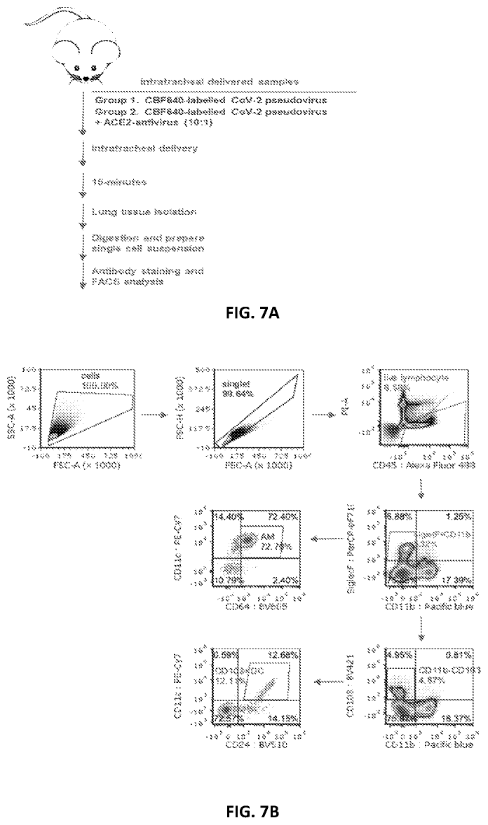

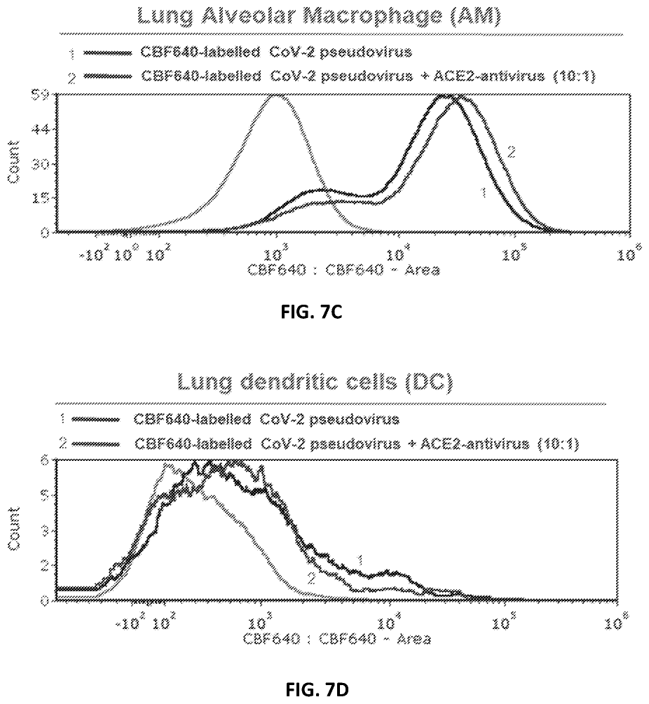

SARS CoV-2 pseudovirus were labeled with fluorescent dyes, and the ability of the formation of ACE2-antivirus and pseudo-virion complexes on facilitating uptake by lung alveolar macrophages was studied (FIG. 7A-7D). FACS analyses clearly showed that ACE2-antivirus and pseudo-virion complexes Were more effectively taken up by the lung alveolar macrophages than the non-complexed pseudo-virions (FIG. 7C), whereas ACE2-antivirus and pseudo-virion complexes and non-complexed pseudo-virions were taken up by lung dendritic cells equally effectively (FIG. 7D). Collectively, the results clearly demonstrated that ACE2-antivirus treatments of SARS CoV-2 infection could lead to the development of robust immunity against broad range of SARS CoV-2 variants, and ACE2-antivirus “inactivated” SARS CoV-2 could be used as a nasal vaccine.

FIG. 7A-7D. FACS analyses to determine the uptake of ACE2-antivirus and SARS CoV-2 pseudovirus complexes by lung alveolar macrophages and dendritic cells upon intranasal delivery. FIG. 7A illustrates the design of uptake analyses of ACE2-antivirus and SARS CoV-2 pseudovirus complexes by lung alveolar macrophages and dendritic cells. Dye-labelled (CBF640) SARS CoV-2 pseudovirus was incubated with unlabeled ACE2-antivirus and delivered into mouse lungs through intratracheal delivery. After 15 minutes, lung tissues were isolated from the mice, digested, and processes into single cells for FACS staining. FIG. 7B shows FACS staining and gating strategy for defining the uptake of ACE2-antivirus and SARS CoV-2 pseudovirus complexes by lung alveolar macrophages and dendritic cells. FIG. 7C shows the relative uptake of dye-labeled SARS CoV-2 pseudovirus alone or ACE2-antivirus and pseudovirus complexes by lung alveolar macrophages. FIG. 7D shows the relative uptake of dye-labelled SARS CoV-2 pseudovirus alone or ACE2-antivirus and pseudovirus complexes by dendritic cells.

### Example 6: Design and Production of Decoy Multivalent Particles Displaying ACE2 Receptors (ACE2-MVPs)

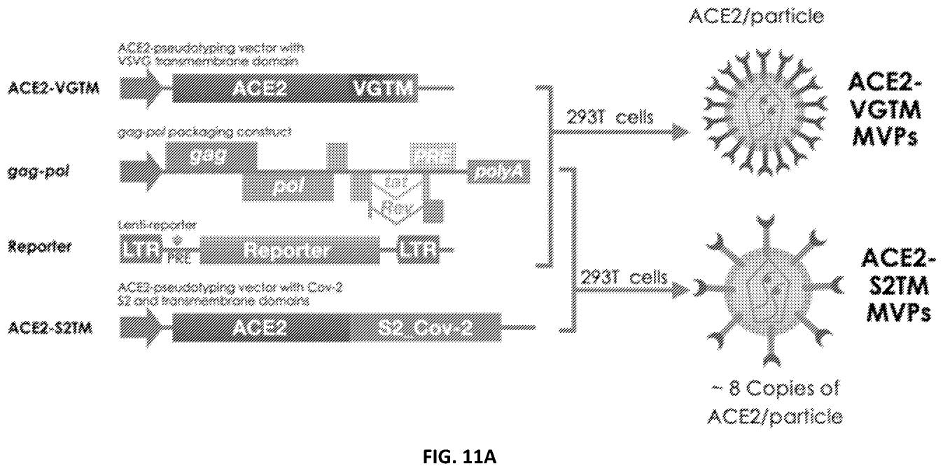

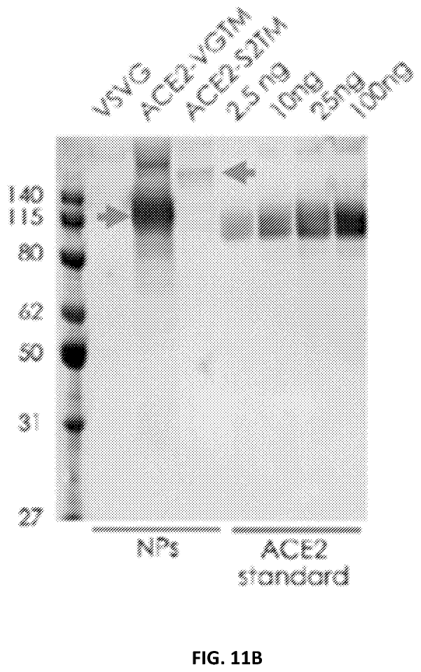

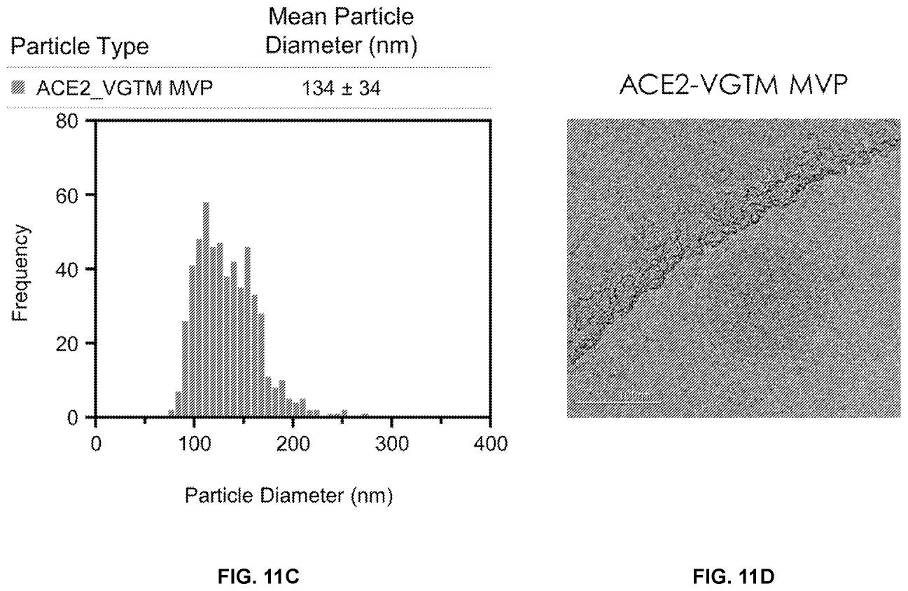

ACE2-MVPs were generated by pseudotyping lentiviral particles with a fusion protein consisting of the ACE2 extracellular domain and the membrane anchoring segment of a viral envelop protein (FIG. 11A). Briefly, ACE2-MVPs were generated by co-transfecting the ACE2 fusion construct with a lentiviral packaging construct expressing essential packaging components, such as Gag-Pol and Rev proteins, and a viral genome transfer vector encoding a GFP/luciferase reporter. ACE2-MVPs without viral genomes were also packaged with no transfer vector. Several viral envelope proteins were tested for anchoring ACE2 protein to the membrane of the pseudo-lentiviral particles, including VSV-G (glycoprotein of Vesicular Stomatitis virus), HCΔ18 (a mutant version of the hemagglutinin envelope protein from measles virus), and S2 (the fusion domain of the SARS CoV-2 spike protein). Also tested were the fusion of ACE2 to full-length VSVG or truncated VSV-G with only a transmembrane region and cytosolic tail.

Among the variations of ACE2 fusion proteins, ACE2 fused with VSVG transmembrane domain (VGTM), and S2 transmembrane domain (S2TM) produced ACE2-MVPs with high copies of the ACE2 fusion protein on the viral-like particle surface as determined by quantitative Western blot analyses (FIG. 11B). These pseudotyped MVPs displayed about eight copies of ACE2-S2TM or 236 copies of ACE2-VGTM on the particles, respectively, providing a basis to test the effects of valency on the neutralizing function of ACE2-MVPs. The average particle diameter of ACE2-VGTM MVPs was 134±34 nm, as determined by tunable resistive pulse sensing analyses (TRPS) using qNano (FIG. 11C). The morphology of ACE2-VGTM MVPs were characterized by cryoEM analyses at nominal magnification of 150,000× (FIG. 11D).

### Example 7: Efficient Inhibition of SARS CoV-2 Virus Infection by ACE2-MVPs

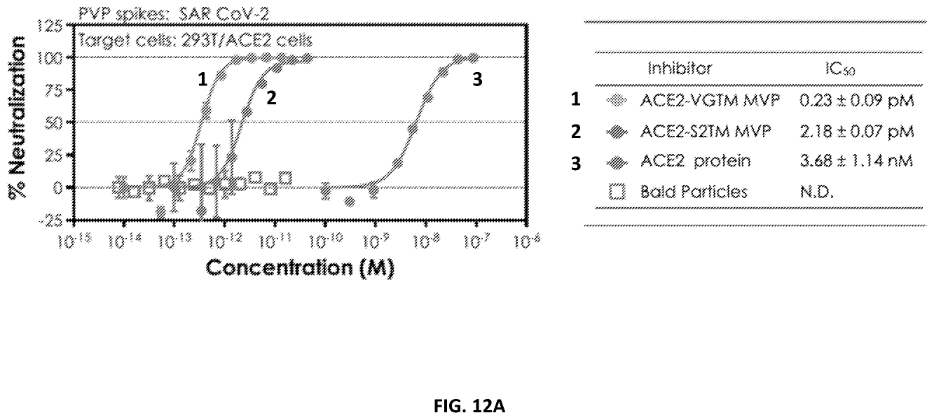

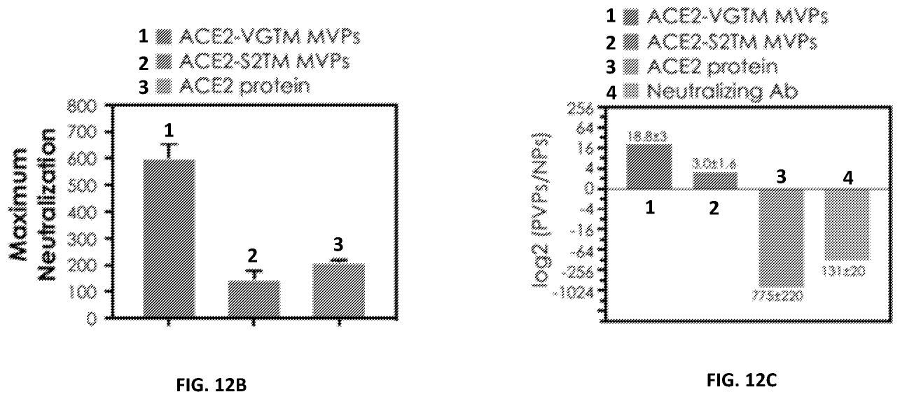

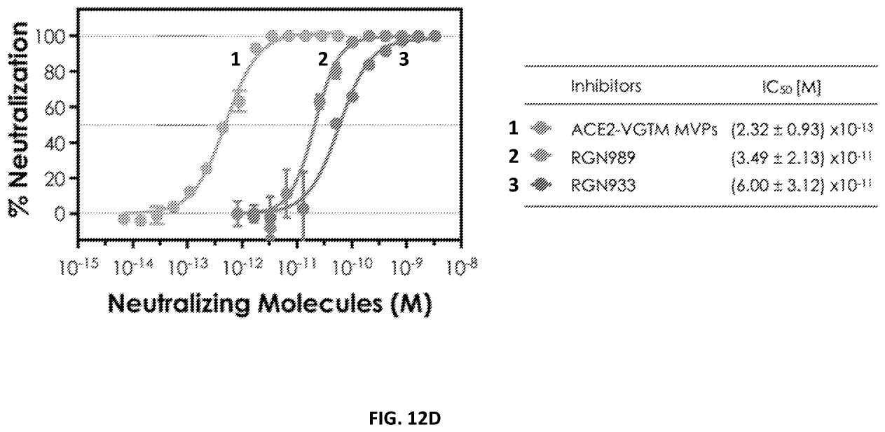

The neutralizing activity of ACE2-MVPs were determined in a microneutralization assay against lentiviruses pseudotyped with SARS CoV-2 spike protein (CoV-2 PVP) using 293 T/ACE2 cells as target cells. Recombinant ACE2 had an IC₅₀ of 3.68±1.14 nM in the pseudovirus neutralization assay, as shown in FIG. 12A. In contrast, the decoy-MVPs displaying ACE2-VGTM or ACE2-S2TM had IC₅₀ values of 0.23±0.09 pM or 2.18±0.07 pM, respectively, which were at least 1000-fold or 10,000-fold more potent than the monovalent ACE2 recombinant protein (FIG. 12A). The results demonstrated that the neutralizing function of the ACE2 decoy receptor were drastically enhanced by increasing valencies. Notably, the ACE2-VGTM MVPs displaying ˜236 copies of ACE2 were about 10-fold more potent than the ACE2-S2TM MVPs displaying ˜8 copies of ACE2. Furthermore, the maximum inhibition of pseudovirus infection was about 600-fold by the ACE2-VGTM MVPs, and about-100 to 200-fold by the ACE2-S2TM MVPs and the ACE2 recombinant proteins (FIG. 12B). Finally, since both ACE2 MVPs and CoV-2 PVPs were pseudotyped lentiviral particles, the stoichiometric ratios between the neutralizing MVPs and the pseudovirus particles were determined by P24 ELISA assays. As shown in FIG. 12C, one particle of ACE2-VGTM MVP or ACE2-S2TM MVP neutralized about 18 or 3 the pseudovirus particles, respectively. About ˜131 copies of recombinant ACE2 proteins were required to neutralize one pseudovirus particle. Notably, the ACE2-VGTM MVPs were nearly 100-fold more potent than two of the antibodies used in the Regeneron antibody cocktails for clinical studies (FIG. 12D).

FIG. 12A-12D show that higher ACE2 valency on the ACE2-MVPs correlated with enhanced neutralization activity. FIG. 12A-12C show the neutralizing activities of various anti-CoV-2 compounds, including ACE2 recombinant protein, and MVPs displaying ACE2-VGTM or ACE2-S2TM were determined in a SARS CoV-2 pseudovirus infection assay using 293T/ACE2 cells as target cells. FIG. 12A shows IC₅₀ values of ACE2-VGTM MVP; ACE2-S2TM MVP; ACE2 protein; and bald particles. FIG. 12B shows maximum neutralization and total fold repression of ACE2-VGTM MVP; ACE2-S2TM MVP; and ACE2 protein. FIG. 12C shows the molecular ratio of viral particle to antiviral compounds of ACE2-VGTM MVP; ACE2-S2TM MVP; and ACE2 protein. FIG. 12D shows the neutralizing activities of the decoy-MVPs displaying ACE2-VGTM and clinical stage neutralizing antibodies, which were determined using a SARS CoV-2 pseudovirus infection assay with 293T/ACE2 cells as target cells.

### Example 8: ACE2-MVPs are Broadly Neutralizing Against ACE2-Targeting Coronaviruses

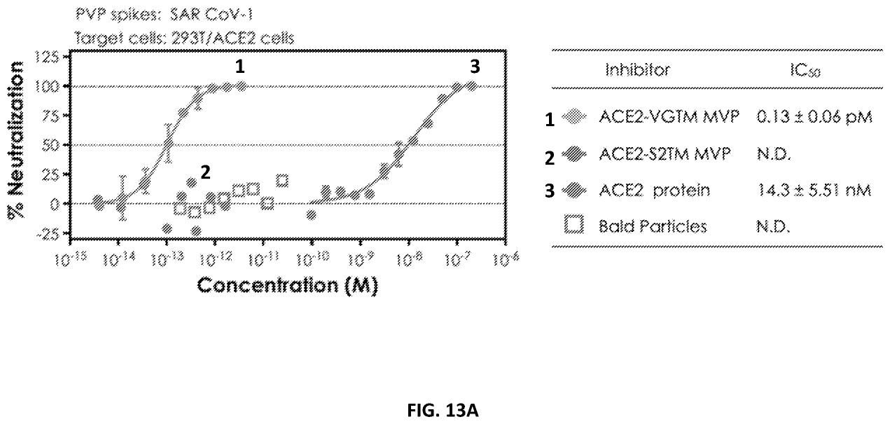

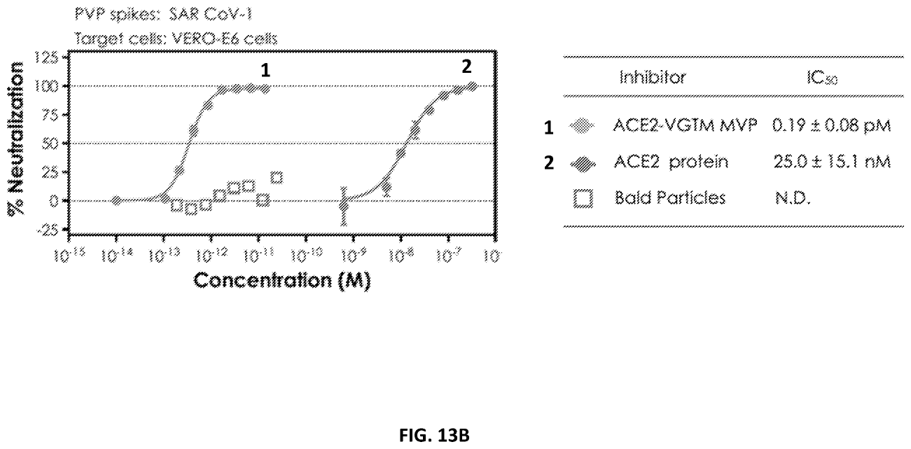

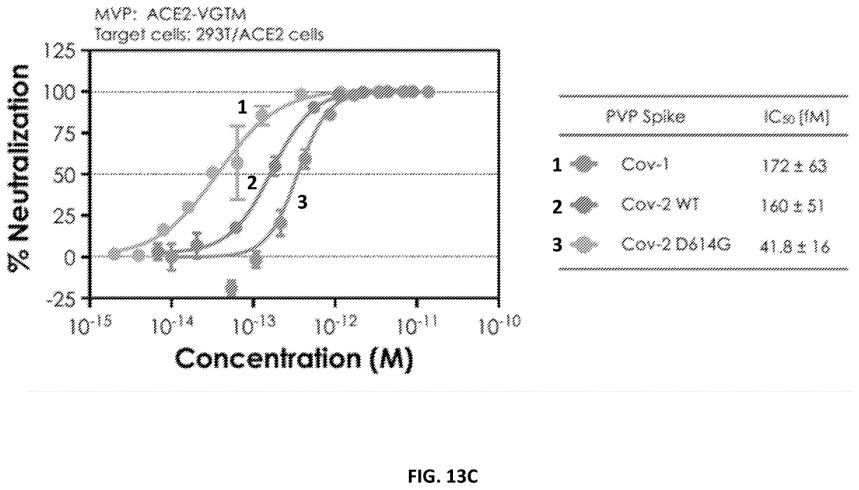

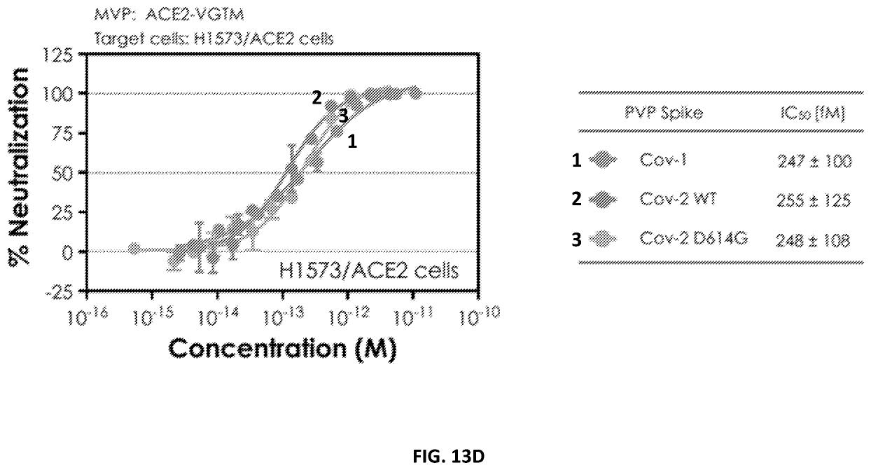

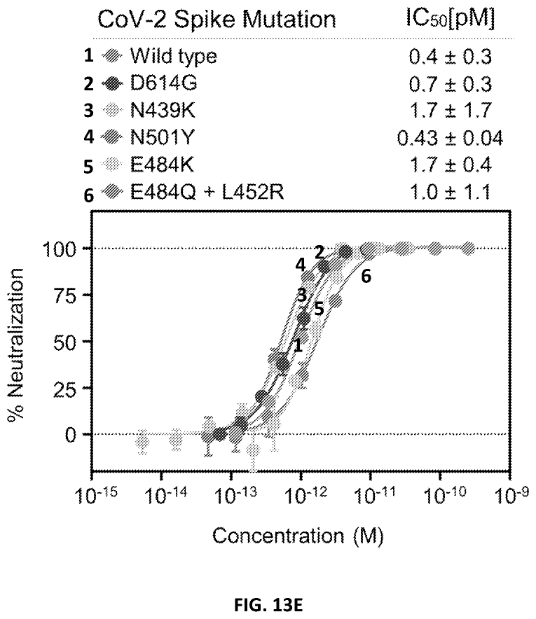

ACE2 is used as an entry receptor by SARS CoV-1 and evolving SARS CoV-2, and thus the ACE2-VGTM MVPs may have broad neutralizing activity against these viruses. The neutralizing activities of ACE2-MVPs were tested against lentiviruses pseudotyped with SARS CoV-1 spike (CoV-1 PVPs) in a microneutralization assay using 293 T/ACE2 cells as target cells. Recombinant ACE2 had an IC₅₀ of 14.3±5.51 nM (FIG. 13A) in a pseudovirus neutralization assay, about 4-5 folds less potent than its activity against CoV-2 PVPs (FIG. 12A). In contrast, the ACE2-VGTM MN/VP had an IC₅₀ of 0.13±0.06 pM, which was comparable to its neutralizing activity against CoV-2 PVPs (FIG. 12A). These results demonstrated that the ACE2-VGTM MVP was a highly potent neutralizing compound against both CoV-1 and CoV-2 viruses, whereas ACE2 recombinant protein showed much less potent neutralizing compound against CoV-1. Furthermore, the ACE2-S2TM MVP, which had ˜8 copies of ACE2 per particle, had no detectable neutralizing activity when comparable concentrations to the ACE2-VGTM MVP was used in the neutralization assay (FIG. 13A). The neutralizing activities of ACE2-MVPs were also tested in a CoV-1 PVP neutralization using VERO-E6 cells as target cells and observed similar IC₅₀ values for the ACE2 recombinant protein and the ACE2-VGTM MVPs (FIG. 13B). In summary, the high-valent ACE2-VGTM MVPs were equally potent in neutralizing both CoV-1 and CoV-2 pseudoviruses, and moreover, higher ACE2 valency on the MVPs appears to overcome the lower affinity between the spike and entry receptor.

SARS CoV-2 is also rapidly mutating, and some of the mutations are more transmissible and more pathogenic. Interestingly, the ACE2-S2TM MVP had an IC₅₀ of 41.8±16 fM in neutralizing D614G spike pseudotyped viruses (D614G-PVP) in a microneutralization assay using 293T/ACE2 cells as target cells, which was at least 3-5 folds more potent against CoV-1 PVP and CoV-2 PVP (FIG. 13C). The ACE2-S2TM MVP had comparable neutralizing activities against CoV-1, CoV-2, and D614G-PVPs in a microneutralization assay using H1573/ACE2 cells as target cells (FIG. 13D). Finally, the ACE2-VGTM MVP was equally potent against CoV-2 variants with N439K, N501Y, E484K, and E484Q+L452R mutations (FIG. 13E). These results support that high-valency ACE2-VGTM MVPs are equally potent in neutralizing both SARS CoV-1, SARS CoV-2, and a variety of CoV-2 variant pseudoviruses, and moreover, that higher ACE2 valency on MVPs is critical to overcoming lower binding affinities between viral spike and host cell entry receptor, demonstrating that ACE2-MVPs are potent neutralizing compounds against all emerging coronaviruses utilizing ACE2 as an entry receptor.

FIG. 13A-13E show the efficient neutralization of SARS-CoV-1 viruses by the ACE2-MVPs, and that neutralization depends on the copies of ACE2 molecules displayed on the particle surfaces. FIG. 13A shows the neutralization activities of the decoy-MVPs displaying ACE2-VGTM or ACE2-S2TM in a SARS CoV-1 pseudovirus infection assay using 293T/ACE2 cells. FIG. 13B shows the neutralization activities of the ACE2-MVPs displaying ACE2-VGTM or ACE2-S2TM in a SARS CoV-1 pseudovirus infection assay using VERO-E6 cells. FIG. 13C shows the neutralization activities of ACE2-VGTM MVPs against CoV-1, CoV-2, and D614G CoV-2 in pseudovirus infection assay using 293 T/ACE2 cells. FIG. 13D shows the neutralization activities of ACE2-VGTM MVPs against CoV-1, CoV-2, and D614G CoV-2 in pseudovirus infection assay using H1573/ACE2 cells. FIG. 13E shows a comparison of the neutralizing activities of the ACE2-VGTM MVPs against a variety of SARS CoV-2 variants in pseudovirus infection assay using 293T/ACE2 cells as target cells.

### Example 9: Efficient Inhibition of MERS Coronavirus Infection by DPP4-MVPs

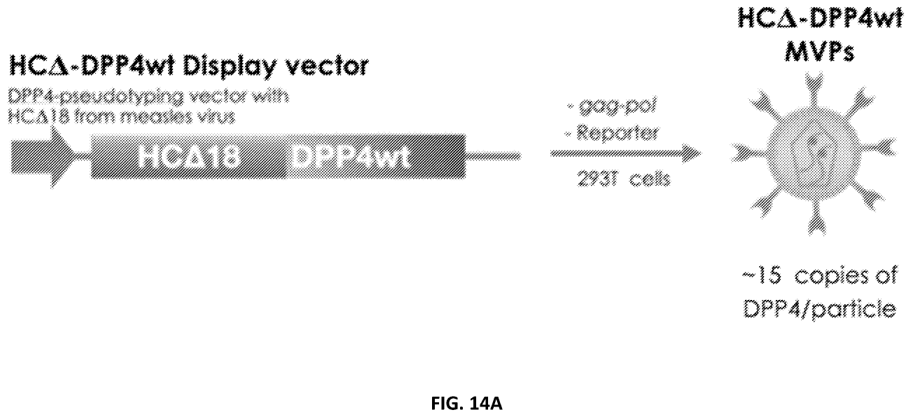

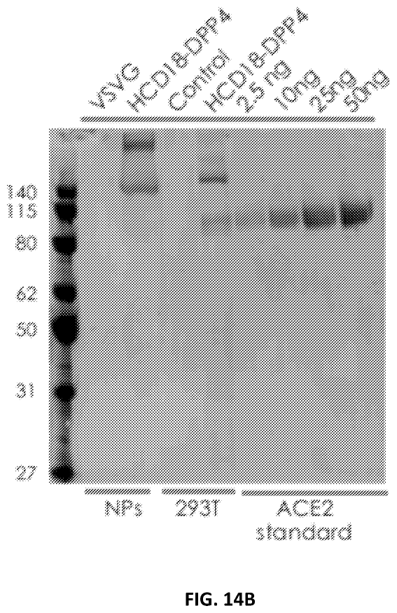

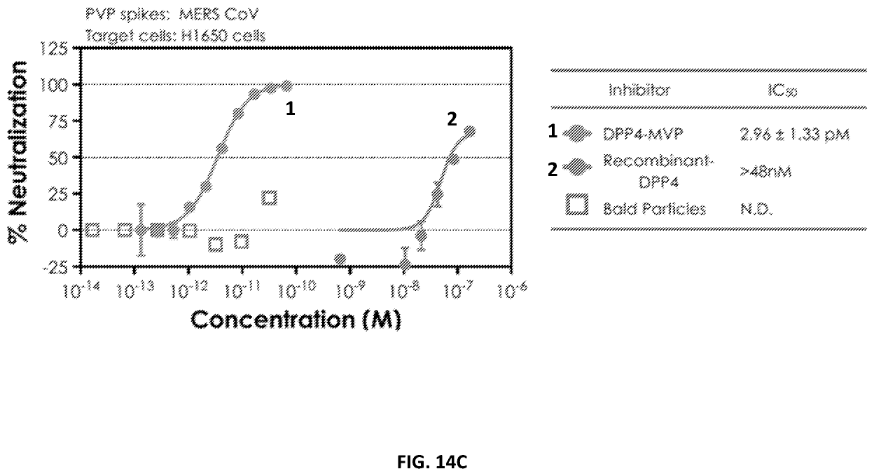

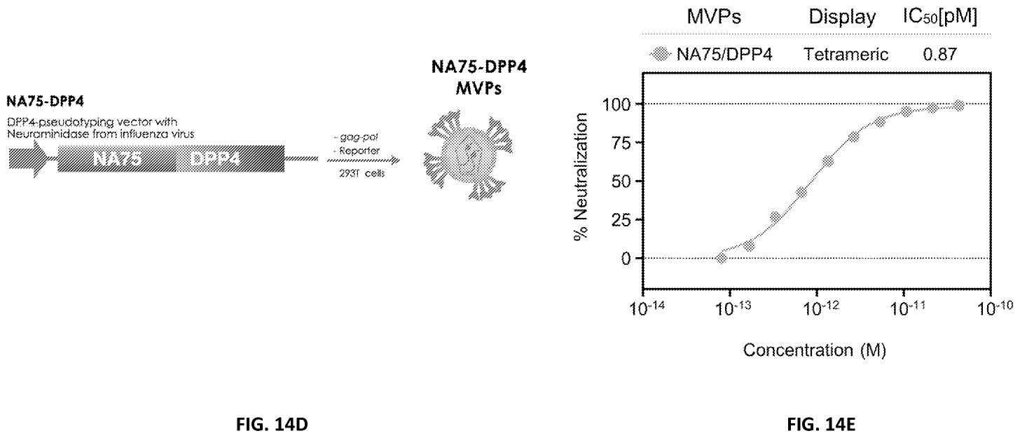

The MERS coronavirus utilizes DPP4 as an entry receptor. To test whether a similar decoy-MVP strategy may be used to neutralize MERS viruses, DPP4-MVPs were generated by pseudotyping lentiviral particles with a fusion protein consisting of the membrane anchoring segment of a mutant version of hemagglutinin envelope protein from measles virus (HCΔ18) and the DPP4 extracellular domain (FIG. 14A). Both the measles envelope protein and DPP4 are type II transmembrane proteins. In the HCΔ-DPP4 pseudotyping construct, the N-terminus and transmembrane region of HCΔ18 were retained and the C-terminal region (extra-membrane) was replaced with the corresponding region of the DPP4. DPP4-MVPs were generated by co-transfecting 293 T cells with the HCΔ-DPP4 pseudotyping construct and a lentiviral packaging construct expressing essential lentiviral packaging components, such as Gag-Pol and Rev proteins, and a lentiviral genome transfer vector encoding a GFP/luciferase reporter. As determined by quantitative Western blot analyses (FIG. 14B), DPP4-MVPs displayed about 15 copies of HCΔ-DPP4 on the particles.

The neutralizing activities of DDP4-MVPs were tested against lentiviruses pseudotyped with MERS spike (MERS-PVPs) in a microneutralization assay using H1650 cells as target cells. The DPP4-MVP had an IC50 of 2.96±1.33 pM in the pseudovirus neutralization assay, whereas recombinant DPP4 had an IC50 of more than 48 nM (FIG. 14C). These results demonstrate that highly potent neutralizing MVPs against IMERS coronaviruses can be generated by displaying multiple copies of a low-affinity type II entry receptor. Furthermore, the IC₅₀ of DPP4-MVPs in neutralizing live MERS coronavirus infection in a microneutralization assay will be assessed.

Finally, to further optimize the display of type II viral entry receptors, the display of DPP4 on lentiviral VLPs was tested by fusing the neuraminidase N-terminus and transmembrane regions with the DPP4 extracellular domain (FIG. 14D) to generate NA75-DPP4 MVPs accordingly. The NA75-DPP4 MVP had an IC₅₀ of 0.87 pM in pseudovirus neutralization assays (FIG. 14E). The results demonstrated that highly potent neutralizing decoy-MVPs could be generated against MERS coronaviruses by displaying multiple copies of a low-affinity type II entry receptor.

FIG. 14A-14E show the design and activity of DPP4-MVPs displaying multiple copies of decoy DPP4 receptors. FIG. 14A shows the design and production of HCΔ-DPP4-MVPs. The schematic illustrates the DPP4-displaying constructs with DPP4 extracellular domain fused to the HCΔ18 transmembrane domain from measles virus. HCΔ-DPP4 MVPs were generated by co-transfecting DPP4-displaying constructs with a lentiviral packaging construct and lentiviral reporter construct. FIG. 14B shows quantitative Western-blot analysis used to determine the copy number of DPP4 molecules on the HCΔ-DPP4 MVPs. FIG. 4C shows the neutralizing activities of various anti-MERS compounds, including DPP4 recombinant protein, and HCΔ-DPP4 MVPs were determined in a MERS pseudovirus infection assay using H1650 cells as target cells. FIG. 14D shows the design and production of NA75-DPP4 MVPs. The schematic illustrates the DPP4-displaying constructs with DPP4 extracellular domain fused to the neuraminidase transmembrane domain from influenzavirus. NA75-DPP4 MVPs were generated by co-transfecting NA75-DPP4-displaying constructs with a lentiviral packaging construct and lentiviral reporter construct. FIG. 14E shows the neutralizing activities of NA75-DPP4 MVPs determined in a MERS pseudovirus infection assay using H1650 cells as target cells.

### Example 10: The Reduced Neutralizing Potency of Decoy-MVPs Displaying Enzymatically Inactive ACE2

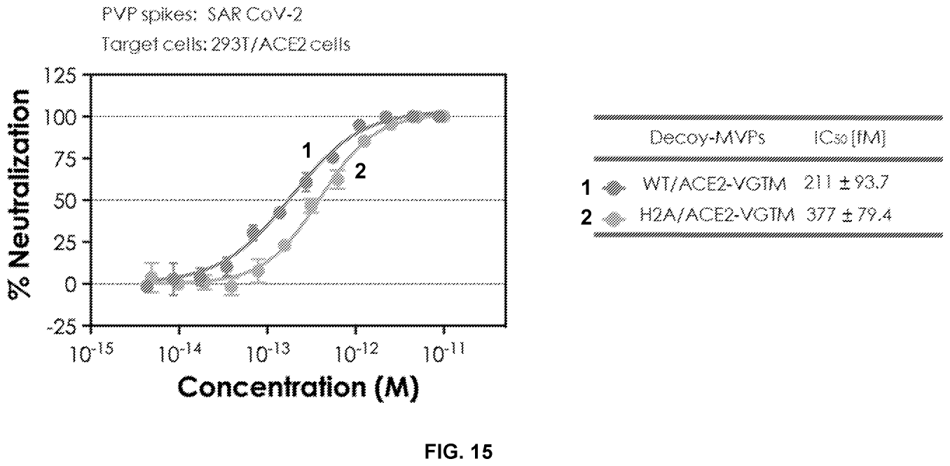

ACE2 is a critical regulator of human angiotensin systems. It lowers blood pressure by catalyzing the hydrolysis of angiotensin II, a vasoconstrictor, into angiotensin (1-7), a vasodilator. Human recombinant ACE2 has been tested in 89 healthy volunteers in a Phase I study and in patients with acute respiratory distress syndrome (ARDS) in a phase II study. Although a safety window can be established, the acute effects of active ACE2 on angiotensin (1-7) production and blood pressure present safety concerns for using ACE2 protein as a SARS CoV-2 neutralizing therapeutics. Moreover, mutations that disrupt the ACE2 catalytic function, such as H374A and H378A, also significantly reduce ACE2 binding to CoV-2 spike protein, thus potentially compromising the neutralization potential ACE2 neutralizing decoys against SARS CoV-2. To this end, decoy-MVPs displaying monomeric H2A/ACE2-VGTM was demonstrated to have an IC₅₀ of 377±79.4 fM, whereas decoy-MVPs displaying monomeric WT/ACE2-VGTM has an IC₅₀ of 211±93.7 fM, in a pseudovirus neutralization assay (FIG. 15). This result confirmed that the inactivating mutations do have some detrimental effects on the neutralizing function of ACE2-MVPs.

FIG. 15 shows that multivalent particles displaying enzymatic-inactive H2A-ACE2, designated as H2A/ACE2 MVPs, have a reduced neutralizing activity against CoV-2 pseudovirus. The neutralizing activities of the H2A/ACE2 MVPs and wild-type ACE2-MVPs were determined in a SARS CoV-2 pseudovirus infection assay using 293T/ACE2 cells as target cells.

### Example 11: Decoy-MVPs with Oligomerized ACE2 Display have Enhanced Neutralizing Potency

Notably, the ACE2-VGTM construct (FIG. 11A) could be used to display multiple copies of monomeric ACE2 molecules on the surface of MVPs based on Western-blot analyses. Although ACE2 MVP with monomeric ACE-VGTM was highly efficacious in neutralizing CoV-1 and CoV-2, the monomeric ACE2 display pattern did not match the trimeric display pattern of the spike protein. The effect of increasing the neutralizing potency of ACE2-MVPs was examined by generating MVPs displaying multivalent ACE2 with trimeric patterns matching to the spike proteins. Such design could further enhance the local avidity and multivalent interaction between spike trimers on the virus and ACE2 trimers on the decoy-MVP. With enhanced local avidity and binding, the detrimental effects of H2A mutations on the neutralizing function of ACE2 decoy-MVPs could be overcome.

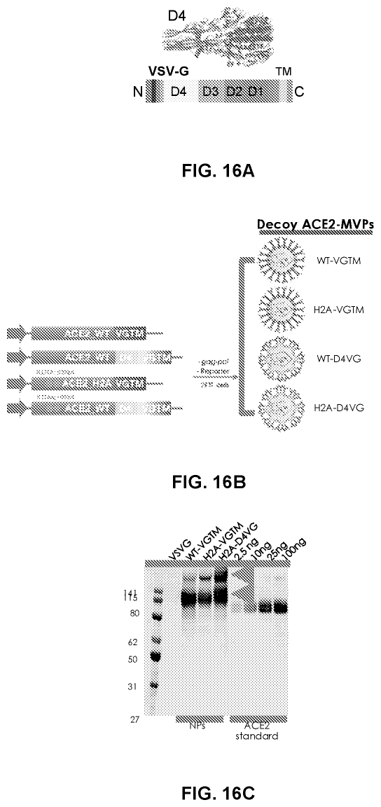

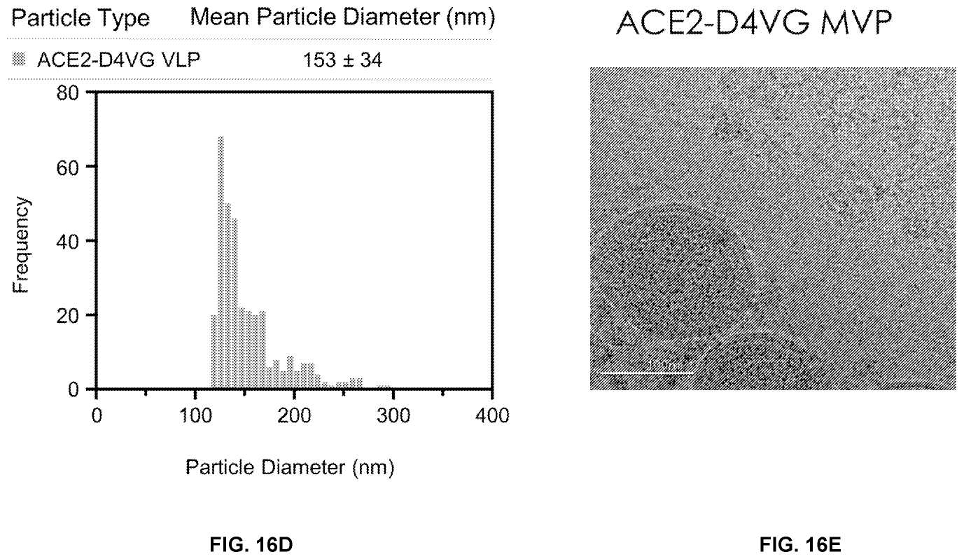

A D4 post-fusion trimerization domain from VSV-G protein (FIG. 16A) was used. A trimeric ACE2 display construct, designated as ACE2-D4VG, was designed to produce a fusion protein with the extracellular domain of ACE2, D4 trimerization domain, and VSVG transmembrane and cytosolic domain (FIG. 16B). Trimeric display constructs were generated that express with wild-type and enzymatically inactive ACE2 fusion proteins, designated as WT/ACE2-D4VG and H2A/ACE2-D4VG, respectively (FIG. 16B). Decoy-MVPs were produced by pseudotyping lentiviral viral-like particles (VLPs) with WT/ACE2-D4VG or H2A/ACE2-D4VG constructs via co-transfection of 293 T cells with a ACE2-display construct, lentiviral packaging constructs encoding structural components, and a lentiviral genome transfer vector encoding a GFP reporter (FIG. 16B). ACE2-MVPs were purified, and their concentrations were determined by p24 ELISA analysis. Copy numbers and oligomeric configurations of ACE2 fusion proteins on MVPs were determined via quantitative Western blot and PAGE analysis (FIG. 16C). Trimeric ACE2-display constructs were found to be highly effective in displaying both wild-type ACE2 and H2A/ACE2. Notably, the ACE2-VGTM display constructs pseudotyped VLPs with primarily monomeric ACE2 fusion proteins, whereas ACE2-D4VG display constructs pseudotyped VLPs with high levels of oligomerized ACE2 (FIG. 16C). The average particle diameter of ACE2-D4VG MVPs was 153±34 nm as determined by tunable resistive pulse sensing analyses (TRPS) using qNano (FIG. 16D). The morphology of ACE2-D4VG MVPs were characterized by cryoEM analyses at nominal magnification of 150,000× (FIG. 16E).

FIG. 16A-16E shows the design, generation, and activity of oligomerized display of wild-type and enzymatically inactive ACE2 on multivalent particles. FIG. 16A shows the structure of post-fusion VSV-G with D4 domain as the trimerization domain. FIG. 16B shows a schematic illustrating the oligomerized ACE2-displaying constructs with ACE2 extracellular domain fused to the VSVG transmembrane domain (ACE2-VGTM) for monomeric display or fused to the D4 post-fusion trimerization domain and VSVG transmembrane domain (ACE2-D4VG) for trimeric display. Multivalent particles display constructs with wild-type ACE2 (WT-ACE2) and enzymatic-inactive ACE2 (H2A/ACE2) were generated by co-transfecting corresponding ACE2-displaying constructs with a lentiviral packaging construct and lentiviral reporter construct. FIG. 16C shows the copy number of ACE2 molecules on the ACE2-MVPs, which were determined by quantitative Western-blot analyses. FIG. 16D shows representative TRPS analysis of ACE2-D4VG MVPs. FIG. 16E shows a representative Electron Microscopy image of H2A/ACE2-D4VG MVPs at nominal magnification of 150,000×.

### Example 12: Decoy-MVP Displaying Trimeric H2a/ACE2 is the Most Potent Inhibitors of SARS CoV-2 Viruses in Pseudovirus Neutralization Assays

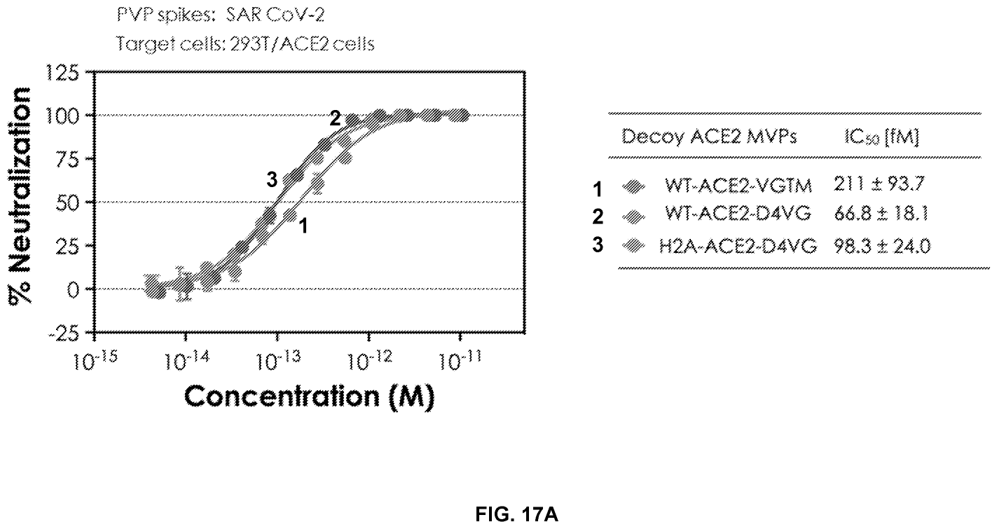

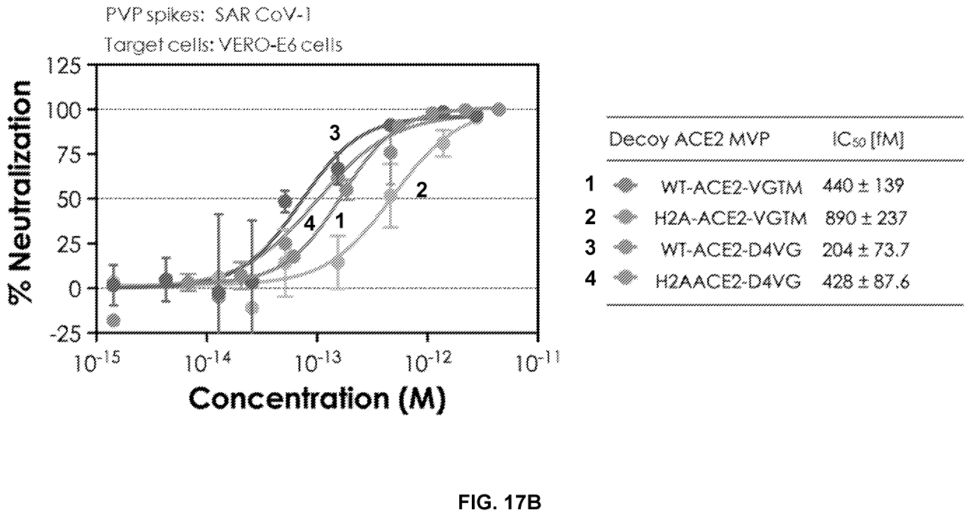

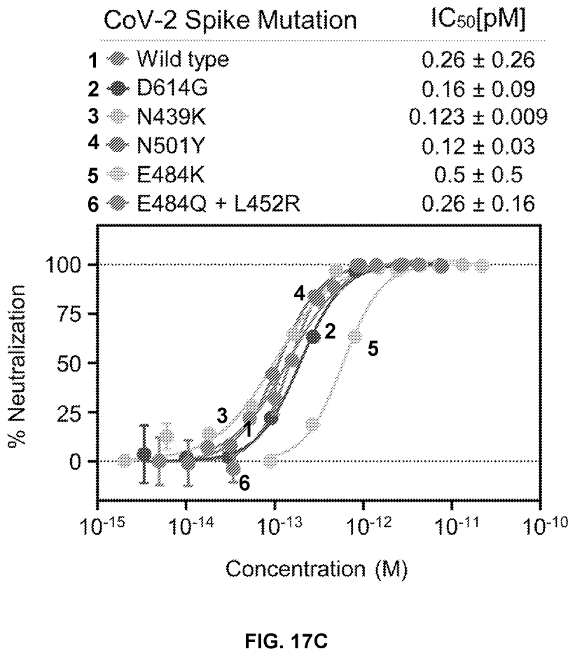

The neutralizing activity of both trimeric and monomeric ACE2-MVPs pseudotyped with wild-type and mutant H2A/ACE2 against SARS CoV-2 or CoV-1 in a pseudovirus neutralization assay was demonstrated, using 293T/ACE2 or VERO-E6 cells as target cells (FIG. 17A, B). Decoy-MVPs displaying trimeric WT/ACE2 and H2A/ACE were found to be both highly potent inhibitors, neutralizing CoV-2 pseudovirus at IC₅₀s of 66.8±18.1 fM and 98.3±24.0 fM, respectively. In contrast, decoy-MVPs pseudotyped with monomeric WT/ACE2 neutralize CoV-2 pseudovirus with IC₅₀s of 211±93.7 fM, an over 3-fold decrease in potency in comparison to their corresponding trimeric ACE2-MVPs (FIG. 15, FIG. 17A). MVPs displaying trimeric WT/ACE2 and H2A/ACE were shown to be both highly potent inhibitors, neutralizing CoV-1 pseudovirus at IC₅₀s of 204±73.7 fM and 428±87.6 fM, respectively. In contrast, MVPs pseudotyped with monomeric WT/ACE2 and H2A/ACE neutralize CoV-1 pseudovirus with IC₅₀s of 440±139 fM or 890±237 fM, an over 2-fold decrease in potency in comparison to their corresponding trimeric ACE2-MVPs (FIG. 15, FIG. 17B). These results demonstrated that MVPs with trimeric ACE2 display could further increase the neutralizing potency of WT and H2A mutant ACE2-MVPs against both CoV-2 and CoV-1 pseudoviruses.

FIG. 17A-17C show the neutralizing activity of enzymatically inactive ACE2 through oligomerized display H2A/ACE2 on MVPs. FIG. 17A shows the neutralizing activities of ACE2-MVPs displaying wild-type ACE2 or enzymatically inactive H2A/ACE2-MVPs in monomeric or trimeric form, which were determined in a SARS CoV-2 pseudovirus infection assay using 293 T/ACE2 cells as target cells. FIG. 17B shows the neutralizing activities of ACE2-MVPs displaying wild-type ACE2 or enzymatically inactive H2A/ACE2-MVPs in the monomeric or trimeric forms, which were determined in a SARS CoV-1 pseudovirus infection assay using VERO-E6 cells as target cells. FIG. 17C compares the neutralizing activities of the H2A/ACE2-D4VG MVPs against a variety of SARS CoV-2 variants in pseudovirus infection assay using 293 T/ACE2 cells as target cells.

### Example 13: Decoy-MVPs Displaying H2a/ACE2 are Potent Inhibitors Against Live SARS CoV-2 Viruses

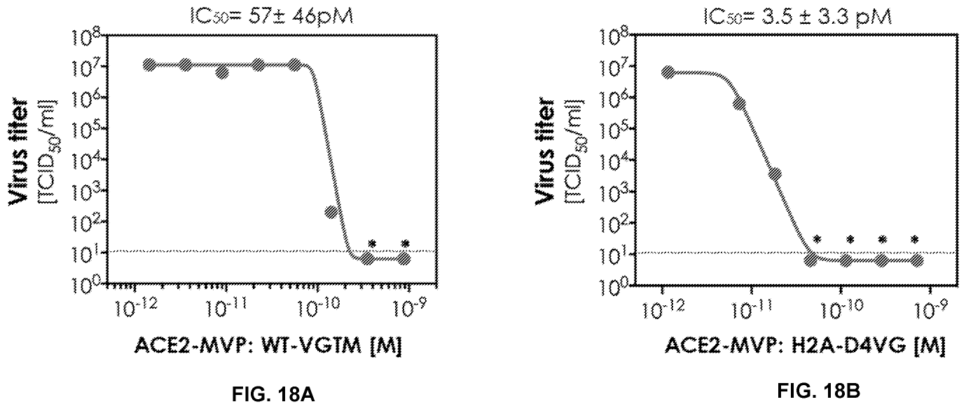

The neutralizing function of monomeric and trimeric ACE2-MVPs against live CoV-2 viruses was further characterized. Both monomeric and trimeric ACE2-MVPs were shown to reduce viral titer over six logs to an undetectable level in this microneutralization assay (FIG. 18A-18B). Notably, monomeric WT/ACE2-MVPs neutralize live CoV-2 virus at an IC₅₀ of 57±46 pM (FIG. 18A), whereas trimeric HA/ACE2-MVPs neutralize live CoV-2 virus at an IC₅₀ of 3.5±3.3 pM (FIG. 18B). These results demonstrated that oligomerized H2A/ACE2-MVPs were significantly more potent against live CoV-2 virus infection. Nevertheless, monomeric ACE2-MVPs were still highly potent inhibitors.

FIG. 18A-18B show the antiviral activity of ACE2-MVPs in a premixed live CoV-2 virus neutralization assay. FIG. 18A the neutralizing activities of monomeric wild-type ACE2-MVP (ACE2WT-VGTM MVP) determined using a SARS CoV-2 live virus neutralization assay. FIG. 18B shows the neutralizing activities of trimeric, enzymatically inactive H2A/ACE2-MVPs (H2A/ACE2-D4VG MVPs) determined using a SARS CoV-2 live virus neutralization assay.

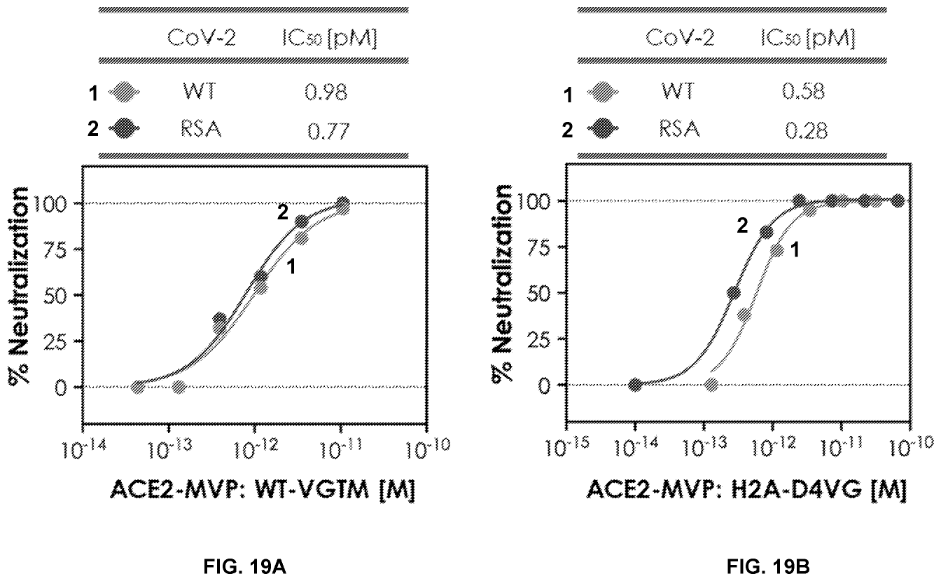

H2A/ACE2 Decoy-MVPs effectively neutralize B.1.351 South Africa variant in PRNT: Whether monomeric WT/ACE2-MVPs and trimeric H2A/ACE2-MVPs could effectively neutralize the B.1.351 South Africa strain of live CoV-2 containing E484K and N501Y mutations in a PRNT assay was further examined (FIG. 19A-19B). Monomeric WT/ACE2-MVPs neutralized both the original USA-WA1/2020 strain and South Africa B.1.351 strain at IC₅₀s of 0.98 pM and 0.77 pM, respectively (FIG. 19A). In comparison, trimeric H2A/ACE2-MVPs neutralized both the original USA-WA1/2020 strain and South Africa B.1.351 stain at 0.58 pM and 0.28 pM, respectively (FIG. 19B). Notably, both monomeric WT/ACE2-MVPs and trimeric H2A/ACE2-MVPs were comparable or have slightly higher potency against the South Africa B.1.351 strain in the PRNT assay. Moreover, trimeric H2A/ACE2-MVPs consistently outperform monomeric WT/ACE2-MVPs in the live virus neutralization assay. Clearly, both monomeric ACE2-MVPs and trimeric H2A/ACE2-MVPs were highly potent inhibitors against the original USA-WA1/2020 strain and South Africa B.1.351 strain—one of the key variants of concern in the ongoing pandemic, offering another critical advantage over neutralizing antibodies.

FIG. 19A shows the neutralizing activities of ACE2-MVPs displaying monomeric wild-type ACE2 against the original Washington strain of SARS CoV-2 or the South Africa variant of SARS CoV-2 in a live virus PRNT assay. FIG. 19B shows the neutralizing activities of ACE2-MVPs displaying trimeric H2A/ACE2 against the original Washington strain of SARS CoV-2 or the South Africa variant of SARS CoV-2 in a live virus PRNT assay.

### Example 14: Decoy-MVPs Displaying H2a/ACE2 are Potent Inhibitors of Live SARS CoV-2 Viruses in Hamsters

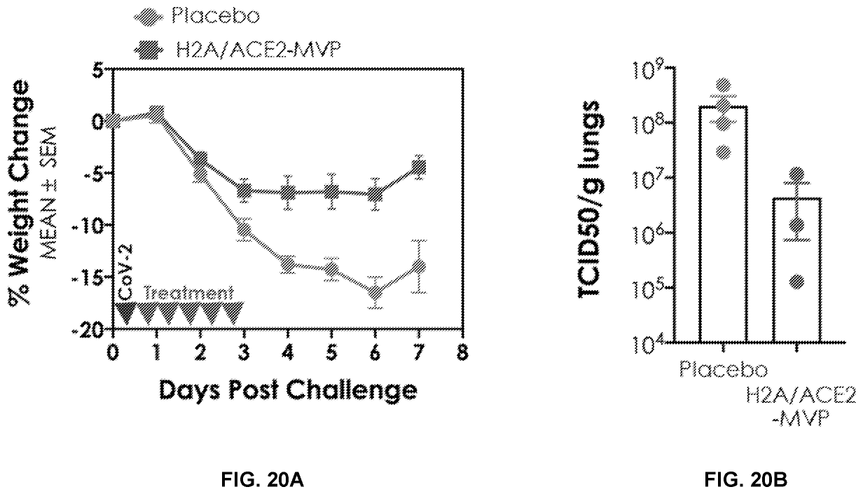

Golden hamsters inoculated with CoV-2 virus closely mimic more severe disease in humans. Affected hamsters develop readily observable clinical symptoms, including rapid weight loss accompanied by a very high viral load in the lungs and severe lung histology. To evaluate the ability of H2A/ACE2-MVPs to treated infected animals, hamsters were challenged with 2.3×10⁴ Pfu SARS CoV-2 virus and then treated hamsters with 1×10¹¹ particles of H2A/ACE2-MVPs through IN delivery. The treatments were started at 4 hours post virus challenging and given twice/day for a total of five doses. H2A/ACE2-MVPs treatments were observed to have significantly reduced weight loss from the challenged hamsters (FIG. 20A) and furthermore reduced viral load in lungs by more than one log (FIG. 20B). In summary, the hamster study demonstrated that H2A/ACE2-MVPs have potent neutralizing and therapeutic effects against CoV-2 infection in hamsters.

FIG. 20A shows the effect of trimeric H2A/ACE-MVPs in post-exposure treatment of SARS CoV-2 live virus infection on weight loss in hamsters. FIG. 20B shows the effect of trimeric H2A/ACE-MVPs in post-exposure treatment of SARS CoV-2 live virus infection on viral loads in lungs in hamsters.

### Example 15: Treatment with ACE2-MVPs Effectively Rescue Mice from Lethal Infection by SARS CoV-2

SARS CoV-2 infection causes lethality in the K18-hACE2 transgenic mice and induces symptoms and pathology recapitulating many of the defining features of severe COVID-19 in humans. High viral titer in lungs, with spread to brain and other organs, is observed in infected mice, coinciding with massive upregulation of inflammatory cytokines and infiltration of monocytes, neutrophils and activated T cells. This model has been used to test the efficacy of vaccine and therapeutics in preventing SARS-CoV-2 induced lethal infection.

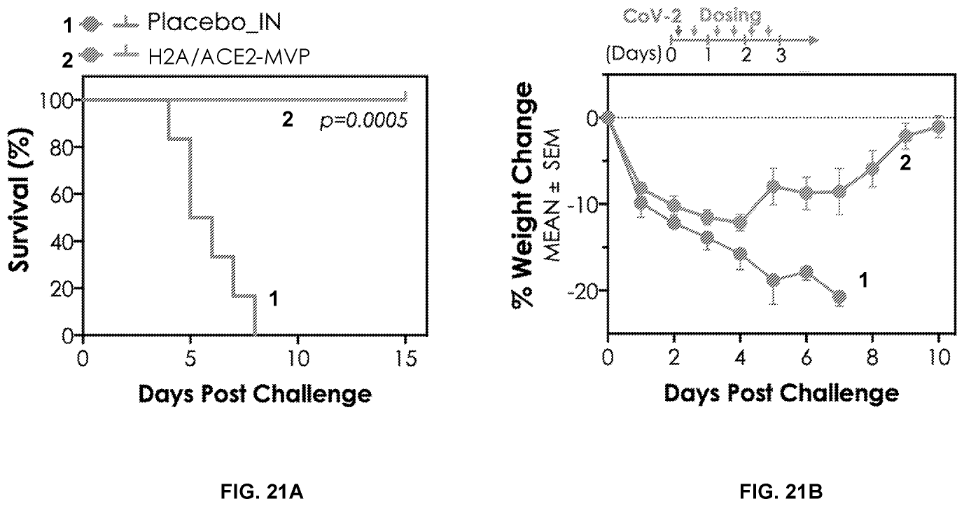

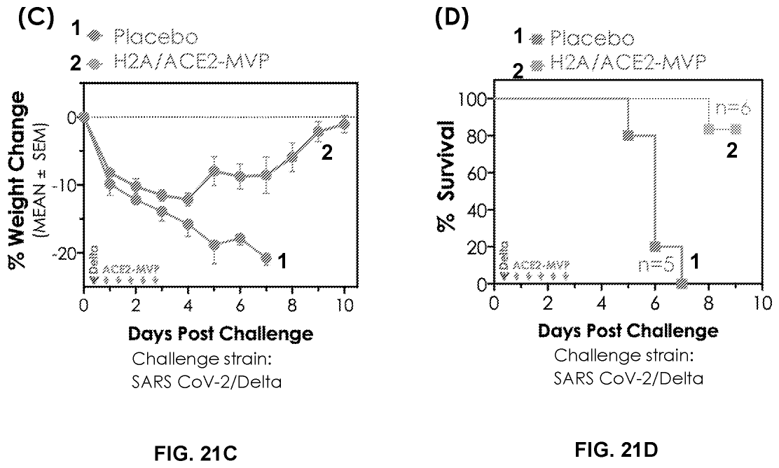

Whether IN delivery of ACE2-MVPs could protect ACE2 transgenic mice from SARS CoV-2 infection-related symptoms and lethality was investigated. K18-hACE2 mice were challenged with 2800 pfu of SARS CoV-2 (Strain USA-WA1/2020) and treated with 5 doses of H2A/ACE2-D4VG MVPs (lx 10¹¹ particles per dose) delivered IN. Dosing began 4-hours post-infection, and subsequent doses were given twice a day at day 1 and day 2 post-infection. Mice in the treatment group exhibited no respiratory symptoms and all survived the infection (FIG. 21A), whereas all mice in the placebo group succumbed to infection at approximately day 6 post-infection. Moreover, in comparison to the placebo group, mice in the treatment group experienced modest or no respiratory symptoms and only transitory weight loss (FIG. 21B). The results demonstrated that H2A/ACE2 MVPs could rescue lethal SARS CoV-2 infection and completely prevent respiratory symptoms in the K18-hACE2 transgenic mouse model, a model recapitulating severe COVID-19 in humans.

Furthermore, whether IN delivery of ACE2-MVPs protected ACE2 transgenic mice from Delta variant infection-related symptoms and lethality was also investigated. K18-hACE2 mice were challenged with 800 pfu of SARS CoV-2 (Delta variant NR55674) and treated with 5 doses of H2A/ACE2-D4VGMVPs (1× 10¹¹ particles per dose) delivered IN. Again, dosing began 4-hours post-infection, and subsequent doses were given twice a day at day 1 and day 2 post-infection. Mice in the treatment group exhibited no respiratory symptoms and all but one survived the infection (FIG. 21C), whereas all mice in the placebo group succumbed to infection at approximately day 6 post-infection. Moreover, in comparison to the placebo group, the five surviving mice in the treatment group experienced modest or no respiratory symptoms and only transitory weight loss (FIG. 21D). Thus, H2A/ACE2 MVPs rescued lethal SARS CoV-2 Delta variant infection and largely prevented respiratory symptoms in the K18-hACE2 transgenic mouse model, demonstrating that ACE2-MVPs potentially can be used as therapeutics against all SARS CoV-2 variants utilizing ACE2 as an entry receptor.

FIG. 21A-21B show the efficacy of trimeric H2A/ACE-MVPs in post-exposure treatment of SARS CoV-2 live virus infection in the hACE2 transgenic mice. FIG. 21A shows the effect of trimeric H2A/ACE-MVPs treatment on survival in ACE2 mice challenged with the WA strain of SARS CoV-2. FIG. 21B shows the effect of trimeric H2A/ACE2 MVPs treatment on weight loss in ACE2 mice challenged with the WA strain of SARS CoV-2. FIG. 21C depicts the effect of trimeric H2A/ACE-MVPs treatment on survival of SARS CoV-2 Delta variant infected hACE2 transgenic mice. FIG. 21D shows the effects of the weight loss in hACE2 transgenic mice infected with the SARS CoV-2 Delta variant.

### Example 16: ACE2-MVP Treatment of SARS CoV-2 Infection Induces Robust Immunity Against Dominant Delta Variant

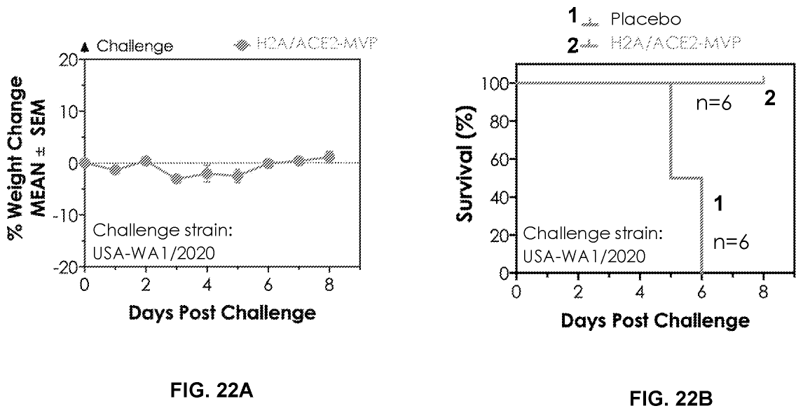

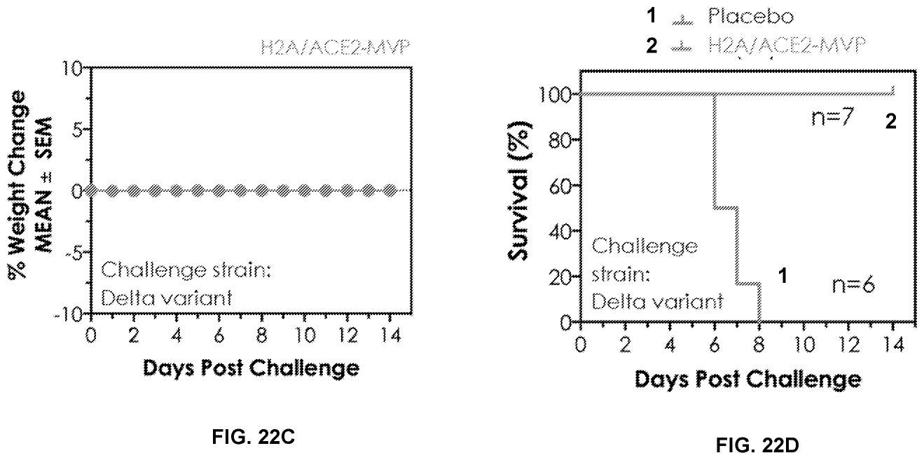

To examine how decoy-MVP treatment of SARS CoV-2 may affect the development of antiviral immunity post-infection, we re-challenged the hACE2 mice rescued from primary infection with various strains of SARS CoV-230 days after the initial infection. First, mice were challenged with the original SARS CoV-2 strain, the same virus strain using in the primary infection. No noticeable respiratory symptoms, weight loss (FIG. 22A), or death (FIG. 22B) were observed in the re-challenged survivors. Further, another group of hACE2 mice rescued from primary infection with the Delta variant of SARS CoV-2 were re-challenged at about 9000 Pfu, a viral dosage that was at least three times higher than virus dosage used in the primary infection. Again, no noticeable respiratory symptoms, weight loss (FIG. 22C), or death (FIG. 22D) were observed. Notably, ACE2-MVP treatment of SARS CoV-2 infected hACE2 mice not only rescued these mice from lethal infection and eliminated all respiratory symptoms through drastically reduction peak viral load in these mice. Nevertheless, these mice developed robust immunity against both the original SARS CoV-2 strain as well as the Delta variant. Thus, hACE2 mice surviving primary challenge as a result of ACE2-MVP treatment developed robust immunity against the original SARS CoV-2 strain as well as the Delta variant.

FIG. 22A-22D show that ACE2 mice rescued by the H2A/ACE2-D4VG MVP were resistant to re-challenge with the original SARS CoV-2 strain as well as the Delta variant. ACE2 mice survived from primary SARS CoV-2 challenge with trimeric H2A/ACE2 MVPs were challenged again with the original SARS CoV-2 strain as well as the Delta variant. FIG. 22A shows the effect of SARS CoV-2 re-challenge on body weight of ACE2 transgenic mice. FIG. 22B shows the effect of SARS CoV-2 re-challenge on survival of ACE2 transgenic mice. FIG. 22C shows the effect of Delta variant re-challenge on body weight of ACE2 transgenic mice. FIG. 22D shows the effect of Delta variant re-challenge on survival of ACE2 transgenic mice.

### Example 17: EV-Based ACE2-MVPs are Highly Potent Inhibitors Against Live CoV-2 Viruses

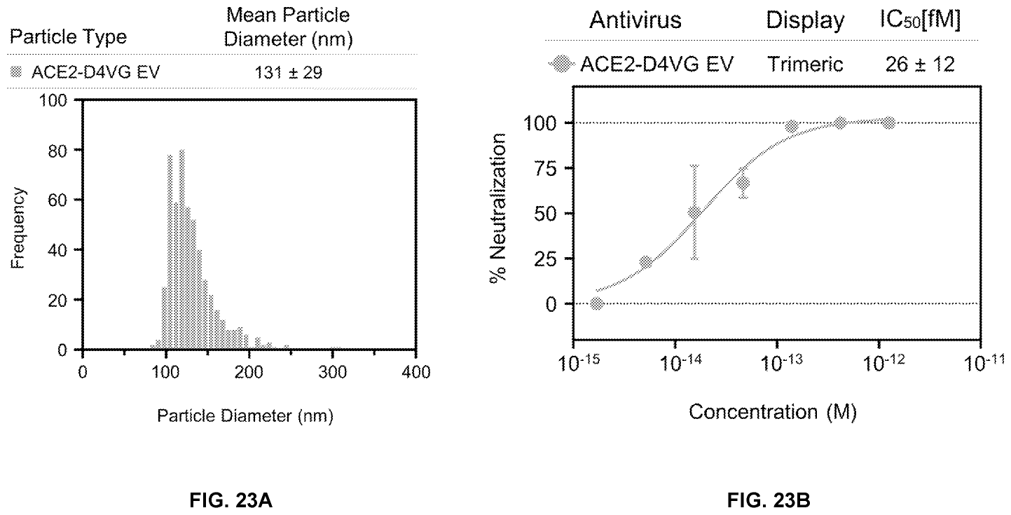

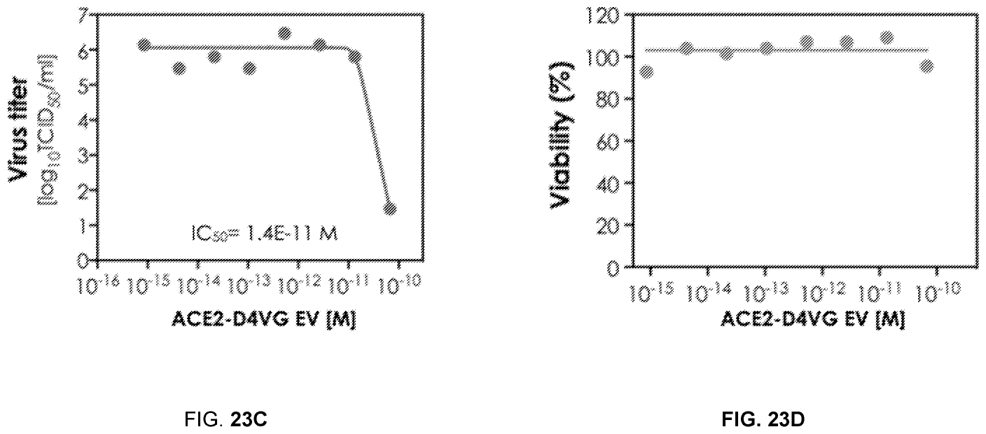

By transfecting 293 T cells with only the trimeric decoy-receptor displaying vector (FIG. 16A) without the lentiviral packaging vector, EVs displaying multiple copies of ACE2 were generated, designated EV-based ACE2-MVPs. The mean diameter of EV-based ACE2-MVPs was 131±29 nm as determined by TRPS analysis (FIG. 23A). Moreover, EVs displaying trimeric H2A/ACE2 were highly potent inhibitors, neutralizing CoV-2 pseudovirus at IC₅₀s of 26±12 fM. Furthermore, trimeric EV-based ACE2-MVPs neutralized live CoV-2 virus at an IC₅₀ of 14 pM in post-infection live CoV-2 microneutralization assays and reduced viral titer by over five logs (FIG. 23C) without noticeable cytotoxicity (FIG. 23D). The results demonstrate that EV-based ACE2-MVPs were highly potent neutralizers of SARS CoV-2.

FIG. 23A-23D show particle analysis and in vitro neutralizing efficacy of EV-based ACE2-MVPs. FIG. 23A shows particle size distribution of EV-based ACE2-MVPs as determined by Tunable Resistive Pulse Sensing Analysis using a qNano instrument. FIG. 23B shows the neutralizing activity of EV-based ACE2-MVPs determined in a SARS CoV-2 pseudovirus infection assay using 293T/ACE2 cells as target cells. FIG. 23C shows the neutralizing activity of EV-based ACE2-MVPs as determined in a SARS CoV-2 live virus neutralization assay. FIG. 23D shows the cytotoxicity of EV-based ACE2-MVPs as determined in a SARS CoV-2 live virus neutralization assay.

### Example 18: Design Strategy of Decoy-MVP Display Vector

The results presented above demonstrate that decoy-MVPs are a novel class of highly potent antivirals against pandemic viruses. Decoy-MVPs were designed to be the mirror image of its targeting virus and display the viral-entry receptors that match to the oligomeric multivalent spike proteins on the virus envelope. Two different types of enveloped particle display vectors were prepared for efficient protein display on VLP and extracellular vesicles (EV).

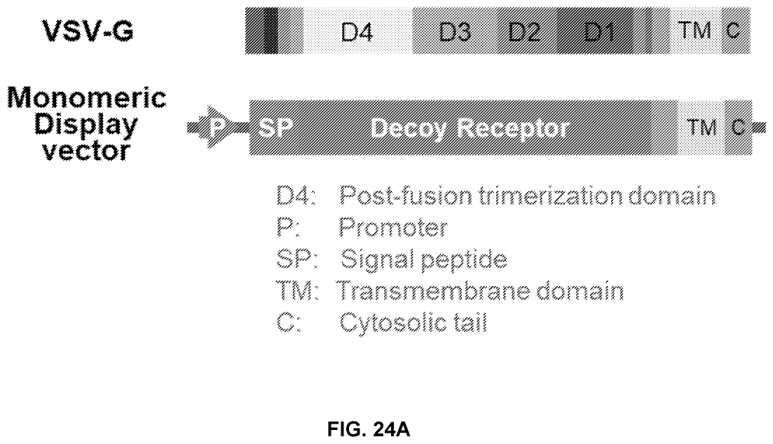

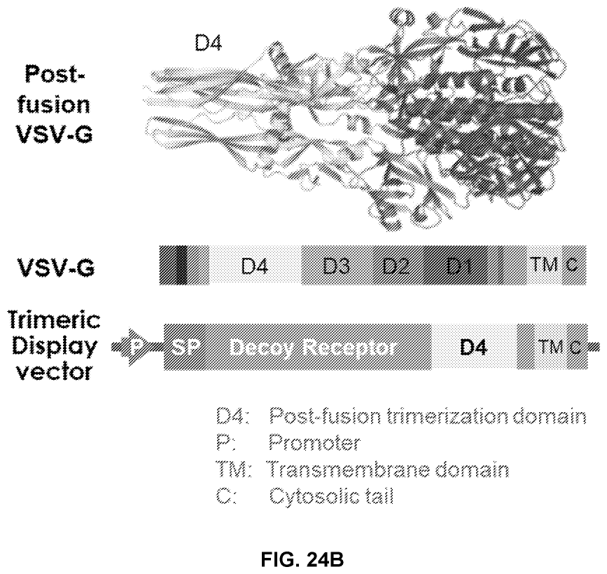

A monomeric display vector expressing a fusion protein consisting of the extracellular domain of a viral entry receptor decoy linked to the VSVG transmembrane and intracellular domains is designed as shown in FIG. 24A to display hundreds of copies of monomeric proteins on the surface of VLPs and EVs. Aside from the use of monomeric formats that are suited to form high avidity interactions with similarly multivalently displayed patterned viral spike proteins, enveloped particles are made to match oligomeric display formats of viral spike proteins to further enhance avidity at the level of individual oligomeric binding partners. To this end, a trimeric display vector expressing a fusion protein consisting of the extracellular domain of a viral entry receptor decoy linked to the D4 post-fusion trimerization domain of VSVG, followed by the transmembrane and intracellular domains of VSVG is designed as shown in FIG. 24B. The vector is used to display hundreds of copies of trimeric proteins on the surface of VLPs and EVs and are well suited to form high avidity interactions with similarly oligomeric proteins on the viral envelope.

FIG. 24A-24B show vectors for multivalent displaying of decoy viral entry receptors on enveloped particles in varied oligomeric format. FIG. 24A shows a monomeric display of viral entry receptors on enveloped particles by using a vector expressing a fusion protein consisting of a decoy viral entry receptor linked to the VSVG transmembrane and intracellular domains. FIG. 24B shows a trimeric or oligomeric display of viral entry receptors on enveloped particles by using a vector expressing a fusion protein consisting of a protein linked to the D4 post-fusion trimerization domain of VSVG, followed by the transmembrane and intracellular domains of VSVG.

### Example 19: Generation of Monomeric Decoy-MVPs

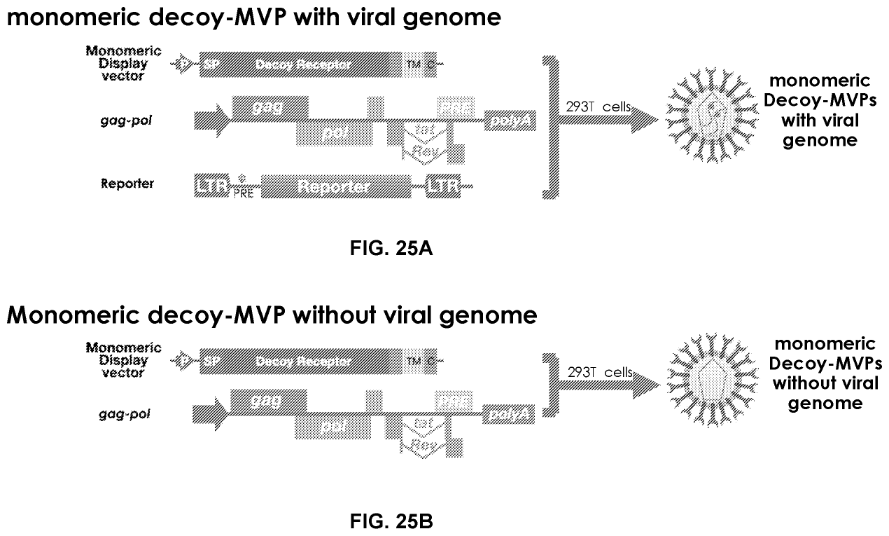

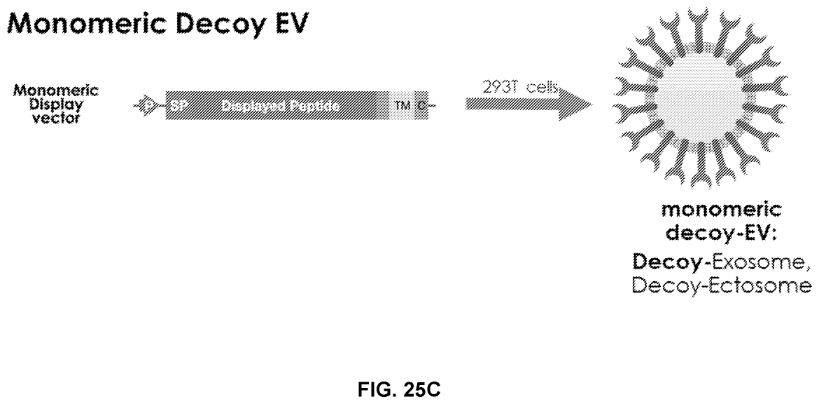

Multivalent decoy receptors are displayed as monomers on the surface of a VLP and an extracellular vesicle using a monomeric display vector. The monomeric VLP-based decoy-MVP is produced with viral RNA genomes in which the monomeric peptide display construct with a lentiviral packaging construct expresses essential packaging components including Gag-Pol and Rev proteins and a viral genome transfer encoding a GFP/luciferase reporter as shown in FIG. 25A. The monomeric VLP-based decoy-MVP without RNA genome is produced by co-transfecting displaying vector with only a lentiviral packaging construct but not the viral genome transfer vector as shown in FIG. 25B. The monomeric EV-based decoy-MVP which includes decoy-exosome and decoy-ectosome is produced by transfecting only monomeric peptide displaying vector in 293T cells as shown in FIG. 25C.

FIG. 25A shows monomeric decoy-MVP production by pseudo-typing ACE2 receptors on the lentiviral-based viral-like particles with viral genome. FIG. 25B shows Monomeric decoy-MVP production by pseudo-typing ACE2 receptors on the lentiviral-based viral-like particles without viral genome. FIG. 25C shows monomeric decoy-MVP production by pseudo-typing extracellular vesicles with ACE2 receptors.

### Example 20: Generation of Trimeric Decoy-MVPs

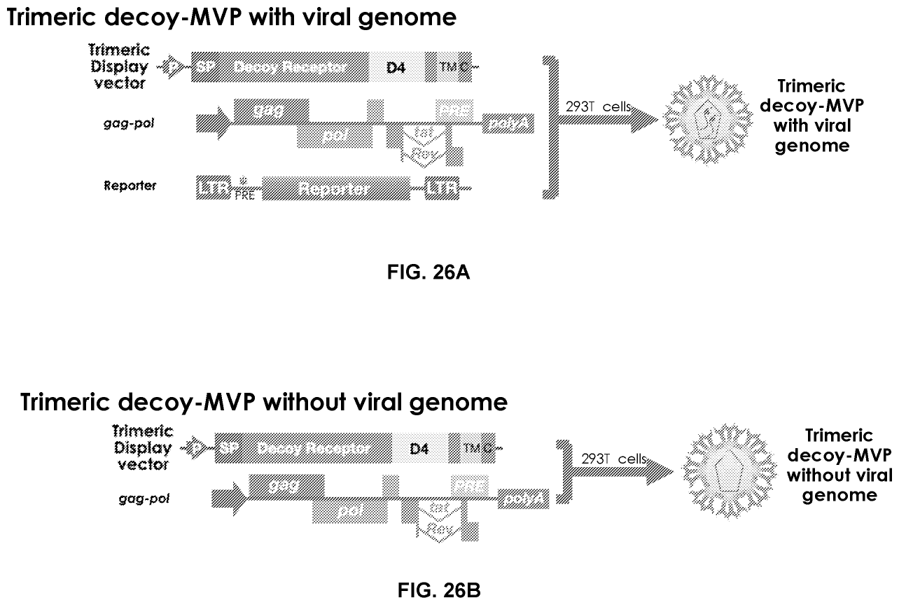

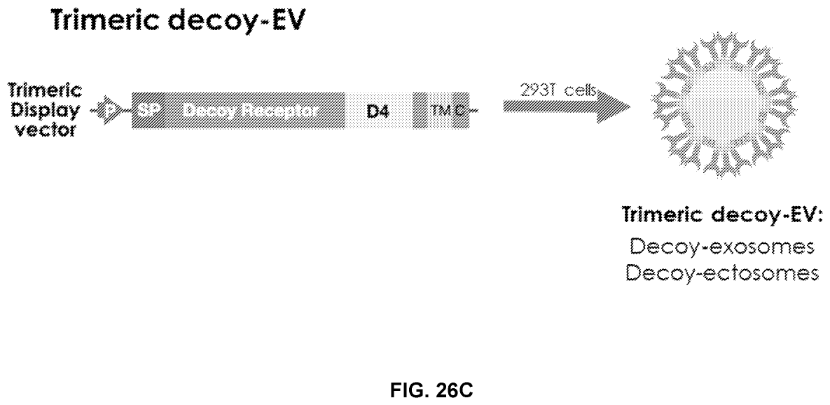

Multivalent decoy receptors are displayed as trimers on the surface of a VLP and an extracellular vesicle using a trimeric display vector. The trimeric VLP-based decoy-MVP is produced with viral RNA genomes in which the trimeric peptide display construct with a lentiviral packaging construct expresses essential packaging components including Gag-Pol and Rev proteins and a viral genome transfer encoding a GFP/luciferase reported as shown in FIG. 26A. The trimeric VLP-based enveloped particle without RNA genome is produced by co-transfecting displaying vector together with only a lentiviral packaging construct but not the viral genome transfer vector as shown in FIG. 26B. The trimeric EV-based decoy-MVP which includes decoy-exosome and decoy-ectosome is produced by transfecting only the trimeric peptide displaying vector in 293T cells as shown in FIG. 26C.

FIG. 26A-26C show in vitro production of trimeric decoy-MVPs. FIG. 26A shows trimeric decoy-MVP production by pseudo-typing ACE2 receptors onto the lentiviral-based viral-like particles with viral genome. FIG. 26B shows trimeric decoy-MVP production by pseudo-typing ACE2 receptors onto the lentiviral-based viral-like particles without viral genome. FIG. 26C shows trimeric decoy-MVP production by pseudo-typing extracellular vesicles with ACE2 receptors.

### Example 21: Generation of Mixed Monomeric and Trimeric Decoy-MVP

To further increase decoy display density, enveloped particles displaying mixed monomeric and trimeric decoy receptor are generated by co-transfecting monomeric and trimeric decoy display constructs. Such design is used to increase the density of the displayed peptide or to create combinatorial of distinct displayed decoys. Mixed monomeric and trimeric decoy-MVPs are built with VLPs and EVs by co-transfecting monomeric and trimeric display vectors.

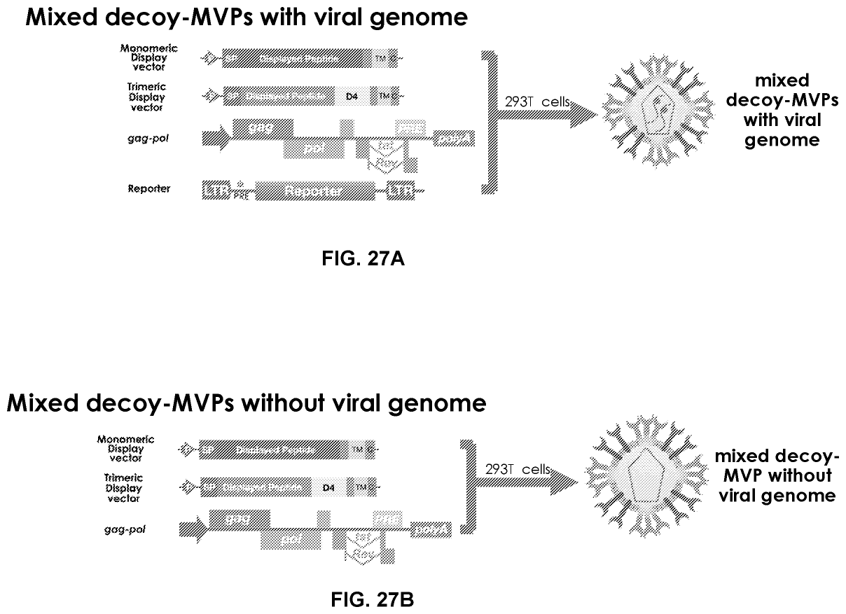

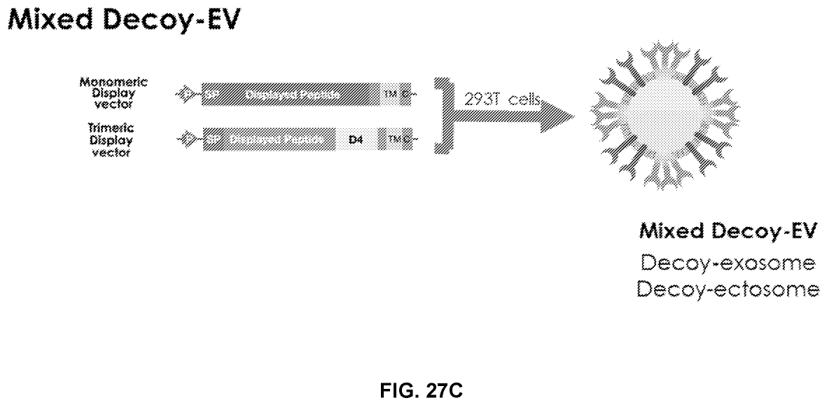

To produce mixed decoy-MVPs with viral RNA genomes, the mixed monomeric and trimeric decoy receptor display constructs are co-transfected with a lentiviral packaging construct expressing essential packaging components, such as Gag-Pol and Rec proteins, and viral genome transfer vector encoding a GFP/luciferase reported as shown in FIG. 27A. The mixed decoy-MVPs without RNA genome are produced by co-transfecting the mixed monomeric and trimeric display vector together with only a lentiviral packaging construct but not the viral genome transfer vector as shown in FIG. 27B. The mixed EV-based decoy-MVPs which includes mixed decoy-exosome and decoy-ectosome is produced by transfecting the mixed monomeric and trimeric display peptide constructs into 293T cells as shown FIG. 27C.

FIG. 27A-27C show in vitro production of mixed monomeric and trimeric decoy-MVPs. FIG. 27A shows mixed monomeric and trimeric decoy-MVP production by pseudo-typing viral-entry receptors onto the lentiviral-based viral-like particles with viral genome. FIG. 27B shows mixed monomeric and trimeric decoy-MVP production by pseudo-typing viral-entry receptors onto the lentiviral-based viral-like particles without viral genome.

FIG. 27C shows mixed monomeric and trimeric decoy-MVP production by pseudo-typing extracellular vesicles with viral-entry receptors.

### Example 22: Oligomerization Configurations for the Decoy Proteins Displayed on Decoy-MVPs

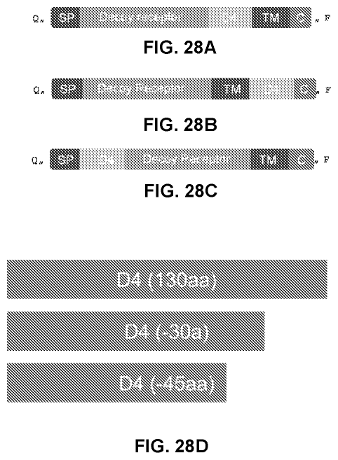

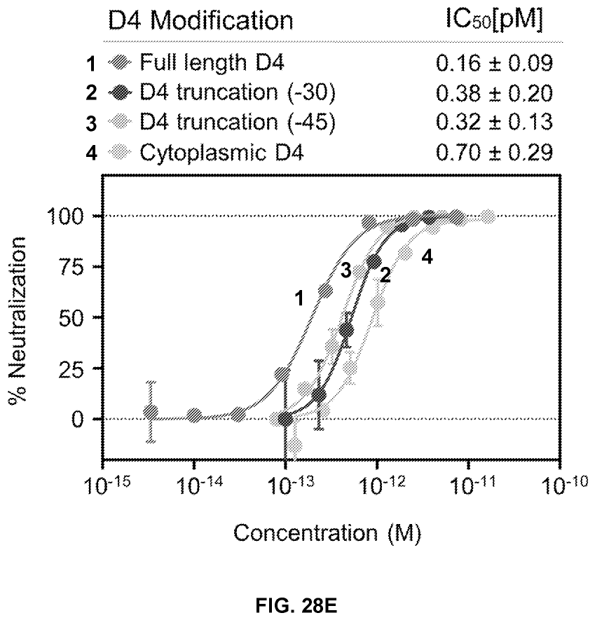

Decoy-MVPs were genetically programmed to display peptides of interest in various configurations by modifying the display vector as shown in FIGS. 28A-28C. The VSVG D4 trimerization domain were placed at various positions of the fusion peptide: (1) extracellular and juxtaposed to the transmembrane domain; (2) intracellular and juxtaposed to the transmembrane domain; (3) extracellular and after the signal peptide (FIGS. 28A-28C). Moreover, the length of D4 trimerization domain was varied from 85 to 100 to 130 amino acids (FIG. 28D). H2A/ACE2-D4VG MVPs with varied D4 location and length were shown to be highly potent inhibitors, neutralizing CoV-2 pseudovirus at IC₅₀s below 1 pM (FIG. 28E).

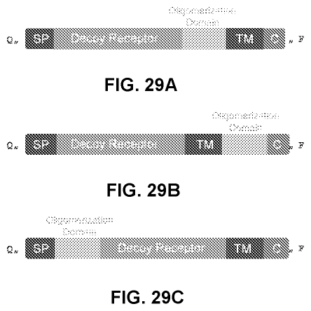

Furthermore, various oligomerization domains may be used for distinct surface display patterns that are suitable for the function of decoy receptors (FIGS. 29A-19C). In addition to the VSVG D4 trimerization domain, the Dengue E protein fusion domain or a foldon domain are used to create trimeric display patterns on the surface of VLPs and EVs. Leucine zipper domains and the influenza neuraminidase stem domain are used to create dimeric and tetrameric display patterns on the surface of VLPs and EVs, respectively. Exemplary oligomerization domains and valence are summarized in Table 4. With these display configurations, it is possible to program combinatorial decoy-MVPs with mixed monomeric, dimeric, trimeric, and tetrameric display patterns optimized to their function in target cell regulation or virus neutralization.

TABLE 4
Exemplary oligomerization domains and valence

| Oligomerization Domain | Valence |
|---|---|
| VSV-G protein D4 | Trimer |
| Dengue E protein fusion protein | Trimer |
| Foldon | Trimer |
| human C-propeptide of α1(I) collagen | Trimer |
| Leucine Zipper | Dimer |
| Influenza Neuraminidase stem | Tetramer |

### Example 23: Exemplary Sequences

TABLE 5
Sequences

| | SEQ | | |
|---|---|---|---|
| | ID | Accession | |
| Name | NO | Number | Amino Acid Sequence |
| ACE2 | 1 | NP_001358344 | MSSSSWLLLSLVAVTAAQSTIEEQAKTFLDKFNHEA |
| | | | EDLFYQSSLASWNYNTNITEENVQNMNNAGDKWS |
| | | | AFLKEQSTLAQMYPLQEIQNLTVKLQLQALQQNGSS |
| | | | VLSEDKSKRLNTILNTMSTIYSTGKVCNPDNPQECLL |
| | | | LEPGLNEIMANSLDYNERLWAWESWRSEVGKQLRP |
| | | | LYEEYVVLKNEMARANHYEDYGDYWRGDYEVNG |
| | | | VDGYDYSRGQLIEDVEHTFEEIKPLYEHLHAYVRAK |
| | | | LMNAYPSYISPIGCLPAHLLGDMWGRFWTNLYSLTV |
| | | | PFGQKPNIDVTDAMVDQAWDAQRIFKEAEKFFVSV |
| | | | GLPNMTQGFWENSMLTDPGNVQKAVCHPTAWDLG |
| | | | KGDFRILMCTKVTMDDFLTAHHEMGHIQYDMAYA |
| | | | AQPFLLRNGANEGFHEAVGEIMSLSAATPKHLKSIG |
| | | | LLSPDFQEDNETEINFLLKQALTIVGTLPFTYMLEKW |
| | | | RWMVFKGEIPKDQWMKKWWEMKREIVGVVEPVP |
| | | | HDETYCDPASLFHVSNDYSFIRYYTRTLYQFQFQEA |
| | | | LCQAAKHEGPLHKCDISNSTEAGQKLFNMLRLGKSE |
| | | | PWTLALENVVGAKNMNVRPLLNYFEPLFTWLKDQ |
| | | | NKNSFVGWSTDWSPYADQSIKVRISLKSALGDKAYE |
| | | | WNDNEMYLFRSSVAYAMRQYFLKVKNQMILFGEE |
| | | | DVRVANLKPRISFNFFVTAPKNVSDIIPRTEVEKAIR |
| | | | MSRSRINDAFRLNDNSLEFLGIQPTLGPPNQPPVSIW |
| | | | LIVFGVVMGVIVVGIVILIFTGIRDRKKKNKARSGEN |
| | | | PYASIDISKGENNPGFQNTDDVQTSF |
| DPP4 | 2 | NP_001926 | MKTPWKVLLGLLGAAALVTIITVPVVLLNKGTDDA |
| | | | TADSRKTYTLTDYLKNTYRLKLYSLRWISDHEYLYK |
| | | | QENNILVFNAEYGNSSVFLENSTFDEFGHSINDYSISP |
| | | | DGQFILLEYNYVKQWRHSYTASYDIYDLNKRQLITE |
| | | | ERIPNNTQWVTWSPVGHKLAYVWNNDIYVKIEPNL |
| | | | PSYRITWTGKEDIIYNGITDWVYEEEVFSAYSALWW |
| | | | SPNGTFLAYAQFNDTEVPLIEYSFYSDESLQYPKTVR |
| | | | VPYPKAGAVNPTVKFFVVNTDSLSSVTNATSIQITAP |
| | | | ASMLIGDHYLCDVTWATQERISLQWLRRIQNYSVM |
| | | | DICDYDESSGRWNCLVARQHIEMSTTGWVGRFRPSE |
| | | | PHFTLDGNSFYKIISNEEGYRHICYFQIDKKDCTFITK |
| | | | GTWEVIGIEALTSDYLYYISNEYKGMPGGRNLYKIQ |
| | | | LSDYTKVTCLSCELNPERCQYYSVSFSKEAKYYQLR |
| | | | CSGPGLPLYTLHSSVNDKGLRVLEDNSALDKMLQN |
| | | | VQMPSKKLDFIILNETKFWYQMILPPHFDKSKKYPLL |
| | | | LDVYAGPCSQKADTVFRLNWATYLASTENIIVASFD |
| | | | GRGSGYQGDKIMHAINRRLGTFEVEDQIEAARQFSK |
| | | | MGFVDNKRIAIWGWSYGGYVTSMVLGSGSGVFKC |
| | | | GIAVAPVSRWEYYDSVYTERYMGLPTPEDNLDHYR |
| | | | NSTVMSRAENFKQVEYLLIHGTADDNVHFQQSAQIS |
| | | | KALVDVGVDFQAMWYTDEDHGIASSTAHQHIYTH |
| | | | MSHFIKQCFSLP |
| | 3 | NP_955548 | KFTIVFPHNQKGNWKNVPSNYHYCPSSSDLNWHND |
| | | | LIGTALQVKMPKSHKAIQADGWMCHASKWVTTCD |
| | | | FRWYGPKYITHSIRSFTPSVEQCKESIEQTKQGTWLN |
| | | | PGFPPQSCGYATVIDAEAVIVQVTPHHVLVDEYTGE |
| | | | WVDSQFINGKCSNYICPTVHNSTTWHSDYKVKGLC |
| | | | DSNLISMDITFFSEDGELSSLGKEGTGFRSNYFAYET |
| | | | GGKACKMQYCKHWGVRLPSGVWFEMADKDLFAA |
| | | | ARFPECPEGSSISAPSQTSVDVSLIQDVERILDYSLCQ |
| | | | ETWSKIRAGLPISPVDLSYLAPKNPGTGPAFTIINGTL |
| | | | KYFETRYIRVDIAAPILSRMVGMISGTTTERELWDD |
| | | | WAPYEDVEIGPNGVLRTSSGYKFPLYMIGHGMLDS |
| | | | DLHLSSKAQVFEHPHIQDAASQLPDDESLFFGDTGLS |
| | | | KNPIELVEGWFSSWKSSIASFFFIIGLIIGLFLVLRVGI |
| | | | HLCIKLKHTKKRQIYTDIEMNRLGK |
| | 4 | QJF75467 | MFVFLVLLPLVSSQCVNLTTRTQLPPAYTNSFTRGV |
| | | | YYPDKVFRSSVLHSTQDLFLPFFSNVTWFHAIHVSGT |
| | | | NGTKRFDNPVLPFNDGVYFASTEKSNIIRGWIFGTTL |
| | | | DSKTQSLLIVNNATNVVIKVCEFQFCNDPFLGVYYH |
| | | | KNNKSWMESEFRVYSSANNCTFEYVSQPFLMDLEG |
| | | | KQGNFKNLREFVFKNIDGYFKIYSKHTPINLVRDLPQ |
| | | | GFSALEPLVDLPIGINITRFQTLLALHRSYLTPGDSSS |
| | | | GWTAGAAAYYVGYLQPRTFLLKYNENGTITDAVDC |
| | | | ALDPLSETKCTLKSFTVEKGIYQTSNFRVQPTESIVRF |
| | | | PNITNLCPFGEVENATRFASVYAWNRKRISNCVADY |
| | | | SVLYNSASFSTFKCYGVSPTKLNDLCFTNVYADSFVI |
| | | | RGDEVRQIAPGQTGKIADYNYKLPDDFTGCVIAWNS |
| | | | NNLDSKVGGNYNYLYRLFRKSNLKPFERDISTEIYQ |
| | | | AGSTPCNGVEGFNCYFPLQSYGFQPTNGVGYQPYR |
| | | | VVVLSFELLHAPATVCGPKKSTNLVKNKCVNFNFN |
| | | | GLTGTGVLTESNKKFLPFQQFGRDIADTTDAVRDPQ |
| | | | TLEILDITPCSFGGVSVITPGTNTSNQVAVLYQDVNC |
| | | | TEVPVAIHADQLTPTWRVYSTGSNVFQTRAGCLIGA |
| | | | EHVNNSYECDIPIGAGICASYQTQTNSPRRARSVASQ |
| | | | SIIAYTMSLGAENSVAYSNNSIAIPTNFTISVTTEILPV |
| | | | SMTKTSVDCTMYICGDSTECSNLLLQYGSFCTQLNR |
| | | | ALTGIAVEQDKNTQEVFAQVKQIYKTPPIKDFGGFN |
| | | | FSQILPDPSKPSKRSFIEDLLFNKVTLADAGFIKQYGD |
| | | | CLGDIAARDLICAQKFNGLTVLPPLLTDEMIAQYTSA |
| | | | LLAGTITSGWTFGAGAALQIPFAMQMAYRENGIGVT |
| | | | QNVLYENQKLIANQFNSAIGKIQDSLSSTASALGKLQ |
| | | | DVVNQNAQALNTLVKQLSSNFGAISSVLNDILSRLD |
| | | | KVEAEVQIDRLITGRLQSLQTYVTQQLIRAAEIRASA |
| | | | NLAATKMSECVLGQSKRVDFCGKGYHLMSFPQSAP |
| | | | HGVVFLHVTYVPAQEKNFTTAPAICHDGKAHFPREG |
| | | | VFVSNGTHWFVTQRNFYEPQIITTDNTFVSGNCDVVI |
| | | | GIVNNTVYDPLQPELDSFKEELDKYFKNHTSPDVDL |
| | | | GDISGINASVVNIQKEIDRLNEVAKNLNESLIDLQEL |
| | | | GKYEQYIKWPWYIWLGFIAGLIAIVMVTIMLCCMTS |
| | | | CCSCLKGCCSCGSCCKFDEDDSEPVLKGVKLHYT |

### Example 24: Discussion

A novel strategy to neutralize emerging coronaviruses was established using decoy-multivalent particles displaying high copy numbers of decoy viral-entry receptors. MVPs displaying ACE2 or DPP4 effectively neutralized SARS CoV-1/CoV-2, emerging CoV-2 variants and MERS coronaviruses. The decoy-MVPs were remarkably potent, often neutralizing their target viruses at sub-picomolar IC₅₀s and completely eliminating viral titers in live virus neutralization and antiviral assays. Furthermore, decoy-MVPs were over 10,000-fold more potent than their corresponding mono or low-valency recombinant entry receptor proteins. This enhancement correlated with the number of copies viral entry receptors displayed on the surface of these particles. With as low as ten receptor copies per MVP, the decoy-MVPs were already more potent than many clinical-stage neutralizing antibodies, and their potency were further increased by orders of magnitude by displaying more decoy receptors. The results demonstrate that the high valency of viral receptors on MVPs was the key to enabling maximum neutralizing efficacy of decoy-MVPs.

Multivalent interactions result in potent neutralization by decoy-MVPs: SARS CoV-2 virions, as well as many other enveloped and non-enveloped viruses, display hundreds of copies of large spike proteins and utilize multivalent interactions between spike and host-cell proteins for attachment and entry. The boost in functional affinity that viruses receive through multivalent interactions is exponential, and nearly all enveloped and non-enveloped viruses use this multivalent strategy for attachment and host-cell entry. This provides a tremendous advantage to viruses. Most notably, the multivalent strategy enabled viruses to turn relatively weak monovalent interactions with millimolar binding affinities into super-strong multivalent interactions with functional affinities in the nanomolar to picomolar range, in turn creating a high threshold for low or monovalent binders, such as neutralizing antibodies and recombinant protein inhibitors, to overcome.

In contrast to neutralizing antibodies, ACE2-MVPs and DPP4-MVPs were designed to function as decoy-target cells and readily formed multivalent interactions with the spike proteins of corresponding SARS Coronavirus virions. Both ACE2-MVPs and DPP4-MVPs had picomolar range IC50s and were considerably more potent than many neutralizing antibodies being tested in clinic. At over 200 copies of ACE2 molecules per particle, which was comparable to the number of Spike proteins per virion, ACE2-MVPs effectively competed with target cells for virus binding at a comparable functional affinity. Notably, ACE2-MVPs effectively blocked viral entry after viruses bound to target cells, indicating that decoy-MVPs latched onto viruses attached to cells through multivalent interaction and prevent them from fusing with target cells. Taken together, these findings illustrate that multivalent interaction underlies the potent neutralization by decoy-MVPs.

Decoy-MVPs create variant-proof multivalent traps for viruses: Viruses harness high mutation rates and multivalent binding to host cells to gain an advantage in targeted cell entry and immune evasion. Spike mutagenesis and novel glycosylation patterns can effectively disrupt the neutralizing function of antibodies and other low-valency viral-blocking agents, enabling viruses to win the cat-and-mouse game with our immune system. It is likely that mutations that are resistant to current combinations of clinically tested neutralization antibodies will emerge and render these therapies less effective. Not surprisingly, it remains a challenge to develop effective low-valency neutralizing compounds against viruses or to generate universal vaccines by using highly potent antibodies.

In contrast, a virus would not be able to escape neutralization control by a corresponding decoy-MVP without losing or significantly altering its original tropism. Mutations abolishing spike and ACE2 binding abolish virion interaction with ACE2-MVP and target cells, whereas mutations enhancing spike and ACE2 binding augment virion interaction with ACE2-MVP and target cells. The ACE2-MVPs of the disclosure neutralized D614G CoV-2 viruses at comparable or higher efficiency than the original SARS CoV-1 and CoV-2 viruses. Thus, ACE2-MVPs, which were broadly neutralizing against all SARS coronaviruses utilizing ACE2 as a host cell receptor, created multivalent traps that were difficult for viruses to escape. In the event that SARS CoV-2 evolved to adopt a new host cell receptor or a new zoonotic coronavirus jumped to humans, decoy-MVPs could be readily developed once their host cell receptors are identified. As a proof of the adaptability of the decoy-MVP strategy, DPP4-MVPs for MERS were created, which demonstrated that these decoy-MVPs were highly potent in neutralizing MERS viruses. In addition to high potency, the decoy-MVP strategy effectively countered existing strategies of viral immune evasion, offering another critical advantage over neutralizing antibodies.

Decoy-MVPs as building blocks for modular antivirals: The demonstration of decoy-MVPs as potent antivirals illustrated a modular approach to block viruses from entering cells by building MVPs displaying universal features required for viral attachment and entry. The approach enabled the development of antivirals using a relative constant for viral pathogenesis—host cell receptors. The advantage of the approach was evident in comparison to developing neutralizing antibodies for a constantly evolving spike or surface glycoprotein. Decoy-MVPs can be built to precisely mimic target cells so that the virus cannot distinguish the two in terms of molecular identity and multivalent functional affinity.

The decoy-MVPs of the disclosure displayed a single type of viral entry receptor, such as wild-type ACE2 for SARS-CoV-1/2 and wild-type DPP4 for MERS coronavirus. Conceivably, such decoy-MVPs could be further modified to display mutated viral entry receptors with improved affinity to viral envelope proteins, reduced size for ease of production, and inactivated physiological function to avoid undesirable impacts on normal physiology. For example, ACE2 has enzymatic activities required for angiotensin processing. Thus, delivering large amounts of functional ACE2-MVPs may cause a dramatic decrease in Angiotensin II levels and increase of angiotensin (1-5/7). Therefore, enzymatically inactive ACE2 or DPP4 may be displayed on MVPs to eliminate other functions of the decoy-MVPs that are unrelated to antiviral function.

Many viruses utilize both host cell attachment receptors and entry receptors for infection. For example, while ACE2 is essential for virus infection, SARS CoV-2 entry of target cells may also be facilitated by TMPRSS2, DPP4, and sialic acid. Decoy-MVPs were generated by displaying viral decoy receptors, such as ACE2 and DPP4, on the lentiviral particles. With this design, decoy-MVPs could be modified by co-transfecting to ACE2-displaying vector together with displaying vectors for host-cell entry receptors, attachment receptors, and other molecules important for viral infection. Ratios can be tuned to maximize their neutralizing potential and accurately recapitulate a typical target cell membrane. Decoy receptors could also be displayed on other types of viruses with or without lipid envelopes or on the surface of synthetic nanoparticles. Beyond decoy-MVPs, other types of multivalent particles can be generated by displaying spike-specific antibodies or other engineered spike-binding proteins alone or together with decoy receptors for enhanced neutralization function. Finally, decoy-MVPs can be armed with additional regulatory molecules on their surfaces or inside nanoparticles to deliver additional cargo for immune modulation, targeted degradation, and vaccination.

The decoy-MVP strategy enables preemptive development of antivirals: Viral zoonoses, the transmission of viral diseases between animals to humans, has and continues to be a significant public health risk with epidemic, endemic, and pandemic potential. However, because of high mutation rates during virus replication, humans have been playing catchup to develop effective antiviral therapeutics in an effort to control outbreaks of influenza, coronaviruses, and other zoonotic viruses. Since nearly all enveloped and non-enveloped viruses use their multivalent surface envelope proteins for attachment and host-cell entry, our results suggest that decoy-MVPs can be used as modular antiviral therapeutics for all viruses that utilize host cell receptors for cell attachment and entry. The decoy-MVP strategy of the disclosure suggests a novel approach to preemptively develop modular decoy-MVPs for any human and animal virus with zoonotic potential. Instead of chasing elusive super-antibodies for rapidly evolving viruses, host cell entry receptors can be identified for pathogenic human viruses and animal viruses with zoonotic potential, and decoy-MVPs therapeutics can be pre-emptively developed. This approach will provide an important arsenal for fighting against many pathogenic human viruses, such as influenza, coronaviruses, hepatitis viruses, dengue virus, and HIV.

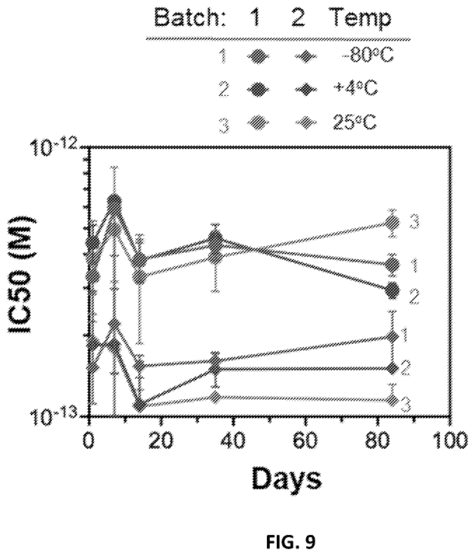

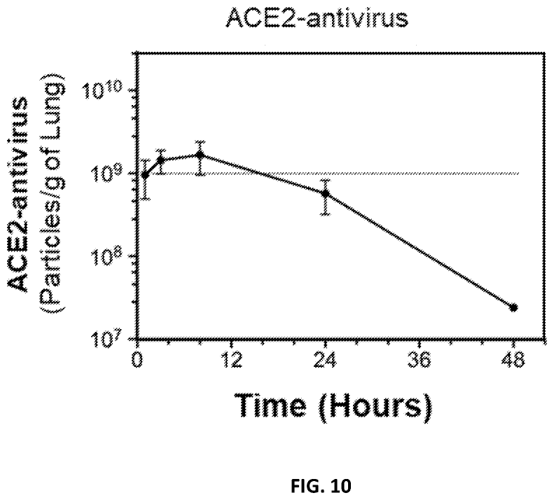

To emulate and counter the spike multivalence of SARS CoV-2, an “ACE2-antivirus” was designed, which was a multivalent neutralizing therapeutic displaying thousands copies ACE2 molecules in trimeric/oligomeric patterns on enveloped viral-like particles. Methods to genetically program lentiviral VLPs to accomplish a patterned display was developed (FIG. 1A-1B). ACE2-antiviruses displaying higher copy numbers of ACE2 in trimeric/oligomeric patterns demonstrated picomolar/sub-picomolar IC₅₀s against SARS CoV-2, and emerging variants (FIG. 1A-1G, FIG. 2A-2C, and FIG. 3A-3F). Intranasal delivery of ACE2-antiviruses effectively rescued human ACE2 transgenic mice from lethal infection by live SARS CoV-2 variants (FIG. 4A-4F, FIG. 5A-5H), and facilitated the development of robust immunity against reinfection by live SARS CoV-2 variants (FIG. 6A-6M). Notably, ACE2-antivirus had many favorable properties as a therapeutic. The ACE2-antivirus was stable at room temperature for at least three months (FIG. 9), and could be efficiently aerosolized for localized delivery (FIG. 10). These findings demonstrated that the ACE2-antivirus was a highly potent variant-proof therapeutic against all SARS coronaviruses utilizing ACE2 as an entry receptor.

FIG. 9 shows the stability of H2A-D4VG antivirus stored at −80° C., +4° C., and 25° C. The neutralizing activities of two batches of H2A-D4VG antivirus stored at −80° C., +4° C., 25° C. were determined in a SARS CoV-2 pseudovirus infection assay using 293T/ACE2 cells as target cells. FIG. 10 shows the PK of the ACE2-antivirus following intranasal dosing. A dose of 2×10¹¹ ACE2-antivirus was delivered into the lung of Balbc mice through intranasal delivery. At various time points after dosing, the lungs of the dosed mice were collected and analyzed by P24 ELISA analyses to determine the amount of ACE2-antivirus in the lung.

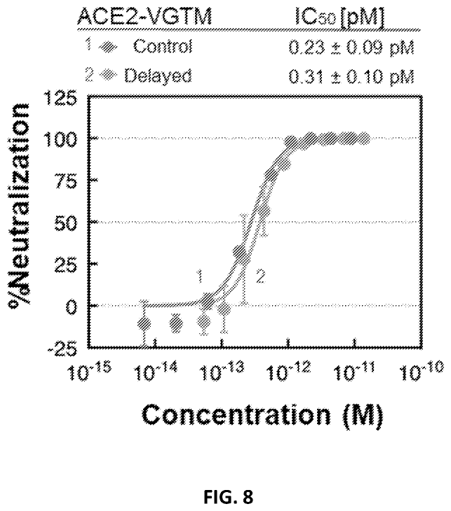

ACE2-antivirus valency determines neutralization potency: The multivalent nature of viruses was mimicked to compete against spike multivalency. To establish a robust VLP display technology, various transmembrane display anchors and oligomerization domains derived from virus surface proteins for effective display of ACE2 protein on VLPs were tested (FIG. 1A-1B). ACE2-antiviruses were generated with varied ACE2 valency from as low as 85±3 copies per particle for ACE2-S2TM antivirus to over thousand copies for the ACE2-VGTM antiviruses (FIG. 1A, 1B). ACE2-VGTM antiviruses with significantly higher ACE2 valency Were more potent than corresponding antiviruses with lower ACE2 valency in both pseudovirus and live virus neutralizing assays (FIG. 1A-1G, FIG. 2A-2C, and FIG. 3A-3F). Notably, the differences in neutralizing potency were more drastic in the total fold of repression and in the neutralizing stoichiometry determined in PNA assays, as disclosed herein. For example, an ACE2-S2TM antivirus reduced a viral infection signal by 100-fold, whereas an ACE2-VGTM antivirus reduced a viral infection signal by 600-fold to the background level. Moreover, each ACE2-S2TM antivirus neutralized about three pseudovirus particles, whereas ACE2-VGTM antivirus sequestered about 18 pseudovirus particles. The data suggests that ACE2-antiviruses may block virus from entering cells through a simple sequestration mechanism without blocking virus interaction with target cells. In a delayed PNA assay, pseudovirus was pre-incubated with target cells for 30 minutes, and ACE2-VGTM antivirus was added (FIG. 8). The data showed that ACE2-MVPs effectively blocked viral entry after viruses bound to target cells, indicating that decoy-MVPs could latch onto viruses that have already attached to cells through multivalent interaction and prevent them from fusing with target cells. The results demonstrated that multivalent ACE2-antiviruses could function as a molecular Velcro to soak up multiple SARS CoV-2 virions, and higher valency could facilitate more effective capture of target viral particles.

FIG. 8 shows the neutralizing potentials of ACE2-VGTM antivirus in a delayed pseudovirus neutralization assay (PNA). In the control PNA, pseudovirus was mixed with ACE2VGTM antivirus and then incubated with target cells. In the delayed PNA, pseudovirus was incubated with target cells for one hour and allowed to attached to target cells prior to the introduction of ACE2VGTM antivirus. The analyses were used to infer whether ACE2-antivirus can block the virus entry after viruses have attached to the target cells.

Oligomerized ACE2-antivirus exhibits enhanced neutralization potency: Beyond higher valency, the effect of oligomerization patterns of ACE2 molecules in enhancing local avidity during interaction with spike trimers and exhibiting neutralization of ACE2-antiviruses was investigated. An ACE2-D4VG antivirus was designed and generated, which displayed higher copies of ACE2 in trimeric/oligomeric formats, using a display carrier comprising a D4 trimerization domain from the post-fusion VSV-G protein, the VSV-G transmembrane, and cytosolic domains, as disclosed herein. The design reflected the trimeric or tetrameric display of spike proteins shared by nearly all enveloped viruses. The trimeric or tetrameric display was hypothesized to induce a multivalent engagement between spikes and viral entry receptor and for selected enrichment of viral spike proteins during virus biogenesis. The ACE2-D4VG antiviruses displayed over 2340±640 copies of trimeric/oligomeric ACE2, whereas ACE2-VGTM antiviruses displayed about 1002±317 copies of monomeric ACE2. The ACE2-D4VG antiviruses, which displayed higher copies of ACE2 in trimeric/oligomeric formats, was more potent than the ACE2-VGTM antivirus, which displayed slightly fewer copies of ACE2 in monomeric format, as determined by various neutralization assays (FIGS. 1-3). The data also illustrated that oligomerized display and increased valency helped rescue the reduced neutralization potency caused by the ACE2 inactivating mutations (FIG. 2). Finally, the trimeric H2A-D4VG antivirus was significantly more potent than monomeric ACE2-VGTM antivirus in a live virus neutralization assay against live SARS CoV-2 (FIG. 3A, 3C) and the delta variant (FIG. 3E, 3F). Collectively, the data demonstrate that ACE2-antivirus with higher valency and trimeric/oligomeric display were more potent neutralizers of live SARS CoV-2 variants (FIGS. 1-3), supporting the notion that more dominant antiviral multivalency may be the key to beating spike multivalence.

ACE2-antivirus as a potential variant-proof therapeutic: By design, the ACE2-antiviruses disclosed herein should be effective against all SARS coronaviruses utilizing ACE2 as entry receptor. Mutations abolishing spike proteins, and ACE2 binding abolishes virion interaction with ACE2-antivirus and target cells. Mutations enhancing spike and ACE2 binding augment virion interaction with ACE2-MVP and target cells. Supporting this notion, the trimeric H2A-D4VG-antivirus was found to be equally potent against SARS CoV-1 and SARS CoV-2 variants with D416G, or N439K, or N501Y, or E484K, or E484Q+L452R spike mutations in PNA analyses (FIG. 2, and other data disclosed herein). Moreover, in live virus neutralization analyses, the trimeric H2A-D4VG-antivirus had low picomolar or sub-picomolar IC₅₀s against the original, beta, and delta SARS CoV-2 variants, albeit with slight varied neutralizing potencies (FIG. 3A-3F). Notably, the trimeric/oligomeric H2A-D4VG-antivirus with higher valency was more robust against SARS CoV-2 mutagenesis than the monomeric H2A-VGTM antivirus (FIG. 3A-3F). Finally, the trimeric H2A-D4VG-antivirus effectively rescued hACE2 transgenic mice from lethal infection by the original SARS CoV-2 (FIG. 5A-5H) and the delta variant at nearly 8-times of challenge dose (FIG. 6A-6M). The results provide strong in vitro and in vivo support that the ACE2-antiviruses of the disclosure are a potent variant-proof neutralizing therapeutic for SARS coronaviruses utilizing ACE2 entry receptor.

Decoy-antiviruses as multivalent therapeutics for pandemic viruses: As exemplified by ACE2-antiviruses of the disclosure, highly potent variant-proof neutralizing decoy-antiviruses may be rapidly developed to target novel viruses. The decoy-antivirus strategy effectively counters spike mutagenesis and viral immune evasion, offering another critical advantage over neutralizing antibody-based antivirals. The results illustrate that the decoy-antivirus platform may be employed to generate highly potent, variant-proof neutralizing therapeutics against existing and future pandemic viruses.

Decoy-antiviruses as modular multivalent therapeutics: The experiments disclosed herein illustrate a programmable multivalent therapeutic platform and therapeutic modality. The decoy-antiviruses described herein display a single type of viral entry receptor, such as ACE2 for SARS-CoV-1/2. Decoy-antiviruses can be further modified to display mutated viral entry receptors with improved affinity to viral spike proteins, reduced size for ease of production, and inactivated physiological function to avoid undesirable impacts on normal physiology. ACE2-antiviruses can be programmed to display ACE2 and co-receptors to further improve their resilience against spike mutagenesis. The multivalent ACE2-antiviruses can be built on VLPs based on other viruses, exosomes, or synthetic nanoparticles.

Use of decoy-antiviruses as treatment vaccine or “inactivated” vaccine for the pandemic viruses: The methods disclosed herein describe a novel strategy for vaccination. First, ACE2-antivirus treatment protected mice from lethal infection and led to the development of broad protective immunity against multiple SARS CoV-2 variants (FIG. 6A-6M). The data suggest a novel approach for vaccination by treatment. Specifically, infected patients can be treated with ACE2-antivirus and consequently vaccinated. Such a vaccination approach would combine treatment and vaccination and thus eliminate the need to vaccinate the entire population. Second, mice were vaccinated with intranasally delivered ACE2-antivirus “inactivated” live SARS CoV-2 (FIGS. 6I-6M), and the data suggest a novel approach to rapidly producing “inactivated” vaccine by using decoy-antivirus. ACE2-antivirus was a highly potent variant-proof neutralizing compound against all SARS Coronaviruses utilizing ACE2 as an entry receptor. Live SARS Coronavirus could be mixed with an excess amount of ACE2-antivirus to rapidly generate ACE2-antivirus “inactivated” SARS CoV-2 vaccines. If high-affinity entry receptors cannot be defined for the pandemic viruses, highly potent neutralizing antibody-based antiviruses (Ab-antivirus) can be rapidly developed by displaying antibodies recognizing viral surface proteins on the enveloped vesicles. Similarly, “inactivated” vaccines can be generated by mixing live viruses with an Ab-antivirus, and the mixture can be used to immunize patients.

ACE2-antivirus can also be designed to be immune-modulating to further enhance the immune responses during vaccination. For example, ACE2-antiviruses can be built on lentiviral-based VLPs carrying double-stranded viral genomes, which can serve as an agonist for TLR3. ACE2-antivirus can also be programmed to display immune-modulating molecules, such as OX40 ligand and interferon, to further enhance the immune responses against the ACE2-antivirus “inactivated” live SARS CoV-2. Finally, our finding demonstrated that ACE2-antivirus “inactivated” can be delivered intranasally for effective vaccination, illustrating anew approach for intranasal vaccination. Nasal vaccination offers some advantages over traditional injected vaccines. Such vaccination may help the development of mucosal immunity in the respiratory tracks, which is the primary route of entry for respiratory viruses, thus providing fast and more effective protection. Nasal vaccination are also easier to administer and may help with widespread adoption of a vaccine.

The data demonstrate that the ACE2-Antiviruses disclosed herein are variant-proof and orders of magnitude more potent than some clinical neutralizing antibodies. Moreover, treatment through intranasal delivery of ACE2-Antivirus prevented the development of respiratory symptoms and rescued mice from lethal infection by both the original and delta strains of SARS CoV-2. Notably, following ACE2-antivirus treatment of SARS CoV-2 infection facilitated the development of robust immunity against multiple SARS CoV-2 variants. Together, the results establish the decoy-Antivirus as novel, variant-proof neutralizing therapeutic platform targeting existing and new pandemic coronaviruses by counteracting multivalent interactions between viral spike and entry receptors.

### Example 25: Methods and Materials

Design and production of spike-pseudotyped viral-like particles: Codon-optimized synthetic DNAs encoding the SARS CoV-1, CoV-2, and MERS Coronavirus spike proteins were cloned into a mammalian expression vector placing under the control of a CMV promoter. For improved CoV-2 spike expression and pseudotyping, a construct expressing a chimeric protein containing the extracellular spike domain fused to VSV-G transmembrane and cytosolic tails was also generated. The expression of spike proteins after transfecting into 293 T cells was validated by Western-blots using specific antibodies against respective spike protein and the VSV-G tag.

To produce spike-pseudotyped viral-like particles, spike expression construct, psPAX2 lentiviral packaging vector, and a lentiviral transfer vector with luciferase reporter were co-transfected into 293T cells using a polyethyenimine (PEI) transfection protocol. psPAX2 is a generation 2 lentiviral vector packaging vector expressing gag, pol, rev proteins. Briefly, eight million 293 T cells were seeded onto a 10 cm plate 16-24 hour before transfection and cultured overnight. Cells should reach ˜90% of confluency at the time of transfection. A transfection mix was prepared by adding 30 μg of diluted PEI solution to a DNA cocktail containing 1.25 μg of spike expression construct, 5 μg of psPAX2 lentiviral packaging vector, and 7.5 μg of lentiviral luciferase reporter vector. The transfection mix was incubated at room temperature for 15 minutes and then added to the cells. At 5-6 hours post transfection, cell culture medium was changed to virus production medium containing 0.1% sodium butyrate. Coronavirus pseudovirions were collected twice at 24- and 48-hour post medium change, concentrated by PEG precipitation, and further purified through a gel-filtration column.

Design and production of decoy-multivalent particles displaying ACE2 receptors (ACE2-MVPs): Synthetic DNAs encoding the ACE2 ectodomain were fused to various viral displaying anchor molecules and were cloned into a mammalian expression vector under the control of a CMV promoter. Viral envelope proteins were chosen as displaying anchor molecules because they are integral to viral biogenesis and are highly efficient at targeting viral membrane. ACE2 displaying constructs were generated expressing the ACE2 ectodomain fused to full-length VSV-G, or the truncated VSV-G with only transmembrane and cytosolic domains, or the truncated CoV-2 spike with S1 domain deleted. Synthetic DNAs encoding DPP4 ectodomain were fused to the HCΔ18 transmembrane domain from measles virus.

To produce decoy multivalent-particles, ACE2 or DPP4 displaying construct, psPAX2 lentiviral packaging vector, and a lentiviral transfer vector with GFP reporter were co-transfected into 293T cells using a polyethyenimine (PEI) transfection protocol. Briefly, eight million 293 T cells were seeded onto a 10 cm plate 16-24 hour before transfection and cultured overnight. Cells were expected to reach ˜90% of confluency by the time of transfection. A transfection mix was prepared by adding 30 ug of diluted PEI solution to a DNA cocktail containing 1.25 μg of ACE2 or DPP4 expression construct, 5 μg of psPAX2 lentiviral packaging vector, and 7.5 μg of GFP reporter vector. The transfection mix was incubated at room temperature for 15 minutes and then added to the cells. At 5-6 hours post transfection, cell culture medium was changed to virus production medium containing 0.1% sodium butyrate. ACE2-MVPs were collected twice at 24- and 48-hour post medium change, concentrated by PEG precipitation, and further purified through a gel-filtration column.

ACE2-MVPs without viral genome could also be packaged with no transfer vector without compromising ACE2-MVP yields and function. Several viral envelope proteins were tested for anchoring ACE2 protein to the membrane of the pseudo-lentiviral particles, including VSV-G (glycoprotein of Vesicular Stomatitis virus), HCΔ18 (a mutant version of hemagglutinin envelope protein from measles virus), and S2 (the fusion domain of SARS CoV-2 spike protein). Fusions of ACE2 to the full-length VSVG and truncated VSV-G with only transmembrane region and cytosolic tail were also tested.

Western blot analysis of decoy-MVPs: Expression of fusion proteins on decoy-MVPs are confirmed via western blot analysis of purified particles. Samples of purified MVPs are lysed at 4° C. for 10 minutes with cell lysis buffer (Cell Signaling) before being mixed with NuPage LDS sample buffer and boiled at 95° C. for 5 minutes. Differences in oligomerization are determined by running samples in reducing and non-reducing conditions. Under reducing conditions, 5% 2-Mercaptoethanol are added to samples to dissociate oligomerized MVP-ICs. Protein samples are then separated on NuPAGE 4-12% Bis-Tris gels and transferred onto a polyvinylidene fluoride (PVDF) membrane. PVDF membranes are blocked with TRIS-buffered saline with Tween-20 (TBST) and 5% skim milk for 1 hour, prior to overnight incubation with primary antibody diluted in 5% milk. For display fusion constructs expressing VSVG-tag, an anti-VSV-G epitope tag rabbit polyclonal antibody are used at a 1:2000 dilution. The following day, the PVDF membrane are washed 3 times with 1×TBST and stained with a goat-anti-rabbit secondary antibody (IRDye 680) at a 1:5000 dilution for 60 minutes in 5% milk. Post-secondary antibody staining, the PVDF membrane are again washed 3 times with TBST before imaging on a Licor Odyssey scanner.

Alternatively, western blot analyses are performed using an automated Simple Western size-based protein assay (Protein Simple) following the manufacturer's protocols. Unless otherwise mentioned, all reagents used here are from Protein Simple. Concentrated samples are lysed as described above, before being diluted 1:10 in 0.1× sample buffer for loading on capillaries. Displayed fusion protein expression levels are identified using the same primary rabbit polyclonal antibody at a 1:400 dilution and an HRP conjugated anti-rabbit secondary antibody (Protein Simple). Chemiluminescence signal analysis and absolute quantitation are performed using Compass software (Protein Simple).

Quantitative western blot analyses of decoy-MVPs: Quantitative western blot analyses were carried out to determine the copies of ACE2 and DPP4 on the lentiviral particles. P24 ELISA assays were used to determine the lentiviral particle concentration of the ACE2-MVPs. Samples containing decoy-MVP samples (2-3×10⁸ particles, ˜20 μg protein) were mixed with loading buffer and boiled at 100° C. for 5 minutes. The proteins and their corresponding serial-diluted recombinant protein standards were separated on 12% sodium dodecyl sulfate polyacrylamide (SDS-PAGE) and transferred onto a polyvinylidene fluoride (PVDF) membrane. The membrane was blocked with phosphate buffered saline with Tween-20 (PBST) and 5% skim milk at room temperature for 2 hours and subsequently incubated overnight at room temperature with the primary goat-anti-human ACE2 antibodies. The membrane was incubated at room temperature for one hour with the secondary antibodies (IRDye 680 anti-goat secondary) and quantified on a Licor Odyssey scanner. The copies of ACE2 or DPP4 proteins on respective decoy-MVPs were calculated by using the standard curves generated by corresponding ACE2 and DPP4 recombinant proteins.

Viral-like particle quantification by p24 ELISA: P24 concentrations in pseudovirus samples of pseudotyped coronaviruses, influenza viruses and decoy-multivalent particles are determined using an HIV p24 SimpleStep ELISA kit. Concentrations of lentiviral pseudovirus particles are extrapolated from the assumption that each lentiviral particle contains approximately 2000 molecules of p24, or 1.25×10⁴ pseudovirus particles per picogram of p24 protein.

Quantify decoy-MVPs by Tunable Resistive Pulse Sensing: The sizes and concentrations of VLP_based or extracellular vesicle-based decoy-MVPs are determined by tunable resistive pulse sensing (TRPS, qNano, IZON). Purified pseudovirus collections are diluted in 0.2 μm filtered PBS with 0.03% Tween-20 prior to qNano analysis. Concentration and size distributions of MVP-ICs are then determined using an NP200 nanopore at a 45.5 mm stretch, and applied voltages between 0.5 and 0.7V were used to achieve a stable current of 130 nA through the nanopore. Measurements for each pseudovirus sample are taken at pressures of 3, 5 and 8 mbar, and considered valid if at least 500 events were recorded, particle rate was linear and root mean squared signal noise was maintained below 10 pA. MVPs concentrations are then determined by comparison to a standardized multi-pressure calibration using CPC200 (mode diameter: 200 nm) (IZON) carboxylated polystyrene beads diluted 1:200 in 0.2 M filtered PBS from their original concentration of 7.3×1011 particles per/mL. Measurements are analyzed using IZON Control Suite 3.4 software to determine original sample concentrations.

Characterization of Proteins Displayed on Enveloped Particles: The concentration of VLP- or EV-based decoy-MVPs are measured by P24 ELISA or tunable resistive pulse sensing (TRPS, qNano), respectively. Then, copies of displayed peptides on enveloped particles are determined by quantitative Western-blot analyses. Finally, the oligomerization patterns of displayed peptides on the enveloped particles were discerned by non-reducing PAGE analyses. Enveloped particles are expected to display at least 10 copies of protein molecules on surface of VLPs and EVs with monomeric or trimeric configurations.

Target cells for coronavirus pseudovirus infection: A large panel of cell lines was screened to identify target cell lines that were effectively infected by spike pseudovirions. Candidate target cells were infected with saturated doses of coronavirus spike pseudovirions carrying a luciferase reporter, and luciferase activity of the infected cells was measured at 48 hours post-infection. Target cells that yielded at least 1,000-fold luciferase signals above the background infection were considered infectable. The cell lines tested included native cell lines, such as VERO, VERO E6, large panel of human lung cancer cell lines, and ACE2 overexpression cell lines. H1650 cells were effective target cells for the MERS spike pseudovirions (>10,000-fold increase in luciferase signals), 293T/ACE2 and H1573/ACE2 cells were effective target cells for the CoV-2 spike pseudovirions (10,000 to 100,000-fold increase in luciferase signals), and 293T/ACE2 and VERO E6 were effective target cells for the CoV-1 spike pseudovirions (1,000 to 10,000-fold increase in luciferase signals). TCID₅₀ (Fifty-percent tissue culture infective dose) were then determined for CoV-1, CoV-2, and MERS spike pseudovirions by titrating the dose-dependent infection in respective target cell lines. The TCID₅₀ doses were used in the pseudovirus neutralization assay to determine the inhibitory activities of decoy-MVPs.

IC₅₀ Pseudovirus neutralization assay: Respective target cells were seeded in 96-well, flat-bottom, clear, tissue-culture treated plates at 25,000 cells/well with 6 μg/mL polybrene in the appropriate base medium supplemented with 10% fetal bovine serum and 1% Penicillin Streptomycin. RPMI media with glucose, HEPES Buffer, L-Glutamine, sodium bicarbonate and sodium pyruvate served as base medium for H1573/ACE2 cells and H1650 cells, while 293 T Growth Media was used as base medium for 293 T/17 cells. Pseudovirus was then added to wells at TCID₅₀ concentrations, along with titrated decoy anti-Virus MVP in 9×2-fold serial dilutions, yielding a 10-point dilution curve. In delayed pseudovirus neutralization assays, pseudovirus was added to wells in TCID₅₀ concentrations and incubated with cells for 60 minutes prior to the addition of titrated anti-Virus MVP. Plates containing cells, pseudovirus and decoy-MVP were then centrifuged at 800×g, 25° C. for 60 minutes to maximize infection efficiency. 48 hours post-infection, cells were lysed using Firefly Luciferase Lysis Buffer and lysis was transferred to 96-well, white assay plates before luciferase activity was analyzed via GLOMAX multi-detection system. Titrated infection data was then plotted and fitted to a 4-parameter, logistic curve in order to calculate the half maximal inhibitory concentration (IC₅₀) of various decoy anti-Virus MVPs neutralizing their respective pseudoviruses.

Plaque reduction neutralization test with SARS CoV-2 virus: Vero E6 cells (ATCC: CRL-1586) were seeded at 175,000 cells/well using DMEM media supplemented with 10% fetal bovine serum (FBS) and Gentamicin in 24-well, tissue-culture treated plates. Cells were then incubated overnight at 37° C. in 5% CO₂ until reaching 80-100% confluence the next day. The following day, anti-Virus MVP samples in serum were heat inactivated at 56° C. for 30 minutes before preparing serial dilutions. All dilutions were made using DMEM supplemented with 2% FBS and Gentamicin (referred to as “diluent”). Anti-Virus MVP serial dilutions, to a total volume of 300 μL, were made using diluent, and 300 μL empty diluent served as a virus positive control. Next, 300 μL diluent containing SARS CoV-2 (30 PFU/well) was added to anti-Virus MVP serial dilutions and to the virus-only positive control, to a final volume of 600 μL. Mixtures of anti-Virus MVP and SARS CoV-2 were incubated at 37° C. in 5.0% CO₂ for 60 minutes, before serial dilutions and virus positive control were added to cells. Cells were incubated with mixtures for 1 hour to allow for infection, and virus titers for each serial dilution were then determined by plaque assay. Percent neutralization data was plotted and a 4-parameter logistic curve was fitted to data to determine the 50% plaque reduction neutralization titer (PRNT₅₀) of various anti-Virus MVPs neutralizing live SARS CoV-2 virus (GraphPad Prism 9.0.0).

In vivo live virus neutralization efficacy of ACE2-MVP in hamsters: Eight golden hamsters, male and female, 6-8 weeks old were used in each cohort. Animals were weighed prior to the start of the study. Animals were challenged with 2.3×10∝PFU of USA-WA1/2020 (NR-52281; BEI Resources) through intranasal delivery (IN) administration of 50 μL of viral inoculum into each nostril. At various time points after infection, hamsters are treated with decoy-MVPs through intranasal delivery. The animals were monitored twice daily for signs of COVID-19 disease (ruffled fur, hunched posture, labored breathing) during the study period. Body weights were measured once daily during the study period. Lung tissues were collected and sampled for viral load assays by PRNT. Tissues were stored at 80° C. for histology and viral load analysis by qPCR or PRNT analyse s.

In vivo live virus neutralization efficacy of ACE-MVP in ACE2 mice: Six ACE2 transgenic mice, male and female, 6-8 weeks old were used in each cohort. Animals were weighed prior to the start of the study. Animals were challenged with 2.3×10⁴ PFU of USA-WA1/2020 (NR-52281; BEI Resources) through intranasal administration of 50 μL of viral inoculum into each nostril. At various time points after infection, hamsters are treated with decoy-MVPs through intranasal delivery. The animals were monitored twice daily for signs of COVID-19 disease (ruffled fur, hunched posture, labored breathing) and survival during the study period. Body weights were measured once daily during the study period. Lung tissues were collected and sampled for viral load assays by PRNT. Tissues were stored at 80° C. for histology and viral load analysis by qPCR or PRNT analyses.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A composition of multivalent particle that comprises a virus-capturing polypeptide on a surface of the particle wherein the displayed virus-capturing polypeptide is expressed at a valency of at least 10 copies on the surface of the particle and in an oligomerized format wherein the displayed virus-capturing polypeptide forms multivalent interactions with a viral spike protein which provide tighter binding through avidity than a soluble version of the displayed virus-capturing polypeptide to the viral spike protein.

2. The composition of claim 1, wherein the viral spike protein is from severe acute respiratory syndrome coronavirus 1 (SARS-CoV-1), severe acute respiratory syndrome coronavirus 2 (SARS-CoV-2), Middle East respiratory syndrome-related coronavirus (MERS-CoV), respiratory syncytial virus (RSV), hepatitis B virus (HBV), hepatitis D virus (HDV), human immunodeficiency virus (HIV), or combinations thereof.

3. The composition of claim 1 or 2, wherein the displayed virus-capturing polypeptide comprises a receptor for the virus entry, a ligand, a secreted protein, an antibody, or an engineered protein.

4. The composition of claim 3, wherein the receptor comprises a viral entry receptor or a viral attachment receptor.

5. The composition of claim 3, wherein the receptor is both a viral entry receptor and a viral attachment receptor.

6. The composition of claim 3, wherein the displayed virus-capturing polypeptide comprises an extracellular domain of the receptor.

7. The composition of any one of claims 1-6, wherein the displayed virus-capturing polypeptide comprises angiotensin-converting enzyme 2 (ACE2), transmembrane serine protease 2 (TRMPSS2), dipeptidyl peptidase 4 (DPP4), cluster of differentiation 4 (CD4), C-C chemokine receptor type 5 (CCR5), C-X-C chemokine receptor type 4 (CXCR4), cluster of differentiation 209 (CD209), or C-type lectin domain family 4 member M (CLEC4M).

8. The composition of any one of claims 1-7, wherein the displayed virus-capturing polypeptide comprises an amino acid sequence of at least 90% sequence identity to the amino acid sequence according to any one of SEQ ID NOs: 1-4.

9. The composition of any one of claims 1-8, wherein the displayed virus-capturing polypeptide is part of a fusion protein, wherein the fusion protein comprises a transmembrane polypeptide.

10. The composition of claim 9, wherein the transmembrane polypeptide comprises a spike glycoprotein transmembrane region, a mammalian membrane protein, an envelope protein, a nucleocapsid protein, or a cellular transmembrane protein.

11. The composition of claim 9 or 10, wherein the transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD 114, Baboon endogenous virus (BaEV), glycoprotein (GP41), or glycoprotein 120 (GP120).

12. The composition of claim 11, wherein the VSVG transmembrane region comprises full length VSVG transmembrane region or a truncated VSVG transmembrane region.

13. The composition of claim 11, wherein the transmembrane polypeptide comprises the VSVG transmembrane region and a VSVG cytoplasmic tail.

14. The composition of any one of claims 9-13, wherein the transmembrane polypeptide comprises an amino acid sequence at least about 90% identical to the amino acid sequence according to any one of SEQ ID NOs: 19-27.

15. The composition of any one of claims 9-14, wherein the fusion protein further comprises an oligomerization domain.

16. The composition of claim 15, wherein the oligomerization domain comprises a dimerization domain, a trimerization domain, or a tetramerization domain.

17. The composition of claim 15 or 16, wherein the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain.

18. The composition of any one of claims 15-17, wherein the oligomerization domain comprises an amino acid sequence that has at least 90% sequence identity to an amino acid sequence according to any one of SEQ ID NOs: 5-18, and 28.

19. The composition of any one of claims 9-18, wherein the fusion protein is expressed at least about 50 copies on a surface of the multivalent particle.

20. The composition of any one of claims 9-18, wherein the fusion protein is expressed at least about 75 copies on a surface of the multivalent particle.

21. The composition of any one of claims 9-18, wherein the fusion protein is expressed at least about 100 copies on a surface of the multivalent particle.

22. The composition of any one of claims 9-18, wherein the fusion protein is expressed at least about 150 copies on a surface of the multivalent particle.

23. The composition of any one of claims 9-18, wherein the fusion protein is expressed at least about 200 copies on a surface of the multivalent particle.

24. The composition of any one of claims 1-23, wherein the multivalent particle further comprises a second displayed virus-capturing polypeptide on a surface of the particle that binds to the viral protein, wherein the second displayed virus-capturing polypeptide is expressed at least about 10 copies on the surface of the multivalent particle.

25. The composition of claim 24, wherein the second displayed virus-capturing polypeptide comprises a receptor for the viral protein.

26. The composition of claim 25, wherein the receptor comprises a viral entry receptor or a viral attachment receptor.

27. The composition of claim 25, wherein the receptor is both a viral entry receptor and a viral attachment receptor.

28. The composition of claim 25, wherein the second displayed virus-capturing polypeptide comprises an extracellular domain of the receptor.

29. The composition of claim 24, wherein the second displayed virus-capturing polypeptide comprises a ligand or a secreted protein.

30. The composition of claim 24, wherein the second displayed virus-capturing polypeptide comprises ACE2, TRMPSS2, DPP4, CD4, CCR5, CXCR4, CD209, or CLEC4M.

31. The composition of any one of claims 24-30, wherein the second displayed virus-capturing polypeptide comprises an amino acid sequence of at least 90% sequence identity to the amino acid sequence according to any one of SEQ ID NOs: 1-4.

32. The composition of any one of claims 24-31, wherein the second displayed virus-capturing polypeptide is part of a second fusion protein, wherein the second fusion protein comprises a second transmembrane polypeptide.

33. The composition of claim 32, wherein the second transmembrane polypeptide comprises a spike glycoprotein transmembrane region, a mammalian membrane protein, an envelope protein, a nucleocapsid protein, or a cellular transmembrane protein.

34. The composition of claim 32 or 33, wherein the second transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD114, Baboon endogenous virus (BaEV), glycoprotein (GP41), or glycoprotein 120 (GP120).

35. The composition of claim 34, wherein the VSVG transmembrane region of the second transmembrane polypeptide comprises full length VSVG transmembrane region or a truncated VSVG transmembrane region.

36. The composition of claim 34, wherein the second transmembrane polypeptide comprises the VSVG transmembrane region and a VSVG cytoplasmic tail.

37. The composition of any one of claims 24-36, wherein the second transmembrane polypeptide comprises an amino acid sequence at least about 90% identical to the amino acid sequence according to any one of SEQ ID NOs: 19-27.

38. The composition of any one of claims 32-37, wherein the second fusion protein further comprises a second oligomerization domain.

39. The composition of claim 38, wherein the second oligomerization domain comprises a dimerization domain, a trimerization domain, or a tetramerization domain.

40. The composition of claim 38 or 39, wherein the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain.

41. The composition of any one of claims 38-40, wherein the oligomerization domain comprises an amino acid sequence that has at least 90% sequence identity to an amino acid sequence according to any one of SEQ ID NOs: 5-18, and 28.

42. The composition of any one of claims 32-41, wherein the second fusion protein is expressed at least about 50 copies on a surface of the multivalent particle.

43. The composition of any one of claims 32-41, wherein the second fusion protein is expressed at least about 75 copies on a surface of the multivalent particle.

44. The composition of any one of claims 32-41, wherein the second fusion protein is expressed at least about 100 copies on a surface of the multivalent particle.

45. The composition of any one of claims 32-41, wherein the second fusion protein is expressed at least about 150 copies on a surface of the multivalent particle.

46. The composition of any one of claims 32-41, wherein the second fusion protein is expressed at least about 200 copies on a surface of the multivalent particle.

47. The composition of any one of claims 1-46, wherein the multivalent particle does not comprise viral genetic material.

48. The composition of any one of claims 1-46, wherein the multivalent particle is a viral-like a particle.

49. The composition of any one of claims 1-46, wherein the multivalent particle is an extracellular vesicle.

50. The composition of any one of claims 1-46, wherein the multivalent particle is an exosome.

51. The composition of any one of claims 1-46, wherein the multivalent particle is an ectosome.

52. A particle that comprises a ACE2 polypeptide on a surface of the particle wherein the ACE2 polypeptide is expressed at a valency of at least 10 copies on the surface of the particle and in an oligomerized format wherein the ACE2 polypeptide forms multivalent interactions with a viral spike protein which provide tighter binding through avidity than a soluble version of the ACE2 polypeptide to the viral spike protein.

53. The particle of claim 52, wherein the ACE2 polypeptide comprises a wildtype ACE2, or a H2A mutant of ACE2.

54. The particle of claim 52 or 53, wherein the viral spike protein comprises a wildtype SARS CoV-1 spike protein, a mutant of SARS CoV-1 spike protein, a wildtype SARS CoV-2 spike protein, or a mutant of SARS CoV-2 spike protein that binds to ACE2.

55. The particle of claim 54, wherein the viral spike protein comprises the spike protein of original SARS CoV-2, the spike protein of Beta variant of SARS CoV-2, the spike protein of Delta variant of SARS CoV-2, the spike protein of B.1.351 Beta variant of SARS CoV-2, the spike protein of B.1.617.2 Delta variant of SARS CoV-2, the spike protein of SARS CoV-2 USA-WA1/2020 strain, the D614G mutant of SARS CoV-2 spike protein, the N439K mutant of SARS CoV-2 spike protein, the N501Y mutant of SARS CoV-2 spike protein, the E484K mutant of SARS CoV-2 spike protein, the E484Q+L452R mutant of SARS CoV-2 spike protein, the spike protein of Omicron variant of SARS CoV-2, or the E484K+N501Y mutant of spike protein of the B.1.351 South Africa strain of SARS CoV-2.

56. The particle of any one of claims 52-55, wherein the particle binds to the viral spike protein to effectively neutralize a virus comprising the viral spike protein.

57. The particle of claim 56, wherein the virus comprises a SARS CoV-1 virus, or a SARS CoV-2 virus.

58. The particle of claim 56 or 57, wherein the virus comprises the original SARS CoV-2 virus, Delta variant of SARS CoV-2, Beta variant of SARS CoV-2, B.1.351 Beta variant of SARS CoV-2, B.1.617.2 Delta variant of SARS CoV-2, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant of SARS CoV-2, or B.1.351 South Africa strain of SARS CoV-2 comprising the E484K+N501Y mutant of spike protein.

59. The particle of any one of claims 52-58, wherein the ACE2 polypeptide is part of a fusion protein, wherein the fusion protein comprises a transmembrane polypeptide.

60. The particle of claim 59, wherein the transmembrane polypeptide comprises a spike glycoprotein transmembrane region, a mammalian membrane protein, an envelope protein, a nucleocapsid protein, or a cellular transmembrane protein.

61. The particle of claim 59 or 60, wherein the transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD 114, Baboon endogenous virus (BaEV), glycoprotein (GP41), or glycoprotein 120 (GP120).

62. The particle of claim 61, wherein the VSVG transmembrane region comprises full length VSVG transmembrane region or a truncated VSVG transmembrane region.

63. The particle of claim 61, wherein the transmembrane polypeptide comprises the VSVG transmembrane region and a VSVG cytoplasmic tail.

64. The particle of any one of claims 59-63, wherein the transmembrane polypeptide comprises an amino acid sequence at least about 90% identical to the amino acid sequence according to any one of SEQ ID NOs: 19-27.

65. The particle of any one of claims 59-64, wherein the fusion protein further comprises an oligomerization domain.

66. The particle of claim 65, wherein the oligomerization domain comprises a dimerization domain, a trimerization domain, or a tetramerization domain.

67. The particle of claim 65 or 66, wherein the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain.

68. The particle of any one of claims 65-67, wherein the oligomerization domain comprises an amino acid sequence that has at least 90% sequence identity to an amino acid sequence according to any one of SEQ ID NOs: 5-18, and 28.

69. The particle of any one of claims 52-68, wherein the particle is a viral-like a particle.

70. The particle of any one of claims 52-68, wherein the particle is an extracellular vesicle.

71. The particle of any one of claims 52-68, wherein the particle is an exosome.

72. The particle of any one of claims 52-68, wherein the particle is an ectosome.

73. A particle that comprises a DPP4 polypeptide on a surface of the particle wherein the DPP4 polypeptide is expressed at a valency of at least 10 copies on the surface of the particle and in an oligomerized format wherein the DPP4 polypeptide forms multivalent interactions with a viral protein which provide tighter binding through avidity than a soluble version of the DPP4 polypeptide to the viral protein.

74. The particle of claim 73, wherein the viral protein comprises a MERS coronavirus spike protein.

75. The particle of claim 73 or 74, wherein the particle binds to the viral protein to effectively neutralize a virus comprising the viral protein.

76. The particle of claim 75, wherein the virus comprises a MERS coronavirus.

77. The particle of any one of claims 73-76, wherein the DPP4 polypeptide is part of a fusion protein, wherein the fusion protein comprises a transmembrane polypeptide.

78. The particle of claim 77, wherein the transmembrane polypeptide comprises a spike glycoprotein transmembrane region, a mammalian membrane protein, an envelope protein, a nucleocapsid protein, or a cellular transmembrane protein.

79. The particle of claim 77 or 78, wherein the transmembrane polypeptide comprises Vesicular stomatitis virus G (VSVG) transmembrane region, spike protein S1, spike protein S2, Sindbis virus envelope (SINDBIS) protein, hemagglutinin envelope protein from measles virus, envelope glycoprotein of measles virus fusion (F) protein, RD 114, Baboon endogenous virus (BaEV), glycoprotein (GP41), or glycoprotein 120 (GP120).

80. The particle of any one of claims 77-79, wherein the transmembrane polypeptide comprises an amino acid sequence at least about 90% identical to the amino acid sequence according to any one of SEQ ID NOs: 19-27.

81. The particle of any one of claims 77-80, wherein the fusion protein further comprises an oligomerization domain.

82. The particle of claim 81, wherein the oligomerization domain comprises a dimerization domain, a trimerization domain, or a tetramerization domain.

83. The particle of claim 81 or 82, wherein the oligomerization domain comprises a leucine zipper dimerization domain, a D4 post-fusion trimerization domain of VSV-G protein, a Dengue E protein post-fusion trimerization domain, a foldon trimerization domain, human C-propeptide of α1(I) collagen, or an influenza neuraminidase stem domain.

84. The particle of any one of claims 81-83, wherein the oligomerization domain comprises an amino acid sequence that has at least 90% sequence identity to an amino acid sequence according to any one of SEQ ID NOs: 5-18, and 28.

85. The particle of any one of claims 73-84, wherein the particle is a viral-like a particle.

86. The particle of any one of claims 73-82, wherein the particle is an extracellular vesicle.

87. The particle of any one of claims 73-82, wherein the particle is an exosome.

88. The particle of any one of claims 73-82, wherein the particle is an ectosome.

89. A multivalent particle that comprises at least 10 copies of a ACE2 polypeptide on the surface of the particle wherein the particle is self-assembled from the expression of a fusion protein that contains the ACE2 polypeptide sequence and an oligomerization domain sequence from a vector that is transfected into a cell.

90. A multivalent particle that comprises at least 10 copies of a DPP4 polypeptide on the surface of the particle wherein the particle is self-assembled from the expression of a fusion protein that contains the DPP4 polypeptide sequence and an oligomerization domain sequence from a vector that is transfected into a cell.

91. A method of treating a viral infection in a subject in need thereof comprising a multivalent particle displaying a ACE2 polypeptide on a surface of a particle, wherein the displayed ACE2 molecule on the particle forms multivalent interaction to a viral protein which provides tighter binding through avidity than a soluble version of the display polypeptide to the viral protein and administering the multivalent particle to the subject.

92. A method of treating a viral infection in a subject in need thereof comprising a multivalent particle displaying a DPP4 polypeptide on a surface of a particle, wherein the displayed DPP4 molecule on the particle forms multivalent interaction to a viral protein which provides tighter binding through avidity than a soluble version of the display polypeptide to the viral protein and administering the multivalent particle to the subject.

93. A method of inducing immunity against a viral infection in a subject comprising administering to the subject the composition of any one of claims 1-51 or the particle of any one of claims 52-90.

94. The method of claim 93, wherein the administering further comprises administering to the subject a virus comprising the viral protein, wherein the viral infection comprises an infection by the virus.

95. The method of claim 94, further comprising mixing the composition of any one of claims 1-51 or the particle of any one of claims 52-90 with the virus before the administering.

96. The method of claim 95, further comprising, before the administering, incubating the composition of any one of claims 1-51 or the particle of any one of claims 52-90 with the virus after the mixing, wherein the composition of any one of claims 1-51 or the particle of any one of claims 52-90 neutralizes the virus.

97. The method of any one of claims 95-96, wherein the composition of any one of claims 1-51 or the particle of any one of claims 52-90 forms a complex with the virus.

98. The method of claim 97, wherein the complex is in an immune cell after the administering.

99. The method of claim 98, wherein the immune cell comprises a macrophage.

100. The method of claim 98, wherein the immune cell comprises a dendritic cell.

101. The method of any one of claims 93-100, wherein the viral infection comprises an infection by SARS CoV-2, SARS CoV-1, MERS CoV, RSV, HBV, HDV, or HIV.

102. The method of any one of claims 93-101, wherein the viral infection comprises an infection by the original SARS CoV-2 virus, Delta variant of SARS CoV-2, Beta variant of SARS CoV-2, SARS CoV-2 B.1.351 Beta variant, SARS CoV-2 B.1.617.2 Delta variant, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant SARS CoV-2, or the B.1.351 South Africa strain of SARS CoV-2 comprising the E484K+N501Y mutant of spike protein.

103. The composition of any one of claims 1-51, wherein the multivalent particle neutralizes a virus comprising the viral spike protein when the displayed virus-capturing polypeptide forms multivalent interactions with the viral spike protein.

104. The composition of claim 103, wherein the neutralizing potency of the multivalent particle against the virus increases when the displayed virus-capturing polypeptide form oligomers on the surface of the multivalent particle.

105. The composition of claim 103, wherein the neutralizing potency of the multivalent particle against the virus is correlated with the copy number of the displayed virus-capturing polypeptide expressed on the surface of the multivalent particle.

106. The composition of claim 105, wherein the neutralizing potency of the multivalent particle against the virus increases when the copy number of the displayed virus-capturing polypeptide expressed on the surface of the multivalent particle increases.

107. The composition of claim 100, wherein the neutralizing potency comprises:
(1) a suppression of a viral infection; and/or
(2) a number of viral particles that bind to a single multivalent particle.

108. A method for immunizing a subject against a viral infection comprising administering to the subject:
(a) the composition of any one of claims 1-51 or the particle of any one of claims 52-90; and
(b) a virus comprising the viral spike protein, wherein the viral infection comprises an infection by the virus.

109. The method of claim 108, wherein the virus comprises a live virus.

110. The method of claim 108, wherein the virus comprises an inactivated or dead virus.

111. The method of any one of claims 108-110, wherein the viral infection comprises an infection by SARS CoV-2, SARS CoV-1, MERS CoV, RSV, HBV, HDV, or HIV.

112. The method of any one of claims 108-110, wherein the viral infection comprises an infection by the original SARS CoV-2 virus, Delta variant of SARS CoV-2, Beta variant of SARS CoV-2, B.1.351 Beta variant of SARS CoV-2, B.1.617.2 Delta variant of SARS CoV-2, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant of SARS CoV-2, or B.1.351 South Africa strain of SARS CoV-2 comprising the E484K+N501Y mutant of spike protein.

113. The method of any one of claims 108-112, wherein the administering comprises administering through inhalation.

114. The method of any one of claims 108-112, wherein the administering comprises administering intranasally or intravenously.

115. A method for treating a viral infection in a subject comprising administering to the subject the composition of any one of claims 1-51 or the particle of any one of claims 52-90.

116. The method of claim 115, wherein the administering induces immunity in the subject against the viral infection.

117. The method of claim 115 or 116, wherein the viral infection comprises an infection by SARS CoV-2, SARS CoV-1, MERS CoV, RSV, HBV, HDV, or HIV.

118. The method of claim 115 or 116, wherein the viral infection comprises an infection by the original SARS CoV-2 virus, Delta variant of SARS CoV-2, Beta variant of SARS CoV-2, B.1.351 Beta variant of SARS CoV-2, B.1.617.2 Delta variant of SARS CoV-2, SARS CoV-2 USA-WA1/2020 strain, SARS CoV-2 comprising the D614G mutant of spike protein, SARS CoV-2 comprising the N439K mutant of spike protein, SARS CoV-2 comprising the N501Y mutant of spike protein, SARS CoV-2 comprising the E484K mutant of spike protein, SARS CoV-2 comprising the E484Q+L452R mutant of spike protein, Omicron variant of SARS CoV-2, or B.1.351 South Africa strain of SARS CoV-2 comprising the E484K+N501Y mutant of spike protein.

119. A multivalent particle comprising a fusion protein that comprises a mammalian polypeptide that binds to a viral protein and a transmembrane polypeptide wherein the fusion protein is expressed at least about 10 copies on a surface of the multivalent particle.

120. The multivalent particle of claim 119, wherein the viral protein is from SARS-CoV-1, SARS-CoV-2, MERS-CoV, RSV, HBV, HDV, HIV, or combinations thereof.

121. The multivalent particle of claim 119 or 120, wherein the mammalian polypeptide comprises a receptor that has binding specificity for the viral protein.

122. The multivalent particle of claim 121, wherein the receptor comprises a viral entry receptor or a viral attachment receptor.

123. The multivalent particle of claim 121, wherein the receptor is both a viral entry receptor and a viral attachment receptor.

124. The multivalent particle of claim 121, wherein the mammalian polypeptide comprises an extracellular domain of the receptor.

125. The multivalent particle of claim 119 or 120, wherein the mammalian polypeptide comprises a ligand or a secreted protein.

126. The multivalent particle of claim 119 or 120, wherein the mammalian polypeptide comprises ACE2, TRMPSS2, DPP4, CD4, CCR5, CXCR4, CD209, or CLEC4M.

127. The multivalent particle of claim 119 or 120, wherein the mammalian polypeptide comprises an amino acid sequence of at least 90% sequence identity to an amino acid sequence according to SEQ ID NO: 1.

128. The multivalent particle of claim 119 or 120, wherein the mammalian polypeptide comprises an amino acid sequence of at least 90% sequence identity to an amino acid sequence according to SEQ ID NO: 2.

129. The multivalent particle of any one of claims 119-128, wherein the transmembrane polypeptide anchors the fusion protein to a bilayer of the multivalent particle.

130. The multivalent particle of any one of claims 119-128, wherein the transmembrane polypeptide comprises a spike glycoprotein transmembrane region, a mammalian membrane protein, an envelope protein, a nucleocapsid protein, or a cellular transmembrane protein.

131. The multivalent particle of any one of claims 119-129, wherein the transmembrane polypeptide comprises a VSVG transmembrane region, spike protein S1 transmembrane region, spike protein S2 transmembrane region, Sindbis virus envelope (SINDBIS) protein, hemagglutinin envelope protein from measles virus, envelope glycoprotein of measles virus fusion (F) protein, RD114, BaEV, GP41, or GP120.

132. The multivalent particle of claim 131, wherein the VSVG transmembrane region comprises full length VSVG transmembrane region or a truncated VSVG transmembrane region.

133. The multivalent particle of claim 131, wherein the transmembrane polypeptide comprises the VSVG transmembrane region and a VSVG cytoplasmic tail.

134. The multivalent particle of any one of claims 119-129, wherein the transmembrane polypeptide comprises an amino acid sequence at least about 90% identical to that set forth in SEQ ID NO: 3.

135. The multivalent particle of any one of claims 119-129, wherein the transmembrane polypeptide comprises an amino acid sequence at least about 90% identical to that set forth in SEQ ID NO: 4.

136. The multivalent particle of any one of claims 119-135, wherein the fusion protein is expressed at least about 50 copies on a surface of the multivalent particle.

137. The multivalent particle of any one of claims 119-135, wherein the fusion protein is expressed at least about 75 copies on a surface of the multivalent particle.

138. The multivalent particle of any one of claims 119-135, wherein the fusion protein is expressed at least about 100 copies on a surface of the multivalent particle.

139. The multivalent particle of any one of claims 119-135, wherein the fusion protein is expressed at least about 150 copies on a surface of the multivalent particle.

140. The multivalent particle of any one of claims 119-135, wherein the fusion protein is expressed at least about 200 copies on a surface of the multivalent particle.

141. The multivalent particle of claim 119, wherein the mammalian polypeptide comprises ACE2 and the transmembrane polypeptide comprises a VSVG transmembrane region.

142. The multivalent particle of claim 119, wherein the mammalian polypeptide comprises ACE2 and the transmembrane polypeptide comprises a spike protein S2 transmembrane region.

143. The multivalent particle of claim 119, wherein the mammalian polypeptide comprises ACE2 and the transmembrane polypeptide comprises a surface glycoprotein transmembrane region of an enveloped virus.

144. The multivalent particle of claim 119, wherein the mammalian polypeptide comprises DPP4 and the transmembrane polypeptide comprises hemagglutinin envelope protein from measles virus.

145. The multivalent particle of claim 144, wherein the hemagglutinin envelope protein from measles virus is a variant of the hemagglutinin envelope protein from measles virus.

146. The multivalent particle of any one of claims 119-145, wherein the multivalent particle further comprises a second fusion protein that comprises a second mammalian polypeptide that binds to the viral protein and a second transmembrane polypeptide wherein the second fusion protein is expressed at least about 10 copies on the surface of the multivalent particle.

147. The multivalent particle of claim 146, wherein the second mammalian polypeptide comprises a receptor that has binding specificity for the viral protein.

148. The multivalent particle of claim 147, wherein the receptor comprises a viral entry receptor or a viral attachment receptor.

149. The multivalent particle of claim 147, wherein the receptor is both a viral entry receptor and a viral attachment receptor.

150. The multivalent particle of claim 147, wherein the second mammalian polypeptide comprises an extracellular domain of the receptor.

151. The multivalent particle of claim 146, wherein the second mammalian polypeptide comprises a ligand or a secreted protein.

152. The multivalent particle of claim 146, wherein the second mammalian polypeptide comprises ACE2, TRMPSS2, DPP4, CD4, CCR5, CXCR4, CD209, or CLEC4M.

153. The multivalent particle of claim 146, wherein the second mammalian polypeptide comprises an amino acid sequence of at least 90% sequence identity to an amino acid sequence according to SEQ ID NO: 1.

154. The multivalent particle of claim 146, wherein the second mammalian polypeptide comprises an amino acid sequence of at least 90% sequence identity to an amino acid sequence according to SEQ ID NO: 2.

155. The multivalent particle of any one of claims 146-154, wherein the second transmembrane polypeptide comprises a transmembrane anchoring protein.

156. The multivalent particle of any one of claims 146-154, wherein the second transmembrane polypeptide comprises a spike glycoprotein transmembrane region, a mammalian membrane protein, an envelope protein, a nucleocapsid protein, or a cellular transmembrane protein.

157. The multivalent particle of any one of claims 146-154, wherein the second transmembrane polypeptide comprises VSVG transmembrane region, spike protein S1 transmembrane region, spike protein S2 transmembrane region, Sindbis virus envelope (SINDBIS) protein, hemagglutinin envelope protein from measles virus, envelope glycoprotein of measles virus fusion (F) protein, RD114, BaEV, GP41, or GP120.

158. The multivalent particle of claim 157, wherein the VSVG transmembrane region comprises full length VSVG transmembrane region or a truncated VSVG transmembrane region.

159. The multivalent particle of claim 157, wherein the transmembrane polypeptide comprises a VSVG transmembrane region and a VSVG cytoplasmic tail.

160. The multivalent particle of any one of claims 146-154, wherein the second transmembrane polypeptide comprises an amino acid sequence at least about 90% identical to that set forth in SEQ ID NO: 3.

161. The multivalent particle of any one of claims 146-154, wherein the second transmembrane polypeptide comprises an amino acid sequence at least about 90% identical to that set forth in SEQ ID NO: 4.

162. The multivalent particle of any one of claims 146-161, wherein the second fusion protein is expressed at least about 50 copies on a surface of the multivalent particle.

163. The multivalent particle of any one of claims 146-161, wherein the second fusion protein is expressed at least about 75 copies on a surface of the multivalent particle.

164. The multivalent particle of any one of claims 146-161, wherein the second fusion protein is expressed at least about 100 copies on a surface of the multivalent p article.

165. The multivalent particle of any one of claims 146-161, wherein the second fusion protein is expressed at least about 150 copies on a surface of the multivalent particle.

166. The multivalent particle of any one of claims 146-161, wherein the second fusion protein is expressed at least about 200 copies on a surface of the multivalent particle.

167. The multivalent particle of claim 146, wherein the second mammalian polypeptide comprises ACE2 and the second transmembrane polypeptide comprises VSVG transmembrane region.

168. The multivalent particle of claim 146, wherein the second mammalian polypeptide comprises ACE2 and the second transmembrane polypeptide comprises spike protein S2 transmembrane region.

169. The multivalent particle of claim 146, wherein the second mammalian polypeptide comprises ACE2 and the second transmembrane polypeptide comprises a surface glycoprotein of an enveloped virus.

170. The multivalent particle of claim 146, wherein the second mammalian polypeptide comprises DPP4 and the second transmembrane polypeptide comprises hemagglutinin envelope protein from measles virus.

171. The multivalent particle of claim 170, wherein the hemagglutinin envelope protein from measles virus is a variant of the hemagglutinin envelope protein from measles virus.

172. The multivalent particle of claim 146, wherein the mammalian polypeptide comprises a viral entry receptor and the second mammalian polypeptide comprises a viral attachment receptor.

173. The multivalent particle of claim 146, wherein the mammalian polypeptide comprises ACE2, the transmembrane polypeptide comprises VSVG transmembrane region, spike protein S1 transmembrane region, spike protein S2 transmembrane region, or a surface glycoprotein of an enveloped virus, the second mammalian polypeptide comprises a heparan sulfate proteoglycan, and the second transmembrane polypeptide comprises VSVG transmembrane region, spike protein S1 transmembrane region, spike protein S2 transmembrane region, or a surface glycoprotein of an enveloped virus.

174. The multivalent particle of claim 146, wherein the mammalian polypeptide comprises CD4 and the second mammalian peptide comprises, CCR5, CXCR4, or both.

175. The multivalent particle of any one of claims 119-174, wherein the multivalent particle comprises an IC50 of less than 5 picomolar (pM) in a neutralization assay.

176. The multivalent particle of any one of claims 119-174, wherein the multivalent particle comprises an IC50 of less than 2.5 picomolar (pM) in a neutralization assay.

177. The multivalent particle of any one of claims 119-174, wherein the multivalent particle comprises an IC50 of less than 1 picomolar (pM) in a neutralization assay.

178. The multivalent particle of any one of claims 119-177, wherein the multivalent particle does not comprise viral genetic material.

179. The multivalent particle of any one of claims 119-178, wherein the multivalent particle is synthetic.

180. The multivalent particle of any one of claims 119-178, wherein the multivalent particle is recombinant.

181. The multivalent particle of any one of claims 119-178, wherein the multivalent particle is a viral-like a particle.

182. The multivalent particle of any one of claims 119-178, wherein the multivalent particle is an extracellular vesicle.

183. The multivalent particle of any one of claims 119-178, wherein the multivalent particle is an exosome.

184. The multivalent particle of any one of claims 119-178, wherein the multivalent particle is an ectosome.

185. The multivalent particle of any one of claims 119-183, wherein the fusion protein further comprises an oligomerization domain.

186. The multivalent particle of claim 184, wherein the oligomerization domain is a dimerization domain.

187. The multivalent particle of claim 186, wherein the dimerization domain comprises a leucine zipper dimerization domain.

188. The multivalent particle of claim 184, wherein the oligomerization domain is a trimerization domain.

189. The multivalent particle of claim 188, wherein the trimerization domain comprises a post-fusion oligomerization domain of viral surface protein.

190. The multivalent particle of claim 188, wherein the trimerization domain comprises a D4 post-fusion trimerization domain of VSV-G protein.

191. The multivalent particle of claim 188, wherein the trimerization domain comprises a Dengue E protein post-fusion trimerization domain.

192. The multivalent particle of claim 188, wherein the trimerization domain comprises a foldon trimerization domain.

193. The multivalent particle of claim 187, wherein the trimerization domain comprises human C-propeptide of α1(I) collagen.

194. The multivalent particle of claim 184, wherein the oligomerization domain is a tetramerization domain.

195. The multivalent particle of claim 193, wherein the tetramerization domain comprises an influenza neuraminidase stem domain.

196. The multivalent particle of claim 184, wherein the oligomerization domain comprises an amino acid sequence that has at least 95% sequence identity to an amino acid sequence according to SEQ ID NOs: 5-18, or 28.

197. The multivalent particle of any one of claims 184-196, wherein when the fusion protein is expressed on the surface of the multivalent particle, the oligomerization domain is outside of the multivalent particle.

198. The multivalent particle of any one of claims 184-196, wherein when the fusion protein is expressed on the surface of the multivalent particle, the oligomerization domain is outside of the multivalent particle and adjacent to a signal peptide.

199. The multivalent particle of any one of claims 184-196, wherein when the fusion protein is expressed on the surface of the multivalent particle, the oligomerization domain is inside of the multivalent particle.

200. The multivalent particle of any one of claims 184-196, wherein when the fusion protein is expressed on the surface of the multivalent particle, the oligomerization domain is inside of the multivalent particle and adjacent to the transmembrane polypeptide.

201. The multivalent particle of any one of claims 184-200, wherein the fusion protein comprises a signal peptide.

202. The multivalent particle of any one of claims 184-200, wherein domains of the fusion protein are arranged from the N-terminus to the C-terminus in the following orders:
(a) signal peptide, extracellular domain of a viral entry receptor which binds to a surface protein of a virus, oligomerization domain, transmembrane polypeptide, and cytosolic domain;
(b) signal peptide, extracellular domain of a viral entry receptor which binds to a surface protein of a virus, transmembrane polypeptide, oligomerization domain, and cytosolic domain; or
(c) signal peptide, oligomerization domain, extracellular domain of a viral entry receptor, transmembrane polypeptide, and cytosolic domain.

203. A composition comprising a first nucleic acid sequence encoding a multivalent particle comprising a fusion protein that comprises an extracellular domain of a viral entry receptor that binds to a viral protein and a transmembrane polypeptide wherein the fusion protein is expressed at least about 10 copies on a surface of the multivalent particle when the multivalent particle is expressed; and an excipient.

204. The composition of claim 203, wherein the viral protein is from SARS-CoV-1, SARS-CoV-2, MERS-CoV, respiratory syncytial virus, HBV, HDV, HIV, or combinations thereof.

205. The composition of claim 203 or 204, further comprising a second nucleic acid sequence that encodes one or more packaging viral proteins.

206. The composition of claim 205, wherein the one or more packaging viral proteins is a lentiviral protein, a retroviral protein, an adenoviral protein, or combinations thereof.

207. The composition of claim 205, wherein the one or more packaging viral proteins comprises gag, pol, pre, tat, rev, or combinations thereof.

208. The composition of any one of claims 203-207, further comprising a third nucleic acid sequence that encodes a replication incompetent viral genome, a reporter, a therapeutic molecule, or combinations thereof.

209. The composition of claim 208, wherein the viral genome is derived from vesicular stomatitis virus, measles virus, Hepatitis virus, influenzavirus, or combinations thereof.

210. The composition of claim 208, wherein the reporter is a fluorescent protein or luciferase.

211. The composition of claim 210, wherein the fluorescent protein is green fluorescent protein.

212. The composition of claim 208, wherein the therapeutic molecule is an immune modulating protein, a cellular signal modulating molecule, a proliferation modulating molecule, a cell death modulating molecule, or combinations thereof.

213. The composition of any one of claims 203-212, wherein the mammalian polypeptide comprises a receptor that has binding specificity for the viral protein.

214. The composition of claim 213, wherein the receptor comprises a viral entry receptor or a viral attachment receptor.

215. The composition of claim 213, wherein the receptor is both a viral entry receptor and a viral attachment receptor.

216. The composition of claim 213, wherein the mammalian polypeptide comprises an extracellular domain of the receptor.

217. The composition of any one of claims 203-212, wherein the mammalian polypeptide comprises a ligand or a secreted protein.

218. The composition of any one of claims 203-212, wherein the mammalian polypeptide comprises ACE2, TRMPSS2, DPP4, CD4, CCR5, CXCR4, CD209, or CLEC4M.

219. The composition of any one of claims 203-212, wherein the mammalian polypeptide comprises an amino acid sequence of at least 90% sequence identity to an amino acid sequence according to SEQ ID NO: 1.

220. The composition of any one of claims 203-212, wherein the mammalian polypeptide comprises an amino acid sequence of at least 90% sequence identity to an amino acid sequence according to SEQ ID NO: 2.

221. The composition of any one of claims 203-220, wherein the transmembrane polypeptide comprises a transmembrane anchoring protein.

222. The composition of any one of claims 203-220, wherein the transmembrane polypeptide comprises a spike glycoprotein transmembrane region, a mammalian membrane protein, an envelope protein, a nucleocapsid protein, or a cellular transmembrane protein.

223. The composition of any one of claims 203-220, wherein the transmembrane polypeptide comprises VSVG transmembrane region, spike protein S1 transmembrane region, spike protein S2 transmembrane region, Sindbis virus envelope (SINDBIS) protein, hemagglutinin envelope protein from measles virus, envelope glycoprotein of measles virus fusion (F) protein, RD114, BaEV, GP41, or GP120.

224. The composition of claim 223, wherein the VSVG transmembrane region comprises full length VSVG transmembrane region or a truncated VSVG transmembrane region.

225. The composition of claim 223, wherein the transmembrane polypeptide comprises a VSVG transmembrane region and a VSVG cytoplasmic tail.

226. The composition of any one of claims 203-220, wherein the transmembrane polypeptide comprises an amino acid sequence at least about 90% identical to that set forth in SEQ ID NO: 3.

227. The composition of any one of claims 203-220, wherein the transmembrane polypeptide comprises an amino acid sequence at least about 90% identical to that set forth in SEQ ID NO: 4.

228. The composition of any one of claims 119-183, wherein the fusion protein further comprises an oligomerization domain.

229. The composition of claim 184, wherein the oligomerization domain is a dimerization domain.

230. The composition of claim 186, wherein the dimerization domain comprises a leucine zipper dimerization domain.

231. The composition of claim 184, wherein the oligomerization domain is a trimerization domain.

232. The composition of claim 188, wherein the trimerization domain comprises a post-fusion oligomerization domain of viral surface protein.

233. The composition of claim 188, wherein the trimerization domain comprises a D4 post-fusion trimerization domain of VSV-G protein.

234. The composition of claim 188, wherein the trimerization domain comprises a Dengue E protein post-fusion trimerization domain.

235. The composition of claim 188, wherein the trimerization domain comprises a foldon trimerization domain.

236. The composition of claim 187, wherein the trimerization domain comprises human C-propeptide of α1(I) collagen.

237. The composition of claim 184, wherein the oligomerization domain is a tetramerization domain.

238. The composition of claim 193, wherein the tetramerization domain comprises an influenza neuraminidase stem domain.

239. The composition of claim 184, wherein the oligomerization domain comprises an amino acid sequence that has at least 95% sequence identity to an amino acid sequence according to SEQ ID NOs: 5-18, or 28.

240. The composition of any one of claims 184-196, wherein when the fusion protein is expressed on the surface of the multivalent particle, the oligomerization domain is outside of the multivalent particle.

241. The composition of any one of claims 184-196, wherein when the fusion protein is expressed on the surface of the multivalent particle, the oligomerization domain is outside of the multivalent particle and adjacent to a signal peptide.

242. The composition of any one of claims 184-196, wherein when the fusion protein is expressed on the surface of the multivalent particle, the oligomerization domain is inside of the multivalent particle.

243. The composition of any one of claims 184-196, wherein when the fusion protein is expressed on the surface of the multivalent particle, the oligomerization domain is inside of the multivalent particle and adjacent to the transmembrane polypeptide.

244. The composition of any one of claims 203-227, wherein the fusion protein is expressed at least about 50 copies on a surface of the multivalent particle when itis expressed.

245. The composition of any one of claims 203-227, wherein the fusion protein is expressed at least about 75 copies on a surface of the multivalent particle when itis expressed.

246. The composition of any one of claims 203-227, wherein the fusion protein is expressed at least about 100 copies on a surface of the multivalent particle when it is expressed.

247. The composition of any one of claims 203-227, wherein the fusion protein is expressed at least about 150 copies on a surface of the multivalent particle when it is expressed.

248. The composition of any one of claims 203-227, wherein the fusion protein is expressed at least about 200 copies on a surface of the multivalent particle when it is expressed.

249. The composition of claim 203, wherein the mammalian polypeptide comprises ACE2 and the transmembrane polypeptide comprises VSVG transmembrane region.

250. The composition of claim 203, wherein the mammalian polypeptide comprises ACE2 and the transmembrane polypeptide comprises spike protein S2 transmembrane region.

251. The composition of claim 203, wherein the mammalian polypeptide comprises ACE2 and the transmembrane polypeptide comprises a surface glycoprotein of an enveloped virus.

252. The composition of claim 203, wherein the mammalian polypeptide comprises DPP4 and the transmembrane polypeptide comprises hemagglutinin envelope protein from measles virus.

253. The composition of claim 236, wherein the hemagglutinin envelope protein from measles virus is a variant of the hemagglutinin envelope protein from measles virus.

254. The composition of any one of claims 208-237, wherein the composition further comprises a fourth nucleic acid sequence encoding a second fusion protein that comprises a second mammalian polypeptide that binds to the viral protein and a second transmembrane polypeptide wherein the second fusion protein is expressed at least about 10 copies on the surface of the multivalent particle when it is expressed.

255. The composition of claim 238, wherein the second mammalian polypeptide comprises a receptor that has binding specificity for the viral protein.

256. The composition of claim 239, wherein the receptor comprises a viral entry receptor or a viral attachment receptor.

257. The composition of claim 239, wherein the receptor is both a viral entry receptor and a viral attachment receptor.

258. The composition of claim 239, wherein the second mammalian polypeptide comprises an extracellular domain of the receptor.

259. The composition of claim 238, wherein the second mammalian polypeptide comprises a ligand or a secreted protein.

260. The composition of claim 238, wherein the second mammalian polypeptide comprises ACE2, TRMPSS2, DPP4, CD4, CCR5, CXCR4, CD209, or CLEC4M.

261. The composition of claim 238, wherein the second mammalian polypeptide comprises an amino acid sequence of at least 90% sequence identity to an amino acid sequence according to SEQ ID NO: 1.

262. The composition of claim 238, wherein the second mammalian polypeptide comprises an amino acid sequence of at least 90% sequence identity to an amino acid sequence according to SEQ ID NO: 2.

263. The composition of any one of claims 238-246, wherein the second transmembrane polypeptide comprises a transmembrane anchoring protein.

264. The composition of any one of claims 238-246, wherein the second transmembrane polypeptide comprises a spike glycoprotein transmembrane region, a mammalian membrane protein, an envelope protein, a nucleocapsid protein, or a cellular transmembrane protein.

265. The composition of any one of claims 238-246, wherein the second transmembrane polypeptide comprises VSVG transmembrane region, spike protein S1 transmembrane region, spike protein S2 transmembrane region, Sindbis virus envelope (SINDBIS) protein, hemagglutinin envelope protein from measles virus, envelope glycoprotein of measles virus fusion (F) protein, RD114, BaEV, GP41, or GP120.

266. The composition of claim 249, wherein the VSVG transmembrane region comprises full length VSVG transmembrane region or a truncated VSVG transmembrane region.

267. The composition of claim 249, wherein the VSVG transmembrane region comprises a VSVG transmembrane region and a VSVG cytoplasmic tail.

268. The composition of any one of claims 238-246, wherein the second transmembrane polypeptide comprises an amino acid sequence at least about 90% identical to that set forth in SEQ ID NO: 3.

269. The composition of any one of claims 238-246, wherein the second transmembrane polypeptide comprises an amino acid sequence at least about 90% identical to that set forth in SEQ ID NO: 4.

270. The composition of any one of claims 119-183, wherein the second fusion protein further comprises an oligomerization domain.

271. The composition of claim 184, wherein the oligomerization domain is a dimerization domain.

272. The composition of claim 186, wherein the dimerization domain comprises a leucine zipper dimerization domain.

273. The composition of claim 184, wherein the oligomerization domain is a trimerization domain.

274. The composition of claim 188 wherein the trimerization domain comprises a post-fusion oligomerization domain of viral surface protein.

275. The composition of claim 188, wherein the trimerization domain comprises a D4 post-fusion trimerization domain of VSV-G protein.

276. The composition of claim 188, wherein the trimerization domain comprises a Dengue E protein post-fusion trimerization domain.

277. The composition of claim 188, wherein the trimerization domain comprises a foldon trimerization domain.

278. The composition of claim 187, wherein the trimerization domain comprises human C-propeptide of α1(I) collagen.

279. The composition of claim 184, wherein the oligomerization domain is a tetramerization domain.

280. The composition of claim 193, wherein the tetramerization domain comprises an influenza neuraminidase stem domain.

281. The composition of claim 184, wherein the oligomerization domain comprises an amino acid sequence that has at least 95% sequence identity to an amino acid sequence according to SEQ ID NOs: 5-18, or 28.

282. The composition of any one of claims 184-196, wherein when the second fusion protein is expressed on the surface of the multivalent particle, the oligomerization domain is outside of the multivalent particle.

283. The composition of any one of claims 184-196, wherein when the second fusion protein is expressed on the surface of the multivalent particle, the oligomerization domain is outside of the multivalent particle and adjacent to a signal peptide.

284. The composition of any one of claims 184-196, wherein when the second fusion protein is expressed on the surface of the multivalent particle, the oligomerization domain is inside of the multivalent particle.

285. The composition of any one of claims 184-196, wherein when the second fusion protein is expressed on the surface of the multivalent particle, the oligomerization domain is inside of the multivalent particle and adjacent to the transmembrane polypeptide.

286. The composition of any one of claims 238-253, wherein the second fusion protein is expressed at least about 50 copies on a surface of the multivalent particle when itis expressed.

287. The composition of any one of claims 238-253, wherein the second fusion protein is expressed at least about 75 copies on a surface of the multivalent particle when itis expressed.

288. The composition of any one of claims 238-253, wherein the second fusion protein is expressed at least about 100 copies on a surface of the multivalent particle when it is expressed.

289. The composition of any one of claims 238-253, wherein the second fusion protein is expressed at least about 150 copies on a surface of the multivalent particle when it is expressed.

290. The composition of any one of claims 238-253, wherein the second fusion protein is expressed at least about 200 copies on a surface of the multivalent particle when it is expressed.

291. The composition of claim 238, wherein the second mammalian polypeptide comprises ACE2 and the second transmembrane polypeptide comprises VSVG transmembrane region.

292. The composition of claim 238, wherein the second mammalian polypeptide comprises ACE2 and the second transmembrane polypeptide comprises spike protein S2 transmembrane region.

293. The composition of claim 238, wherein the second mammalian polypeptide comprises ACE2 and the second transmembrane polypeptide comprises a surface glycoprotein of an enveloped virus.

294. The composition of claim 238, wherein the second mammalian polypeptide comprises DPP4 and the second transmembrane polypeptide comprises hemagglutinin envelope protein from measles virus.

295. The composition of claim 262, wherein the hemagglutinin envelope protein from measles virus is a variant of the hemagglutinin envelope protein from measles virus.

296. The composition of claim 238, wherein the mammalian polypeptide comprises a viral entry receptor and the second mammalian polypeptide comprises a viral attachment receptor.

297. The composition of claim 238, wherein the mammalian polypeptide comprises ACE2, the transmembrane polypeptide comprises VSVG transmembrane region, spike protein S1 transmembrane region, spike protein S2 transmembrane region, or a surface glycoprotein of an enveloped virus, the second mammalian polypeptide comprises a heparan sulfate proteoglycan, and the second transmembrane polypeptide comprises VSVG transmembrane region, spike protein S1 transmembrane region, spike protein S2 transmembrane region, or a surface glycoprotein of an enveloped virus.

298. The composition of claim 238, wherein the mammalian polypeptide comprises CD4 and the second mammalian peptide comprises, CCR5, CXCR4, or both.

299. The composition of claim 208, wherein the first nucleic acid sequence, the second nucleic acid sequence, and the third nucleic acid sequence are within a same vector.

300. The composition of claim 208, wherein the first nucleic acid sequence, the second nucleic acid sequence, and the third nucleic acid sequence are within different vectors.

301. The composition of claim 238, wherein the first nucleic acid sequence, the second nucleic acid sequence, the third nucleic acid sequence, and the fourth nucleic acid sequence are within a same vector.

302. The composition of claim 238, wherein the first nucleic acid sequence, the second nucleic acid sequence, third nucleic acid sequence, and the fourth nucleic acid sequence are within different vectors.

303. The composition of claim 236, wherein the nucleic acid sequence that encodes the first fusion protein and the second fusion protein and the second nucleic acid sequence and the third nucleic acid sequence are mRNAs.

304. The composition of claim 236, wherein the nucleic acid sequence that encodes the first fusion protein and the second fusion protein and the second nucleic acid sequence and the third nucleic acid sequence are DNA.

305. The composition of any one of claim 267, wherein the composition comprises a vector, wherein the vector is a lentivirus vector, an adenovirus vector, or an adeno-associated virus vector.

306. A pharmaceutical composition comprising the multivalent particle of any one of claims 119-202 and a pharmaceutically acceptable excipient.

307. A method of treating a viral infection in a subject in need thereof, comprising administering to the subject the multivalent particle of any one of claims 119-202 or the composition of any one of claims 203-305.

308. The method of claim 307, wherein the multivalent particle is administered intravenously.

309. The method of claim 307, wherein the multivalent particle is administered through inhalation or intranasal delivery.

310. The method of claim 307, wherein the multivalent particle is administered by an intraperitoneal injection.

311. The method of claim 307, wherein the multivalent particle is administered by a subcutaneous injection.

312. The method of claim 307, wherein the viral infection comprises an infection by SARS CoV-2, SARS CoV-1, MERS CoV.

313. The method of claim 307, wherein the composition is administered intravenously.

314. The method of claim 307, wherein the composition is administered through inhalation.

315. The method of claim 307, wherein the composition is administered by an intraperitoneal injection.

316. The method of claim 307, wherein the composition is administered by a subcutaneous injection.

317. The method of claim 307, wherein the composition comprises a liposome.

318. The method of claim 307, wherein the composition comprises an adeno-associated virus (AAV)

319. The method of claim 307, wherein the composition comprises a lipid nanoparticle.

320. The method of claim 307, wherein the composition comprises a polymer.

321. The method of claim 312, wherein the SARS CoV-2, SARS CoV-1, MERS CoV are effectively neutralized in vivo by the multivalent particle or the composition.

322. The method of claim 307, wherein the multivalent particle or the composition inhibits a respiratory symptom of the viral infection.

323. The method of claim 307, wherein the multivalent particle or the composition induces robust immunity against different strains of the viral infection.

324. The method of claim 307, wherein the viral infection comprises infection by SARS CoV-2, and the multivalent particle or the composition induces robust immunity against Delta variant of SARS CoV-2.

325. A method of producing immunity against a viral infection in a subject in need thereof, comprising administering to the subject the multivalent particle of any one of claims 119-202 or the composition of any one of claims 203-305 and a virus of the viral infection.

326. The method of claim 325, wherein the multivalent particle is administered intravenously.

327. The method of claim 325, wherein the multivalent particle is administered through inhalation or intranasal delivery.

328. The method of claim 325, wherein the multivalent particle is administered by an intraperitoneal injection.

329. The method of claim 325, wherein the multivalent particle is administered by a subcutaneous injection.

330. The method of any one of claims 325-329, wherein the viral infection comprises an infection by SARS CoV-2, SARS CoV-1, MERS CoV, RSV, HBV, HDV, or HIV.

331. The method of any one of claims 325-330, wherein the composition is administered intravenously.

332. The method of any one of claims 325-330, wherein the composition is administered through inhalation.

333. The method of any one of claims 325-330, wherein the composition is administered by an intraperitoneal injection.

334. The method of any one of claims 325-330, wherein the composition is administered by a subcutaneous injection.

335. The method of any one of claims 325-334, wherein the composition comprises a liposome.

336. The method of any one of claims 325-335, wherein the composition comprises an adeno-associated virus (AAV)

337. The method of any one of claims 325-336, wherein the composition comprises a lipid nanoparticle.

338. The method of any one of claims 325-337, wherein the composition comprises a polymer.

339. The method of any one of claims 325-338, wherein the SARS CoV-2, SARS CoV-1, MERS CoV, RSV, HBV, HDV, or HIV are effectively neutralized in vivo by the multivalent particle or the composition.

340. The method of any one of claims 325-339, wherein the multivalent particle or the composition inhibits a respiratory symptom of the viral infection.

341. The method of any one of claims 325-340, wherein the multivalent particle or the composition induces robust immunity against different strains of the viral infection.

342. The method of any one of claims 325-341, wherein the viral infection comprises infection by SARS CoV-2, and the multivalent particle or the composition induces robust immunity against variants of SARS CoV-2.


SEQUENCE LISTING (35 sequences)

SEQ ID NO: 1 - protein, 805 aa, Homo sapiens [sequence omitted]

SEQ ID NO: 2 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 3 - protein, 495 aa, Vesiculovirus indiana
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 4 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 150 aa, Vesiculovirus indiana
IGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDI

SEQ ID NO: 6 - protein, 130 aa, Vesiculovirus indiana
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNL

SEQ ID NO: 7 - protein, 115 aa, Vesiculovirus indiana
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTT

SEQ ID NO: 8 - protein, 100 aa, Vesiculovirus indiana
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFING

SEQ ID NO: 9 - protein, 84 aa, Vesiculovirus indiana
IQADGWMCHASKWVTTCDFRWYGPKYITHSIRSFTPSVEQCKESIEQTKQGTWLNPGFPPQSCGYATVTDAEAVIVQVTPHHVL

SEQ ID NO: 10 - protein, 27 aa, synthetic construct
GYIPEAPRDGQAYVRKDGEWVLLSTFL

SEQ ID NO: 11 - protein, 33 aa, synthetic construct
RMKQLEDKVEELLSKQYHLENEVARLKKLVGER

SEQ ID NO: 12 - protein, 33 aa, synthetic construct
RMKQLEDKVEELLSKNYHLENEVARLKKLVGER

SEQ ID NO: 13 - protein, 40 aa, Influenza A virus
MNPNQKIITIGSICLVVGLISLILQIGNIISIWISHSIQT

SEQ ID NO: 14 - protein, 75 aa, Influenza A virus
MNPNQKIITIGSICMVTGIVSLMLQIGNMISIWVSHSIHTGNQHQSEPISNTNFLTEKAVASVKLAGNSSLCPIN

SEQ ID NO: 15 - protein, 68 aa, Dengue virus
KLCIEAKISNTTTDSRCPTQGEATLVEEQDTNFVCRRTFVDRGHGNGCGLFGKGSLITCAKFKCVTKL

SEQ ID NO: 16 - protein, 83 aa, Dengue virus
IELLKTEVTNPAVLRKLCIEAKISNTTTDSRCPTQGEATLVEEQDTNFVCRRTFVDRGHGNGCGLFGKGSLITCAKFKCVTKL

SEQ ID NO: 17 - protein, 83 aa, Dengue virus
KLCIEAKISNTTTDSRCPTQGEATLVEEQDTNFVCRRTFVDRGHGNGCGLFGKGSLITCAKFKCVTKLEGKIVQYENLKYSVI

SEQ ID NO: 18 - protein, 59 aa, Dengue virus
EAKISNTTTDSRCPTQGEATLVEEQDTNFVCRRTFVDRGHGNGCGLFGKGSLITCAKFK

SEQ ID NO: 19 - protein, 22 aa, Vesiculovirus indiana
IASFFFIIGLIIGLFLVLRVGI

SEQ ID NO: 20 - protein, 37 aa, Vesiculovirus indiana
PIELVEGWFSSWKSSIASFFFIIGLIIGLFLVLRVGI

SEQ ID NO: 21 - protein, 53 aa, Vesiculovirus indiana
DDESLFFGDTGLSKNPIELVEGWFSSWKSSIASFFFIIGLIIGLFLVLRVGIH

SEQ ID NO: 22 - protein, 81 aa, Vesiculovirus indiana
GMLDSDLHLSSKAQVFEHPHIQDAASQLPDDESLFFGDTGLSKNPIELVEGWFSSWKSSIASFFFIIGLIIGLFLVLRVGI

SEQ ID NO: 23 - protein, 25 aa, Vesiculovirus indiana
HLCIKLKHTKKRQIYTDIEMNRLGK

SEQ ID NO: 24 - protein, 23 aa, Influenza A virus
IITIGSVCMTIGMANLILQIGNI

SEQ ID NO: 25 - protein, 23 aa, Influenza A virus
LAIYSTVASSLVLVVSLGAISFW

SEQ ID NO: 26 - protein, 52 aa, Dengue virus
AYGVLFSGVSWTMKIGIGILLTWLGLNSRSTSLSMTCIAVGMVTLYLGVMVQ

SEQ ID NO: 27 - protein, 21 aa, Human immunodeficiency virus 1
FIMIVGGLVGLRIVFAVLSIV

SEQ ID NO: 28 - protein, 256 aa, Homo sapiens
ETGHHHHHHSADEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQQITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 29 - protein, 848 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 30 - protein, 1344 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSDIGGGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTYTDIEMNRLGK

SEQ ID NO: 31 - protein, 1355 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRSFSYFYPFRLPIKGVPIELQVECFTWDQKLWCRHFCVLADSESGGHITHSGMVGMGVSCTVTREGGGSKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPAAARGSGLNDIFEAQKIEWHE

SEQ ID NO: 32 - protein, 737 aa, synthetic construct
KGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 33 - protein, 848 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHAEMGAIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 34 - protein, 978 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSRIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 35 - protein, 978 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHAEMGAIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSRIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK